(19) United States
(12) Patent Application Publication
(10) Pub. No.: US 2019/0055212 A1
(43) Pub. Date: Feb. 21, 2019

(54) HISTONE DEMETHYLASE INHIBITORS

(71) Applicant: Stephen Gwaltney, Escondido, CA (US)

(72) Inventor: Stephen Gwaltney, Escondido, CA (US)

(21) Appl. No.: 16/077,864

(22) PCT Filed: Feb. 16, 2017
(86) PCT No.: PCT/US2017/018094

§ 371 Date: Aug. 14, 2018

Related U.S. Application Data
(60) Provisional application No. 62/295,608, filed on Feb. 16, 2016.

(51) Int. Cl. C07D 401/04; A61P 35/00; C07D 401/14; C07D 491/113; C07D 405/14; C07D 471/04

(52) U.S. Cl. CPC C07D 401/04; A61P 35/00; C07D 471/04; C07D 491/113; C07D 405/14; C07D 401/14

(57) ABSTRACT

This disclosure relates to compounds that inhibit histone demethylase activity. In particular, the disclosure relates to compounds that inhibit histone lysine demethylase KDM5B, pharmaceutical compositions and methods of use, such as methods of treating cancer using the compounds and pharmaceutical compositions disclosed herein.

### FIELD

This invention relates to the field of cancer treatment.

### BACKGROUND

Histone methylation plays an important role in the epigenetic regulation of a number of diverse biological processes and diseases. Histone lysine demethylases are a class of enzymes that remove methyl groups from mon-, di- or tri-methylated lysine residues of histones to regulate gene expression and modulate chromatin structure.

Histone lysine demethylases are classified into two separate superfamilies based on sequence homology and mechanism of action. The members of the KDM1 (Lysine (K) demethylase 1) superfamily are FAD-dependent amine oxidases, which act on mono-/di-methylated lysine residues, whereas the other histone demethylase superfamily members are Fe(II) and 2-oxoglutarate-dependent enzymes, and share the signature Jumonji C (JmjC) domain. Members of the latter histone lysine demethylase superfamily have been further classified into separate groups based on JmjC sequence homology and other associated motifs (see, e.g., Pedersen and Helin (2010) Trends in Cell Biol. 20:672-677).

KDM5B (JARID1B) is a member of the JmjC histone lysine demethylase superfamily and acts on di- and trimethylated lysine residues of histones, particularly di- and trimethylated lysine 4 in the N-terminal tail of histone H3. KDM5B has been reported to be overexpressed in a number of cancers, including breast, prostate, testicular, ovarian, leukemia and bladder carcinoma, and KDM5B activity is reported to be required for continued growth of melanoma (see, e.g., H\acute{ø}fedlt et al., (2013) Nature Rev Drug Disc., Published on line Nov. 13, 2013 doi:10.1038/nrd4154).

With increasing evidence that histone lysine demethylases, including KDM5B, play a critical role in a diverse set of cancers and diseases, a variety of histone demethylase inhibitors have been reported in the literature (e.g., see Lizcano and Garcia (2012) Pharmaceuticals 5:963-990). Inhibitors of KDM5B and other Jumonji C superfamily members compete with the 2-oxoglutrate co-factor and bind to the catalytic region containing Fe(II) to block demethylation. KDM5B inhibitors have yet to successfully advance into human clinical trials.

### SUMMARY

In certain aspects, compounds are provided that inhibit KDM5B activity. In certain embodiments, the compounds are represented by formula (I):

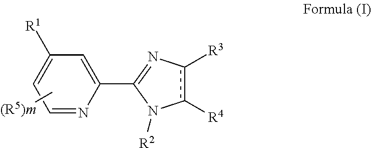

or a pharmaceutical salt thereof:

wherein R¹ is —COOR⁶, —C(O)N(H)CN, —C(O)N(H)OH, or tetrazolyl; R² is C₀-C₆ alkyl-R⁷; R³ and R⁴ are each independently selected from hydrogen, halogen, alkyl, alkoxy, —(CH2)ₙOH, —(CH2)ₙC(O)NHR⁶, —(CH2)ₙC(O)NHR⁵, —(CH2)ₙC(O)NHR⁷, —(CH2)ₙC(O)NR⁶R⁷, —(CH2)ₙN(R⁶)C(O)R⁵, (CH2)ₙN(R⁶)C(O)R⁷, or —(CH2)ₙNHC(O)R⁵, —(CH2)ₙNHC(O)R⁷, carbocyclyl, heterocyclyl, aryl, heteroaryl, alkylcarbocyclyl, alkylheterocyclyl, alkylaryl, alkylheteroaryl, wherein each of the carbocyclyl, heterocyclyl, aryl, heteroaryl, alkylcarbocyclyl, alkylheterocyclyl, alkylaryl, alkylheteroaryl is optionally substituted with one or more R⁵; or R³ and R⁴ and the carbon atoms to which they are attached form a 5-7 membered unsaturated, partial unsaturated or saturated ring system optionally containing 1-3 heteroatoms selected from N, O or S, and further optionally substituted with one or more R⁵;

R⁵ is alkyl, alkenyl, alkynyl, halogen, haloalkyl, alkoxy, cyano, amino, —COOR⁶, C(O)NHR⁶, C(O)N(R⁶)₂, N(R⁶)C(O)R³, NHC(O)R³, aryloxy or optionally substituted heterocyclyl. R⁶ is hydrogen or alkyl; R⁷ is hydrogen, carbocyclyl, heterocyclyl, aryl, or heteroaryl, wherein each of the carbocyclyl, heterocyclyl, aryl, or heteroaryl may be optionally substituted with one or more R⁵; or
R⁶ and R⁷ and the nitrogen atom to which they are attached form a 4-7 membered unsaturated, partial unsaturated or saturated ring system optionally containing 1-3 heteroatoms selected from N, O or S, and further optionally substituted with one or more R⁵; and m and n are each independently zero or an integer between 1 and 3.

In other aspects, pharmaceutical compositions are provided comprising a therapeutically effective amount of a compound disclosed herein and a pharmaceutically acceptable excipient.

In yet other aspects, methods for inhibiting histone demethylase activity in a cell or methods for treating cancer in a patient are provided comprising administering a therapeutically effective amount of a compound or pharmaceutical composition disclosed herein to a cell or to a patient in need thereof.

Numerous other aspects are provided in accordance with these and other aspects of the invention. Other features and aspects of the present invention will become more fully apparent from the following detailed description and the appended claims.

### DETAILED DESCRIPTION

As used herein, the word “a” or “plurality” before a noun represents one or more of the particular noun. For example, the phrase “a mammalian cell” represents “one or more mammalian cells.”

As used herein, “KDM5B” refers to a mammalian Jumonji C superfamily histone lysine demethylase which removes methyl groups from tri- and dimethylated lysine4 of the histone H3 protein.

As used herein, a “KDM5B inhibitor” refers to compounds disclosed herein that are represented by formula (I) as described herein. These compounds are able to negatively modulate or to inhibit all or a portion of the enzymatic activity of KDM5B.

The KDM5B can be from any animal that has KDM5B, including from a human.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)ₐ-B—, wherein a is 0 or 1. In such instances, when a is 0 the moiety is B— and when a is 1 the moiety is A-B—. Also, a number of moieties disclosed herein exist in multiple tautomeric forms, all of which are intended to be encompassed by any given tautomeric structure.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃-hydrocarbyl” includes a covalent bond, methyl, ethyl, propyl, isopropyl, and cyclopropyl.

The term “alkyl” as employed herein refers to straight and branched chain aliphatic groups having from 1 to 12 carbon atoms (1, 2, 3, 4, 5, 6, 7, 8, 9, 10, 11, or 12), which is optionally substituted with one, two or three substituents. Exemplary alkyl groups include, without limitation, methyl, ethyl, propyl, isopropyl, butyl, isobutyl, sec-butyl, tert-butyl, pentyl, and hexyl. A “C₀” alkyl (as in “C₀-C₃-alkyl”) is a covalent bond (like “C₀” hydrocarbyl).

The term “alkenyl” as used herein means an unsaturated straight or branched chain aliphatic group with one or more carbon-carbon double bonds, having from 2 to 12 carbon atoms (2, 3, 4, 5, 6, 7, 8, 9, 10, 11, or 12), which is optionally substituted with one, two or three substituents. Exemplary alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms (2, 3, 4, 5, 6, 7, 8, 9, 10, 11, or 12), which is optionally substituted with one, two or three substituents. Exemplary alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Exemplary alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “alkoxy” refers to —O-alkyl.

The term “cycloalkyl” as employed herein includes saturated and partially unsaturated cyclic hydrocarbon groups having 3 to 12 carbons (3, 4, 5, 6, 7, 8, 9, 10, 11, or 12), wherein the cycloalkyl group additionally is optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are replaced by a heteratom selected from the group consisting of O, S, and N.

An “aryl” group is a C₅-C₁₄ aromatic moiety comprising one to three aromatic rings, which is optionally substituted. The aryl group can be a C₆-C₁₀ aryl group. Exemplary aryl groups include, without limitation, phenyl, naphthyl, anthracenyl, and fluorenyl. An “aralkyl” or “arylalkyl” group comprises an aryl group covalently linked to an alkyl group, either of which may independently be optionally substituted or unsubstituted. Preferably, the aralkyl group is (C₁-C₆)alk(C₆-C₁₀)aryl, including, without limitation, benzyl, phenethyl, and naphthylmethyl.

A “heterocyclyl” or “heterocyclic” group is a ring structure having from about 3 to about 8 atoms, preferably 4 to 7 atoms, wherein one or more atoms are selected from the group consisting of N, O, and S. The heterocyclic group is optionally substituted on carbon at one or more positions. The heterocyclic group is also independently optionally substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Exemplary heterocyclic groups include, without limitation, epoxy, azetidinyl, aziridinyl, tetrahydrofuranyl, pyrrolidinyl, piperidinyl, piperazinyl, thiazolidinyl, oxazolidinyl, oxazolidinonyl, and morpholino. Specifically excluded from the scope of this term are compounds having adjacent annular O and/or S atoms.

As used herein, the term “heteroaryl” refers to groups having 5 to 14 ring atoms, preferably 5, 6, 9, or 10 ring atoms; having 6, 10, or 14 pi electrons shared in a cyclic array; and having, in addition to carbon atoms, from one to three heteroatoms per ring selected from the group consisting of N, O, and S. A “heteroaralkyl” or “heteroarylalkyl” group comprises a heteroaryl group covalently linked to an alkyl group, either of which is independently optionally substituted or unsubstituted. Preferred heteroalkyl groups comprise a C₁-C₆ alkyl group and a heteroaryl group having 5, 6, 9, or 10 ring atoms. Examples of heteroaralkyl groups include pyridylmethyl, pyridylethyl, pyrrolylmethyl, pyrrolylethyl, imidazolylmethyl, imidazolylethyl, thiazolylmethyl, thiazolylethyl, benzimidazolylmethyl, benzimidazolylethyl, quinazolinylmethyl, quinolinylmethyl, quinolinylethyl, benzofuranylmethyl, indolinylethyl, isoquinolinylmethyl, isoindolylmethyl, cinnolinylmethyl, and benzothiophenylethyl. Specifically excluded from the scope of this term are compounds having adjacent annular O and/or S atoms.

An “arylene,” “heteroarylene,” or “heterocyclylene” group is an aryl, heteroaryl, or heterocyclyl group, as defined hereinabove, that is positioned between and serves to connect two other chemical groups.

Exemplary heterocyclyls and heteroaryls include, but are not limited to, acridinyl, azocinyl, azetidinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

As employed herein, when a moiety (e.g., cycloalkyl, hydrocarbyl, aryl, heteroaryl, heterocyclic, urea, etc.) is described as “optionally substituted” it is meant that the group optionally has from one to four (1, 2, 3, or 4) non-hydrogen substituents. Suitable substituents include, without limitation, halo, hydroxy, oxo (e.g., an annular —CH— substituted with oxo is —C(O)—) nitro, halohydrocarbyl, hydrocarbyl, aryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

A “halohydrocarbyl” is a hydrocarbyl moiety in which from one to all hydrogens have been replaced with one or more halo.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally substituted. The term “sulfonamido” refers to a sulfonamide substituent attached by either the sulfur or the nitrogen atom. The term “amino” is meant to include NR₃₀, R₃₁, alkylamino, arylamino, and cyclic amino groups. The term “ureido” as employed herein refers to a substituted or unsubstituted urea moiety.

The term “radical” as used herein means a chemical moiety comprising one or more unpaired electrons.

A moiety that is substituted is one in which one or more hydrogens have been independently replaced with another chemical substituent. As a non-limiting example, substituted phenyls include 2-fluorophenyl, 3,4-dichlorophenyl, 3-chloro-4-fluoro-phenyl, 2-fluor-3-propylphenyl. As another non-limiting example, substituted n-octyls include 2,4-dimethyl-5-ethyl-octyl and 3-cyclopentyl-octyl. Included within this definition are methylenes (—CH₂—) substituted with oxygen to form carbonyl —CO—).

As used herein, an “unsubstituted” moiety as defined above (e.g., unsubstituted cycloalkyl, unsubstituted heteroaryl, etc.) means that moiety as defined above that does not have any of the optional substituents for which the definition of the moiety (above) otherwise provides. Thus, for example, while an “aryl” includes phenyl and phenyl substituted with a halo, “unsubstituted aryl” does not include phenyl substituted with a halo.

As used herein, a therapeutically effective amount of a compound is an amount that is sufficient to ameliorate, or in some manner reduce, a symptom or stop or reverse progression of a condition, or negatively modulate or inhibit the activity of KDM5B. Such amount may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Compounds

In certain aspects, compounds are provided that inhibit KDM5 activity. In certain embodiments, the compounds are represented by formula (I):

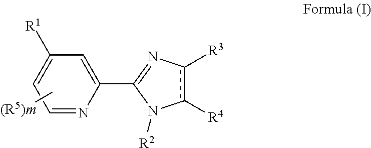

or a pharmaceutical salt thereof:

wherein R¹ is —COOR⁶, —C(O)N(H)CN, —C(O)N(H)OH, or tetrazolyl; R² is C₀-C₆ alkyl-R¹; R³ and R⁴ are each independently selected from hydrogen, halogen, alkyl, alkoxy, —(CH₂)ₙOH, —(CH₂)ₙC(O)NHR⁶, —(CH₂)ₙC(O)NHR⁷, —(CH₂)ₙC(O)NHR⁷, —(CH₂)ₙC(O)NR⁶R⁷, —(CH₂)ₙN(R⁶)C(O)R³, (CH₂)ₙN(R⁶)C(O)R⁷, or —(CH₂)ₙNHC(O)R⁵, —(CH₂)ₙNHC(O)R⁷, carbocyclyl, heterocyclyl, aryl, heteroaryl, alkylcarbocyclyl, alkylheterocyclyl, alkylaryl, alkylheteroaryl, wherein each of the carbocyclyl, heterocyclyl, aryl, heteroaryl, alkylcarbocyclyl, alkylheterocyclyl, alkylaryl, alkylheteroaryl is optionally substituted with one or more R; or R³ and R⁴ and the carbon atoms to which they are attached form a 5-7 membered unsaturated, partial unsaturated or saturated ring system optionally containing 1-3 heteroatoms selected from N, O or S, and further optionally substituted with one or more R⁵;

R⁵ is alkyl, alkenyl, alkynyl, halogen, haloalkyl, alkoxy, cyano, amino, —COOR⁶. C(O)NHR⁶, C(O)N(R⁶)₂, N(R⁶)C(O)R³, NHC(O)R³, aryloxy or optionally substituted heterocyclyl. R⁶ is hydrogen or alkyl; R⁷ is hydrogen, carbocyclyl, heterocyclyl, aryl, or heteroaryl, wherein each of the carbocyclyl, heterocyclyl, aryl, or heteroaryl may be optionally substituted with one or more R⁵; or

R⁶ and R⁷ and the nitrogen atom to which they are attached form a 4-7 membered unsaturated, partial unsaturated or saturated ring system optionally containing 1-3 heteroatoms selected from N, O or S, and further optionally substituted with one or more R⁵; and m and n are each independently zero or an integer between 1 and 3.

In certain embodiments, R¹ is —COOR⁶ or —C(O)N(H)CN. In certain preferred embodiments, R¹ is —COOH. In other embodiments, R² is C₀-C₃ alkyl-R⁷. In certain other embodiments, R⁷ is aryl or heteroaryl each of which may be optionally substituted with one or more R⁷. In certain preferred embodiments, the aryl is heterocyclyl substituted tetrahydronaphthlanyl or phenyl, which is optionally substituted with alkyl, halogen, haloalkyl, alkoxy or cyano.

In other embodiments, R³ is heteroaryl, preferably pyridyl. In still other embodiments, R⁴ is hydrogen, —C(O)NH₂ or —(CH₂)ₙOR⁶ and, in certain preferred embodiments, R⁴ is —C(O)NH₂ or —CH₂OH.

In certain embodiments, exemplary compounds of formula (I) are:

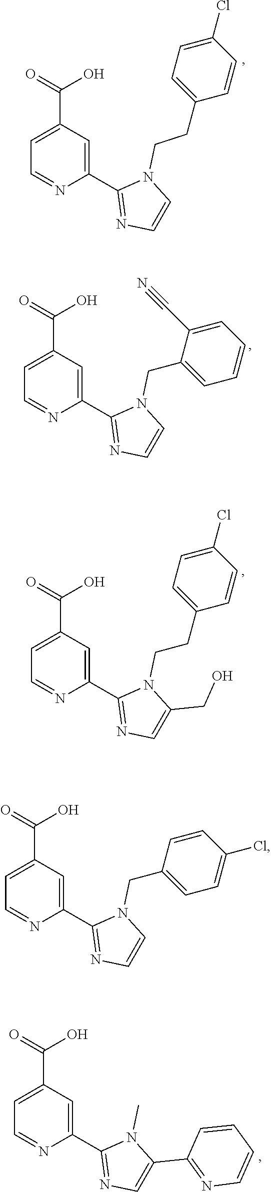 [SMILES: O=C(O)c1ccnc(-c2nccn2CCc2ccc(Cl)cc2)c1 ; N#Cc1ccccc1Cn1ccnc1-c1cc(C(=O)O)ccn1 ; O=C(O)c1ccnc(-c2ncc(CO)n2CCc2ccc(Cl)cc2)c1 ; O=C(O)c1ccnc(-c2nccn2Cc2ccc(Cl)cc2)c1 ; Cn1c(-c2ccccn2)cnc1-c1cc(C(=O)O)ccn1]

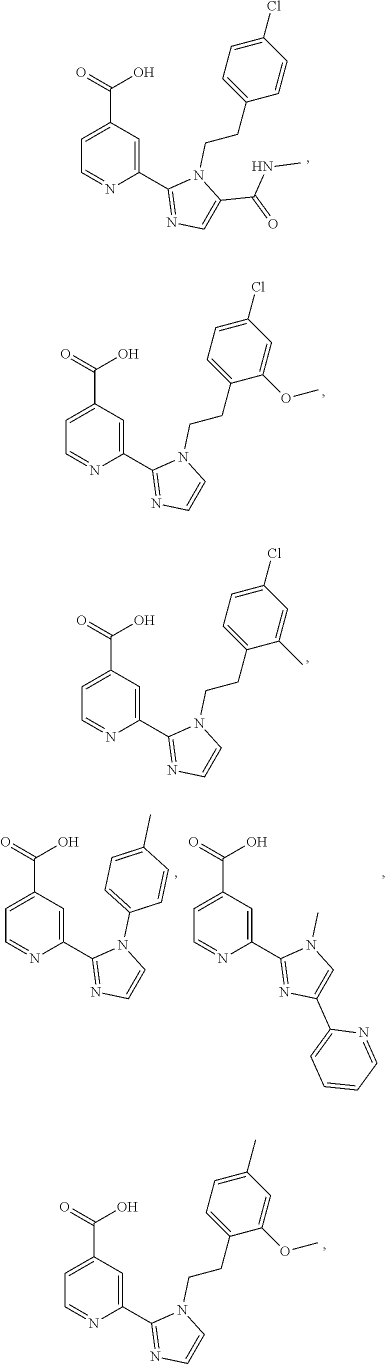

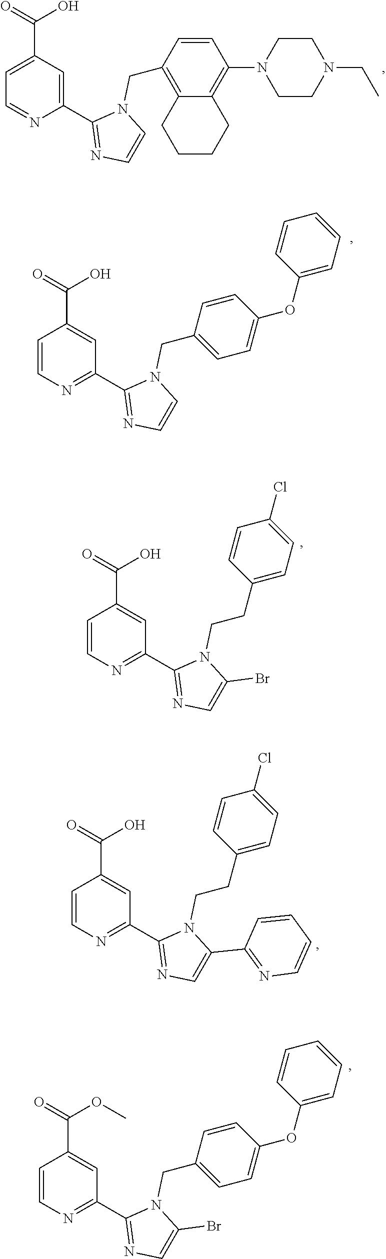 [SMILES: CCN1CCN(c2ccc(Cn3ccnc3-c3cc(C(=O)O)ccn3)c3c2CCCC3)CC1 ; O=C(O)c1ccnc(-c2nccn2Cc2ccc(Oc3ccccc3)cc2)c1 ; O=C(O)c1ccnc(-c2ncc(Br)n2CCc2ccc(Cl)cc2)c1 ; O=C(O)c1ccnc(-c2ncc(-c3ccccn3)n2CCc2ccc(Cl)cc2)c1 ; COC(=O)c1ccnc(-c2ncc(Br)n2Cc2ccc(Oc3ccccc3)cc2)c1]

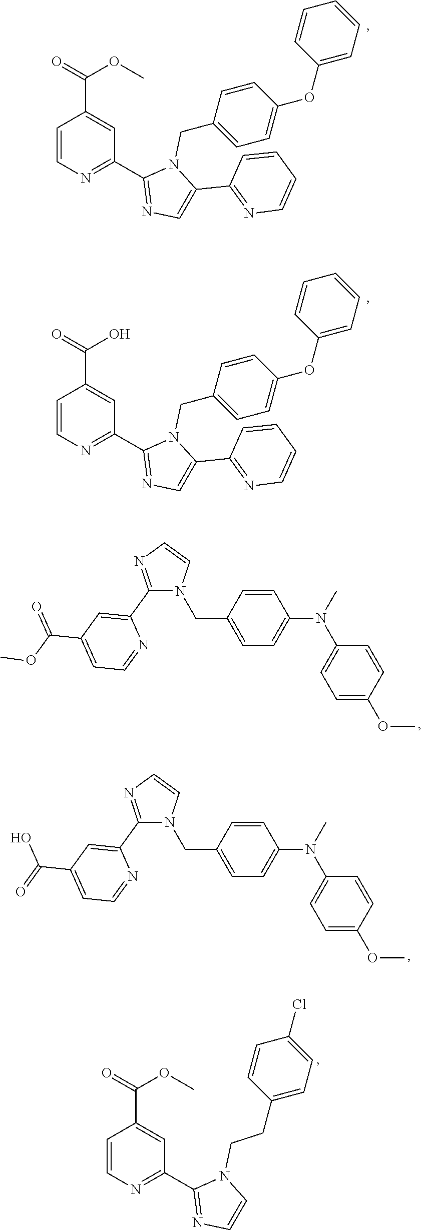

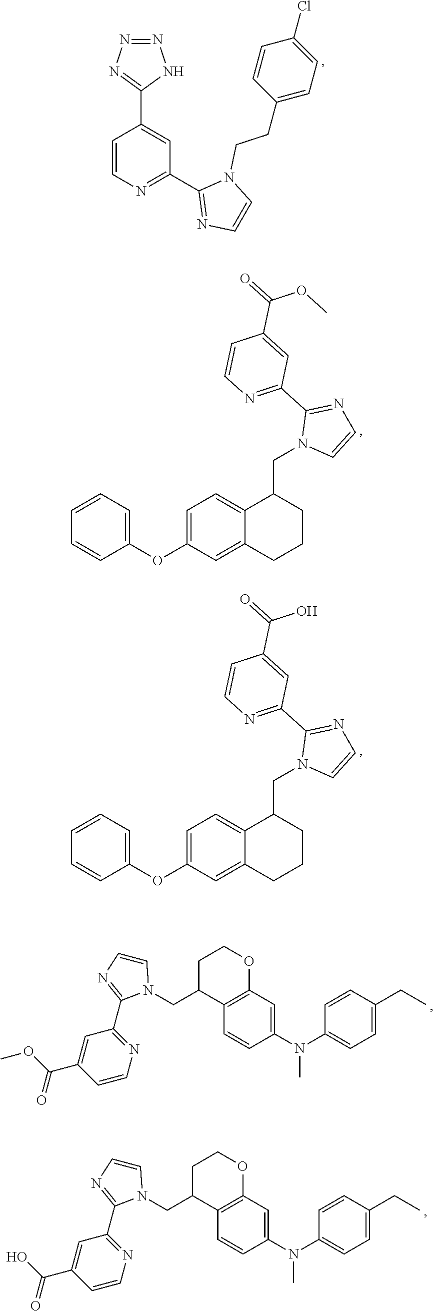 [SMILES: Clc1ccc(CCn2ccnc2-c2cc(-c3nnn[nH]3)ccn2)cc1 ; COC(=O)c1ccnc(-c2nccn2CC2CCCc3cc(Oc4ccccc4)ccc32)c1 ; O=C(O)c1ccnc(-c2nccn2CC2CCCc3cc(Oc4ccccc4)ccc32)c1 ; CCc1ccc(N(C)c2ccc3c(c2)OCCC3Cn2ccnc2-c2cc(C(=O)OC)ccn2)cc1 ; CCc1ccc(N(C)c2ccc3c(c2)OCCC3Cn2ccnc2-c2cc(C(=O)O)ccn2)cc1]

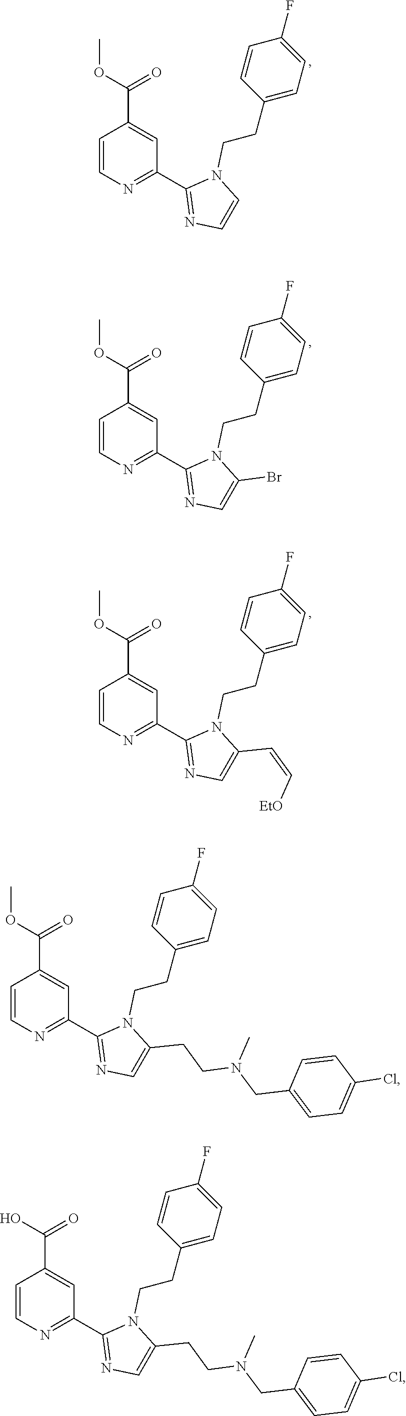 [SMILES: COC(=O)c1ccnc(-c2nccn2CCc2ccc(F)cc2)c1 ; COC(=O)c1ccnc(-c2ncc(Br)n2CCc2ccc(F)cc2)c1 ; CCO/C=C\c1cnc(-c2cc(C(=O)OC)ccn2)n1CCc1ccc(F)cc1 ; COC(=O)c1ccnc(-c2ncc(CCN(C)Cc3ccc(Cl)cc3)n2CCc2ccc(F)cc2)c1 ; CN(CCc1cnc(-c2cc(C(=O)O)ccn2)n1CCc1ccc(F)cc1)Cc1ccc(Cl)cc1]

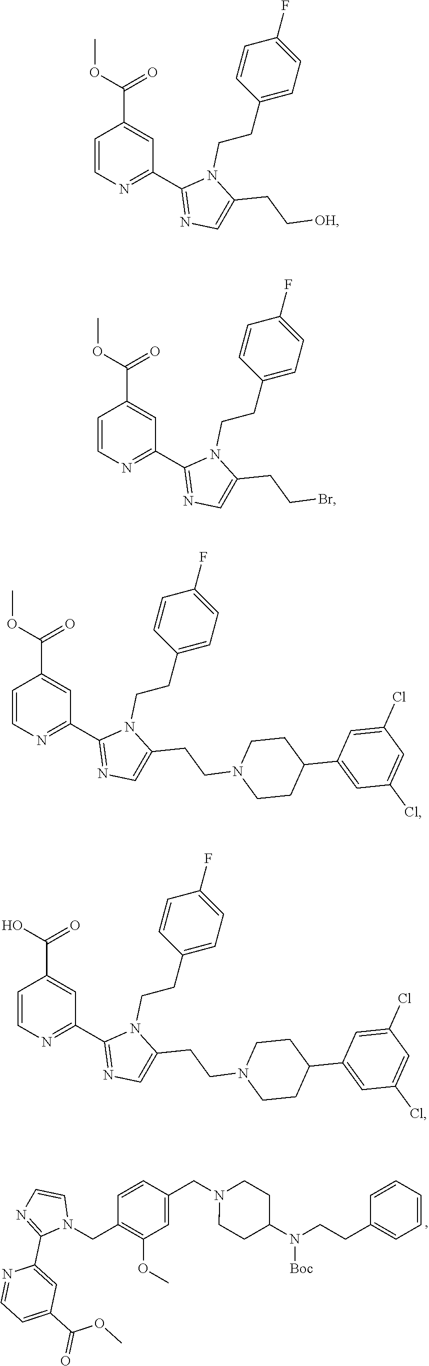 [SMILES: COC(=O)c1ccnc(-c2ncc(CCO)n2CCc2ccc(F)cc2)c1 ; COC(=O)c1ccnc(-c2ncc(CCBr)n2CCc2ccc(F)cc2)c1 ; COC(=O)c1ccnc(-c2ncc(CCN3CCC(c4cc(Cl)cc(Cl)c4)CC3)n2CCc2ccc(F)cc2)c1 ; O=C(O)c1ccnc(-c2ncc(CCN3CCC(c4cc(Cl)cc(Cl)c4)CC3)n2CCc2ccc(F)cc2)c1 ; COC(=O)c1ccnc(-c2nccn2Cc2ccc(CN3CCC(N(CCc4ccccc4)C(=O)OC(C)(C)C)CC3)cc2OC)c1]

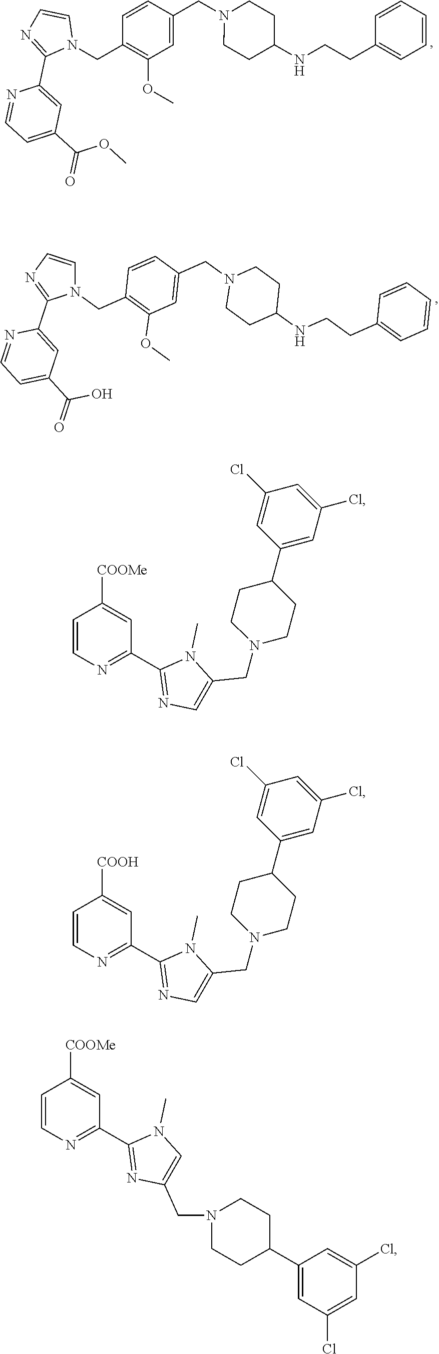 [SMILES: COC(=O)c1ccnc(-c2nccn2Cc2ccc(CN3CCC(NCCc4ccccc4)CC3)cc2OC)c1 ; COc1cc(CN2CCC(NCCc3ccccc3)CC2)ccc1Cn1ccnc1-c1cc(C(=O)O)ccn1 ; COC(=O)c1ccnc(-c2ncc(CN3CCC(c4cc(Cl)cc(Cl)c4)CC3)n2C)c1 ; Cn1c(CN2CCC(c3cc(Cl)cc(Cl)c3)CC2)cnc1-c1cc(C(=O)O)ccn1 ; COC(=O)c1ccnc(-c2nc(CN3CCC(c4cc(Cl)cc(Cl)c4)CC3)cn2C)c1]

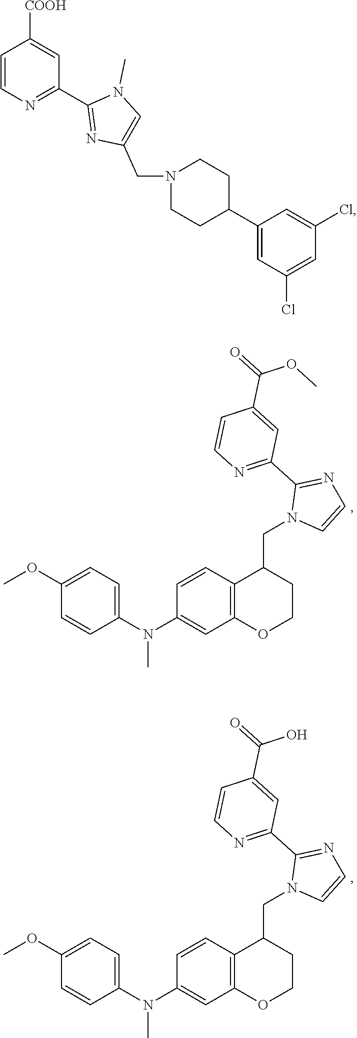 [SMILES: Cn1cc(CN2CCC(c3cc(Cl)cc(Cl)c3)CC2)nc1-c1cc(C(=O)O)ccn1 ; COC(=O)c1ccnc(-c2nccn2CC2CCOc3cc(N(C)c4ccc(OC)cc4)ccc32)c1 ; COc1ccc(N(C)c2ccc3c(c2)OCCC3Cn2ccnc2-c2cc(C(=O)O)ccn2)cc1]

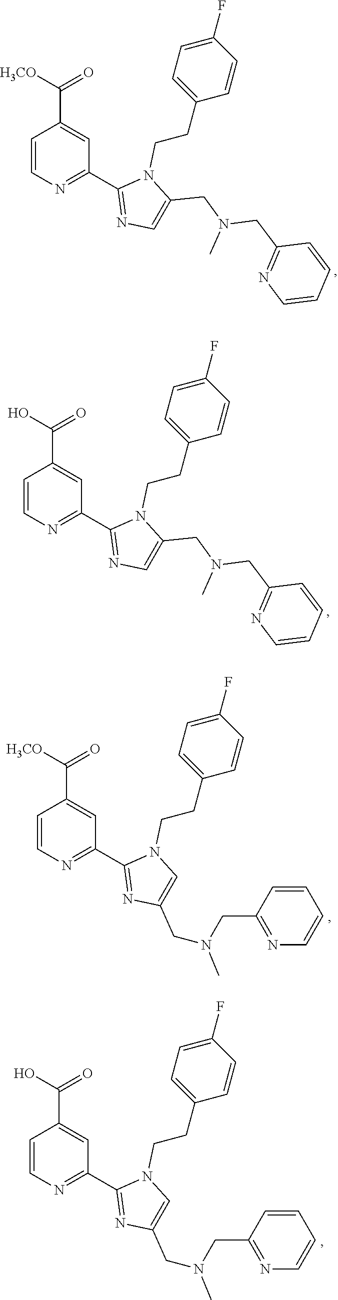

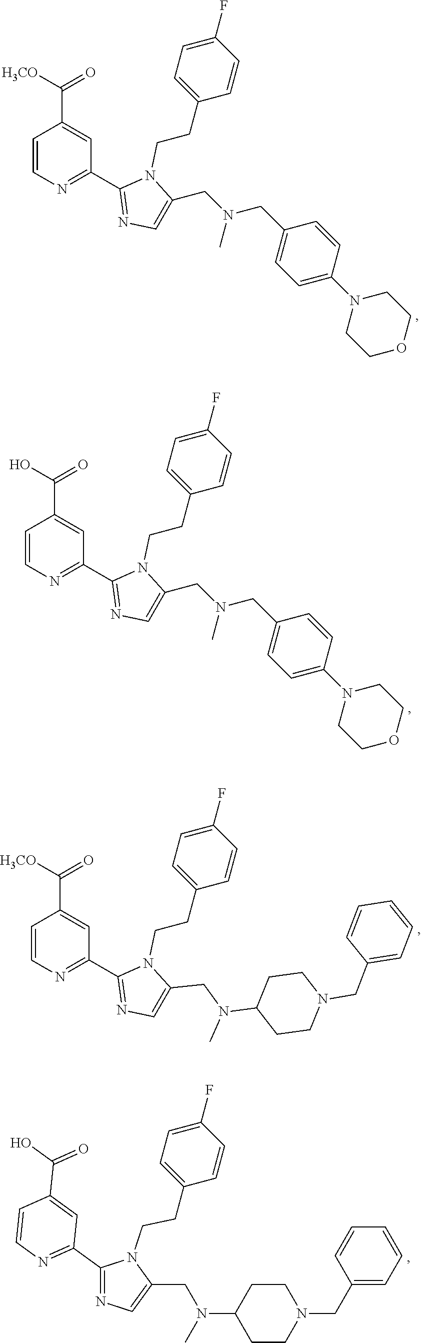 [SMILES: COC(=O)c1ccnc(-c2ncc(CN(C)Cc3ccc(N4CCOCC4)cc3)n2CCc2ccc(F)cc2)c1 ; CN(Cc1ccc(N2CCOCC2)cc1)Cc1cnc(-c2cc(C(=O)O)ccn2)n1CCc1ccc(F)cc1 ; COC(=O)c1ccnc(-c2ncc(CN(C)C3CCN(Cc4ccccc4)CC3)n2CCc2ccc(F)cc2)c1 ; CN(Cc1cnc(-c2cc(C(=O)O)ccn2)n1CCc1ccc(F)cc1)C1CCN(Cc2ccccc2)CC1]

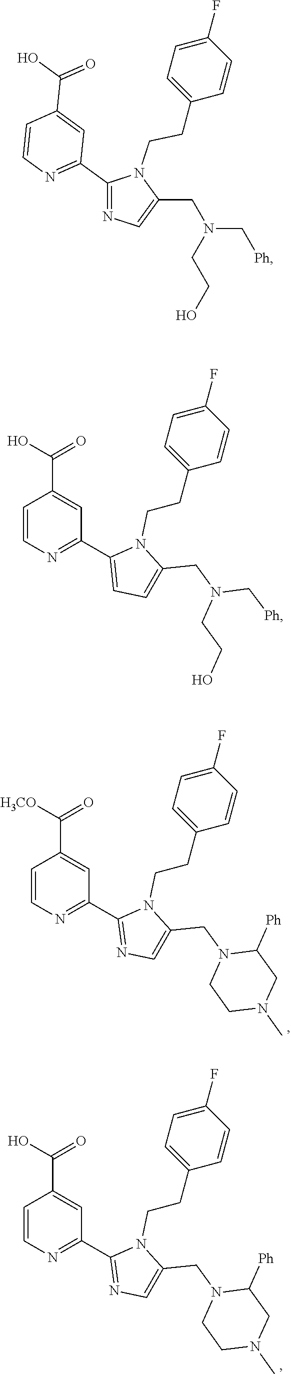 [SMILES: O=C(O)c1ccnc(-c2ncc(CN(CCO)Cc3ccccc3)n2CCc2ccc(F)cc2)c1 ; O=C(O)c1ccnc(-c2ccc(CN(CCO)Cc3ccccc3)n2CCc2ccc(F)cc2)c1 ; COC(=O)c1ccnc(-c2ncc(CN3CCN(C)CC3c3ccccc3)n2CCc2ccc(F)cc2)c1 ; CN1CCN(Cc2cnc(-c3cc(C(=O)O)ccn3)n2CCc2ccc(F)cc2)C(c2ccccc2)C1]

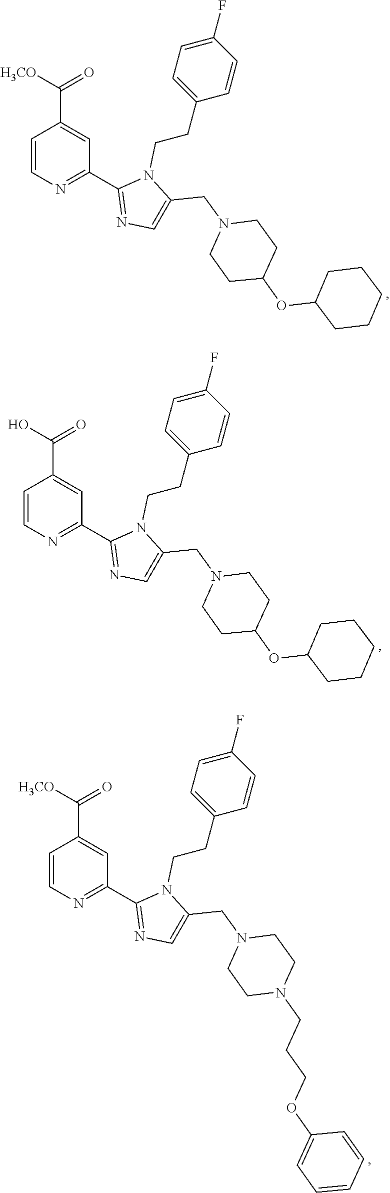 [SMILES: COC(=O)c1ccnc(-c2ncc(CN3CCC(OC4CCCCC4)CC3)n2CCc2ccc(F)cc2)c1 ; O=C(O)c1ccnc(-c2ncc(CN3CCC(OC4CCCCC4)CC3)n2CCc2ccc(F)cc2)c1 ; COC(=O)c1ccnc(-c2ncc(CN3CCN(CCCOc4ccccc4)CC3)n2CCc2ccc(F)cc2)c1]

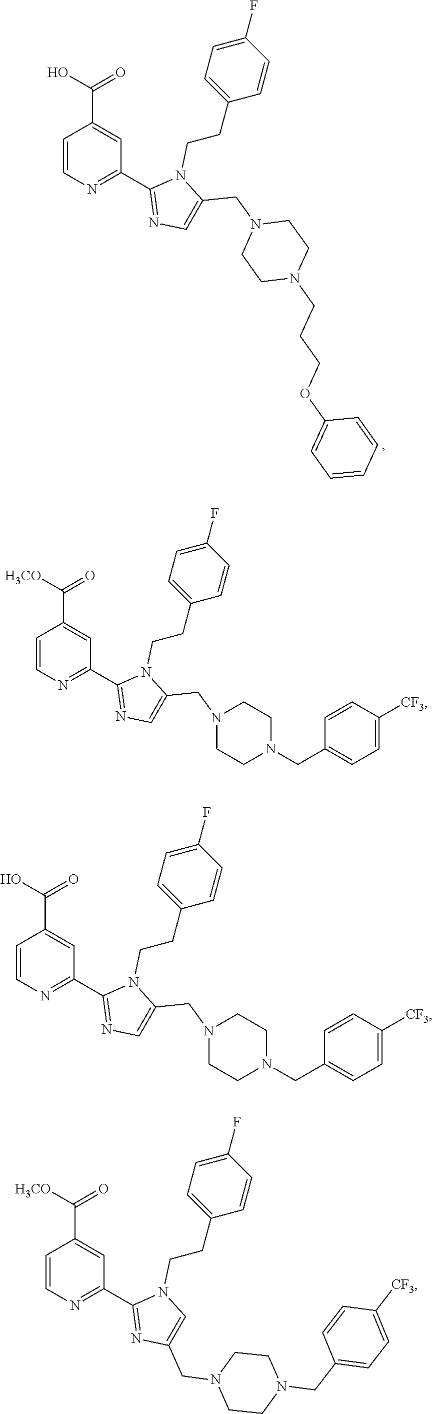

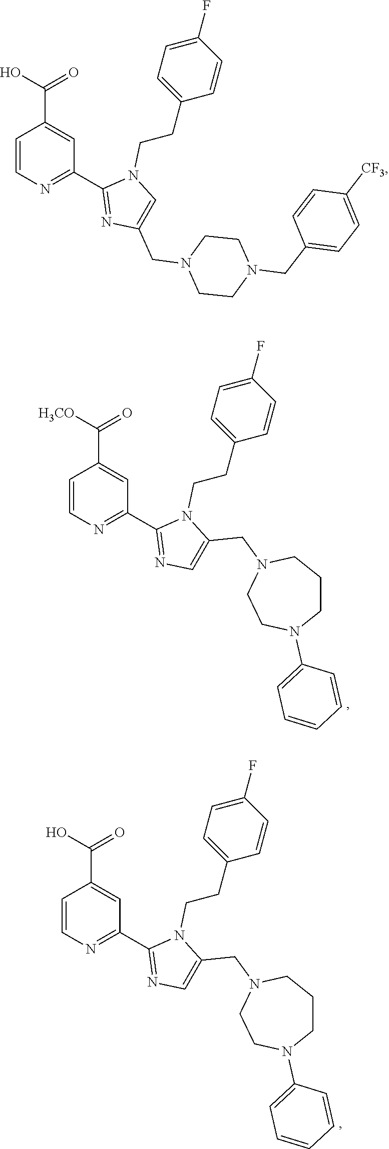 [SMILES: O=C(O)c1ccnc(-c2nc(CN3CCN(Cc4ccc(C(F)(F)F)cc4)CC3)cn2CCc2ccc(F)cc2)c1 ; COC(=O)c1ccnc(-c2ncc(CN3CCCN(c4ccccc4)CC3)n2CCc2ccc(F)cc2)c1 ; O=C(O)c1ccnc(-c2ncc(CN3CCCN(c4ccccc4)CC3)n2CCc2ccc(F)cc2)c1]

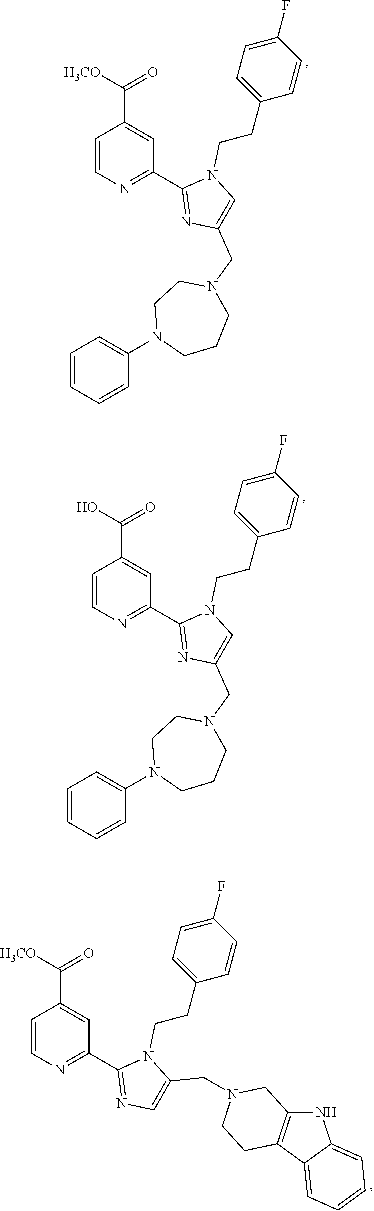 [SMILES: COC(=O)c1ccnc(-c2nc(CN3CCCN(c4ccccc4)CC3)cn2CCc2ccc(F)cc2)c1 ; O=C(O)c1ccnc(-c2nc(CN3CCCN(c4ccccc4)CC3)cn2CCc2ccc(F)cc2)c1 ; COC(=O)c1ccnc(-c2ncc(CN3CCc4c([nH]c5ccccc45)C3)n2CCc2ccc(F)cc2)c1]

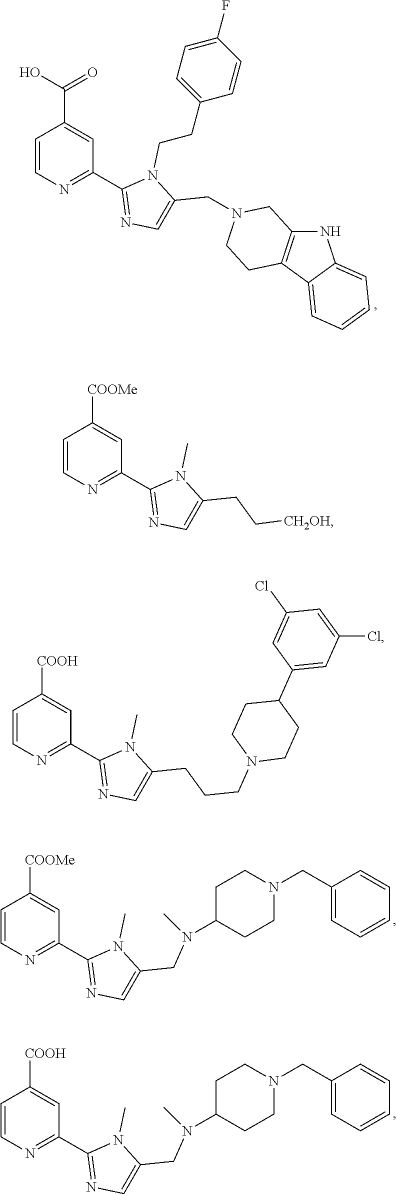 [SMILES: O=C(O)c1ccnc(-c2ncc(CN3CCc4c([nH]c5ccccc45)C3)n2CCc2ccc(F)cc2)c1 ; COC(=O)c1ccnc(-c2ncc(CCCO)n2C)c1 ; Cn1c(CCCN2CCC(c3cc(Cl)cc(Cl)c3)CC2)cnc1-c1cc(C(=O)O)ccn1 ; COC(=O)c1ccnc(-c2ncc(CN(C)C3CCN(Cc4ccccc4)CC3)n2C)c1 ; CN(Cc1cnc(-c2cc(C(=O)O)ccn2)n1C)C1CCN(Cc2ccccc2)CC1]

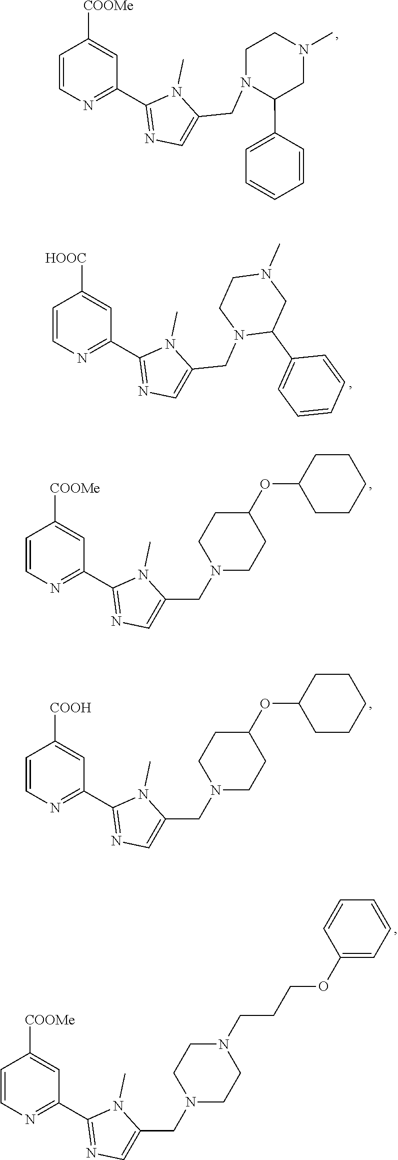

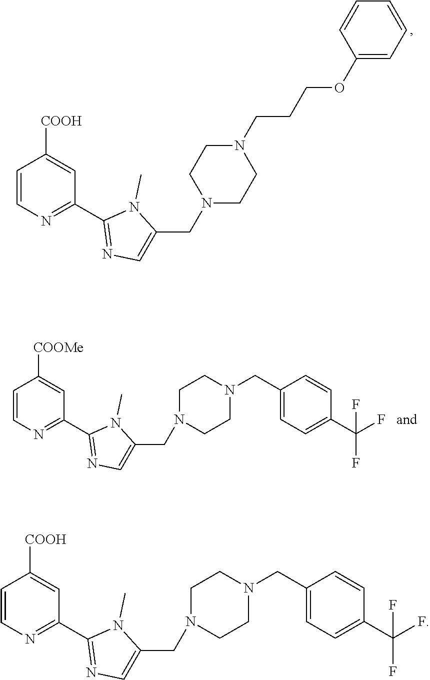 [SMILES: Cn1c(CN2CCN(CCCOc3ccccc3)CC2)cnc1-c1cc(C(=O)O)ccn1 ; COC(=O)c1ccnc(-c2ncc(CN3CCN(Cc4ccc(C(F)(F)F)cc4)CC3)n2C)c1 ; "and" ; Cn1c(CN2CCN(Cc3ccc(C(F)(F)F)cc3)CC2)cnc1-c1cc(C(=O)O)ccn1]

In yet other embodiments, R³ and R⁴ and the carbon atoms to which they are attached form a 5-7 membered unsaturated, partial unsaturated or saturated ring system optionally containing 1-3 heteroatoms selected from N, O or S, and further optionally substituted with one or more R⁵. In certain embodiments, R¹ is —COOR⁶ or —C(O)N(H)CN. In certain preferred embodiments, R¹ is —COOH. In other embodiments, R² is C₁-C₃ alkyl-R⁷. In certain other embodiments, R⁷ is is aryl or heteroaryl each of which may be optionally substituted with one or more R⁵. In certain preferred embodiments, the aryl is phenyl, which is optionally substituted with alkyl, halogen, haloalkyl, alkoxy or cyano.

In other embodiments, exemplary compounds of formula (I) are selected from the group consisting of:

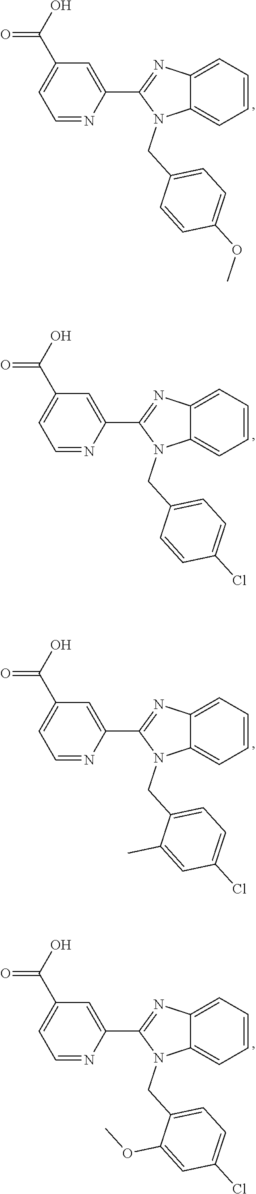 [SMILES: COc1ccc(Cn2c(-c3cc(C(=O)O)ccn3)nc3ccccc32)cc1 ; O=C(O)c1ccnc(-c2nc3ccccc3n2Cc2ccc(Cl)cc2)c1 ; Cc1cc(Cl)ccc1Cn1c(-c2cc(C(=O)O)ccn2)nc2ccccc21 ; COc1cc(Cl)ccc1Cn1c(-c2cc(C(=O)O)ccn2)nc2ccccc21]

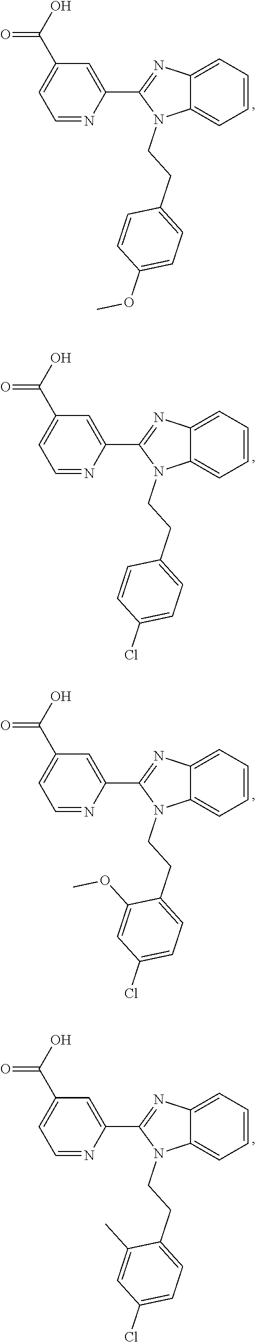 [SMILES: COc1ccc(CCn2c(-c3cc(C(=O)O)ccn3)nc3ccccc32)cc1 ; O=C(O)c1ccnc(-c2nc3ccccc3n2CCc2ccc(Cl)cc2)c1 ; COc1cc(Cl)ccc1CCn1c(-c2cc(C(=O)O)ccn2)nc2ccccc21 ; Cc1cc(Cl)ccc1CCn1c(-c2cc(C(=O)O)ccn2)nc2ccccc21]

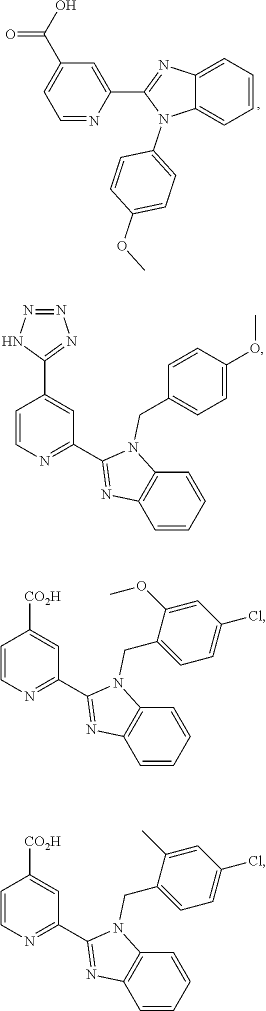 [SMILES: COc1ccc(-n2c(-c3cc(C(=O)O)ccn3)nc3ccccc32)cc1 ; COc1ccc(Cn2c(-c3cc(-c4nnn[nH]4)ccn3)nc3ccccc32)cc1 ; COc1cc(Cl)ccc1Cn1c(-c2cc(C(=O)O)ccn2)nc2ccccc21 ; Cc1cc(Cl)ccc1Cn1c(-c2cc(C(=O)O)ccn2)nc2ccccc21]

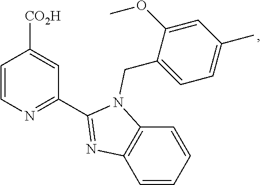 [SMILES: COc1cc(C)ccc1Cn1c(-c2cc(C(=O)O)ccn2)nc2ccccc21]

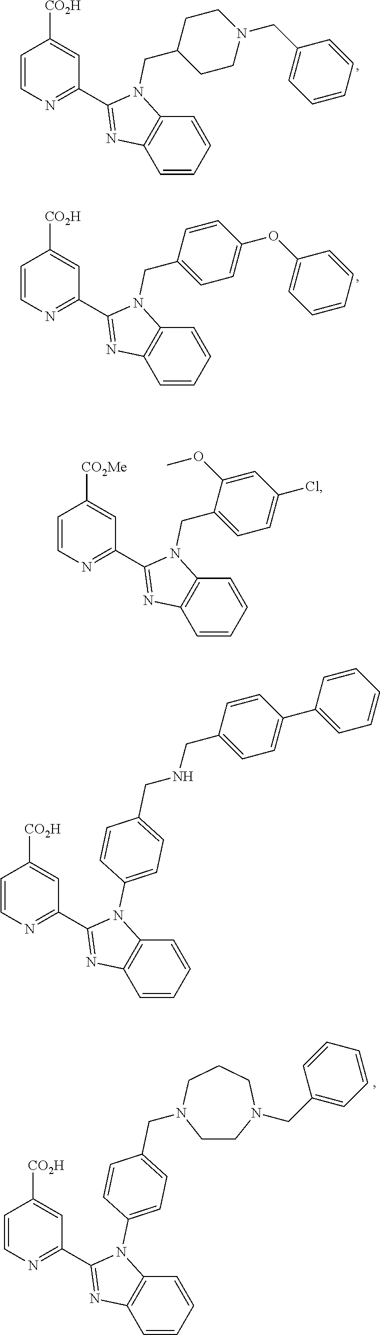 [SMILES: O=C(O)c1ccnc(-c2nc3ccccc3n2CC2CCN(Cc3ccccc3)CC2)c1 ; O=C(O)c1ccnc(-c2nc3ccccc3n2Cc2ccc(Oc3ccccc3)cc2)c1 ; COC(=O)c1ccnc(-c2nc3ccccc3n2Cc2ccc(Cl)cc2OC)c1 ; O=C(O)c1ccnc(-c2nc3ccccc3n2-c2ccc(CNCc3ccc(-c4ccccc4)cc3)cc2)c1 ; O=C(O)c1ccnc(-c2nc3ccccc3n2-c2ccc(CN3CCCN(Cc4ccccc4)CC3)cc2)c1]

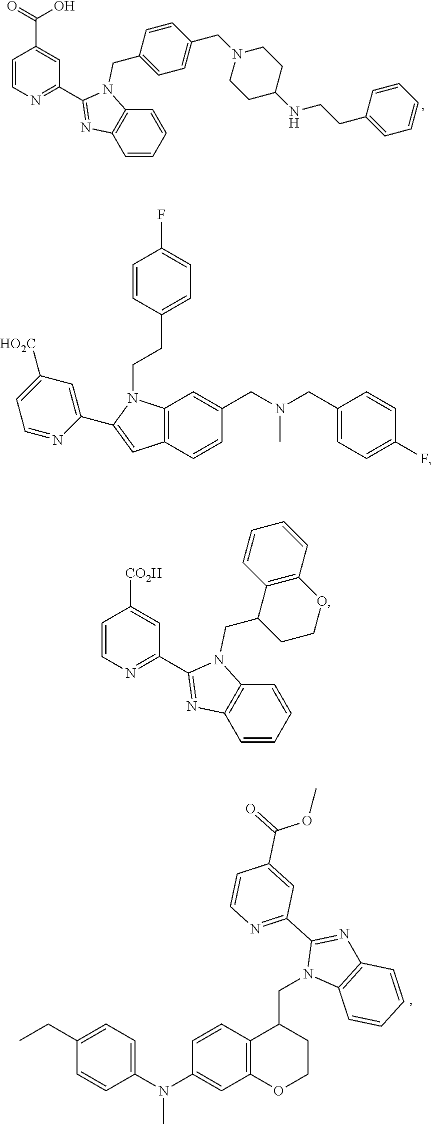 [SMILES: O=C(O)c1ccnc(-c2nc3ccccc3n2Cc2ccc(CN3CCC(NCCc4ccccc4)CC3)cc2)c1 ; CN(Cc1ccc(F)cc1)Cc1ccc2cc(-c3cc(C(=O)O)ccn3)n(CCc3ccc(F)cc3)c2c1 ; O=C(O)c1ccnc(-c2nc3ccccc3n2CC2CCOc3ccccc32)c1 ; CCc1ccc(N(C)c2ccc3c(c2)OCCC3Cn2c(-c3cc(C(=O)OC)ccn3)nc3ccccc32)cc1]

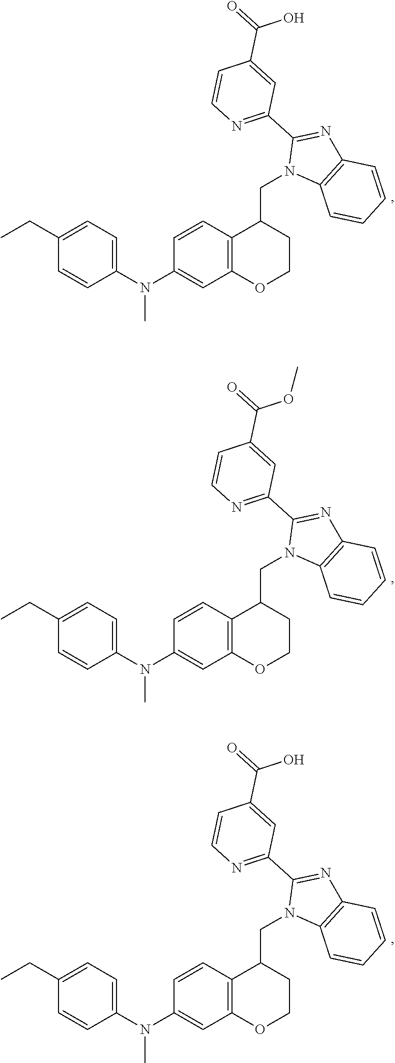 [SMILES: CCc1ccc(N(C)c2ccc3c(c2)OCCC3Cn2c(-c3cc(C(=O)O)ccn3)nc3ccccc32)cc1 ; CCc1ccc(N(C)c2ccc3c(c2)OCCC3Cn2c(-c3cc(C(=O)OC)ccn3)nc3ccccc32)cc1 ; CCc1ccc(N(C)c2ccc3c(c2)OCCC3Cn2c(-c3cc(C(=O)O)ccn3)nc3ccccc32)cc1]

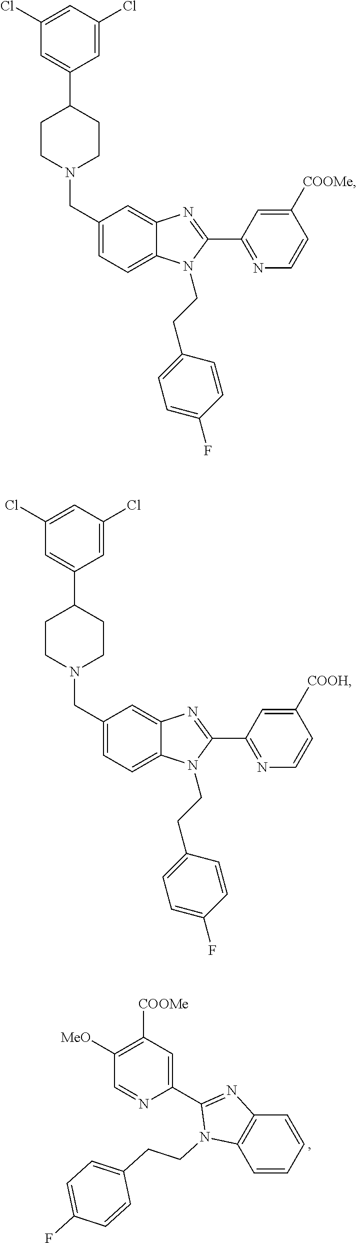 [SMILES: COC(=O)c1ccnc(-c2nc3cc(CN4CCC(c5cc(Cl)cc(Cl)c5)CC4)ccc3n2CCc2ccc(F)cc2)c1 ; O=C(O)c1ccnc(-c2nc3cc(CN4CCC(c5cc(Cl)cc(Cl)c5)CC4)ccc3n2CCc2ccc(F)cc2)c1 ; COC(=O)c1cc(-c2nc3ccccc3n2CCc2ccc(F)cc2)ncc1OC]

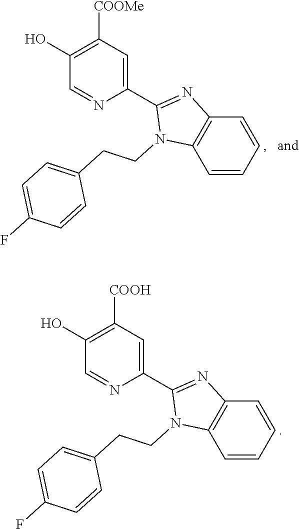 [SMILES: COC(=O)c1cc(-c2nc3ccccc3n2CCc2ccc(F)cc2)ncc1O ; O=C(O)c1cc(-c2nc3ccccc3n2CCc2ccc(F)cc2)ncc1O]

The compounds of formula (I) may be formulated into pharmaceutical compositions.

The compounds disclosed herein may have one or more chiral centers and can be synthesized as stereoisomeric mixtures, isomers of identical constitution that differ in the arrangement of their atoms in space. The compounds may be used as mixtures or the individual components/isomers may be separated using reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds disclosed herein may be synthesized using optically pure, chiral reagents and intermediates to prepare individual isomers or entantiomers. Unless otherwise indicated, all chiral (enantiomeric and diastereomeric) and racemic forms are within the scope of the compounds disclosed herein.

Unless otherwise indicated to the contrary, chemical structures, which include one or more stereocenters, illustrated herein without indicating absolute or relative stereochemistry, encompass all possible stereoisomeric forms of the compound (e.g., diastereomers and enantiomers), and mixtures thereof.

Pharmaceutical Compositions

In another aspect, pharmaceutical compositions are provided comprising a histone demethylase inhibitor disclosed herein and a pharmaceutically acceptable carrier, excipient, or diluent. Compounds disclosed herein may be formulated by any suitable method known in the art and may be prepared for administration by any suitable route, including, without limitation, parenteral, oral, sublingual, transdermal, topical, intranasal, intratracheal, or intrarectal. In certain embodiments, compounds disclosed herein are administered intravenously, such as in a hospital setting. In certain other embodiments, the compounds disclosed herein are administered orally.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions disclosed herein may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term pharmaceutically acceptable salts refers to salts that retain the desired biological activity of the above-identified compounds and exhibit minimal or no undesired toxicological effects. Examples of such salts include, but are not limited to, acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. A preferred dose of the active compound for all of the above-mentioned conditions is in the range from about 0.01 to 300 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds disclosed herein may be used in the methods described herein.

Methods of Use

KDM5B (JARID1B) is a member of the JmjC histone lysine demethylase superfamily and acts on di- and trimethylated lysine residues of histones, particularly di- and trimethylated lysine 4 in the N-terminal tail of histone H3. KDM5B has been reported to be overexpressed in a number of cancers, including breast, prostate, testicular, ovarian, leukemia and bladder carcinoma, and KDM5B activity is reported to be required for continued growth of melanoma (e.g., see H\acute{ø}fedlt et al., (2013) Nature Rev Drug Disc., Published on line Nov. 13, 2013 doi:10. 1038/nrd4154).

With increasing evidence that histone lysine demethylases, including KDM5B, play a critical role in a diverse set of cancers and diseases, a variety of histone demethylase inhibitors have been reported in the literature (e.g., see Lizcano and Garcia (2012) Pharmaceuticals 5:963-990). Inhibitors of KDM5B and other Jumonji C superfamily members compete with the 2-oxoglutrate co-factor and bind to the catalytic region containing Fe(II) to block demethylation.

In yet another aspect, methods are provided for inhibiting KDM5B activity in a cell, comprising contacting the cell in which inhibition of KDM5B activity is desired with a therapeutically effective amount of a compound of formula (I), pharmaceutically acceptable salts thereof or pharmaceutical compositions containing the compound or pharmaceutically acceptable salt thereof.

One use for the compounds, compositions, and methods disclosed herein is for inhibiting KDM5B activity in a cell.

In certain embodiments, a cell in which inhibition of KDM5B activity is desired is contacted with a therapeutically effective amount of a compound of formula (I) to negatively modulate the activity of KDM5B. In other embodiments, a therapeutically effective amount of pharmaceutically acceptable salt or pharmaceutical compositions containing the compound of formula (I) may be used.

By negatively modulating the activity of KDM5B, particularly in cases for cells overexpressing the KDM5B enzyme or somatic mutations that activate the KDM5B enzyme, the methods are designed to restore normal cellular transcription expression patterns, e.g., by altering the methylation pattern of H3K4 to inhibit undesired cellular proliferation resulting from enhanced KDM5B activity and/or expression within the cell. The cells may be contacted in a single dose or multiple doses in accordance with a particular treatment regimen to effect the desired negative modulation of KDM5B. The inhibition of cellular proliferation and KDM5B-dependent demethylation of histone H3K4 may be monitored in the cell using well known methods to assess the effectiveness of treatment and dosages may be adjusted accordingly by the attending medical practitioner.

Methods of determining inhibition of KDM5B are known in the art.

In another aspect, methods are provided of treating cancer comprising administering to a patient having cancer a therapeutically effective amount of a compound of formula (I), pharmaceutically acceptable salts thereof or pharmaceutical compositions comprising the compound or pharmaceutically acceptable salts thereof.

The compositions and methods provided herein may be used for the treatment of a wide variety of cancer, including tumors such as prostate, breast, brain, skin, cervical carcinomas, testicular carcinomas, etc. More particularly, cancers that may be treated by the compositions and methods of the invention include, but are not limited to, tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Breast; Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer.

In certain aspects, methods are provided of treating a patient with a cancer in which at least some of the cancerous cells are inappropriately expressing KDM5B, including over-expressing KDM5B, comprising administering to a patient having a cancer in which at least some of the cancerous cells are inappropriately expressing KDM5B, including over-expressing KDM5B a therapeutically effective amount of a compound of formula (I), a pharmaceutically acceptable salt thereof or a pharmaceutical composition comprising the compound or a pharmaceutically acceptable salt thereof.

Methods for the diagnosis of cancer (primary or metastatic) are known in the art. Methods for determining whether the cancer has reduced or has been eliminated, the patient has improved, etc. are known in the art.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively. The inhibition of cellular proliferation and KDM5B-dependent demethylation of histone H3K4 may be monitored in the cell using well known methods to assess the effectiveness of treatment, along with other prognostic or biological factors, and dosages may be adjusted accordingly by the attending medical practitioner.

The term “effective amount” or “a therapeutically effective amount” refers to an amount of a compound or composition that provides the desired biological, therapeutic, and/or prophylactic result. That result can be reduction, amelioration, palliation, lessening, delaying, and/or alleviation of one or more of the signs, symptoms, or causes of a disease, such as cancer, in a patient, or any other desired alteration of a biological system. An effective amount can be administered in one or more administrations. In certain other embodiments, an “effective amount” or “a therapeutically effective amount” is the amount of a compound or composition disclosed herein that improves the life expectancy of a patient by any amount of time, including at least one day, at least one week, at least two weeks, at least three weeks, at least one month, at least two months, at least three months, at least 6 months, at least one year, at least 18 months, at least two years, at least 30 months, or at least three years, or the duration of treatment. An effective amount can be an amount that causes a cancer to shrink or to be eliminated from a patient. Whether a desired result has been achieved can be determined by methods known in the art.

A compound or a composition disclosed herein can be administered to a patient as a monotherapy. In some embodiments, the methods described herein can include administering to the patient one or more additional treatments, such as one or more additional therapeutic agents.

The additional treatment can be any additional treatment, including experimental treatments. The other treatment can be any treatment, any therapeutic agent, that improves or stabilizes the patient's health. An additional therapeutic agent can be administered prior to, concurrently, or after administration of a compound or composition disclosed herein. An additional agent and a compound or composition disclosed herein can be administered using the same delivery method or route or using a different delivery method or route.

In some embodiments, a compound or composition disclosed herein can be formulated with one or more additional active agents useful for treating cancer in a patient.

When a compound or composition disclosed herein is to be used in combination with a second active agent, the agents can be formulated separately or together. For example, the respective pharmaceutical compositions can be mixed, e.g., just prior to administration, and administered together or can be administered separately, e.g., at the same or different times, by the same route or different route.

In some embodiments, a composition can be formulated to include a sub-therapeutic amount of a compound or composition disclosed herein and a sub-therapeutic amount of one or more additional active agents such that the components in total are therapeutically effective for treating a cancer. Methods for determining a therapeutically effective dose of an agent are known in the art.

A patient includes a human patient.

### Reaction Schemes and Examples

The compounds disclosed herein may be prepared using commercially available reagents using the synthetic methods and reaction schemes described herein, or using other reagents and conventional methods well known to those skilled in the art.

For instance, pyridyl imidazole compounds of the present invention may be prepared according to the General Reaction Schemes I-IV whereas pyridyl benzimidazoles compounds of the present invention may be prepared according to the General Reaction Schemes I and II.

General Reaction Schemes

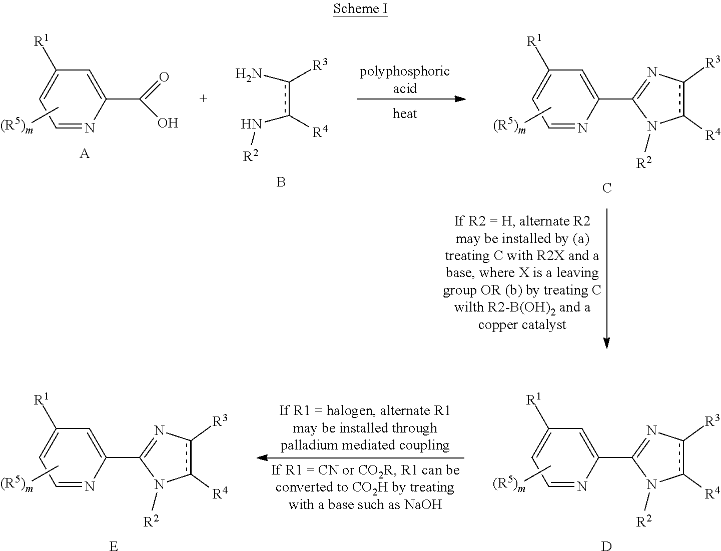

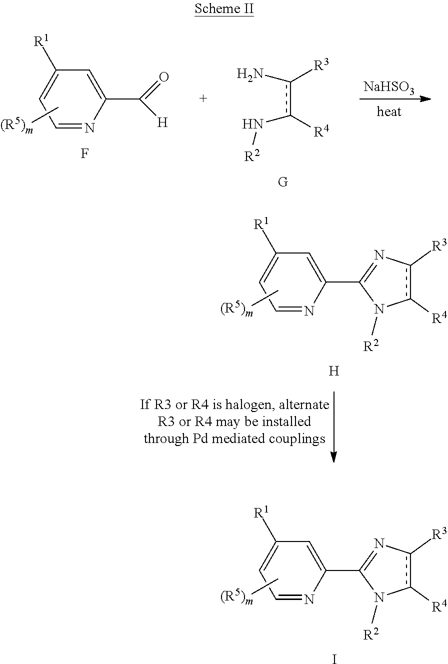

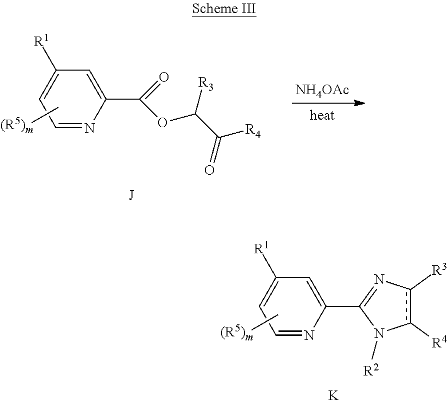

### EXAMPLES

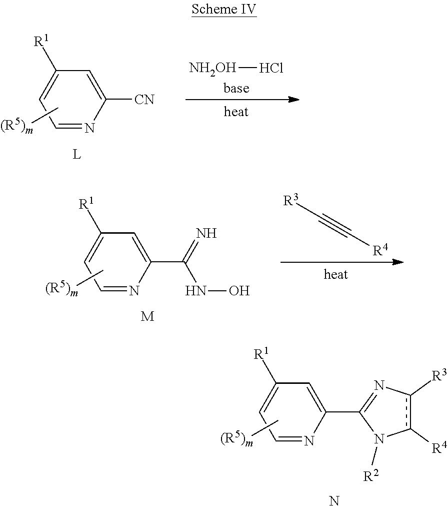

The following Examples are intended to illustrate further certain preferred embodiments of the invention and are not intended to limit the scope of the invention.

### Example 1

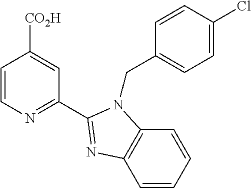 [SMILES: O=C(O)c1ccnc(-c2nc3ccccc3n2Cc2ccc(Cl)cc2)c1]

### Example 1A

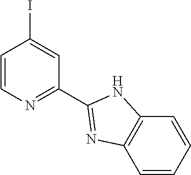 [SMILES: Ic1ccnc(-c2nc3ccccc3[nH]2)c1]

### Example 1A

4-iodopicolinic acid (1.02 g, 4.1 mmol, 1.00 eq) and 1,2-phenylene diamine (0.442 g, 4.1 mmol, 1.0 eq) were stirred together with polyphosphoric acid (5 mL) at 180° C. under N₂ for 5 hrs. The mixture was cooled to 90° C. and diluted with water. The mixture was made basic with 2M Na₂CO₃. A precipitate formed. The precipitate was collected, washed with water and dried under vacuum to give 0.805 g of a brown solid that was used directly in the next step. LCMS MH⁺ calculated, 322.0, found 322.1.

### Example 1B

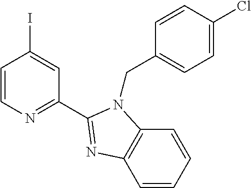 [SMILES: Clc1ccc(Cn2c(-c3cc(I)ccn3)nc3ccccc32)cc1]

### Example 1B

Example 1A (157 mg, 0.49 mmol) was stirred in DMF (2 mL) at 0° C. Sodium hydride (14 mg, 0.6 mmol, 1.2 eq) was added. After 30 minutes (min), 4-chloro benzyl bromide (111 mg, 0.54 mmol, 1.1 eq) was added. The reaction was stirred overnight as the cooling bath warmed to RT. Water was added to the reaction and a precipitate formed. The precipitate was collected by filtration, washed with water and dried under vacuum to give 106 mg of a tan solid (49%). LCMS MH⁺ calculated, 446.0, found 446.0.

### Example 1C

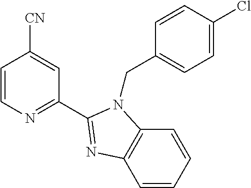 [SMILES: N#Cc1ccnc(-c2nc3ccccc3n2Cc2ccc(Cl)cc2)c1]

### Example 1C

Example 1B (90 mg, 0.2 mmol, 1 eq), zinc cyanide (234 mg, 2 mmol, 10 eq) and PdCl₂-dppf(15 mg, 0.02 mmol, 0.1 eq) were stirred together in DMA (2 mL). The reaction mixture was deoxygenated by alternately evacuating the reaction flask and then introducing N₂. This procedure was repeated two more times. The reaction was heated at 120° C. overnight. The mixture was cooled to RT. A solution of saturated sodium bicarbonate was added. A precipitate formed which was collected by filtration and washed with water. Chromatography (SiO₂, 10%→20%→30% ethyl acetate in pentane) gave 66 mg of the product (96%). LCMS MH⁺ calculated, 345.1, found 345.2. ¹H NMR (DMSO-d6, 500 MHz) δ 8.91 (dd, J=1.1, 5.5 Hz, 1H), 8.71 (dd, J=0.5, 1.5 Hz, 1H), 7.96 (dd, J=1.5, 5 Hz, 1H), 7.80 (m, 1H), 7.62 (m, 1H), 7.32 (m, 4H), 7.17 (d, J=8.5 Hz, 2H), 6.16 (s, 2H).

### Example 1

Example 1C (57 mg, 0.166 mmol, 1 eq) was stirred in EtOH (1 mL). 5 M NaOH (0.5 mL) was added and the reaction heated at 85° C. overnight. The reaction was cooled to RT and neutralized with 3N HCl. A precipitate formed and was collected by filtration. The precipitate was washed with water and dried under vacuum to give 45 mg of an off-white solid (750). LCMS MH⁺ calculated, 364.1, found 364.0. ¹H NMR (DMSO-d6, 500 MHz) δ 8.85 (d, J=5 Hz, 1H), 8.80 (s, 1H), 7.89 (dd, J=1.5, 5 Hz, 1H), 7.79 (m, 1H), 7.60 (m, 1H), 7.31 (m, 4H), 7.18 (d, J=8.5 Hz, 2H), 6.21 (s, 2H).

### Example 2

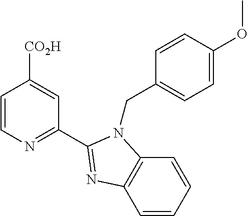 [SMILES: COc1ccc(Cn2c(-c3cc(C(=O)O)ccn3)nc3ccccc32)cc1]

### Example 2A

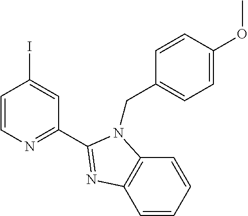 [SMILES: COc1ccc(Cn2c(-c3cc(I)ccn3)nc3ccccc32)cc1]

### Example 2A

Following the procedure for Example 1B, Example 1A (283 mg, 0.88 mmol) and 4-methoxybenzyl chloride (0.14 mL, 0.97 mmol) were reacted to provide the target compound that was used directly in the next step. LCMS MH⁺ calculated, 442.0, found 442.0.

### Example 2B

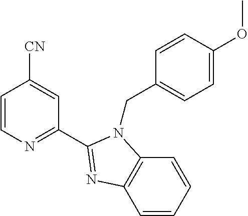 [SMILES: COc1ccc(Cn2c(-c3cc(C#N)ccn3)nc3ccccc32)cc1]

### Example 2B

Following the procedure for Example 1C, Example 2A (304 mg, 0.69 mmol) was converted to the target compound (117 mg, 50%). LCMS MH⁺ calculated, 341.1, found 341.2. ¹H NMR (DMSO-d6, 500 MHz) δ 8.96 (m, 1H), 8.69 (s, 1H), 7.97 (dd, J=1.5, 5 Hz, 1H), 7.78 (dd, J=0.5, 7 Hz, 1H), 7.65 (dd, J=1.5, 6.5 Hz, 1H), 7.31 (m, 2H), 7.11 (d, J=9 Hz, 2H), 6.80 (dd, J=1.5, 6.5 Hz, 2H), 6.10 (s, 2H), 3.33 (s, 3H).

### Example 2

Following the procedure for Example 1, Example 2B (64 mg, 0.19 mmol) was converted to the target compound (51 mg, 75%). LCMS MH⁺ calculated, 360.1, found 360.2. ¹H NMR (DMSO-d6, 500 MHz) δ 8.90 (m, 1H), 8.79 (dd, J=1, 1.5 Hz, 1H), 7.91 (dd, J=1.5, 5 Hz, 1H), 7.77 (m, 1H), 7.63 (m, 1H), 7.29 (m, 2H), 7.18 (d, J=8.5 Hz, 2H), 6.80 (dd, J=2, 6.5 Hz, 2H), 6.15 (s, 2H), 3.40 (s, 3H).

### Example 3

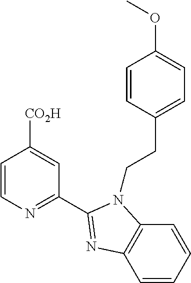 [SMILES: COc1ccc(CCn2c(-c3cc(C(=O)O)ccn3)nc3ccccc32)cc1]

### Example 3A

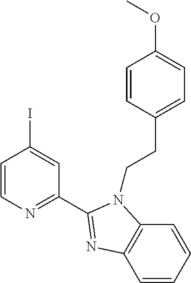 [SMILES: COc1ccc(CCn2c(-c3cc(I)ccn3)nc3ccccc32)cc1]

### Example 3A

Following the procedure of Example 1B, Example 1A (166 mg, 0.52 mmol) and 4-methoxyphenethyl bromide (0.1 mL, 0.62 mmol) were reacted to provide the target compound (58 mg, 25%). LCMS MH⁺ calculated, 456.1, found 456.1.

### Example 3B

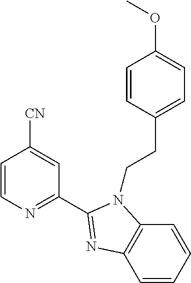 [SMILES: COc1ccc(CCn2c(-c3cc(C#N)ccn3)nc3ccccc32)cc1]

### Example 3B

Following the procedure of Example 1C, Example 3A (55 mg, 0.12 mmol) was converted to the target compound (26 mg, 61%). LCMS MW calculated, 355.2, found 355.2.

### Example 3

Following the procedure of Example 1, Example 3B (26 mg, 0.073 mmol) was converted to the target compound (17 mg, 62%). LCMS MH⁺ calculated, 374.1, found 374.2. ¹H NMR (DMSO-d6, 500 MHz) δ 13.83 (s, 1H), 8.90 (d, J=5 Hz, 1H), 8.43 (s, 1H), 7.87 (dd, J=1.5, 5 Hz, 1H), 7.74 (dd, J=4.5, 8 Hz, 2H), 7.32 (m, 2H), 6.86 (d, J=8.5 Hz, 2H), 6.64 (d, J=8.5 Hz, 2H), 5.03 (t, I=7.5 Hz, 2H), 3.64 (s, 3H), 2.96 (t, J=7.5 Hz, 2H).

### Example 4

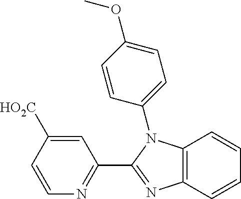 [SMILES: COc1ccc(-n2c(-c3cc(C(=O)O)ccn3)nc3ccccc32)cc1]

### Example 4A

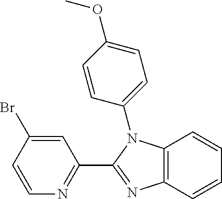 [SMILES: COc1ccc(-n2c(-c3cc(Br)ccn3)nc3ccccc32)cc1]

### Example 4A

1-N-(4-methoxyphenyl)benzene-1,2-diamine (258 mg, 1.2 mmol) and 4-bromo-pyridine-2-carbaldehyde (224 mg, 1.2 mmol) were stirred together in DMA (3 mL). Sodium bisulfite (187 mg, 1.8 mmol) was added and the mixture heated to 150° C. for 2 hours. The mixture was cooled and then partitioned between ethyl acetate and brine. The organic layer was further washed once with brine, dried over sodium sulfate and concentrated. Chromatography (SiO₂, 50% ethyl acetate in pentane with 1% triethylamine) gave 227 mg of the product (61%). LCMS MH⁺ calculated, 380.0, found 380.0.

### Example 4B

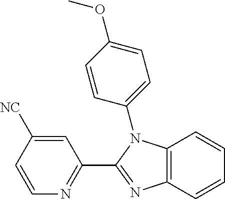 [SMILES: COc1ccc(-n2c(-c3cc(C#N)ccn3)nc3ccccc32)cc1]

### Example 4B

Following the procedure of Example 1C, Example 4A (104 mg, 0.27 mmol) was converted to the target compound (23 mg, 26%). LCMS MH⁺ calculated, 327.1, found 327.2.

### Example 4

Following the procedure of Example 1, Example 4B (23 mg, 0.071 mmol) was converted to the target compound (15 mg, 62%). LCMS MH⁺ calculated, 346.1, found 346.1. ¹H NMR (DMSO-d6, 500 MHz) δ 13.84 (s, 1H), 8.60 (dd, J=0.5, 1.5 Hz, 1H), 8.54 (dd, J=1.0, 5 Hz, 1H), 7.84 (dd, J=1.0, 7 Hz, 1H), 7.78 (dd, J=1.5, 5 Hz, 1H), 7.31 (m, 4H), 7.18 (dd, J=1.5, 7 Hz, 1H), 7.05 (m, 2H), 3.83 (s, 3H).

### Example 5

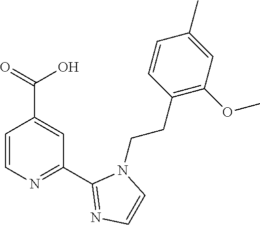 [SMILES: COc1cc(C)ccc1CCn1ccnc1-c1cc(C(=O)O)ccn1]

### Example 5A

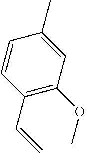

### Example 5A

To a solution of methyl(triphenyl)phosphonium bromide (4.745 g, 13.32 mmol, 1 eq) in THF (20 mL) was added t-BuONa (1.28 g, 13.32 mmol, 4 eq) at 0° C. under N₂ atmosphere. The mixture was stirred at 0° C. for 0.5 hr. To the mixture was added 2-methoxy-4-methyl-benzaldehyde (500 mg, 3.33 mmol, 1 eq) dropwise over 15 min, then the mixture was stirred at 0° C. for 0.5 hr. To this reaction was added H₂O (20 mL) and the mixture was extracted with ethyl acetate (20 mL*2). The combined organic phase was washed with brine (20 mL), dried over anhydrous sodium sulfate, filtered and concentrated in vacuo. The residue was purified by flash silica gel chromatography to give the target compound (490 mg, 3.31 mmol, 99% yield) as a white oil. ¹H NMR (400 MHz, CHLOROFORM-d) δ 2.36 (3H, s) 3.85 (3H, s) 5.19-5.23 (1H, m) 5.70 (1H, dd, J=17.82, 1.51 Hz) 6.70 (1H, s) 6.74-6.79 (1H, m) 6.97-7.06 (1H, m) 7.37 (1H, d, J=7.53 Hz).

### Example 5B

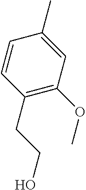

### Example 5B

To a solution of Example 5A (500 mg, 3.37 mmol, 1 eq) in THF (20 mL) was added BH₃-Me₂S (10 M, 3.37 mL, 10 eq) at 0° C. The mixture was stirred at 15° C. for 3 hr and then cooled to 0° C. again. A mixture of NaOH (2.70 g, 67 mmol, 20 eq) dissolved in H₂O (800 μl) and H₂O₂ (7.64 g, 67 mmol, 6.48 mL, 30% purity, 20 eq) was added to the mixture and stirring was continued for 3 hr at 15° C. The reaction mixture was diluted with H₂O (10 mL) and extracted with ethyl acetate (20 mL*2). The combined organic layers were washed with brine (20 mL*2), dried over sodium sulfate, filtered and concentrated under reduced pressure to give a residue. The residue was purified by column chromatography to give the target compound (350 mg, 2.11 mmol, 62% yield) as a colorless oil. ¹H NMR (400 MHz, CHLOROFORM-d) δ 2.35 (3H, s) 2.85-2.91 (2H, m) 3.80-3.83 (2H, m) 3.83 (3H, s) 6.69-6.76 (2H, m) 7.05 (1H, s).

### Example 5C

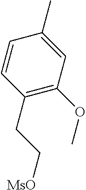 [SMILES: COc1cc(C)ccc1CCOS(C)(=O)=O]

### Example 5C

To a solution of Example 5B (350 mg, 2.11 mmol, 1 eq) in DCM (10 mL) was added methanesulfonyl chloride (290 mg, 2.53 mmol, 196 μL, 1.20 eq) and Et₃N (427 mg, 4.22 mmol, 585 μl, 2 eq) at 0° C. The reaction was stirred at 20° C. for 0.5 hour. The reaction was diluted with DCM (10 mL) and washed with H₂O (10 mL). The organic phase was washed with brine (20 mL), dried over anhydrous sodium sulfate, filtered and concentrated in vacuo to give the target compound (400 mg, 1.64 mmol, 78% yield) as a white oil. ¹H NMR (400 MHz, CHLOROFORM-d) δ 2.35 (3H, s) 2.85 (3H, s) 3.03 (2H, s) 3.83 (3H, s) 4.40 (2H, s) 6.69 (2H, s) 7.03-7.07 (1H, m).

### Example 5D

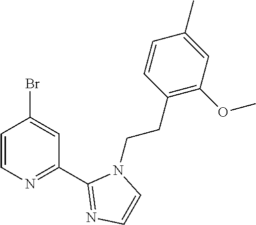 [SMILES: COc1cc(C)ccc1CCn1ccnc1-c1cc(Br)ccn1]

### Example 5D

To a solution of 4-bromo-2-(1H-imidazol-2-yl)pyridine ([1211579-82-6], 200 mg, 893 μmol, 1 eq) in DMF (20 mL) was added Example 5C (327 mg, 1.34 mmol, 1.50 eq) and K₂CO₃ (2.68 mmol, 2 eq). The reaction was stirred at 80° C. for 1 hour. To this reaction was added H₂O (50 mL) and the mixture was extracted with ethyl acetate (50 mL*2). The combined organic phase was washed with brine (50 mL), dried over anhydrous sodium sulfate, filtered and concentrated under vacuum. The residue was purified by column chromatography to give the target compound (230 mg, 592 μmol, 66% yield, 96% purity) as a white solid. LCMS: ESI m/z 371.9 [M+1].

### Example 5E

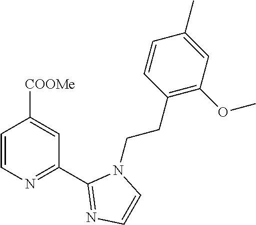 [SMILES: COC(=O)c1ccnc(-c2nccn2CCc2ccc(C)cc2OC)c1]

### Example 5E

To a solution of Example 5D (230 mg, 618 μmol, 1 eq) in methanol (10 mL) was added Et₃N (938 mg, 9.27 mmol, 1.28 mL, 15 eq) and Pd(dppf)Cl₂ (45 mg, 62 μmol, 0.10 eq). The reaction was stirred at 80° C. under CO (50 psi) atmosphere for 4 hours. The reaction was filtered and concentrated in vacuo. The residue was purified by prep-TLC to give the target compound (150 mg, 390 μmol, 63% yield, 91% purity) as a white solid. LCMS: ESI m/z 351.9 [M+1].

### Example 5

To a solution of Example 5E (150 mg, 427 μmol, 1 eq) in MeOH (5 mL) was added LiOH.H₂O (2 M, 213 μl, 1 eq) at 20° C. The reaction was stirred at 20° C. for 12 hours. The reaction was concentrated in vacuo and the pH adjusted to 5 with HCl (1 M; 0.1 mL). The solid was collected by filtration, and the filter cake was washed with water and dried in vacuo to give the target compound (52 mg, 150 μmol, 35% yield, 97% purity) as a pink solid. LCMS: ESI m/z 338.1 [M+1]. ¹H NMR (400 MHz, METHANOL-d4) δ 2.19 (3H, s) 2.93-2.98 (2H, m) 3.56-3.60 (3H, m) 4.87-4.90 (2H, m) 6.34 (1H, s) 6.46 (2H, s) 7.31 (1H, s) 7.45 (1H, s) 7.85-7.95 (2H, m) 8.70-8.78 (1H, m).

### Example 6

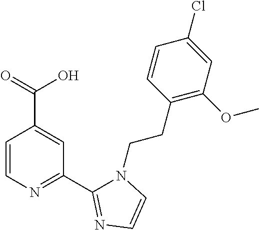 [SMILES: COc1cc(Cl)ccc1CCn1ccnc1-c1cc(C(=O)O)ccn1]

### Example 6A

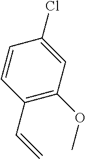

### Example 6A

To a solution of methyl-(triphenyl)phosphonium bromide (2.51 g, 7.03 mmol, 4 eq) in THF (10 mL) was added t-BuONa (672 mg, 7.00 mmol, 3.98 eq) at 0° C. under N₂ atmosphere. The mixture was stirred at 0° C. for 30 min. Then to the mixture was added 4-chloro-2-methoxybenzaldehyde (300 mg, 1.76 mmol, 1 eq) dropwise over 15 min. The mixture was warmed to 15° C. and stirred for 30 min. The reaction mixture was diluted with H₂O (20 mL) and extracted with EtOAc (30 mL*2). The combined organic layers were washed with brine (20 mL*2), dried over sodium sulfate, filtered and concentrated in vacuo. The residue was purified by prep-TLC (SiO₂, petroleum ether/Ethyl acetate=4:1). The target compound (250 mg, 1.48 mmol, 84% yield) was obtained as a yellow oil. ¹H NMR (400 MHz, CHLOROFORM-d) δ 3.85 (s, 3H), 5.28 (dd, J=11.2, 1.12 Hz, 1H), 5.72 (dd, J=17.6, 1.12 Hz, 1H), 6.86 (d, J=1.6 Hz, 1H), 6.91-6.95 (m, 1H), 6.95-7.02 (m, 1H), 7.39 (d, J=8.0 Hz, 1H).

### Example 6B

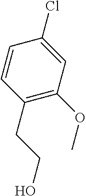

### Example 6B

To a solution of Example 6A (250 mg, 1.48 mmol, 1 eq) in THF (10

mL) was added BH₃-Me₂S (10 M, 1.48 mL, 10 eq) dropwise at 0° C. The mixture was stirred at 20° C. for 3 hours (hr) and then cooled to 0° C. again. A mixture of NaOH (1.18 g, 29.6 mmol) dissolved in H₂O (800 μl) and H₂O₂ (3.36 g, 29.60 mmol, 2.84 mL, 30% purity, 20 eq) was added to the mixture and stirring was continued for 3 hr at 20° C. The reaction mixture was diluted with H₂O (10 mL) and extracted with EtOAc (20 mL*2). The combined organic layers were washed with brine (20 mL*3), dried over sodium sulfate, filtered and concentrated in vacuo. The residue was purified by prep-TLC (SiO₂, Petroleum ether/Ethyl acetate=5:2). The target compound (170 mg, 911 μmol, 61% yield) was obtained as a yellow oil. ¹H NMR (400 MHz, CHLOROFORM-d) δ 2.86 (t, J=6.8 Hz, 2H), 3.78-3.85 (m, 5H), 6.86 (s, 1H), 6.90 (dd, J=8.0, 1.6 Hz, 1H), 7.09 (d, J=8.0 Hz, 1H).

### Example 6C

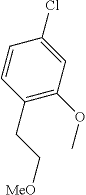

### Example 6C

To a solution of Example 6B (170 mg, 911 μmol, 1 eq) in DCM (10 mL) was added Et₃N (184 mg, 1.82 mmol, 253 μl, 2 eq). The mixture was cooled to 0° C. and MsCl (157 mg, 1.37 mmol, 106 μl, 1.50 eq) was added dropwise. After addition, the mixture was warmed to 15° C. and stirred for 15 min. The reaction mixture was diluted with DCM (20 mL), then washed with brine (15 mL*3), dried over sodium sulfate, filtered and concentrated in vacuo. The crude product was used in the next step without further purification. The target compound (300 mg, crude) was obtained as yellow oil. ESI m/z 169.1 [M-OMs+1]+. ¹H NMR (400 MHz, CHLOROFORM-d) δ 3.03 (t, J=6.8 Hz, 2H), 3.68 (s, 3H), 3.83 (s, 3H), 4.38 (t, J=6.8 Hz, 2H), 6.86 (d, J=1.6 Hz, 1H), 6.90 (dd, J=7.6, 1.6 Hz, 1H) 7.09 (d, J=8.0 Hz, 1H).

### Example 6D

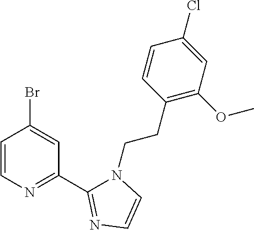 [SMILES: COc1cc(Cl)ccc1CCn1ccnc1-c1cc(Br)ccn1]

### Example 6D

To a solution of Example 6C (300 mg, 1.13 mmol, 1 eq) in DMF (5 mL) was added 4-bromo-2-(1H-imidazol-2-yl)pyridine ([1211579-82-6], 253 mg, 1.13 mmol, 1 eq) and K₂CO₃ (312 mg, 2.26 mmol, 2 eq). The mixture was stirred at 70° C. for 2 hr. The reaction mixture was diluted with H₂O (10 mL) and extracted with EtOAc (20 mL*3). The combined organic layers were washed with brine (20 mL*2), dried over sodium sulfate, filtered and concentrated in vacuo. The residue was purified by prep-TLC (SiO₂, Petroleum ether: Ethyl acetate=1:1). The target compound (70 mg, 178 μmol, 16% yield) was obtained as a yellow solid. ¹H NMR (400 MHz, CHLOROFORM-d) δ 3.05 (t, J=6.8 Hz, 2H), 3.76 (s, 3H), 4.77 (t, J=6.4 Hz, 2H), 6.73-6.87 (m, 4H), 7.10 (s, 1H), 7.40 (dd, J=5.2, 1.6 Hz, 1H), 8.29 (s, 1H), 8.38 (d, J=5.2 Hz, 1H).

### Example 6E

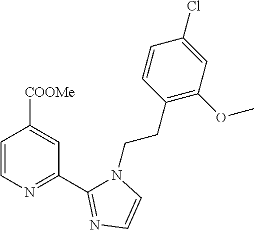 [SMILES: COC(=O)c1ccnc(-c2nccn2CCc2ccc(Cl)cc2OC)c1]

### Example 6E

A mixture of Example 6D (70 mg, 178 μmol, 1 eq), Et₃N (180 mg, 1.78 mmol, 247 μl, 10 eq), Pd(dppf)Cl₂ (13 mg, 17.83 μmol, 0.10 eq) in MeOH (10 mL) was degassed and purged with N₂ 3 times. The mixture was stirred at 70° C. for 4.5 hr under a CO atmosphere (50 psi). The reaction mixture was filtered and concentrated in vacuo. The residue was purified by prep-TLC (SiO₂, Petroleum ether/Ethyl acetate=1:2). The target compound (50 mg, 134 μmol, 75% yield) was obtained as a purple oil. ESI m/z 372.1 [M+11]+

### Example 6

To a solution of Example 6E (50 mg, 134 μmol, 1 eq) in MeOH (2 mL) and H₂O (1 mL) was added NaOH (11 mg, 269 μmol, 2 eq). The mixture was stirred at 50° C. for 1 hr. The reaction mixture was concentrated to remove MeOH. The residue was adjusted to pH=7 with 2M HCl and filtered. The cake was washed with H₂O and dried in vacuo. The target compound (31 mg, 86 μmol, 64% yield) was obtained as a red solid. ESI m/z 358.0 [M+1]+. ¹H NMR (400 MHz, DMSO-d₆) δ 2.92 (t, 2H), 3.67 (s, 3H), 4.75-4.83 (t, 2H), 6.76 (s, 3H), 7.02 (br. s., 1H), 7.27 (br. s., 1H), 7.72 (br. s., 1H), 8.24 (br. s., 1H), 8.71 (br. s., 1H).

### Example 7

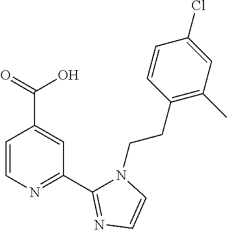 [SMILES: Cc1cc(Cl)ccc1CCn1ccnc1-c1cc(C(=O)O)ccn1]

### Example 7A

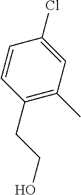

### Example 7A

To a solution of 4-chloro-1-ethenyl-2-methylbenzene ([121135-78-2], 1.00 g, 6.55 mmol, 1 eq) in THF (10 mL) was added BH₃-Me₂S (10 M, 6.55 mL, 10 eq) dropwise at 0° C. The mixture was stirred at 20° C. for 3 hr and then cooled to 0° C. again. A mixture of NaOH (5.24 g, 131 mmol, 20 eq) dissolved in H₂O (1 mL) and H₂O₂ (14.86 g, 131 mmol, 12.59 mL, 30% purity, 20 eq) was added to the mixture and stirring was continued for 3 hr at 20° C. The reaction mixture was diluted with H₂O (10 mL) and extracted with EtOAc (25 mL*3). The combined organic layers were washed with brine (30 mL*2), dried over sodium sulfate, filtered and concentrated in vacuo. The residue was purified by column chromatography (SiO₂, Petroleum ether/Ethyl acetate=10/1 to 4:1). The target compound (570 mg, 3.34 mmol, 51% yield) was obtained as a light yellow oil. ¹H NMR (400 MHz, CHLOROFORM-d) δ 2.32 (s, 3H), 2.86 (t, J=6.8 Hz, 2H), 3.82 (t, J=6.8 Hz, 2H), 7.08-7.15 (m, 2H), 7.16 (s, 1H).

### Example 7B

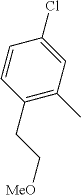

### Example 7B

To a solution of Example 7A (570 mg, 3.34 mmol, 1 eq) and Et₃N (676 mg, 6.68 mmol, 926 μl, 2 eq) in DCM (10 mL) was added MsCl (497 mg, 4.34 mmol, 336 μl, 1.30 eq). The mixture was stirred at 15° C. for 0.5 hr. The reaction mixture was diluted with DCM (20 mL) and washed with brine (20 mL*3), dried over sodium sulfate, filtered and concentrated in vacuo. The crude product was used in the next step directly without further purification. The target compound (800 mg, crude) was obtained as yellow oil. ¹H NMR (400 MHz, CHLOROFORM-d) δ 2.33 (s, 3H), 2.91 (s, 3H), 3.05 (t, J=7.2 Hz, 2H), 4.37 (t, J=7.2 Hz, 2H), 7.09-7.20 (m, 3H).

### Example 7C

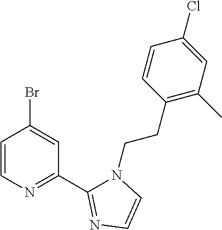 [SMILES: Cc1cc(Cl)ccc1CCn1ccnc1-c1cc(Br)ccn1]

### Example 7C

To a solution of Example 7B (800 mg, 3.22 mmol, 1 eq) and 4-bromo-2-(1H-imidazol-2-yl)pyridine ([1211579-82-6], 433 mg, 1.93 mmol, 0.60 eq) in DMF (6 mL) was added K₂CO₃ (890 mg, 6.44 mmol, 2 eq). The reaction mixture was stirred at 80° C. for 10 hr. The reaction mixture was filtered. The filtrate was diluted with EtOAc (40 mL) and washed with brine (20 mL*3), dried over sodium sulfate, filtered and concentrated in vacuo. The residue was purified by prep-TLC (SiO₂, Petroleum ether/Ethyl acetate=1:1). The target compound (600 mg, 1.59 mmol, 49% yield) was obtained as a yellow oil. ESI m/z 378.1 [M+1]. ¹H NMR (400 MHz, METHANOL-d₄) δ 2.11 (s, 3H), 3.04 (t, J=6.8 Hz, 2H), 4.83 (t, J=6.8 Hz, 2H), 6.71 (d, J=8.0 Hz, 1H), 6.89-6.95 (m, 2H), 7.07 (s, 1H), 7.25 (s, 1H), 7.53 (dd, J=5.2, 1.6 Hz, 1H), 7.85 (d, J=1.2 Hz, 1H), 8.38 (d, J=5.2 Hz, 1H).

### Example 7D

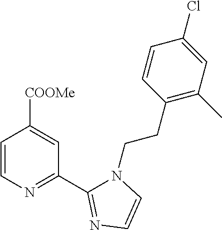 [SMILES: COC(=O)c1ccnc(-c2nccn2CCc2ccc(Cl)cc2C)c1]

### Example 7D

To a solution of Example 7C (600 mg, 1.59 mmol, 1 eq) in MeOH (10 mL) was added Et₃N (2.41 g, 23.85 mmol, 3.31 mL, 15 eq) and Pd(dppf)Cl₂ (116 mg, 159 μmol, 0.10 eq). The mixture was stirred at 70° C. for 5 hr under a CO atmosphere (50 psi). The reaction mixture was filtered and concentrated in vacuo. The residue was diluted with EtOAc (40 mL) and washed with brine (20 mL*3), dried over sodium sulfate, filtered and concentrated under reduced pressure. The residue was purified by prep-TLC (SiO₂, Petroleum ether/Ethyl acetate=1:1). The target compound (450 mg, 1.26 mmol, 79% yield) was obtained as a brown oil. ESI m/z 356.1 [M+1]+

### Example 7

To a solution of Example 7D (450 mg, 1.26 mmol, 1 eq) in MeOH (10 mL) and H₂O (5 mL) was added NaOH (101 mg, 2.52 mmol, 2 eq). The mixture was stirred at 50° C. for 1 hr. The reaction mixture was filtered and concentrated to remove MeOH. The residue was diluted with H₂O (15 mL) and adjusted to pH=7 with 4M HCl and then filtered. The filter cake was washed with MeOH (20 mL) and dried in vacuo. The target compound (400 mg, 1.17 mmol, 93% yield) was obtained as a purple solid. ESI m/z 341.9 [M+1]+. ¹H NMR (400 MHz, DMSO-d₆) δ 2.19 (s, 3H), 3.00 (t, J=6.8 Hz, 2H), 4.78 (t, J=6.8 Hz, 2H), 6.95 (d, J=7.6 Hz, 1H), 7.01-7.09 (m, 3H), 7.39 (s, 1H), 7.74 (d, J=4.0 Hz, 1H), 8.33 (s, 1H), 8.74 (d, J=4.8 Hz, 1H).

### Example 8

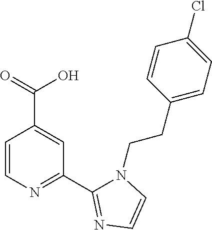 [SMILES: O=C(O)c1ccnc(-c2nccn2CCc2ccc(Cl)cc2)c1]

### Example 8A

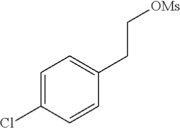 [SMILES: CS(=O)(=O)OCCc1ccc(Cl)cc1]

### Example 8A

To a solution of 2-(4-chlorophenyl)ethanol (250 mg, 1.60 mmol, 215 id, 1 eq) and Et₃N (243 mg, 2.40 mmol, 333 μl, 1.50 eq) in DCM (5 mL) at 0° C. was added methanesulfonyl chloride (340 mg, 2.97 mmol, 230 μl, 1.86 eq). The solution was stirred at 0° C. for 2 hours. Water (30 mL) was added to the solution. The organic layer was separated, dried over anhydrous sodium sulfate, and concentrated to afford the target compound (350 mg, 93% yield). The product can be used directly for the next step without further purification. ¹H NMR (400 MHz, METHANOL-d₄) δ 7.35-7.21 (m, 4H), 4.43 (t, J=6.6 Hz, 2H), 3.04 (t, J=6.6 Hz, 2H), 2.98 (s, 3H).

### Example 8B

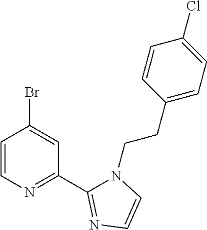 [SMILES: Clc1ccc(CCn2ccnc2-c2cc(Br)ccn2)cc1]

### Example 8B

To a solution of 4-bromo-2-(1H-imidazol-2-yl)pyridine ([1211579-82-6], 100 mg, 446 μmol, 1 eq) and 2-(4-chlorophenyl)ethyl methanesulfonate (157 mg, 669 μmol, 1.50 eq) in DMF (5.00 mL) was added K₂CO₃ (123 mg, 893 μmol, 2 eq). The mixture was heated to 80° C. for 10 hours. The reaction was filtered and the filtrate concentrated in vacuo to afford the crude product. The crude product was purified by chromatography on silica gel to give the product as a yellow oil (150 mg, 93% yield). ESI m/z 363.8 [M+1]+ ¹H NMR (400 MHz, METHANOL-d4) δ 8.35 (d, J=5.2 Hz, 1H), 7.84 (d, J=1.6 Hz, 1H), 7.45 (dd, J=2.0, 5.2 Hz, 1H), 7.10 (d, J=1.2 Hz, 1H), 7.05-7.00 (m, 2H), 6.95 (d, J=1.2 Hz, 1H), 6.83 (d, J=8.4 Hz, 2H), 4.73 (t, J=7.0 Hz, 2H), 2.93 (t, J=7.0 Hz, 2H).

### Example 5C

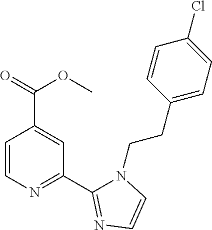 [SMILES: COC(=O)c1ccnc(-c2nccn2CCc2ccc(Cl)cc2)c1]

### Example 8C

To a solution of Example 5B (140 mg, 386 μmol, 1 eq) and Et₃N (390 mg, 3.86 mmol, 535 μL, 10 eq) in MeOH (10 mL) was added Pd(dppf) Cl₂ (42 mg, 58 μmol, 0.15 eq). The mixture was purged with carbon monoxide 6 times, then the mixture was heated to 80° C. under a carbon monoxide (50 psi) atmosphere and stirred for 18 hours. The solution was cooled to RT and filtered. The filtrate was concentrated in vacuo to afford the crude product, which was purified by flash chromatography on silica gel to afford the product as a yellow solid (100 mg, 76% yield). ESI m/z 341.9 [M+1]⁺ ¹H NMR (400 MHz, METHANOL-d₄) δ 8.76 (d, J=5.0 Hz, 1H), 8.21 (s, 1H), 7.84 (dd, J=1.6, 5.2 Hz, 1H), 7.28 (d, J=1.0 Hz, 1H), 7.11-7.04 (m, 3H), 6.92-6.87 (m, 2H), 4.88-4.84 (m, 2H), 4.00 (s, 3H), 3.04 (t, J=6.8 Hz, 2H).

### Example 8

To a solution of Example 5C (130 mg, 380 μmol, 1 eq) in MeOH (1 mL) and water (1 mL) was added LiOH (159 mg, 3.80 mmol, 10 eq). The mixture was stirred at 25° C. for 1 hour. 2 M HCl was added slowly to adjust the pH to 7. A precipitate formed. The solid was filtered and washed with H₂O (5 mL*2), then dried under vacuum to provide the target compound as a white solid (80 mg, 64% yield). ESI m/z 328.0 [M+1]⁺ ¹H NMR (400 MHz, DMSO-d₆) δ 8.80 (d, J=5.0 Hz, 1H), 8.40 (s, 1H), 7.75 (dd, J=1.6, 5.0 Hz, 1H), 7.34 (s, 1H), 7.25 (d, J=8.4 Hz, 2H), 7.12 (d, J=8.4 Hz, 2H), 7.06 (s, 1H), 4.79 (t, J=7.2 Hz, 2H), 3.02 (t, J=7.2 Hz, 2H).

### Example 9

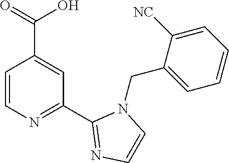 [SMILES: N#Cc1ccccc1Cn1ccnc1-c1cc(C(=O)O)ccn1]

### Example 9A

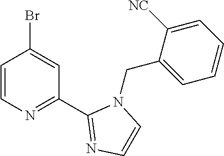 [SMILES: N#Cc1ccccc1Cn1ccnc1-c1cc(Br)ccn1]

### Example 9A

To a solution of 4-bromo-2-(1H-imidazol-2-yl)pyridine ([1211579-82-6], 150 mg, 669 μmol, 1 eq) in DMF (5 mL) was added NaH (134 mg, 3.35 mmol, 60% purity, 5 eq) at 0° C. The reaction was stirred for 15 min, then 2-(bromomethyl) benzonitrile (144 mg, 736 μmol, 1.10 eq) was added to the solution dropwise. The mixture was stirred at 25° C. for 5 hours. Water (10 mL) was added to the solution at 0° C. The mixture was extracted with EtOAc (15 mL*3). The combined the organic layers were dried over anhydrous sodium sulfate and concentrated in vacuo to afford the crude product. The crude product was purified by flash chromatography on silica gel to obtain the target compound as a yellow oil (102 mg, 45% yield). ESI m/z 340.8 [M+1]+ ¹H NMR (400 MHz, METHANOL-d₄) 8.40 (d, J=5.2 Hz, 1H), 8.25 (d, J=1.8 Hz, 1H), 7.75 (dd, J=1.0, 7.8 Hz, 1H), 7.57-7.50 (m, 2H), 7.45 (d, J=1.0 Hz, 1H), 7.44-7.38 (m, 1H), 7.24 (d, J=1.0 Hz, 1H), 6.98 (d, J=8.0 Hz, 1H), 6.12 (s, 2H).

### Example 9B

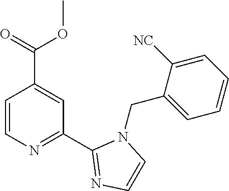 [SMILES: COC(=O)c1ccnc(-c2nccn2Cc2ccccc2C#N)c1]

### Example 9B

To a solution of Example 9A (90 mg, 265 μmol, 1 eq) and Et₃N (268 mg, 2.65 mmol, 367 μL, 10 eq) in MeOH (10 mL) was added Pd(dppf)Cl₂ (29 mg, 40 μmol, 0.15 eq). The mixture was purged with CO and the solution heated at 80° C. under a CO atmosphere (50 psi) for 18 hours. The reaction was filtered and concentrated in vacuo to afford the crude product. The crude product was purified by prep-TLC (DCM/MeOH=20/1) to give the product as a yellow solid (60 mg, 71% yield). ESI m/z 319.1 [M+1]⁺ ¹H NMR (400 MHz, METHANOL-d₄) δ 8.72 (d, J=5.0 Hz, 1H), 8.58 (s, 1H), 7.79 (dd, J=1.6, 5.0 Hz, 1H), 7.75 (dd, J=1.0, 7.8 Hz, 1H), 7.53 (dt, J=1.4, 7.8 Hz, 1H), 7.46 (d, J=1.2 Hz, 1H), 7.43-7.37 (m, 1H), 7.26 (d, J=1.2 Hz, 1H), 6.99 (d, J=7.8 Hz, 1H), 6.14 (s, 2H), 3.98 (s, 3H).

### Example 9

To a solution of Example 98 (60 mg, 188 μmol, 1 eq) in MeOH (3 mL) was added 1 mL LiOH (2 M) solution. The mixture was stirred at 25° C. for 1 hour. The reaction was concentrated in vacuo to remove most of the MeOH. The pH was adjusted to 7 with HCl solution (2 M). A precipitate formed. The solid was filtered and dried under vacuum to give the target compound as a white solid (49 mg, 85% yield). ESI m/z 305.1 [M+1]+¹H NMR (400 MHz, METHANOL-d4) δ 8.72 (d, J=5.0 Hz, 1H), 8.58 (s, 1H), 7.81 (d, J=5.0 Hz, 1H), 7.76 (dd, J=1.0, 7.8 Hz, 1H), 7.54 (m, 1H), 7.48 (s, 1H), 7.44-7.38 (m, 1H), 7.28 (s, 1H), 7.01 (d, J=7.8 Hz, 1H), 6.15 (s, 2H).

### Example 10

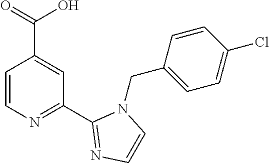 [SMILES: O=C(O)c1ccnc(-c2nccn2Cc2ccc(Cl)cc2)c1]

### Example 10A

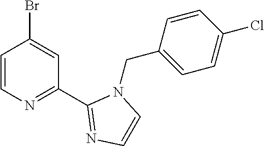 [SMILES: Clc1ccc(Cn2ccnc2-c2cc(Br)ccn2)cc1]

### Example 10A

To a solution of 4-bromo-2-(1H-imidazol-2-yl) pyridine ([1211579-82-6], 160 mg, 714 μmol, 1 eq) in DMF (5 mL) was added NaH (228 mg, 5.71 mmol, 60% purity, 8 eq). The mixture was stirred at 0° C. for 15 min, then 1-chloro-4-(chloromethyl)benzene (172 mg, 1.07 mmol, 1.50 eq) was added. The mixture was warmed to 25° C. and stirred for 5 hours. Water (10 mL) was added. The mixture was extracted with ethyl acetate (25 mL*3), dried over anhydrous sodium sulfate and filtered. The solvent was removed in vacuo to afford the crude product. The crude product was purified by flash chromatography on silica gel (PE/EA=5/1) to give the target product as an oil (180 mg, 72% yield). ESI m/z 350.1 [M+1]+ ¹H NMR (400 MHz, METHANOL-d4) δ 8.42 (d, J=5.4 Hz, 1H), 8.23 (d, J=1.6 Hz, 1H), 7.54 (dd, J=2.0, 5.4 Hz, 1H), 7.34 (d, J=1.0 Hz, 1H), 7.31-7.24 (m, 2H), 7.19-7.10 (m, 3H), 5.87 (s, 2H).

### Example 10B

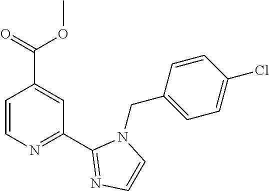 [SMILES: COC(=O)c1ccnc(-c2nccn2Cc2ccc(Cl)cc2)c1]

### Example 10B

To a solution of Example 10A (140 mg, 401 μmol, 1 eq) and Et₃N (406 mg, 4.02 mmol, 556 μl, 10 eq) in MeOH (3 mL) was added Pd(dppf)Cl₂ (44 mg, 60 μmol, 0.15 eq). The mixture was purged with carbon monoxide (50 psi) and heated to 80° C. under a carbon monoxide atmosphere for 6 hours. The solution was cooled to room temperature (RT), filtered and the solvent evaporated to afford the crude product. The crude product was purified by prep-TLC (DCM/MeOH=20/1), to obtain the product as a yellow solid (113 mg, 86% yield). ESI m/z 328.0 [M+1]+ ¹H NMR (400 MHz, METHANOL-d4) δ 8.75 (dd, J=0.8, 5.0 Hz, 1H), 8.56-8.53 (s, 1H), 7.83 (d, J=1.6, 5.0 Hz, 1H), 7.36 (d, J=1.2 Hz, 1H), 7.30-7.26 (m, 2H), 7.21-7.14 (m, 3H), 5.89 (s, 2H), 3.98 (s, 3H).

### Example 10

To a solution of Example 10B (100 mg, 305 μmol, 1 eq) in MeOH (4 mL) was added 1 mL LiOH (2 M) solution. The mixture was stirred at 25° C. for 1 hour. The mixture was concentrated in vacuo to remove most of the methanol. To the residue was added 2 M HCl solution to adjust the pH to 7. A precipitate was formed. The solid was filtered and dried under vacuum to give the target compound as a white solid (85 mg, 89% yield, purity=97%). ESI m/z 313.9 [M+1]+ ¹H NMR (400 MHz, METHANOL-d4) δ 8.68 (d, J=3.6 Hz, 1H), 8.46 (s, 1H), 7.80 (d, J=4.0 Hz, 1H), 7.38-7.26 (m, 3H), 7.17 (d, J=8.2 Hz, 3H), 5.87 (s, 2H).

### Example 11

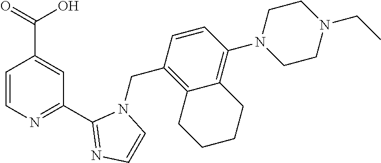 [SMILES: CCN1CCN(c2ccc(Cn3ccnc3-c3cc(C(=O)O)ccn3)c3c2CCCC3)CC1]

### Example 11A

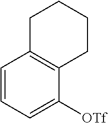 [SMILES: O=S(=O)(Oc1cccc2c1CCCC2)C(F)(F)F]

### Example 11A

To a solution of tetralin-5-ol (5 g, 33.74 mmol, 1 eq) and Et₃N (10.24 g, 101 mmol, 14 mL, 3 eq) in DCM (50 mL) at 0° C. was added dropwise trifluoromethylsulfonyl trifluoromethanesulfonate (10.47 g, 37.11 mmol, 6.12 mL, 1.10 eq). The reaction mixture was warmed to 25° C. and stirred for 4 hours. To the mixture was added water (50 mL) and the mixture extracted with DCM (25 mL*3). The combined organics were dried over anhydrous sodium sulfate and concentrated in vacuo to afford the crude product. The crude product was purified by flash chromatography on silica gel (eluent: petroleum ether) to give the product as an oil (8 g, 85% yield). ¹H NMR (400 MHz, CD₃OD) δ 7.26-7.13 (m, 2H), 7.10 (d, J=7.8 Hz, 1H), 2.84 (t, J=5.6 Hz, 2H), 2.78 (t, J=5.6 Hz, 2H), 1.89-1.76 (m, 4H).

### Example 11B

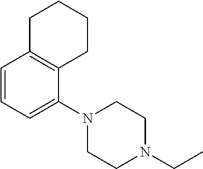 [SMILES: CCN1CCN(c2cccc3c2CCCC3)CC1]

### Example 11B

To a solution of Example 11A (4.90 g, 17.48 mmol, 1 eq), 1-ethylpiperazine (2.79 g, 24.47 mmol, 3.10 mL, 1.40 eq), Cs₂CO₃ (11.39 g, 34.96 mmol, 2 eq) and RuPhos (1.14 g, 2.45 mmol, 0.14 eq) in dioxane (50 mL) was added Pd₂ (dba)₃ (800 mg, 874 μmol, 0.05 eq). The mixture was heated to 100° C. under a N₂ atmosphere for 6 hours. The mixture was cooled to RT and filtered. The filtrate was concentrated in vacuo to afford crude product. The crude product was purified by flash chromatography on silica gel (DCM/MeOH=20/1) to obtain the product as a yellow solid (1.85 g, 43% yield). ESI m/z 245.0 [M+1]⁺ ¹H NMR (400 MHz, CDO₃OD) δ 7.10-7.04 (m, 1H), 6.92 (d, J=8.0 Hz, 1H), 6.84 (d, J=7.8 Hz, 1H), 3.03 (br. s, 8H), 2.87 (q, J=7.2 Hz, 2H), 2.81-2.72 (m, 4H), 1.84-1.73 (m, 4H), 1.28 (t, J=7.4 Hz, 3H).

### Example 11C

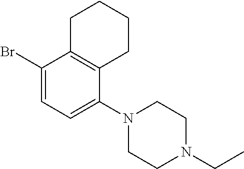 [SMILES: CCN1CCN(c2ccc(Br)c3c2CCCC3)CC1]

### Example 11C

To a solution of Example 11B (2.70 g, 11.05 mmol, 1 eq) in DCM (100 mL) was added NBS (2.16 g, 12.16 mmol, 1.10 eq) portion-wise. The mixture was stirred at 25° C. for 2 hours. The solvent was removed in vacuo to afford the crude product. The crude product was purified by flash chromatography on silica gel (DCM/MeOH=20/1) to obtain the product as a yellow solid (2.6 g, 73% yield). ESI m/z 324.9 [M+1]+ ¹H NMR (400 MHz, CD₃OD) δ 7.37 (d, J=8.4 Hz, 1H), 6.87 (d, J=8.4 Hz, 1H), 3.00-2.96 (m, 4H), 2.78-2.67 (m, 8H), 1.88-1.69 (m, 6H), 1.24-1.20 (m, 3H).

### Example 11D

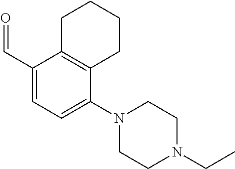 [SMILES: CCN1CCN(c2ccc(C=O)c3c2CCCC3)CC1]

### Example 11D

To a solution of Example 11C (600 mg, 1.86 mmol, 1 eq) in THF (20 mL) was added n-BuLi (2.5 M, 1.86 mL, 2.50 eq) at −78° C. After 30 min, DMF (1.36 g, 18.60 mmol, 1.43 mL, 10 eq) was added to the solution and the reaction stirred at −78° C. for 1 hour, then warmed to 0° C. for 30 min. Ice water was slowly added to the solution at 0° C., and the mixture extracted with EA (50 mL*3). The organics were dried with anhydrous sodium sulfate and concentrated in vacuo to afford the crude product. The crude product was purified by flash chromatography on silica gel (DCM/MeOH=100/1, 0.25% Et₃N) to obtain the product as a yellow oil (0.36 g, 71% yield). ESI m/z 273.0 [M+1]+ ¹H NMR (400 MHz, CD₃OD) δ 10.10 (s, 1H), 7.67 (d, J=8.2 Hz, 1H), 7.05 (d, J=8.2 Hz, 1H), 3.23 (t, J=6.6 Hz, 2H), 3.04 (t, J=4.2 Hz, 4H), 2.80-2.75 (m, 2H), 2.68 (br. s., 4H), 2.57-2.50 (m, 2H), 1.90-1.81 (m, 2H), 1.76-1.69 (m, 2H), 1.20-1.14 (m, 3H).

### Example 11E

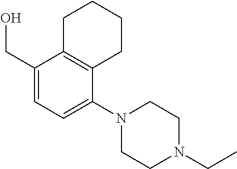 [SMILES: CCN1CCN(c2ccc(CO)c3c2CCCC3)CC1]

### Example 11E

To a solution of Example 11D (350 mg, 1.28 mmol, 1 eq) in MeOH (5 mL) was added NaBH₄ (97 mg, 2.56 mmol, 2 eq) slowly. The reaction was stirred for 2 hours at 25° C. The solvent was removed in vacuo. Water (10 mL) was added. The mixture was extracted with ethyl acetate. The organics were dried with anhydrous sodium sulfate and concentrated in vacou to afford the crude product. The crude product was purified by flash chromatography on silica gel (DCM/MeOH) to obtain the product as an oil. (150 mg, 42.71% yield). ESI m/z 275.0 [M+1]+

### Example 11F

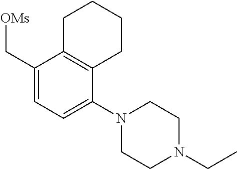 [SMILES: CCN1CCN(c2ccc(COS(C)(=O)=O)c3c2CCCC3)CC1]

### Example 11F

To a solution of Example 11E (180 mg, 656 μmol, 1 eq) and Et₃N (132.76 mg, 1.31 mmol, 182 μl, 2 eq) in DCM (2 mL) was added MsCl (112.71 mg, 984 μmol, 76.16 μl, 1.50 eq) at 0° C. The mixture was stirred at 0° C. for 1 hour. The solvent was removed in vacuo and the residue was used directly for the next step without further purification (200 mg crude target compound).

### Example 11G

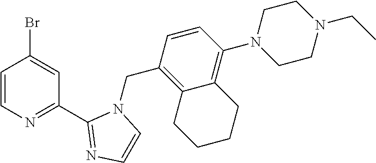 [SMILES: CCN1CCN(c2ccc(Cn3ccnc3-c3cc(Br)ccn3)c3c2CCCC3)CC1]

### Example 11G

To a solution of Example 11F (100 mg, 284 μmol, 1.20 eq) and 4-bromo-2-(1H-imidazol-2-yl)pyridine ([1211579-82-6], 53 mg, 236 μmol, 1 eq) in DMF (5 mL) was added K₂CO₃ (98 mg, 709 μmol, 3 eq). The mixture was stirred at 80° C. for 10 hours. The reaction mixture was filtered. The filtrate was concentrated in vacuo to afford the crude product. The crude product was purified by prep-TLC (DCM/MeOH=10/1) to obtain the target compound as a yellow solid (60 mg, 44% yield). ESI m/z 479.7 [M+1]+¹H NMR (400 MHz, CD₃OD) δ 8.28 (d, J=5.2 Hz, 1H), 8.10 (d, J=1.2 Hz, 1H), 7.42 (dd, J=1.8, 5.2 Hz, 1H), 7.01 (d, J=13.8 Hz, 2H), 6.73 (d, J=8.2 Hz, 1H), 6.44 (d, J=8.0 Hz, 1H), 5.67 (s, 2H), 2.93-2.84 (m, 6H), 2.73 (q, J=7.2 Hz, 2H), 2.65 (t, J=6.0 Hz, 2H), 2.53 (t, J=6.2 Hz, 2H), 1.74-1.66 (m, 2H), 1.65-1.56 (m, 2H), 1.14 (t, J=7.2 Hz, 3H).

### Example 11H

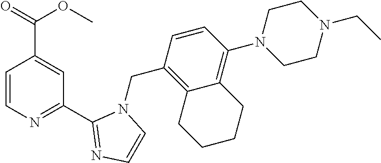 [SMILES: CCN1CCN(c2ccc(Cn3ccnc3-c3cc(C(=O)OC)ccn3)c3c2CCCC3)CC1]

### Example 11H

To a solution of Example 11G (80 mg, 167 μmol, 1 eq) and Et₃N (135 mg, 1.33 mmol, 185 μl, 8 eq) in MeOH (10 mL) was added Pd(dppf)Cl₂ (18 mg, 25 μmol, 0.15 eq). The solution was purged with carbon monoxide 6 times, then the mixture was stirred under a carbon monoxide atmosphere (50 psi) at 80° C. for 12 hours. The mixture was cooled to RT and filtered. The filtrate was concentrated in vacuo to afford the crude product. The crude product was purified by flash chromatography on silica gel (50 mg, 65% yield). ESI m/z 460.0 [M+1]+

### Example 11

To a solution of Example 11H (40 mg, 87 μmol, 1 eq) in MeOH (5 mL) was added 2 mL LiOH solution (2 M) and the mixture was stirred at 25° C. for 2 hours. The pH was adjusted to 7 with 2 M HCl solution. The residue was concentrated in vacuo to afford the crude product. The crude product was purified by Prep-HPLC to obtain the target compound as a yellow solid (28 mg, 72% yield, 99% purity). ESI m/z 446.0 [M+1]+ ¹H NMR (400 MHz, CD₃OD) δ 8.61 (d, J=5.0 Hz, 1H), 8.40 (s, 1H), 7.75 (dd, J=1.5, 5.0 Hz, 1H), 7.11 (d, J=1.3 Hz, 1H), 7.06 (d, J=1.1 Hz, 1H), 6.84 (d, J=8.3 Hz, 1H), 6.63 (d, J=8.2 Hz, 1H), 5.70 (s, 2H), 2.95 (br. s, 4H), 2.91-2.77 (m, 3H), 2.74 (t, J=6.1 Hz, 4H), 2.58 (t, J=6.3 Hz, 2H), 1.82-1.74 (m, 2H), 1.73-1.66 (m, 2H), 1.22 (t, J=7.2 Hz, 3H).

### Example 12

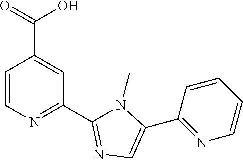 [SMILES: Cn1c(-c2ccccn2)cnc1-c1cc(C(=O)O)ccn1]

### Example 12A

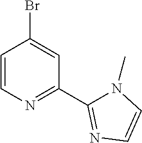

### Example 12A

To a solution of 4-bromo-2-(1H-imidazol-2-yl)pyridine ([1211579-82-6], 400 mg, 1.79 mmol, 1 eq) in THF (5 mL) was added NaH (143 mg, 3.58 mmol, 60% purity, 2 eq) at 0° C. The mixture was stirred at this temperature for 0.5 hr, then to the mixture was added MeI (279 mg, 1.97 mmol, 123 μl, 1.10 eq) dropwise. The mixture was warmed to 15° C. and stirred for 10 hr. The reaction mixture was diluted with H₂O and extracted with EtOAc (20 mL*3). The combined organic layers were washed with brine (20 mL*3), dried over sodium sulfate, filtered and concentrated in vacuo. The crude product was purified by column chromatography (SiO₂, Petroleum ether/Ethyl acetate=10/1 to 6:1). The target compound (340 mg, 1.43 mmol, 80% yield) was obtained as a yellow solid. ¹H NMR indicated the desired product. ¹H NMR (400 MHz, CHLOROFORM-d) δ ppm 8.42 (d, J=1.6 Hz, 1H), 8.38 (d, J=5.2 Hz, 1H), 7.38 (dd, J=5.2, 1.6 Hz, 1H), 7.13 (s, 1H), 6.99 (s, 1H), 4.13 (s, 3H).

### Example 12B

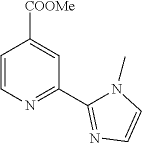 [SMILES: COC(=O)c1ccnc(-c2nccn2C)c1]

### Example 12B

A mixture of Example 12A (150 mg, 630 μmol, 1 eq), Pd(dppf)Cl₂ (46 mg, 63 μmol, 0.10 eq) and Et₃N (956 mg, 9.45 mmol, 1.31 mL, 15 eq) in MeOH (10 mL) was stirred at 70° C. for 5 hr under a CO atmosphere. The reaction mixture was filtered and concentrated in vacuo. The residue was purified by prep-TLC (SiO₂, Petroleum ether/Ethyl acetate=1:1). The target compound (120 mg, 552 μmol, 88% yield) was obtained as a purple solid. ESI m/z 218.1 [M+1]+

### Example 12C

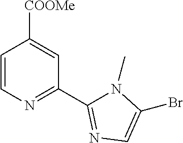 [SMILES: COC(=O)c1ccnc(-c2ncc(Br)n2C)c1]

### Example 12C

To a solution of Example 12B (120 mg, 552 μmol, 1 eq) in DCM (5 mL) was added NBS (98 mg, 552 μmol, 1 eq). The mixture was stirred at 18° C. for 15 min. The reaction mixture was concentrated in vacuo. The residue was purified by prep-TLC (SiO₂, Petroleum ether: Ethyl acetate=1:1). The target compound (150 mg, 506 μmol, 92% yield) was obtained as a light yellow solid. ESI m/z 296.0 [M+1]+ ¹H NMR (400 MHz, CHLOROFORM-d) δ 8.76 (d, J=5.2 Hz, 1H), 8.72 (s, 1H), 7.77-7.86 (m, 1H), 7.21 (s, 1H), 4.15 (s, 3H), 3.99 (s, 3H).

### Example 12D

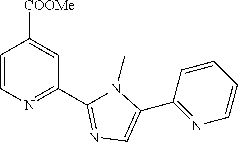 [SMILES: COC(=O)c1ccnc(-c2ncc(-c3ccccn3)n2C)c1]

### Example 12D

A mixture of Example 12C (50 mg, 168.85 μmol, 1 eq), 2-(tributylstannyl)-pyridine ([17997-47-6], 124 mg, 338 μmol, 2 eq) and Pd(PPh₃)₄ (29 mg, 25 μmol, 0.15 eq) in toluene (4 mL) was degassed and purged with N₂ 3 times. The mixture was stirred at 120° C. for 10 hr under a N₂ atmosphere. The reaction mixture was quenched by addition of saturated KF aqueous solution (10 mL) and stirred at 15° C. for 1 hr. The mixture was extracted with EtOAc (20 mL*2). The combined organic phases were washed with brine (20 mL*2), dried with anhydrous sodium sulfate, filtered and concentrated in vacuo. The crude product was purified by prep-TLC (SiO₂, Petroleum ether/Ethyl acetate=4:1). The target compound (20 mg, crude) was obtained as a white solid. ESI m/z 295.2 [M+1]+ ¹H NMR (400 MHz, CHLOROFORM-d) δ 8.80 (d, J=5.2 Hz, 1H), 8.70 (d, J=4.4 Hz, 1H), 7.83 (d, J=3.6 Hz, 1H), 7.77 (td, J=7.6, 1.6 Hz, 1H), 7.62 (d, J=7.6 Hz, 1H), 7.52-7.59 (m, 3H), 4.39 (s, 3H), 3.98 (s, 3H).

### Example 12

To a solution of Example 12D (390 mg, 1.33 mmol, 1 eq) in MeOH (5 mL) and H₂O (3 mL) was added NaOH (106 mg, 2.65 mmol, 2 eq). The mixture was stirred at 60° C. for 15 min. The reaction mixture was concentrated under reduced pressure to remove MeOH. The residue was diluted with H₂O (10 mL) and filtered. The pH of the filtrate was adjusted to 7 with 2M HCl aqueous solution. A precipitate was formed which was filtered and dried in vacuo. The target compound (191 mg, 680 μmol, 51% yield) was obtained as a yellow solid. ESI m/z 281.2 [M+1]⁺ ¹H NMR (400 MHz, DMSO-d₆) δ 9.07 (d, J=4.8 Hz, 1H), 8.80 (d, J=4.4 Hz, 1H), 8.60 (s, 1H), 8.04-8.15 (m, 2H), 8.44 (s, 1H), 7.99 (d, J=8.4 Hz, 1H), 7.57 (dd, J=6.8, 5.2 Hz, 1H), 4.30 (s, 3H).

### Example 13

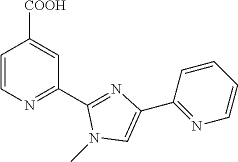 [SMILES: Cn1cc(-c2ccccn2)nc1-c1cc(C(=O)O)ccn1]

### Example 13A

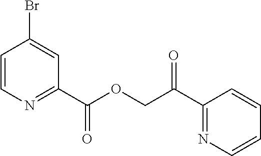 [SMILES: O=C(COC(=O)c1cc(Br)ccn1)c1ccccn1]

### Example 13A

To a solution of 2-bromo-1-(2-pyridinyl)-ethanone hydrobromide ([17570-98-8], 1.11 g, 3.96 mmol, 1 eq) in DMF (10 mL) was added 4-bromo-2-pyridinecarboxylic acid ([30766-03-1], 800 mg, 3.96 mmol, 1 eq) and DIPEA (1.54 g, 11.88 mmol, 2.07 mL, 3 eq). The reaction was stirred at 25° C. for 12 hrs. The mixture was diluted with EtOAc (100 mL), washed with H₂O (30 mL*2) and brine (30 mL). The organic layer was dried over sodium sulfate, filtered and concentrated. The target compound (860 mg, 2.41 mmol, 61% yield) was obtained as a brown oil and used directly in next step. ESI m/z 320.8[M+1]⁺ ¹H NMR (CDCl₃ 400 MHz): δ 8.63 (s, 1H), 8.55 (s, 1H), 8.32 (s, 1H), 8.00-7.98 (d, J=8 Hz, 1H), 7.83-7.81 (m, 1H), 7.63-7.7.62 (m, 1H), 7.49-7.47 (m, 1H), 5.89 (s, 2H).

### Example 13B

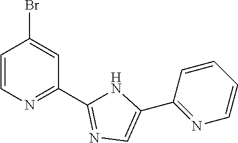 [SMILES: Brc1ccnc(-c2ncc(-c3ccccn3)[nH]2)c1]

### Example 13B

A mixture of Example 13A (321 mg, 1 mmol, 1 eq) and NH₄OAc (1.54 g, 20 mmol, 20 eq) in xylene (10 mL) was heated at 150° C. for 2 hrs in a sealed tube. The mixture was filtered and concentrated. The residue was purified by Prep TLC (EtOAc:PE=1:1). The target compound (78 mg, crude) was obtained as a yellow solid. ESI m/z 303.1 [M+1]⁺ ¹H NMR (CD₃OD 400 MHz): δ 8.78-8.77 (s, 1H), 8.69-8.68 (m, 2H), 8.68-8.66 (m, 1H), 8.61-8.55 (m, 2H), 7.97-7.92 (m, 2H).

### Example 13C

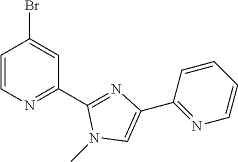 [SMILES: Cn1cc(-c2ccccn2)nc1-c1cc(Br)ccn1]

### Example 13C

To a solution of Example 13B (130 mg, 432 μmol, 1 eq) and Cs₂CO₃ (281 mg, 863 μmol, 2 eq) in DMF (2 mL) was added MeI (74 mg, 518 μmol, 32 Pt, 1.20 eq). The mixture was stirred at RT for 6 hrs. The mixture was diluted with EtOAc (20 mL), washed with H₂O (20 mL*2) and brine (20 mL*2). The organics were dried over sodium sulfate, filtered and concentrated. The residue was purified by prep TLC (EtOAc:PE=1:1). The target compound (80 mg, crude) was obtained as a white solid. ESI m/z 317.1[M+1]⁺ ¹H NMR (CD₃OD 400 MHz): δ 8.70-8.69 (m, 1H), 8.59-8.54 (m, 3H), 8.44-8.41 (m, 2H), 7.88-7.86 (m, 1H), 7.73 (s, 1H), 4.27 (s, 3H).

### Example 13D

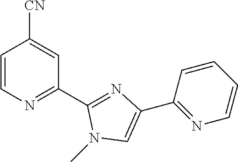 [SMILES: Cn1cc(-c2ccccn2)nc1-c1cc(C#N)ccn1]

### Example 13D

A mixture of Example 13C (40 mg, 127 μmol, 1 eq), Zn (830 μg, 12.69 μmol, 0.10 eq), Zn(CN)₂ (15 mg, 127 μmol, 1 eq), DPPF (14 mg, 25 μmol, 0.20 eq) and Pd₂ (dba)₃ (11.6 mg, 12.7 μmol, 0.10 eq) in DMAC (800 μl) was heated at 120° C. for 2 hrs. The mixture was diluted with EtOAc (30 mL), washed with water (20 mL*2), brine (20 mL*2), dried over sodium sulfate, filtered and concentrated. The residue was purified by prep-TLC (EtOAc:PE=1:1). The target compound (23 mg, crude) was obtained as a grey solid. ESI m/z 262.1 [M+1]⁺

### Example 13

A mixture of Example 13D (23 mg, 88 μmol, 1 eq) and NaOH (7 mg, 176 μmol, 2 eq) in H₂O (1 mL) and MeOH (1 mL) was heated at 80° C. for 10 hrs. The MeOH was removed under reduced pressure and the residue was adjusted to pH=3 with 2 N HCl. The residue was purified by prep-HPLC (TFA buffer), treated with 1 N HCl (1 drop) and lyophilized. The target compound (8 mg, 28 μmol, 32% yield) was obtained as a yellow solid. ESI m/z 281.2[M+1]⁺ ¹H NMR (CD₃OD 400 MHz): δ 8.89-8.87 (m, 2H), 8.66-8.65 (d, J=4 Hz, 1H), 8.47 (s, 1H), 8.36-8.34 (m, 2H), 7.98-7.97 (d, J=4 Hz, 1H), 7.78 (s, 1H), 4.29 (s, 3H).

### Example 14

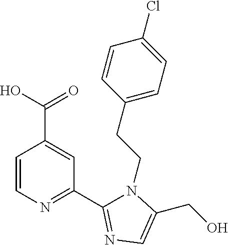 [SMILES: O=C(O)c1ccnc(-c2ncc(CO)n2CCc2ccc(Cl)cc2)c1]

### Example 14A

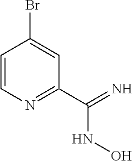 [SMILES: N=C(NO)c1cc(Br)ccn1]

### Example 14A

To a solution of 4-bromo-2-pyridinecarbonitrile ([62150-45-2], 1.75 g, 9.56 mmol, 1 eq) in a mixture of ethanol (25 mL) and H₂O (25 mL) was added hydroxylamine hydrochloride (1.66 g, 24 mmol, 2.50 eq) and K₂CO₃ (2 eq) at 20° C. The reaction was stirred at 80° C. for 12 hours. The reaction was concentrated in vacuo. The residue was dissolved in ethyl acetate (30 mL) and washed with H₂O (30 mL*2). The organic layer was dried over sodium sulfate and concentrated in vacuo to give the target compound (2.00 g, 9.26 mmol, 97% yield) as a white solid. ¹H NMR (400 MHz, CDCl₃) δ 8.38 (d, J=5.3 Hz, 1H), 8.13 (d, J=1.8 Hz, 1H), 7.48 (dd, J=1.8, 5.3 Hz, 1H).

### Example 14B

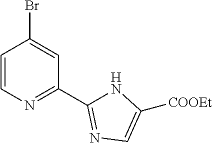 [SMILES: CCOC(=O)c1cnc(-c2cc(Br)ccn2)[nH]1]

### Example 14B

To a solution of Example 14A (2.0 g, 9.35 mmol, 1 eq) in methanol (50 mL) was added ethyl prop-2-ynoate (5.50 g, 56 mmol, 5.5 mL, 6 eq) at 20° C. The reaction was stirred at 70° C. for 12 hours. Then the reaction was concentrated in vacuo. The residue was suspended in Ph₂O (20 mL) and stirred at 200° C. for 1 hour. The mixture was cooled to RT and petroleum ether (20 mL) was added. The crude product was purified by column chromatography to give the target compound (1.05 g, 3.55 mmol, 38% yield) as a yellow solid. ¹H NMR (400 MHz, CD₃OD) δ 8.51-8.47 (d, J=5.2 Hz, 1H), 8.44-8.41 (s, 1H), 7.91-7.88 (m, 1H), 7.65-7.60 (m, 1H), 4.39 (q, J=7.0 Hz, 2H), 1.41 (t, J=7.0 Hz, 3H).

### Example 14C

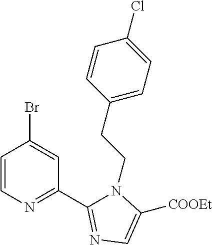 [SMILES: CCOC(=O)c1cnc(-c2cc(Br)ccn2)n1CCc1ccc(Cl)cc1]

### Example 14C

To a solution of Example 14B (1.05 g, 3.55 mmol, 1 eq) in DMF (5 mL) was added 2-(4-chlorophenyl) ethyl methanesulfonate (1.08 g, 4.62 mmol, 1.30 eq) and K₂CO₃ (981 mg, 7.10 mmol, 2 eq) at 20° C. The reaction was stirred at 80° C. for 2 hours. The reaction was diluted with ethyl acetate (20 mL) and washed with brine (20 mL*2). The organic layer was dried with sodium sulfate. The residue was purified by column chromatography to give the target compound (430 mg, 989 μmol, 28% yield) as a white solid. ¹H NMR (400 MHz, CD₃OD) δ 8.52 (d, J=5.3 Hz, 1H), 7.88 (s, 1H), 7.81 (s, 1H), 7.66 (d, J=5.3 Hz, 1H), 7.12 (d, J=8.0 Hz, 2H), 6.92 (d, J=8.3 Hz, 2H), 5.22 (t, J=6.8 Hz, 2H), 4.43-4.38 (m, 2), 3.06 (t, J=6.0 Hz, 2H), 1.42 (t, J=7.0 Hz, 3H).

### Example 14D

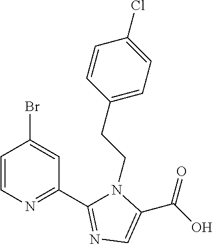 [SMILES: O=C(O)c1cnc(-c2cc(Br)ccn2)n1CCc1ccc(Cl)cc1]

### Example 14D

To a solution of Example 14C (400 mg, 920 μmol, 1 eq) in methanol (5 mL) was added LiOH.H₂O (2 M, 460 μl, 1 eq) at 20° C. The reaction was stirred at 20° C. for 12 hours. Then the reaction was concentrated in vacuo and the pH adjusted to 5 with HCl (1M: 0.5 mL). The mixture was filtered to give the target compound (300 mg, 738 μmol, 80% yield) as a white solid. ¹H NMR (400 MHz, DMSO-d6) δ 8.56 (d, J=5.3 Hz, 1H), 7.96 (d, J=1.8 Hz, 1H), 7.72 (m, 2H), 7.20 (d, J=8.3 Hz, 2H), 7.00 (d, J=8.3 Hz, 2H), 5.15 (t, J=7.0 Hz, 2H), 3.00 (t, J=7.2 Hz, 2H).

### Example 14E

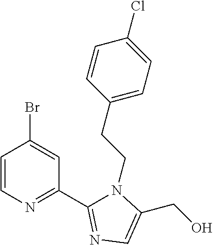 [SMILES: OCc1cnc(-c2cc(Br)ccn2)n1CCc1ccc(Cl)cc1]

### Example 14E

To a solution of Example 14D (140 mg, 344 μmol, 1 eq) in THF (5 mL) was added Et₃N (38 mg, 379 μmol, 53 μl, 1.10 eq) and ethyl chloroformate (41 mg, 379 μmol, 36 μl, 1.10 eq) at 0° C. The reaction was stirred at 0° C. for 1 hour. Then to this reaction mixture was added NaBH₄ (26 mg, 689 μmol, 2 eq) at 0° C. The reaction was stirred at 20° C. for 1 hour. To this reaction was added H₂O (10 mL) and the mixture extracted with ethyl acetate (10 mL*2). The combined organic layers were washed with brine (10 mL), dried over anhydrous sodium sulfate, filtered and concentrated in vacuo. The residue was purified by prep-TLC to give the target compound (20 mg, 43 μmol, 12.6% yield, 85% purity) as a white solid. LCMS: ESI m/z 394.0 [M+1].

### Example 14F

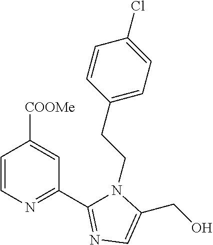 [SMILES: COC(=O)c1ccnc(-c2ncc(CO)n2CCc2ccc(Cl)cc2)c1]

### Example 14F

To a solution of Example 14E (20 mg, 51 μmol, 1.00 eq) in methanol (5 mL) was added Et₃N (77 mg, 764 μmol, 106 μl, 15 eq) and Pd(dppf)Cl₂ (5.59 mg, 7.64 μmol, 0.15 eq). The reaction was stirred at 80° C. under CO (50 psi) for 1 hour. The reaction was concentrated in vacuo. The residue was purified by prep-TLC to give the target compound (20 mg, 47 μmol, 92% yield, 86.8% purity) as a white solid. LCMS: ESI m/z 371.9 [M+1].

### Example 14

To a solution of Example 14F (20 mg, 54 μmol, 1 eq) in methanol (4 mL) was added LiOH₂.H₂O (2 M, 27 μl, 1 eq) at 20° C. The reaction was stirred at 20° C. for 12 hours. The reaction was concentrated in vacuo and the pH adjusted 5 with HCl (1 M; 0.1 mL). The residue was purified by prep-HPLC (TFA condition) to give the target compound (10 mg, 28 μmol, 52% yield) as a white solid. LCMS: ESI m/z 358.2 [M+1]. ¹H NMR (400 MHz, CD₃OD) δ 8.95 (d, J=4.5 Hz, 1H), 8.11 (s, 1H), 8.08 (d, J=5.3 Hz, 1H), 7.64 (s, 1H), 7.09 (d, J=8.0 Hz, 2H), 6.92 (d, J=8.3 Hz, 2H), 5.08 (t, J=6.5 Hz, 2H), 4.79 (s, 2H), 3.20 (t, J=6.5 Hz, 2H).

### Example 15

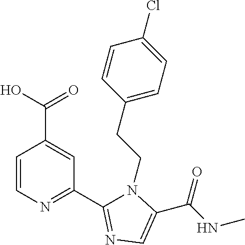 [SMILES: CNC(=O)c1cnc(-c2cc(C(=O)O)ccn2)n1CCc1ccc(Cl)cc1]

### Example 15A

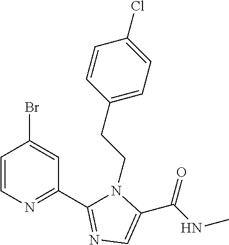 [SMILES: CNC(=O)c1cnc(-c2cc(Br)ccn2)n1CCc1ccc(Cl)cc1]

### Example 15A

To a solution of Example 14D (85 mg, 209 μmol, 1 eq) in DMF (5 mL) was added methylamine hydrochloride (21 mg, 314 μmol, 1.50 eq), HATU (119 mg, 314 μmol, 1.50 eq) and DIPEA (108 mg, 836 μmol, 146 μl, 4 eq). The reaction was stirred at RT for 1 hour. The reaction was diluted with ethyl acetate (20 mL) and washed with brine (20 mL*2). The organic layer was dried over sodium sulfate and concentrated in vacuo to give the target compound (80 mg, 184 μmol, 88% yield, 96% purity) as a white solid. LCMS: ESI m/z 421.0 [M+1].

### Example 15B

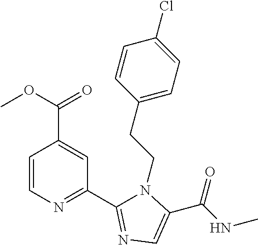

### Example 15B

To a solution of Example 15A (40 mg, 95 μmol, 1 eq) in methanol (5 mL) was added Et₃N (145 mg, 1.43 mmol, 198 μl, 15 eq) and Pd(dppf)Cl₂ (10 mg, 14 μmol, 0.15 eq). The reaction was stirred at 80° C. under CO (50 psi) for 1 hour. The reaction was concentrated in vacuo. The residue was purified by prep-TLC to give the target compound (35 mg, 72 μmol, 75.5% yield, 82% purity) as a white solid. LCMS: ESI m/z 399.0 [M+1].

### Example 15

To a solution of Example 15B (40 mg, 100 μmol, 1 eq) in methanol (4 mL) was added LiOH.H₂O (4.2 mg, 100 μmol, 1 eq) at 20° C. The reaction was stirred at 20° C. for 12 hours. The reaction was concentrated in vacuo and the pH adjusted to 5 with HCl (1 M; 0.1 mL). The residue was purified by prep-HPLC (TFA buffer) to give the target compound (8 mg, 21 μmol, 21% yield, 100% purity) as a white solid. LCMS: ESI m/z 385.2 [M+1]. ¹H NMR (400 MHz, CD₃OD) δ 8.92 (d, J=5.0 Hz, 1H), 8.19 (s, 1-), 8.02 (d, J=4.3 Hz, 1H), 7.84 (s, 1H), 7.09 (d, J=8.3 Hz, 2H), 6.91 (d, J=8.3 Hz, 2H), 5.27 (t, J=6.8 Hz, 2H), 3.12 (t, J=6.7 Hz, 2H), 2.93 (s, 3H).

### Example 16

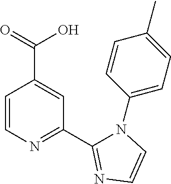 [SMILES: Cc1ccc(-n2ccnc2-c2cc(C(=O)O)ccn2)cc1]

### Example 16A

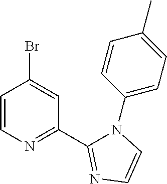 [SMILES: Cc1ccc(-n2ccnc2-c2cc(Br)ccn2)cc1]

### Example 16A

To a solution of 4-bromo-2-(1H-imidazol-2-yl)pyridine ([1211579-82-6], 100 mg, 446 μmol, 1 eq), p-tolylboronic acid (121 mg, 893 μmol, 2 eq) and Et₃N (135 mg, 1.34 mmol, 186 μl, 3 eq) in DCM (5 mL) was added Cu(OAc)₂ (162 mg, 893 μmol, 2 eq). The mixture was stirred under an O₂ atmosphere at 25° C. for 2 hours. To the reaction solution was added 2 mL ammonium hydroxide solution. The mixture was extracted with DCM (20 mL*3). The combined organics were dried over anhydrous sodium sulfate. The solvent was removed in vacuo to afford the crude product. The crude product was purified by prep-TLC (DCM/MeOH=20/1) to give the target compound as a yellow oil (120 mg, 86% yield). ESI m/z 314.1 [M+1]⁺ ¹H NMR (400 MHz, CD₃OD) δ 8.07 (d, J=5.2 Hz, 1H), 7.88 (d, J=1.2 Hz, 1H), 7.42 (dd, J=1.8, 5.2 Hz, 1H), 7.29 (s, 1H), 7.13 (d, J=8.2 Hz, 3H), 7.06-7.00 (m, 2H), 2.28 (s, 3H).

### Example 16B

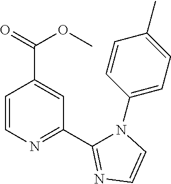 [SMILES: COC(=O)c1ccnc(-c2nccn2-c2ccc(C)cc2)c1]

### Example 16B

To a solution of Example 16A (80 mg, 255 μmol, 1 eq) and Et₃N (258 mg, 2.55 mmol, 353 μl, 10 eq) in MeOH (10 mL) was added Pd(dppf)Cl₂ (28 mg, 38 μmol, 0.15 eq). The mixture was purged with carbon monoxide 6 times, then the solution was heated to 80° C. under a carbon monoxide atmosphere (50 psi) for 18 hours. The solution was cooled to RT and filtered. The filtrate was concentrated in vacuo to afford crude product. The residue was purified by prep-TLC (DCM/MeOH=20/1) to give the product as a yellow solid (30 mg, 40%, yield). ESI w: 294.1 [M+1]⁺

### Example 16

To a solution of Example 16B (40 mg 136 μmol, 1 eq) in MeOH (2 mL) was added 1 mL LiOH (2 M) solution. The mixture was stirred at 25° C. for 1 hour. The pH was adjusted to 7 and the solvent removed in vacuo to afford the crude product. The crude product was purified by prep-HPLC to obtain the product as a white solid (30 mg, 79%, yield). ESI m/z 280.1 [M+1]+ ¹H NMR (400 MHz, CD₃OD) δ 8.93 (dd, J=1.0, 5.0 Hz, 1H), 8.06 (dd, J=1.4, 5.0 Hz, 1H), 7.94 (d, J=2.0 Hz, 1H), 7.91 (d, J=2.0 Hz, 1H), 7.75 (s, 1H), 7.49 (s, 4H), 2.51 (s, 3H).

### Example 17

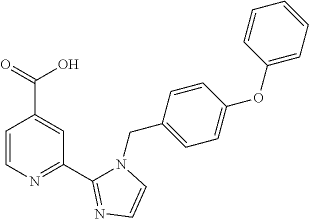 [SMILES: O=C(O)c1ccnc(-c2nccn2Cc2ccc(Oc3ccccc3)cc2)c1]

### Example 17A

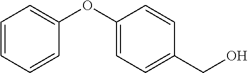 [SMILES: OCc1ccc(Oc2ccccc2)cc1]

### Example 17A

To a solution of 4-phenoxybenzoic acid (1.07 g, 5 mmol, 1 eq) in THF (20 mL) was added BH₃-Me₂S (570 mg, 7.50 mmol, 1.50 eq) dropwise. The mixture was stirred at 0° C. for 2 hours, then warmed to 25° C. for a further 8 hours. The solvent was removed in vacuo. To the residue was added water (30 mL) and the mixture extracted with EA (30 mL*3). The combined organics were dried with sodium sulfate and concentrated in vacuo to afford the target compound as a white solid, which was used directly for the next step without further purification (0.99 g, 99%, yield).

### Example 17B

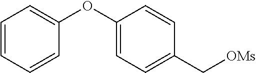 [SMILES: CS(=O)(=O)OCc1ccc(Oc2ccccc2)cc1]

### Example 17B

To a solution of Example 17A (0.99 g, 4.99 mmol, 1 eq) and Et₃N (1.52 g, 15 mmol, 2.08 mL, 3 eq) in DCM (5 mL) at 0° C. was added MsCl (2.50 g, 22 mmol, 1.69 mL, 4.37 eq) dropwise. The mixture was stirred for 1 hour at 0° C. Water (10 m L) was added and the organic layer separated. The aqueous layer was further extracted with DCM (10 mL*2). The combined organic layers were dried with anhydrous sodium sulfate and concentrated to afford the target compound as an oil (1.2 g, 86.4%, yield) which was used directly without further purification.

### Example 17C

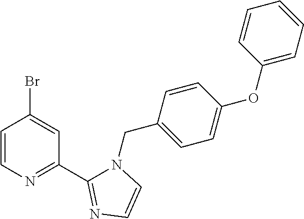 [SMILES: Brc1ccnc(-c2nccn2Cc2ccc(Oc3ccccc3)cc2)c1]

### Example 17C

To a solution of 4-bromo-2-(1H-imidazol-2-yl)pyridine ([1211579-82-6], 150 mg, 669 μmol, 1 eq) and Example 17B (242 mg, 870 μmol, 1.30 eq) in DMF (5 mL) was added K₂CO₃ (231 mg, 1.67 mmol, 2.50 eq). The mixture was heated to 100° C. for 12 hours. The mixture was cooled to RT and filtered. The filtrate was concentrated in vacuo to afford the crude product. The crude product was purified by prep-TLC to obtain the target compound as a white solid (180 mg, 66%, yield). ESI m/z 405.9 [M+I]+

### Example 17D

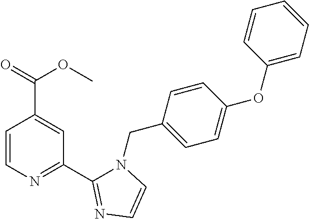 [SMILES: COC(=O)c1ccnc(-c2nccn2Cc2ccc(Oc3ccccc3)cc2)c1]

### Example 17D

To a solution of Example 17C (160 mg, 394 μmol, 1 eq) and Et₃N (319 mg, 3.15 mmol, 437 μl, 8 eq) in MeOH (10 mL) was added Pd(dppf)Cl₂ (43 mg, 59 μmol, 0.15 eq). The mixture was purged with carbon monoxide 6 times, then the mixture was heated under a carbon monoxide atmosphere (50 psi) at 80° C. for 8 hours. The reaction was filtered. The filtrate was concentrated in vacuo to afford the crude product. The crude product was purified by prep-TLC to give the target compound as a yellow solid (100 mg, 66% yield). ESI m/z 386.0 [M+1]+ ¹H NMR (400 MHz, CD₃OD) δ 8.78 (dd, J=0.8, 5.0 Hz, 1H), 8.53 (dd, J=0.9, 1.5 Hz, 1H), 7.83 (dd, J=1.6, 5.0 Hz, 1H), 7.36-7.28 (m, 3H), 7.21-7.16 (m, 3H), 7.13-7.07 (m, 1H), 6.95-6.90 (m, 2H), 6.89-6.84 (m, 2H), 5.86 (s, 2H), 3.98 (s, 3H).

### Example 17

To a solution of Example 17D (50 mg, 130 μmol, 1 eq) in MeOH (2 mL) was added 1 mL NaOH solution (2 M). The mixture was stirred at 25° C. for 2 hours. The pH of the mixture was adjusted to 7 with HCl (2 M) solution. A precipitate formed and was filtered and dried to afford the target compound as a white solid (30 mg, 62%, yield). ESI m/z 372.0 [M+1]+ ¹H NMR (400 MHz, CD₃OD) δ 8.82 (d, J=4.8 Hz, 1H), 8.52 (s, 1H), 7.89 (dd, J=1.4, 5.0 Hz, 1H), 7.41 (s, 1H), 7.38-7.30 (m, 2H), 7.26-7.19 (m, 3H), 7.14-7.08 (m, 1H), 6.97-6.86 (m, 4H), 5.88 (s, 2H).

### Example 18

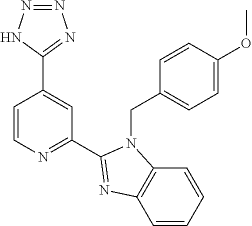 [SMILES: COc1ccc(Cn2c(-c3cc(-c4nnn[nH]4)ccn3)nc3ccccc32)cc1]

### Example 18

Example 2B (129 mg, 0.38 mmol), NaN₃ (86 mg, 1.33 mmol) and NH₄Cl (80 mg, 1.5 mmol) are heated in DMF (3 mL) at 100° C. for 3 hrs. The reaction mixture is then cooled and partitioned between ethyl acetate and brine. The organic layer is further washed once with brine, dried (Na₂SO₄) and concentrated to give 28 mg of the title compound as a white solid. LCMS MH⁺ calculated, 384.2, found 384.6. ¹H NMR (DMSO-d6, 500 MHz) δ 9.02 (s, 1H), 8.95 (d, J=5 Hz, 1H), 8.11 (dd, J=1.5, 5 Hz, 1H), 7.79 (m, 1H), 7.66 (m, 1H), 7.31 (m, 2H), 7.14 (d, J=8.5 Hz, 2H), 6.81 (dd, J=1.5, 6.5 Hz, 2H), 6.19 (s, 2H), 3.65 (s, 3H).

### Example 19

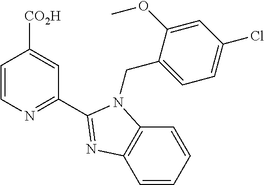 [SMILES: COc1cc(Cl)ccc1Cn1c(-c2cc(C(=O)O)ccn2)nc2ccccc21]

### Example 19A

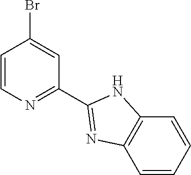 [SMILES: Brc1ccnc(-c2nc3ccccc3[nH]2)c1]

A solution of 4-bromopicolinaldehyde (3.72 g, 20 mmol) and phenylene diamine (4.33 g, 40 mmol) in DMA (20 mL) was heated at 70° C. under N₂ for 4 h. The reaction mixture was cooled and partitioned between ethyl acetate and brine. The organic layer was further washed once with brine, dried (Na₂SO₄) and concentrated. Chromatography (SiO₂, 0→5% MeOH in CH₂Cl₂) gave 2.6 g (47%) of the target compound. LCMS MH⁺ calculated, 274.0, found 274.3.

### Example 19B

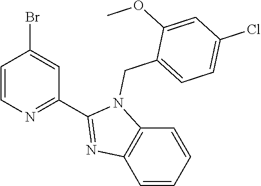 [SMILES: COc1cc(Cl)ccc1Cn1c(-c2cc(Br)ccn2)nc2ccccc21]

To a solution of Example 19A (274 mg, 1 mmol) in DMF (3 mL) at 0° C. was added NaH (51 mg). The mixture was stirred at 0° C. for 1 h. 1-(Bromomethyl)-4-chloro-2-methoxybenzene (306 mg, 1.3 mmol) was added. The cooling bath was removed and the mixture stirred at RT for 4 h. The mixture was partitioned between ethyl acetate and brine. The organic layer was further washed twice with brine, dried (Na₂SO₄) and concentrated to give a yellow solid. The solid was triturated with a mixture of ethyl acetate and pentane, filtered and dried to give the target compound as an off-white solid (355 mg, 83%). LCMS MW calculated, 428.0, found 428.6.

### Example 19C

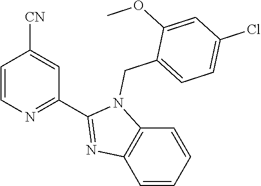 [SMILES: COc1cc(Cl)ccc1Cn1c(-c2cc(C#N)ccn2)nc2ccccc21]

### Example 19C

A mixture of Example 19B (345 mg, 0.81 mmol), Zn(CN)₂ and Pd(dppf)Cl₂ in DMA (2.5 mL) was purged with nitrogen for 5 min. The reaction was heated at 120° C. for 17 h. Water was added. The precipitate that formed was collected by filtration. Chromatography (SiO₂, 10→40% ethyl acetate in pentane) gave the target compound as an off-white solid (180 mg, 60%). LCMS MW calculated, 375.1, found 375.6.

### Example 19

A suspension of Example 19C (165 mg, 0.44 mmol) in a mixture of NaOH (5M, 1.5 mL) and ethanol (1.5 mL) was heated at 85° C. in a pressure vessel overnight. The mixture was concentrated to remove ethanol and the pH adjusted to 7 with HCl (3M). The solid formed was collected by filtration, rinsed with water and dried to give the target compound as a white solid (144 mg, 83%). LCMS MW calculated, 394.1, found 394.1. ¹H NMR (DMSO-d6, 500 MHz) δ 8.68 (s, 1H), 8.58 (d, J=5 Hz, 1H), 7.76 (m, 2H), 7.44 (m, 1H), 7.26 (m, 2H), 7.06 (d, J=2 Hz, 1H), 6.80 (dd, J=2, 8 Hz, 1H), 6.56 (d, J=8 Hz, 1H), 6.11 (s, 2H), 3.77 (s, 3H).

### Example 20

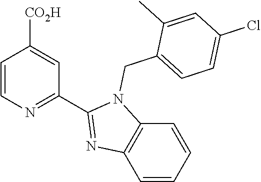 [SMILES: Cc1cc(Cl)ccc1Cn1c(-c2cc(C(=O)O)ccn2)nc2ccccc21]

Example 20 was prepared in a manner analogous to Example 19. LCMS MH⁺ calculated, 378.1, found 378.1. ¹H NMR (DMSO-d6, 500 MHz) δ 8.79 (s, 1H), 8.68 (d, J=2 Hz, 1H), 7.82 (m, 2H), 7.49 (m, 1H), 7.31 (m, 3H), 6.99 (dd, J=2, 8 Hz, 1H), 6.20 (d, J=8.5 Hz, 1H), 6.15 (s, 2H), 2.44 (s, 3H)-.

### Example 21

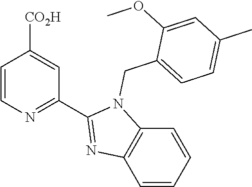 [SMILES: COc1cc(C)ccc1Cn1c(-c2cc(C(=O)O)ccn2)nc2ccccc21]

Example 21 was prepared in a manner analogous to Example 19. LCMS MW calculated, 374.1, found 374.0. ¹H NMR (DMSO-d6, 500 MHz) δ 8.79 (d, J=5 Hz, 1H), 8.72 (s, 1H), 7.86 (dd, J=1.5, 5 Hz, 1H), 7.76 (m, 1H), 7.47 (m, 1H), 7.26 (m, 2H), 6.78 (s, 1H), 6.53 (s, 2H), 6.11 (s, 2H), 3.67 (s, 3H), 2.20 (s, 3H).

### Example 22

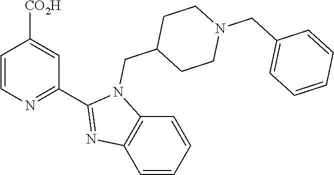 [SMILES: O=C(O)c1ccnc(-c2nc3ccccc3n2CC2CCN(Cc3ccccc3)CC2)c1]

### Example 22A

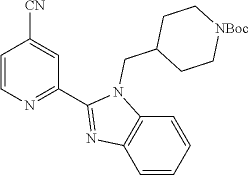 [SMILES: CC(C)(C)OC(=O)N1CCC(Cn2c(-c3cc(C#N)ccn3)nc3ccccc32)CC1]

Example 22A was prepared in a manner analogous to Example 19C. LCMS MW calculated, 418.2, found 418.8.

### Example 22B

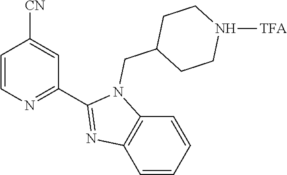

### Example 22B

To a solution of Example 22A (126 mg, 0.30 mmol) in CH₂Cl₂ (0.8 mL) was added TFA (0.8 mL). The reaction was stirred for 3 h. The reaction was concentrated to give a yellow solid. LCMS MH⁺ calculated, 318.2, found 318.5.

### Example 22C

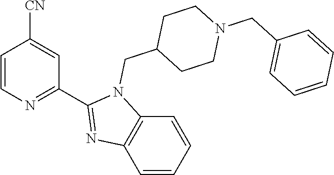 [SMILES: N#Cc1ccnc(-c2nc3ccccc3n2CC2CCN(Cc3ccccc3)CC2)c1]

### Example 22C

A solution of Example 22B (83 mg, 0.2 mmol) and benzaldehyde (64 mg, 0.6 mmol) in MeOH (1 mL) was treated with NaCN(BH₃) (19 mg, 0.3 mmol). The mixture was stirred for 6 h. The reaction mixture was concentrated to remove MeOH and then partitioned between CH₂Cl₂ and brine. The organic layer was dried (Na₂SO₄) and concentrated. PTLC (ethyl acetate) gave the target compound as a white solid (34 mg, 42%). LCMS MH⁺ calculated, 408.2, found 408.6.

Example 22 was prepared from Example 22C in a manner analogous to Example 19. LCMS MW calculated, 427.2, found 427.4. ¹H NMR (DMSO-d6, 500 MHz) δ 8.87 (d, J=5 Hz, 1H), 8.72 (s, 1H), 7.84 (d, J=5 Hz, 1H), 7.73 (t, J=8 Hz, 2H), 7.30 (m, 7H), 4.88 (d, J=6.5 Hz, 2H), 3.60 (br s, 2H), 2.84 (d, J=10.5 Hz, 2H), 2.04 (br s, 21-H), 1.93 (br s, 1H), 1.43 (d, J=11.5 Hz, 2H), 1.33 (m, 2H).

### Example 23

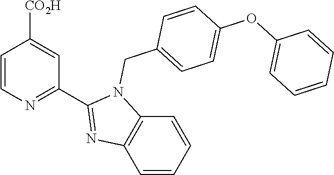 [SMILES: O=C(O)c1ccnc(-c2nc3ccccc3n2Cc2ccc(Oc3ccccc3)cc2)c1]

Example 23 was prepared in a manner analogous to Example 19. LCMS MH⁺ calculated, 422.1, found 422.5. ¹H NMR (DMSO-d6, 500 MHz) δ 8.70 (s, 1H), 8.63 (dd, J=0.5, 5 Hz, 1H), 7.77 (m, 1H), 7.74 (m, 1H), 7.59 (m, 1H), 7.34 (m, 2H), 7.27 (m, 2H), 7.19 (d, J=9 Hz, 2H), 7.09 (t, J=8 Hz, 1H), 6.93 (d, J=7.5 Hz, 2H), 6.88 (d, J=9.5 Hz, 2H), 6.20 (s, 2H).

### Example 24

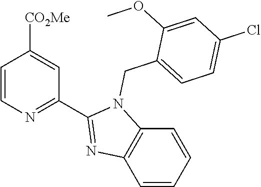 [SMILES: COC(=O)c1ccnc(-c2nc3ccccc3n2Cc2ccc(Cl)cc2OC)c1]

### Example 24

A suspension of Example 19 (59 mg, 0.15 mmol) in MeOH (1 mL) was treated with trimethylsilyldiazomethane (1 mL of a 2 M solution in diethyl ether) at 0° C. The cooling bath was removed and the reaction stirred overnight at RT. The mixture was concentrated. Chromatography (SiO₂, 0→30% ethyl acetate in hexane) gave the target compound (43 mg, 70%) as a white solid. LCMS MH⁺ calculated, 408.1, found 408.4. ¹H NMR (DMSO-d6, 500 MHz) δ 8.84 (d, J=5 Hz, 1H), 8.78 (m, 1H), 7.90 (dd, J=1.5, 5 Hz, 1H), 7.79 (m, 1H), 7.50 (m, 1H), 7.29 (m, 2H), 7.06 (d, J=2 Hz, 2H), 6.80 (dd, J=2, 8 Hz, 1H), 6.60 (d, J=8 Hz, 1H), 6.12 (s, 2H), 3.96 (s, 3H), 3.75 (s, 3H).

### Example 25

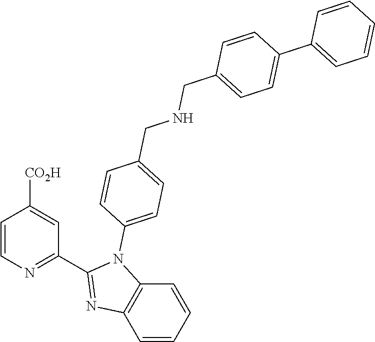 [SMILES: O=C(O)c1ccnc(-c2nc3ccccc3n2-c2ccc(CNCc3ccc(-c4ccccc4)cc3)cc2)c1]

### Example 25A

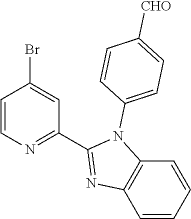 [SMILES: O=Cc1ccc(-n2c(-c3cc(Br)ccn3)nc3ccccc32)cc1]

A mixture of Example 19A (2.19 g, 8 mmol), 4-fluorobenzaldehyde (4.96 g, 40 mmol) and K₂CO, (5.53 g, 40 mmol) in DMF (12 mL) was heated at 120° C. in a pressure vessel for 3 days. The reaction was cooled and partitioned between ethyl acetate and brine. The organic layer was further washed twice with brine, dried (Na₂SO₄) and concentrated. Chromatography (SiO₂, 0→30% ethyl acetate in hexane gave the target compound (1.78 g, 59/0) as a pale yellow solid. LCMS MH⁺ calculated, 378.0, found 378.2.

### Example 25B

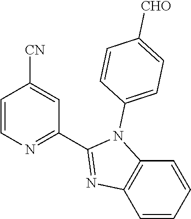 [SMILES: N#Cc1ccnc(-c2nc3ccccc3n2-c2ccc(C=O)cc2)c1]

Example 25B was prepared from Example 25A in a manner analogous to Example 19C. LCMS MH⁺ calculated, 325.1, found 325.2.

### Example 25C

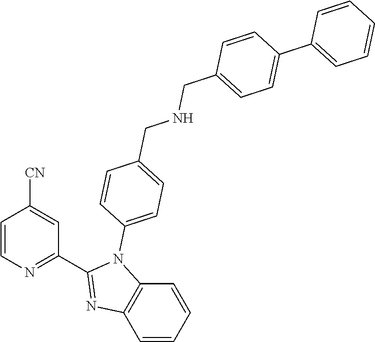 [SMILES: N#Cc1ccnc(-c2nc3ccccc3n2-c2ccc(CNCc3ccc(-c4ccccc4)cc3)cc2)c1]

### Example 25C

A solution of Example 25B (70 mg, 0.22 mmol), [1,1′-biphenyl]-4-ylmethanamine (79 mg, 0.43 mmol) and AcOH (0.1 mL) in MeOH (1 mL) and CH₂Cl₂ (1 mL) was stirred at RT for 1 h. NaCN(BH₃) (27 mg, 0.43 mmol) was added and the reaction stirred for another 4 h. The mixture was concentrated and the residue partitioned between ethyl acetate and saturated NaHCO₃. The organic layer was further washed once with brine, dried (Na₂SO₄) and concentrated. Chromatography (SiO₂, 0→5% MeOH in ethyl acetate) gave the target compound (95 mg, 89%) as a white solid. LCMS MH⁺ calculated, 492.2, found 492.3.

Example 25 was prepared from Example 25C in a manner analogous to that of Example 19. LCMS MH⁺ calculated, 511.2, found 511.3. ¹H NMR (DMSO-d6, 500 MHz) δ 8.58 (s, 1H), 8.38 (d, J=5 Hz, 1H), 7.82 (d, J=7.5 Hz, H), 7.71 (d, J=4.5 Hz, 1H), 7.68 (m, 4H), 7.57 (m, 4H), 7.46 (t, J=7.5 Hz, 2H), 7.37 (m, 3H), 7.29 (m, 2H), 7.21 (d, J=7.5 Hz, 1H), 4.04 (m, 4H), 3.43 (br s, 1H).

### Example 26

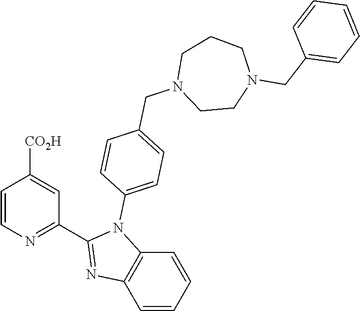 [SMILES: O=C(O)c1ccnc(-c2nc3ccccc3n2-c2ccc(CN3CCCN(Cc4ccccc4)CC3)cc2)c1]

### Example 26A

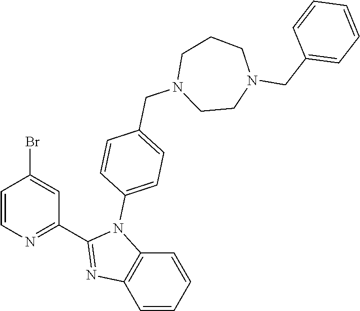 [SMILES: Brc1ccnc(-c2nc3ccccc3n2-c2ccc(CN3CCCN(Cc4ccccc4)CC3)cc2)c1]

A solution of Example 25A (265 mg, 0.7 mmol) and 1-benzyl-1,4-diazepane (267 mg, 1.4 mmol) in MeOH (1.5 mL) and CH₂Cl₂ (0.7 mL) was treated with AcOH (0.08 mL). The reaction was stirred for 1 h. NaCN(BH₃) (88 mg, 1.4 mmol) was added and the reaction stirred for another 3 h. The mixture was concentrated and then partitioned between ethyl acetate and saturated NH₄Cl. The organic layer was dried (Na₂SO₄) and concentrated. Chromatography (SiO₂, 0→15% MeOH in ethyl acetate) gave the target compound (325 mg, 84%) as a white solid. LCMS MH⁺ calculated, 552.2, found 552.3.

### Example 26B

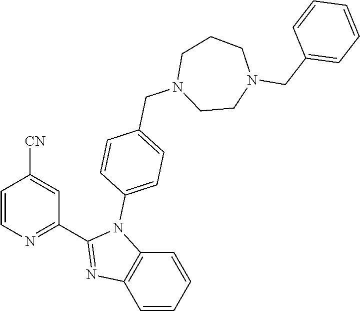 [SMILES: N#Cc1ccnc(-c2nc3ccccc3n2-c2ccc(CN3CCCN(Cc4ccccc4)CC3)cc2)c1]

Example 26B was prepared from Example 26A in a manner analogous to Example 19C. LCMS MH⁺ calculated, 499.3, found 499.4.

Example 26 was prepared from Example 268 in a manner analogous to Example 19. LCMS MH⁺ calculated, 518.3, found 518.4. ¹H NMR (DMSO-d6, 500 MHz) δ 8.50 (d, J=4.5 Hz, 1H), 8.17 (d, J=3 Hz, 1H), 7.83 (d, J=7.5 Hz, 1H), 7.74 (d, J=4.5 Hz, 1H), 7.45 (m, 4H), 7.33 (m, 7H), 7.24 (d, J=7.5 Hz, 1H), 3.91 (br s, 2H), 3.84 (br s, 2H), 2.91 (d, J=4.5 Hz, 4H), 2.78 (d, J=5.5 Hz, 4H), 1.86 (m, 2H).

### Example 27

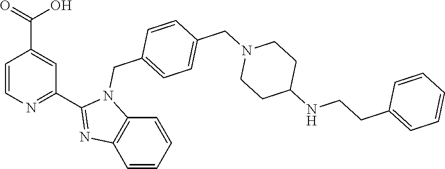 [SMILES: O=C(O)c1ccnc(-c2nc3ccccc3n2Cc2ccc(CN3CCC(NCCc4ccccc4)CC3)cc2)c1]

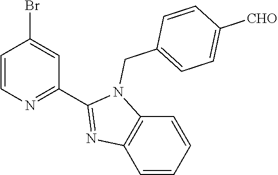 [SMILES: O=Cc1ccc(Cn2c(-c3cc(Br)ccn3)nc3ccccc32)cc1]

Example 27A was prepared in a manner analogous to Example 19B. LCMS MH⁺ calculated, 392.0, found 392.1.

### Example 27B

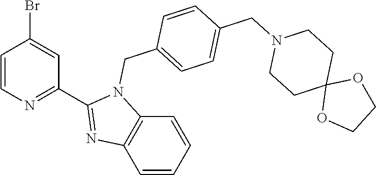 [SMILES: Brc1ccnc(-c2nc3ccccc3n2Cc2ccc(CN3CCC4(CC3)OCCO4)cc2)c1]

Example 27B was prepared from Example 27A in a manner analogous to Example 26A. LCMS MH⁺ calculated, 519.1, found 519.3.

### Example 27C

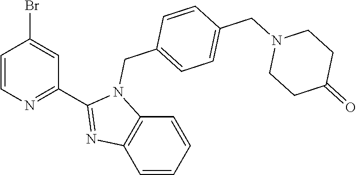 [SMILES: O=C1CCN(Cc2ccc(Cn3c(-c4cc(Br)ccn4)nc4ccccc43)cc2)CC1]

A solution of Example 27B (520 mg, 1 mmol) and p-toluenesulfonic acid monohydrate (1.52 g, 8 mmol) in dioxane (5 mL) and water (3 mL) was heated at 80° C. overnight. The reaction was cooled and partitioned between ethyl acetate and saturated NaHCO₃. The organic layer was further washed once with brine, dried (Na₂SO₄) and concentrated. Chromatography (SiO₂, 30→100% ethyl acetate in hexane) gave the target compound (334 mg, 70%) as a white solid. LCMS MH⁺ calculated, 475.1, found 493.3 (water adduct).

### Example 27D

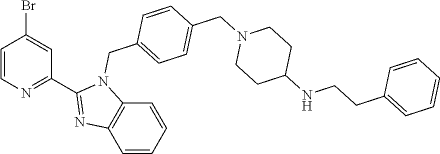 [SMILES: Brc1ccnc(-c2nc3ccccc3n2Cc2ccc(CN3CCC(NCCc4ccccc4)CC3)cc2)c1]

Example 27D was prepared from Example 27C in a manner analogous to Example 26A. LCMS MH⁺ calculated, 580.2, found 580.4.

### Example 27E

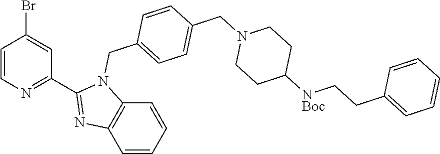 [SMILES: CC(C)(C)OC(=O)N(CCc1ccccc1)C1CCN(Cc2ccc(Cn3c(-c4cc(Br)ccn4)nc4ccccc43)cc2)CC1]

A mixture of Example 27D (380 mg, 0.66 mmol), di-tert-butyl dicarbonate (429 mg, 1.97 mmol) and Na₂CO₃ (209 mg, 1.97 mmol) in THF (2 mL) and water (2 mL) was refluxed for 3 h. The reaction mixture was cooled and partitioned between ethyl acetate and water. The organic layer was dried (Na₂SO₄) and concentrated. Chromatography (SiO₂, 10→50% ethyl acetate in hexane gave the target compound (345 mg, 77%) as a white solid. LCMS MH⁺ calculated, 680.3, found 680.5.

### Example 27F

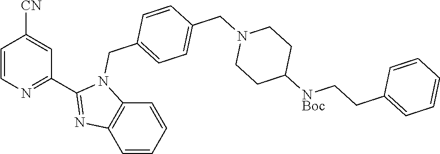 [SMILES: CC(C)(C)OC(=O)N(CCc1ccccc1)C1CCN(Cc2ccc(Cn3c(-c4cc(C#N)ccn4)nc4ccccc43)cc2)CC1]

Example 27F was prepared from Example 27E in a manner analogous to Example 19C. LCMS MH⁺ calculated, 627.3, found 627.6.

### Example 27G

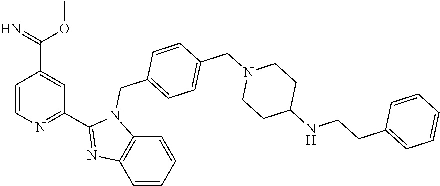 [SMILES: COC(=N)c1ccnc(-c2nc3ccccc3n2Cc2ccc(CN3CCC(NCCc4ccccc4)CC3)cc2)c1]

A solution of Example 27F (200 mg, 0.32 mmol) in MeOH (1 mL) was treated with HCl (2 mL of a 4 M solution in dioxane). The reaction was stirred for 3 h. The mixture was concentrated to give the target compound which was used directly in the next reaction without further purification. LCMS MH⁺ calculated, 559.3, found 559.5.

Example 27 was prepared from Example 27G in a manner analogous to that of Example 19. LCMS MH⁺ calculated, 546.3, found 546.6. ¹H NMR (DMSO-d6, 500 MHz) δ 8.75 (s, 1H), 8.65 (d, J=4.5 Hz, 1H), 7.79 (dd, J=1, 5 Hz, 18), 7.73 (m, 1H), 7.55 (m, 1H), 7.30 (m, 2H), 7.25 (m, 4H), 7.22 (m, 1H), 7.12 (d, J=8.5 Hz, 2H), 7.06 (d, J=8.5 Hz, 2H), 6.23 (s, 2H), 3.35 (br s, 3H), 3.10 (m, 2H), 2.94 (m, 3H), 2.75 (m, 2H), 1.90 (m, 4H), 1.54 (m, 2H).

### Example 28

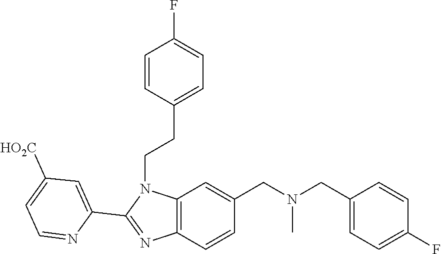 [SMILES: CN(Cc1ccc(F)cc1)Cc1ccc2nc(-c3cc(C(=O)O)ccn3)n(CCc3ccc(F)cc3)c2c1]

### Example 28A

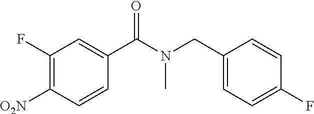 [SMILES: CN(Cc1ccc(F)cc1)C(=O)c1ccc([N+](=O)[O-])c(F)c1]

A suspension of 3-fluoro-4-nitrobenzoic acid (5 g, 27 mmol) in thionyl chloride (16 mL) was refluxed under nitrogen for 6 h during which time the reaction became homogeneous. The reaction was cooled and the mixture concentrated. The mixture was azeotroped with toluene three times to ensure removal of excess thionyl chloride. The residue was dissolved in CH₂Cl₂ (20 mL), cooled to 0° C. and treated with Et₃N (3.64 g, 36 mmol). To this was added a CH₂Cl₂ (50 mL) solution of 4-fluoro-N-methylbenzyl amine (4.88 g, 35 mmol) dropwise. The cooling bath was removed and the reaction stirred overnight at RT. The mixture was thrice with HCl (1 M), twice with saturated NaHCO₃ and twice with brine. The organic layer was dried (Na₂SO₄) and concentrated to give the crude target compound (8.3 g) as a yellow oil. LCMS MH⁺ calculated, 307.1, found 307.3.

### Example 28B

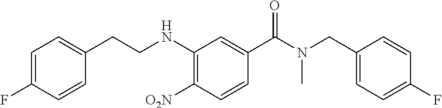 [SMILES: CN(Cc1ccc(F)cc1)C(=O)c1ccc([N+](=O)[O-])c(NCCc2ccc(F)cc2)c1]

A mixture of 4-fluorophenethylamine (2.64 g, 19 mmol), Example 28A (4.47 g, 14.6 mmol) and K₂CO. (6.05 g, 43.8 mmol) in DMA (25 mL) was heated at 70° C. for 4 h. The reaction was cooled and the mixture partitioned between ethyl acetate and brine. The organic layer was further washed thrice with brine, dried (Na₂SO₄) and concentrated to give the crude target compound (6.3 g) as a light brown oil. LCMS MH⁺ calculated, 426.2, found 426.3.

### Example 28C

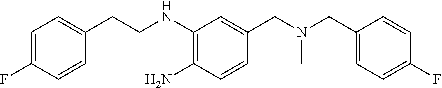 [SMILES: CN(Cc1ccc(F)cc1)Cc1ccc(N)c(NCCc2ccc(F)cc2)c1]

To a suspension of LAH (2.75 g, 72.5 mmol) in THF (40 mL) at 0° C. was added a solution of Example 28B (6.17 g, 14.5 mmol) in THF (30 mL) dropwise. The reaction mixture was allowed to warm to RT and was stirred for an additional 3 h. The mixture was re-cooled to 0° C., treated carefully with water (2.7 mL), then with NaOH (15%, 2.7 mL) and finally with more water (8.2 ml). The precipitate was filtered and the filter cake washed with ethyl acetate. The filtrate was dried (Na₂SO₄) and concentrated. Chromatography (SiO₂, 20→80% ethyl acetate in hexane) gave the target compound (2.1 g, 38%) as a brown oil. LCMS MW calculated, 382.2, found 382.4.

### Example 28D

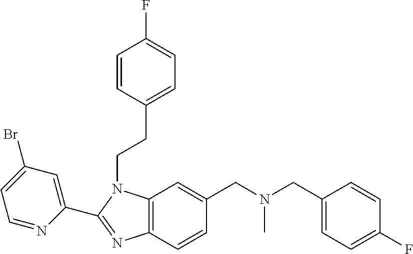 [SMILES: CN(Cc1ccc(F)cc1)Cc1ccc2nc(-c3cc(Br)ccn3)n(CCc3ccc(F)cc3)c2c1]

Example 28D was prepared from Example 28C in a manner analogous to Example 19A. LCMS MW calculated, 547.1, found 547.3.

### Example 28E

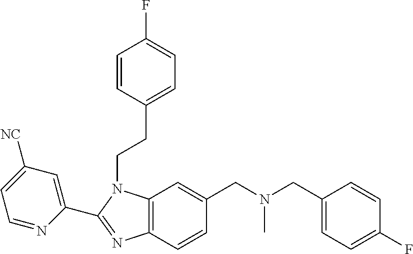 [SMILES: CN(Cc1ccc(F)cc1)Cc1ccc2nc(-c3cc(C#N)ccn3)n(CCc3ccc(F)cc3)c2c1]

Example 28E was prepared from Example 28D in a manner analogous to Example 19C. LCMS MH⁺ calculated, 494.2, found 494.4.

Example 28 was prepared from Example 28E in a manner analogous to Example 19. LCMS MW calculated, 513.2, found 513.4. ¹H NMR (DMSO-d6, 500 MHz) δ 8.71 (d, J=5 Hz, 1H), 8.63 (s, 1H), 7.79 (dd, J=1, 5 Hz, 1H), 7.64 (d, J=8.5 Hz, 1H), 7.44 (s, 1H), 7.41 (m, 2H), 7.25 (d, J=8.5 Hz, 1H), 7.17 (m, 4H), 6.99 (m, 2H), 4.99 (t, J=7 Hz, 2H), 3.61 (s, 2H), 3.50 (s, 2H), 3.08 (t, J=7 Hz, 2H), 2.09 (s, 3H).

### Example 29

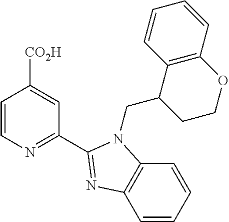 [SMILES: O=C(O)c1ccnc(-c2nc3ccccc3n2CC2CCOc3ccccc32)c1]

### Example 29A

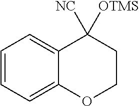

To a solution of 4-chromanone (2.96 g, 20 mmol) in CH₂Cl₂ (25 mL) at 0° C. was added trimethylsilyl cyanide (3.97 g, 40 mmol) as a solution in CH₂Cl₂ (10 mL) dropwise. The cold bath was removed and the reaction stirred at RT for 5 h. The reaction was diluted with additional CH₂Cl₂ (20 mL) and the mixture washed successively with saturated NaHCO₃ and brine. The organic layer was dried (Na₂SO₄) and concentrated to give the crude target compound (4.79 g, 97%0) as a light yellow oil which was used in the next step without further purification.

### Example 29B

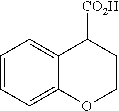 [SMILES: O=C(O)C1CCOc2ccccc21]

A mixture of Example 29A (4.75 g, 19.2 mmol) and SnCl₂ (10.92, 57.6 mmol) in HCl (37%, 20 mL) and AcOH (20 mL) was heated at reflux for 40 h. The residue was concentrated to remove AcOH and then partitioned between CH₂Cl₂ and brine. The organic layer was further washed with brine, dried (Na₂SO₄) and concentrated. Chromatography (SiO₂, 0-40% ethyl acetate in hexane) gave the target compound (3.18 g, 93%) as a white solid.

### Example 29C

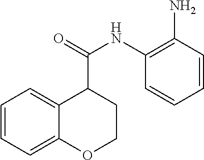 [SMILES: Nc1ccccc1NC(=O)C1CCOc2ccccc21]

To a solution of Example 29B (1.96 g, 11 mmol), o-phenylene diamine (3.57 g, 33 mmol) and N-methylmorpholine (3.34 g, 33 mmol) in DMF (20 mL) at 0° C. was added HATU (5.02 g, 13.2 mmol). The cooling bath was removed and the reaction stirred at RT for 5 h. Water was added to the reaction and a precipitate formed. The precipitate was collected by filtration, rinsed with water and dried in vacuo at 70° C. overnight to give the target compound (2.58 g, 87%) as a light yellow solid. LCMS MW calculated, 269.1, found 269.3.

### Example 29D

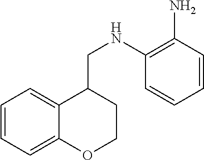 [SMILES: Nc1ccccc1NCC1CCOc2ccccc21]

To a solution of LAH (1.44 g, 37.9 mmol) in THF (35 mL) at 0° C. was added Example 29C portion-wise over a period of 20 min. The cooling bath was removed and the reaction stirred at RT for 5 h. The reaction was then diluted with THF (30 mL) and cooled to 0° C. Water (1.4 mL) was added dropwise followed by NaOH (15%, 1.4 mL) and then additional water (4.2 mL). The resulting precipitate was filtered and the filter cake washed with ethyl acetate. The filtrate was concentrated. Chromatography of the residue (SiO₂, 0→40% ethyl acetate in hexane) gave the target compound (953 mg, 39%) as a brown oil. LCMS MH⁺ calculated, 255.1, found 255.3.

### Example 29E

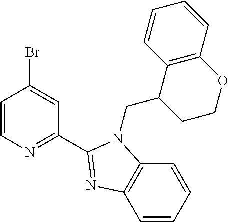 [SMILES: Brc1ccnc(-c2nc3ccccc3n2CC2CCOc3ccccc32)c1]

Example 29E was prepared from Example 29D in a manner analogous to Example 19A. LCMS MW calculated, 420.1, found 420.3.

### Example 29F

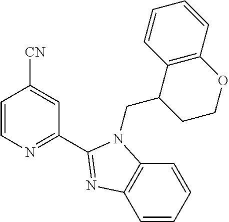 [SMILES: N#Cc1ccnc(-c2nc3ccccc3n2CC2CCOc3ccccc32)c1]

Example 29F was prepared in a manner analogous to Example 19C. LCMS MH⁺ calculated, 367.2, found 367.3.

Example 29 was prepared from Example 29F in a manner analogous to Example 19. LCMS MH⁺ calculated, 386.1, found 386.3. ¹H NMR (DMSO-d6, 500 MHz) δ 8.72 (m, 2H), 7.81 (dd, J=1.5, 5 Hz, 1H), 7.75 (m, 2H), 7.30 (m, 2H), 7.21 (dd, J=1, 7.5 Hz, 1H), 7.08 (t, J=7.5 Hz, 1H), 6.83 (t, J=7.5 Hz, 1H), 6.73 (dd, J=1, 8.5 Hz, 1H), 5.32 (dd, J=5.5, 14 Hz, 1H), 5.06 (dd, J=5.5, 14 Hz, 1H), 4.22 (m, 1H), 4.05 (m, 1H), 3.39 (m, 1H), 1.68 (m, 1H), 1.52 (m, 1H).

### Example 30

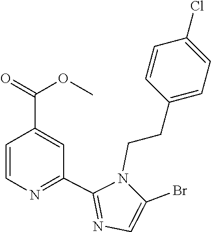 [SMILES: COC(=O)c1ccnc(-c2ncc(Br)n2CCc2ccc(Cl)cc2)c1]

To a solution of Example 8C (100 mg, 292.6 umol, 1 eq) in DCM (5 mL) was added NBS (52 mg, 292.6 umol, 1 eq) at 0° C. The mixture was stirred at 0-25° C. for 12 hours. The reaction was evaporated to remove the solvent to afford the crude product. The crude product was purified by flash chromatography, obtained the target compound as a yellow solid (80 mg, 65%). LCMS MH⁺ calculated, 420.0, found 420.0. ¹H NMR (400 MHz, CD₃OD) ppm 8.65 (d, J=5.2 Hz, 1H), 8.01 (d, J=0.8 Hz, 1H), 7.73 (dd, J=1.6, 5.1 Hz, 1H), 7.07 (s, 1H), 6.95-6.89 (m, 2H), 6.77-6.69 (m, 2H), 4.84 (t, J=6.8 Hz, 2H), 3.88 (s, 3H), 2.90 (t, J=6.8 Hz, 2H).

### Example 31

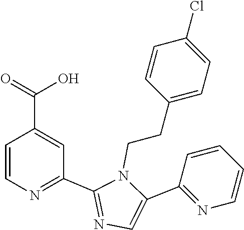 [SMILES: O=C(O)c1ccnc(-c2ncc(-c3ccccn3)n2CCc2ccc(Cl)cc2)c1]

To a solution of Example 30 (70 mg, 166.4 umol, 1 eq), tributyl (2-pyridyl)stannane (122.5 mg, 332.8 umol, 2 eq) in toluene (8 mL) was added Pd(PPh₃)₄ (67.3 mg, 58.2 umol, 0.35 eq). The mixture was heated to 120° C. for 18 hours. The reaction was filtered to remove the solid. The filtrate was concentrated under reduced pressure to remove solvent to afford the crude product. The crude product was isolated by Prep-TLC, then purified by Prep-HPLC to give the target compound as a white solid (15 mg, 25%). LCMS MH⁺ calculated, 405.1, found 405.1. ¹H NMR (400 MHz, CD₃OD) ppm 8.76 (d, J=4.8 Hz, 1H), 8.69 (d, J=4.0 Hz, 1H), 8.44 (s, 1H), 7.89-7.81 (m, 2H), 7.57 (d, J=8.0 Hz, 1H), 7.46 (s, 1H), 7.35 (dd, J=5.2, 7.2 Hz, 1H), 7.09 (d, J=8.2 Hz, 2H), 6.90 (d, J=8.2 Hz, 2H), 5.27 (t, 1=7.2 Hz, 2H), 3.00 (t, J=7.2 Hz, 2H).

### Example 32

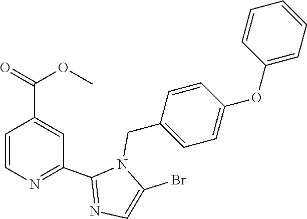 [SMILES: COC(=O)c1ccnc(-c2ncc(Br)n2Cc2ccc(Oc3ccccc3)cc2)c1]

To a solution of Example 17D (100 mg, 259.5 umol, 1 eq) in DCM (5 mL) was added NBS (46.2 mg, 259.5 umol, 1 eq) at 0° C. The reaction was warmed to 25° C. and stirred for 12 hours. The reaction was evaporated to remove the solvent and afford the crude product. The crude product was purified by flash chromatography to obtain the target compound as a white solid (95 mg, 78.9%). LCMS MH+ calculated, 464.1, found 463.9.

### Example 33

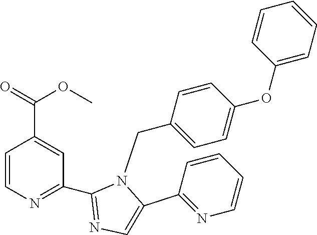 [SMILES: COC(=O)c1ccnc(-c2ncc(-c3ccccn3)n2Cc2ccc(Oc3ccccc3)cc2)c1]

To a solution of Example 32 (70 mg, 150.8 umol, 1 eq) in toluene (6 mL) was added Pd(PPh₃)₄ (52.3 mg, 45.2 umol, 0.30 eq). The mixture was heated to 120° C. for 16 hours. The reaction was filtered to remove the solid. The filtrate was concentrated under vacuum to afford the crude product. The crude product was purified by Prep-TLC, to obtain the target compound as a yellow solid (40 mg, 57.4%). LCMS MH+ calculated, 463.2, found 463.2.

### Example 34

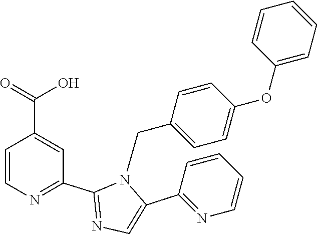 [SMILES: O=C(O)c1ccnc(-c2ncc(-c3ccccn3)n2Cc2ccc(Oc3ccccc3)cc2)c1]

To a solution of Example 33 (40 mg, 86.5 umol, 1 eq) in MeOH (2 mL) was added 1 mL NaOH solution (2 M). The mixture was stirred at 25° C. for 1 hour. The reaction was adjusted to pH 7 and concentrated under vacuum to afford the crude product. The crude product was purified by Prep-HPLC to obtain the target compound as a white solid (15 mg, 38.7%). LCMS MH+ calculated, 449.2, found 449.2. ¹H NMR (400 MHz, CD₃OD) ppm 8.70 (d, J=4.4 Hz, 1H), 8.65 (d, J=4.0 Hz, 1H), 8.47-8.40 (m, 1H), 7.88-7.79 (m, 2H), 7.66 (d, J=8.0 Hz, 1H), 7.54 (s, 1H), 7.36-7.24 (m, 3H), 7.10-7.01 (m, 1H), 6.85-6.74 (m, 4H), 6.67 (d, J=8.8 Hz, 2H), 6.36 (s, 2H).

### Example 35

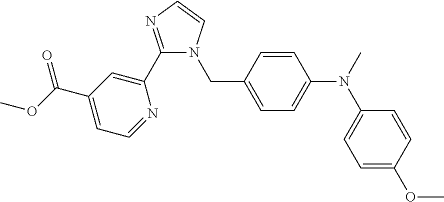

### Example 35A

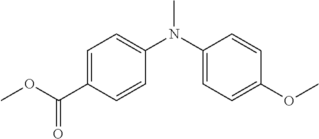

To a solution of methyl 4-bromobenzoate (1.50 g, 6.98 mmol, 1 eq) in dioxane (5 mL) was added 4-methoxy-N-methyl-aniline (976.7 mg, 7.12 mmol, 1.02 eq), Cs₂CO₃ (4.55 g, 13.95 mmol, 2 eq), Pd(OAc)₂ (156.6 mg, 697.54 umol, 0.10 eq) and BINAP (868.7 mg, 1.40 mmol, 0.20 eq) at 20° C. The reaction was stirred at 90° C. for 12 hours. The reaction was filtered and concentrated in vacuo. The residue was purified by column chromatography to give the target compound (1.74 g, 91.9%) as a yellow oil. ¹H NMR (400 MHz, CD₃CL) δ 7.85 (d, J=6.8 Hz, 2H), 7.18-7.10 (m, 2H), 6.95 (d, J=6.0 Hz, 2H), 6.65 (d, J=6.8 Hz, 2H), 3.85 (d, J=4.4 Hz, 6H), 3.32 (s, 3H).

### Example 35B

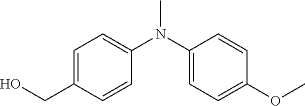 [SMILES: COc1ccc(N(C)c2ccc(CO)cc2)cc1]

To a solution of Example 35A (1.74 g, 6.4 mmol, 1 eq) in THF (10 mL) was added LiAlH₄ (243.3 mg, 6.41 mmol, 1 eq) at 0° C. The reaction was stirred at 0° C. for 1 hour. To this reaction was added H₂O (1 mL), NaOH (15%, 1 mL), H₂O (3 mL) and MgSO₄ and filtered. The organic layer was concentrated in vacuo to give the target compound (1.02 g, 4.19 mmol, 65.4%) as an oil. ¹H NMR (400 MHz, CD₃Cl) δ=7.21 (d, J=8.8 Hz, 2H), 7.10 (d, J=8.8 Hz, 2H), 6.91 (d, J=8.8 Hz, 2H), 6.77 (d, J=8.8 Hz, 2H), 4.58 (s, 2H), 3.83 (s, 3H), 3.27 (s, 3H).

### Example 35C

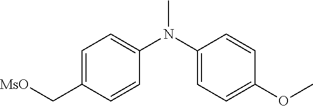 [SMILES: COc1ccc(N(C)c2ccc(COS(C)(=O)=O)cc2)cc1]

To a solution of Example 35B (200 mg, 822 umol, 1 eq) in DCM (2 mL) was added MsCl (113 mg, 986.4 umol, 76.4 uL, 1.20 eq) and Et₃N (166.4 mg, 1.64 mmol, 227.9 uL, 2 eq) at 0° C. The reaction was stirred at 20° C. for 0.5 hours. The reaction was diluted with DCM (10 mL) and washed with H₂O (10 mL). The combined organic phase was dried with anhydrous Na₂SO₄, filtered and concentrated in vacuo to give the target compound (300 mg, crude) as green oil which was used directly in the next step.

### Example 35D

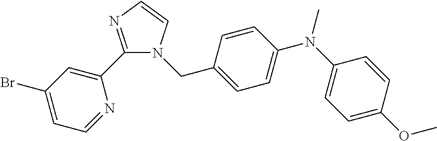 [SMILES: COc1ccc(N(C)c2ccc(Cn3ccnc3-c3cc(Br)ccn3)cc2)cc1]

To a solution of 4-bromo-2-(1H-imidazol-2-yl)pyridine ([1211579-82-6], 110 mg, 490.9 umol, 1 eq) in DMF (2 mL) was added K₂CO₃ (135.7 mg, 981.9 umol, 2 eq) and Example 35C (205.1 mg, 638.2 umol, 1.30 eq) at 20° C. The reaction was stirred at 100° C. for 4 hours. To this reaction was added H₂O (10 mL) and extracted with ethyl acetate (20 mL*2). The combined organic phase was washed with brine (20 mL), dried with anhydrous Na₂SO₄, filtered and concentrated in vacuo. The residue was purified by prep-TLC to give the target compound (80 mg, 36.3%) as yellow oil. ¹H NMR (400 MHz, CD₃Cl) δ 8.41-8.36 (m, 2H), 7.38 (s, 1H), 7.15 (d, J=0.8 Hz, 1H), 7.07 (d, J=9.2 Hz, 2H), 7.05-6.99 (m, 3H), 6.89 (d, J=8.8 Hz, 2H), 6.68 (d, J=8.8 Hz, 2H), 5.75 (s, 2H), 3.81 (s, 3H), 3.23 (s, 3H).

### Example 35

To a solution of Example 35D (80 mg, 178 umol, 1 eq) in methanol (5 mL) was added Et₃N (270.2 mg, 2.67 mmol, 370.2 uL, 15 eq) and Pd(dppf)Cl₂ (19.5 mg, 26.7 umol, 0.15 eq). The resulting mixture was stirred at 80° C. for 12 hours under a CO (50 psi) atmosphere. The reaction was filtered and concentrated in vacuo. The residue was purified by prep-TLC to give the target compound (70 mg, 91.8%) as yellow oil. ¹H NMR (400 MHz, CD₃Cl) 58.72-8.70 (m, 2H), 7.77 (dd, J=1.6, 3.2 Hz, 1H), 7.17 (s, 1H), 7.09-6.99 (m, 5H), 6.88 (d, J=8.8 Hz, 2H), 6.67 (d, J=9.2 Hz, 2H), 5.76 (s, 2H), 3.96 (s, 3H), 3.80 (s, 3H), 3.22 (s, 3H).

### Example 36

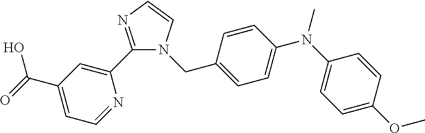 [SMILES: COc1ccc(N(C)c2ccc(Cn3ccnc3-c3cc(C(=O)O)ccn3)cc2)cc1]

To a solution of Example 35 (66 mg, 154 umol, 1 eq) in methanol (2 mL) was added NaOH (2 M, 77 uL, 1 eq) at 20° C. The reaction was stirred at 20° C. for 1 hour. The reaction was adjusted to pH 3 with HCl (2M) and the resulting solid was collected by filtration and dried in vacuo to give the target compound (40 mg, 58.2%) as a yellow solid. LCMS (negative ion mode) M-H calculated, 413.2, found 413.3. ¹H NMR (400 MHz, CD₃OD) δ 8.81 (s, 1H), 8.45 (s, 1H), 7.89 (d, J=4.4 Hz, 1H), 7.35 (s, 1H), 7.22 (s, 1H), 7.05-6.99 (m, 4H), 6.90 (d, J=9.2 Hz, 2H), 6.63 (d, J=8.4 Hz, 2H), 5.72 (s, 2H), 3.78 (s, 3H), 3.17 (s, 3H).

### Example 37

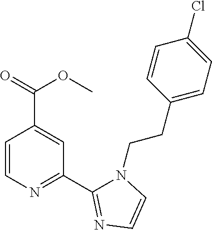 [SMILES: COC(=O)c1ccnc(-c2nccn2CCc2ccc(Cl)cc2)c1]

### Example 37A

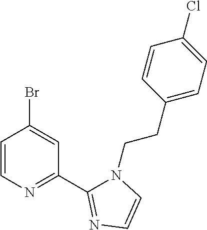 [SMILES: Clc1ccc(CCn2ccnc2-c2cc(Br)ccn2)cc1]

To a solution of 4-bromo-2-(1H-imidazol-2-yl)pyridine ([1211579-82-6], 400 mg, 1.79 mmol, 1 eq) in DMF (20 mL) was added Example 5A (840 mg, 3.58 mmol, 2 eq) and K₂CO₃ (494.79 mg, 3.58 mmol, 2.00 eq). The reaction was stirred at 80° C. for 12 hours. To this reaction was added H₂O (50 mL) and extracted with ethyl acetate (30 mL*2). The residue was purified by column chromatography to give the target compound (400 mg, 61.5%) as yellow oil. ¹H NMR (400 MHz, CD₃OD) 8.47 (d, J=5.2 Hz, 1H), 7.96 (d, J=1.6 Hz, 1H), 7.57 (dd, J=1.6, 3.6 Hz, 1H), 7.22 (d, J=1.2 Hz, 1H), 7.14 (d, J=8.4 Hz, 2H), 7.07 (d, J=1.2 Hz, 1H), 6.95 (d, J=8.4 Hz, 2H), 4.85 (t, J=6.8 Hz, 2H), 4.43 (t, J=6.4 Hz, 2H).

### Example 37

To a solution of Example 37A (200 mg, 551.5 umol, 1 eq) in MeOH (10 mL) was added Pd(dppf)Cl₂ (60.5 mg, 82.7 umol, 0.15 eq) and Et₃N (558 mg, 5.52 mmol, 764.5 uL, 10 eq) at 20° C. The reaction was stirred at 80° C. under a CO (50 psi) atmosphere for 10 hours. The reaction was concentrated in vacuo. The residue was purified by prep-TLC to give the target compound (30 mg, 15.9%) as a yellow solid. LCMS MH+ calculated, 342.1, found 342.0. ¹H NMR (400 MHz, CD₃OD) 8.73 (d, J=4.8 Hz, 1H), 8.20 (s, 1H), 7.79 (d, J=4.8 Hz, 1H), 7.21 (s, 1H), 7.06-7.03 (m, 3H), 6.87 (d, J=8.0 Hz, 2H), 4.81 (t, J=6.8 Hz, 2H), 3.97 (s, 3H), 2.99 (t, J=6.8 Hz, 2H).

### Example 38

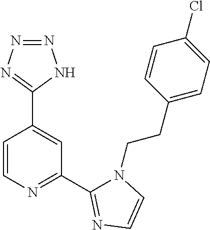 [SMILES: Clc1ccc(CCn2ccnc2-c2cc(-c3nnn[nH]3)ccn2)cc1]

### Example 38A

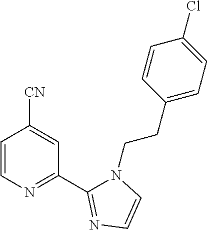 [SMILES: N#Cc1ccnc(-c2nccn2CCc2ccc(Cl)cc2)c1]

To a solution of Example 37A (100 mg, 275.8 umol, 1 eq) in DMF (5 mL) was added Zn(CN)₂ (35.6 mg, 303.3 umol, 19.25 uL, 1.10 eq), Pd₂ (dba)₃ (12.6 mg, 13.8 umol, 0.05 eq) and DPPF (15.3 mg, 27.6 umol, 0.10 eq). The reaction was stirred at 100° C. for 10 hours. To this reaction was added H₂O (20 mL) and extracted with ethyl acetate (20 mL*2). The combined organic phases were washed with brine (20 mL), dried with anhydrous Na₂SO₄, filtered and concentrated in vacuo. The residue was purified by prep-TLC to give the target compound (13.5 mg, 15.8%) as colorless oil. LCMS MH+ calculated, 309.1, found 309.0. ¹H NMR (400 MHz, CD₃OD) δ 8.68 (d, =4.8 Hz, 1H), 7.89 (s, 1H), 7.50 (dd, J=1.6, 3.2 Hz, 1H), 7.14 (d, J=0.8 Hz, 1H), 7.03-6.97 (m, 3H), 6.82 (d, J=8.4 Hz, 2H), 4.74 (t, J=7.2 Hz, 2H), 2.93 (t, J=7.2 Hz, 2H).

### Example 38

To a solution of Example 38A (120 mg, 388.7 umol, 1 eq) in n-PrOH (5 mL) was added NaN₃ (30.3 mg, 466.4 umol, 16.4 uL, 1.20 eq) and ZnCl₂ (53 mg, 388.7 umol, 18.2 uL, 1 eq). The reaction was stirred at 95° C. for 2 hours under N₂. The reaction was concentrated and NaOH (2 mL, 5%) was added. The mixture was filtered and the filtrated was adjusted to pH 3 with HCl (2 M) and then filtered. The residue was purified by prep-HPLC to give the target compound (42 mg, 30.6%) as a white solid. LCMS MH+ calculated, 352.1, found 352.0. ¹H NMR (400 MHz, CD₃OD) δ 9.00 (d, J=4.8 Hz, 1H), 8.28-8.26 (m, 2H), 7.88 (d, J=2.0 Hz, 1H), 7.79 (d, J=1.6 Hz, 1H), 7.07 (d, J=8.4 Hz, 2H), 6.94 (d, J=8.4 Hz, 2H), 5.07 (t, J=6.4 Hz, 2H), 3.19 (t, J=6.4 Hz, 2H).

### Example 39

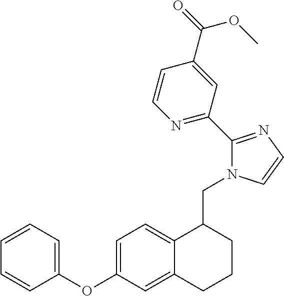 [SMILES: COC(=O)c1ccnc(-c2nccn2CC2CCCc3cc(Oc4ccccc4)ccc32)c1]

### Example 39A

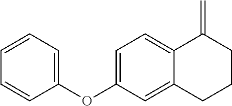 [SMILES: C=C1CCCc2cc(Oc3ccccc3)ccc21]

A mixture of 6-phenoxy-3,4-dihydronaphthalen-1 (2H)-one ([90401-82-4], 723 mg, 3.03 mmol, 1 eq), methyl triphenylphosphonium bromide (1.30 g, 3.64 mmol, 1.20 eq) and t-BuOK (680 mg, 6.06 mmol, 2 eq) in dry THF (10 mL) was heated to 50° C. with stirring for 12 hours under N₂. The reaction mixture was quenched with ice water (20 mL) and the mixture was extracted with ethyl acetate (20 mL*3). The combined organic layers were washed with brine (20 mL) and dried over anhydrous Na₂SO₄. After filtration, the filtrate was concentrated under reduced pressure to afford the crude product (0.9 g) as red oil. The crude product was purified by silica gel chromatography to give the target compound (400 mg, 55.9%) as a colorless oil. 1H NMR (400 MHz, CDCl₃) δ 7.64-7.62 (d, J=8.0 Hz, 1H), 7.37-7.33 (m, 2H), 7.13-7.10 (m, 1H), 7.04-7.02 (m, 62H), 6.84-6.81 (m, 1H), 6.47 (m, 1H), 5.40 (s, 1H), 4.91 (s, 1H), 2.82-2.79 (t, J=10 Hz, 2H), 2.56-2.53 (t, J=6.0 Hz, 2H), 1.91-1.85 (m, 2H).

### Example 39B

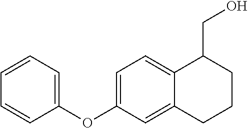 [SMILES: OCC1CCCc2cc(Oc3ccccc3)ccc21]

To a solution of Example 39A (400 mg, 1.69 mmol, 1 eq) in THF (2 mL) was added BH₃-Me₂S (10 M, 1.69 mL, 10 eq) dropwise at −78° C. The mixture was stirred at 25° C. for 3 hr and then cooled to −78° C. again. A mixture of NaOH (1.35 g, 33.8 mmol, 20 eq) dissolved in H₂O (1 mL) and H₂O₂ (3.83 g, 33.8 mmol, 3.25 mL, 30%, 20 eq) was added to the mixture and stirring was continued for 3 hr at 25° C. The reaction mixture was quenched with saturated Na₂SO₃ (15 mL). The mixture was extracted with ethyl acetate (10 mL*3). The combined organic layers were washed with saturated Na₂SO₃ (5 mL*3), water (10 mL), brine (10 mL) and dried over anhydrous Na₂SO₄. After filtration, the filtrate was concentrated under reduced pressure to afford the crude product (0.8 g) as colorless oil. The crude product was purified by Prep-TLC to obtain the target compound (270 mg, 1.06 mmol, 62.8%) as a colorless oil. ¹H NMR (400 MHz, CDC₃) δ 7.35-7.32 (m, 2H), 7.21-7.19 (d, J=8.0 Hz, 1H), 7.02-7.00 (d, J=8.0 Hz, 1H), 6.83-6.80 (m, 1H), 6.76-6.75 (m, 1H), 3.82-3.80 (d, J=8.0 Hz, 2H), 3.00-2.94 (m, 1H), 2.74-2.71 (m, 2H), 1.98-1.70 (m, 4H).

### Example 39C

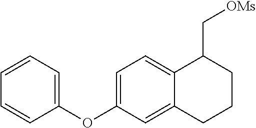 [SMILES: CS(=O)(=O)OCC1CCCc2cc(Oc3ccccc3)ccc21]

To a mixture of Example 39B (270 mg, 1.06 mmol, 1 eq) and Et₃N (322.3 mg, 3.18 mmol, 441.5 uL, 3 eq) in dry dichloromethane (5 mL) was added MsCl (182.4 mg, 1.59 mmol, 123.3 uL, 1.50 eq) below 0° C. After addition, the resulting mixture was allowed to warm to 25° C. with stirring for 3 hours. The reaction mixture was quenched with water (10 mL). The mixture was extracted with dichloromethane (10 mL*3). The combined organic layers were washed with water (10 mL), brine (10 mL) and dried over anhydrous Na₂SO₄. After filtration, the filtrate was concentrated under reduced pressure. The target compound (420 mg, 95.3%) was obtained as yellow oil and used in the next step without further purification.

### Example 39D

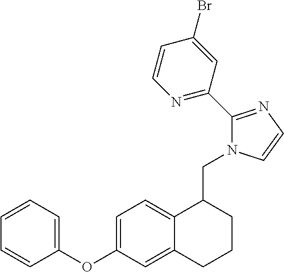 [SMILES: Brc1ccnc(-c2nccn2CC2CCCc3cc(Oc4ccccc4)ccc32)c1]

A suspension of Example 39C (498.6 mg, 1.20 mmol, 1 eq), 4-bromo-2-(1H-imidazol-2-yl)pyridine ([1211579-82-6], 268.9 mg, 1.20 mmol, 1 eq) and K₂CO₃ (331.7 mg, 2.40 mmol, 2 eq) in dry DMF (5 mL) was heated to 80° C. with stirring for 3 hours under N₂. The reaction mixture was cooled to room temperature and quenched with water (15 mL). The mixture was extracted with ethyl acetate (15 mL*3). The combined organic layers were washed with water (10 mL*2), brine (10 mL) and dried over anhydrous Na₂SO₄. After filtration, the filtrate was concentrated under reduced pressure to afford the crude product (0.6 g) as dark oil. The crude product was purified by Prep-TLC to afford the target compound (80 mg, 14.5%) was obtained as a yellow solid. LCMS MH+ calculated, 460.1, found, 460.0. ¹H NMR (400 MHz, CCl₃D) δ 8.33 (d, 1H), 8.28-8.27 (d, J=4.0 Hz, 1H), 7.24-7.16 (m, 4H), 7.06-6.92 (m, 4H), 6.88-6.85 (m, 4H), 6.67-6.59 (m, 3H), 5.08-5.03 (dd, J=8.0 Hz, J₂=16.0 Hz, 1H), 4.25-4.19 (dd, J₁=8.0 Hz, J₂=16.0 Hz, 1H), 3.70-3.60 (m, 1H), 3.24-3.16 (m, 1H), 2.91-2.80 (m, 1H), 2.65-2.51 (m, 3H), 1.77-1.49 (m, 5H).

### Example 39

To a solution of Example 39D (80 mg, 156.4 umol, 1 eq) in MeOH (5 mL) was added Et₃N (316.5 mg, 3.13 mmol, 433.6 uL, 20 eq) and Pd(dppf)C₂.CH₂Cl₂ (12.8 mg, 15.6 umol, 0.10 eq). The reaction was stirred at 80° C. under a CO (50 psi) atmosphere for 4 hours. The reaction mixture was cooled to room temperature and filtered. The filtrate was concentrated under reduced pressure. The target compound (90 mg, crude) was obtained as red solid. LCMS MH+ calculated, 440.2, found, 440.0.

### Example 40

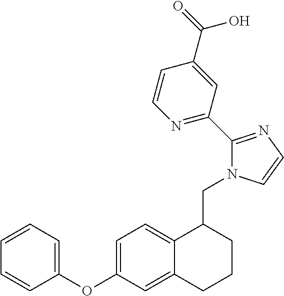 [SMILES: O=C(O)c1ccnc(-c2nccn2CC2CCCc3cc(Oc4ccccc4)ccc32)c1]

### Example 40

To a solution of Example 39 (90 mg, 204.8 umol, 1 eq) in a mixture of MeOH (5 mL) and H₂O (1 mL) was added NaOH (32.8 mg, 819 umol, 4 eq), the mixture was heated at 40° C. with stirring for 1 hour. The reaction mixture was cooled to room temperature and concentrated under reduced pressure. The residue was neutralized with HCl (2 M, 0.4 mL) to pH 7. The residue was purified by Prep-HPLC. The target compound (10 mg, 11%) was obtained as white solid. LCMS MH+ calculated, 426.2, found, 426.0. ¹H NMR (400 MHz, CD₃OD) δ 8.69-8.68 (d, J=4.0 Hz, 1H), 8.44 (s, 1H), 7.79-7.77 (dd, J₁=0.0 Hz, J₂=4.0 Hz, 1H), 7.34-7.29 (m, 2H), 7.27 (d, 1H), 7.09-7.02 (m, 3H), 6.92-6.90 (m, 2H), 6.70-6.68 (dd, J=0.0 Hz, J=2=8.0 Hz, 1H), 6.63-6.62 (m, 1H), 5.10-5.05 (dd, J₁=8.0 Hz, J₂=12.0 Hz, 1H), 4.51-4.45 (dd, J₁=8.0 Hz, J₂=12.0 Hz, 1H), 3.25-3.21 (m, 1H), 2.67-2.66 (m, 21H) 1.81-1.61 (m, 4H).

### Example 41

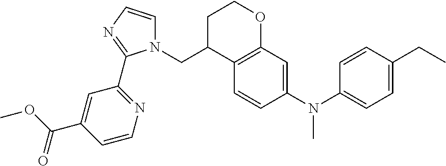 [SMILES: CCc1ccc(N(C)c2ccc3c(c2)OCCC3Cn2ccnc2-c2cc(C(=O)OC)ccn2)cc1]

### Example 41A

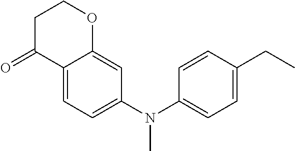 [SMILES: CCc1ccc(N(C)c2ccc3c(c2)OCCC3=O)cc1]

To a solution of N-methyl-4-ethylaniline ([37846-06-3], 2.00 g, 14.8 mmol, 1 eq.) and 7-Bromo-2,3-dihydro-4H-chromen-4-one ([18442-22-3], 4.03 g, 17.75 mmol, 1.20 eq.) in toluene (20 mL) was added Cs₂CO₃ (9.64 g, 29.6 mmol, 2 eq.), BINAP (736.8 mg, 1.18 mmol, 0.08 eq.) and Pd(OAc)₂ (265.7 mg, 1.18 mmol, 0.08 eq.). The mixture was stirred at 100° C. for 12 hours. The solvent was removed in vacuo to give the crude product which was purified by column chromatography to afford the target compound (1.70 g, 40.8%). LCMS MH+ calculated, 282.1, found, 282.2. ¹H NMR (400 MHz, CDC₃) δ 7.71 (d, J=8.8 Hz, 1H), 7.30-7.24 (m, 2H), 7.18-7.10 (m, 2H), 6.36 (d, J=8.8 Hz, 1H), 6.16 (d, J=2.4 Hz, 1H), 4.48 (t, J=6.4 Hz, 2H), 3.35 (s, 3H), 2.78-2.64 (m, 4H), 1.29 (t, J=7.8 Hz, 3H).

### Example 41B

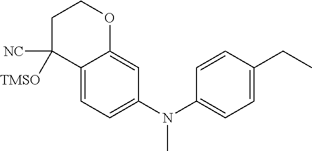

A mixture of Example 41A (1.50 g, 5.33 mmol, 1 eq), zinc iodide (88.5 mg, 277 umol, 0.05 eq) and TMSCN (1.59 g, 15.99 mmol, 2.01 mL, 3 eq) in toluene (15 mL) was stirred at 50° C. for 3 hour under N₂ atmosphere. The reaction was quenched with water, extracted with DCM (50 ml*2) and EtOAc (50 ml). The combine organic layers were concentrated to give the crude target compound (2.00 g) as a yellow solid which was used for the next step without further purification. LCMS MH+ calculated, 381.2, found, 381.1.

### Example 41C

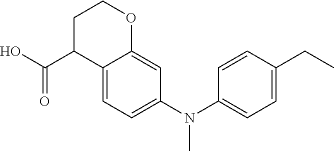 [SMILES: CCc1ccc(N(C)c2ccc3c(c2)OCCC3C(=O)O)cc1]

A mixture of Example 41B (2.00 g, 5.26 mmol, 1 eq) and SnCl₂.2H₂O (4.74 g, 21 mmol, 1.75 mL, 4 eq) in AcOH (5 mL) and concentrated HCl (5 ml) was stirred at 100° C. for 12 hours under N₂ atmosphere. The reaction was quenched with water, extracted with DCM (50 ml*3) and concentrated to give the crude product. The crude product was purified by column to afford the target compound (1.60 g, 97.7%) as a yellow solid. LCMS MH+ calculated, 312.2, found, 312.1.

### Example 41D

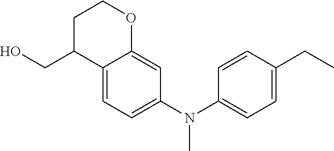 [SMILES: CCc1ccc(N(C)c2ccc3c(c2)OCCC3CO)cc1]

To a solution of Example 41C (2.00 g, 6.38 mmol, 1 eq) in THF (20 mL) was added LAH (975 mg, 25.7 mmol, 4 eq). The mixture was stirred at 25° C. for 2 hours under N₂ atmosphere. The reaction was quenched with water (0.5 ml), extracted with EtOAc (50 ml*3) and concentrated to give the crude product. The crude product was purified by column to afford the target compound (800 mg, 41.9%) as a colorless oil. LCMS MH+ calculated, 298.2, found, 298.0. ¹H NMR (400 MHz, CDCl₃) δ 7.07 (d, J=8.3 Hz, 2H), 6.99-6.88 (m, 3H), 6.37 (dd, J=2.4, 8.4 Hz, 1H), 6.30 (d, J=2.4 Hz, 1H), 4.65 (br s, 1H), 4.09 (dd, J=4.8, 6.1 Hz, 2H), 3.83-3.65 (m, 2H), 3.22-3.22 (m, 1H), 3.18 (s, 3H), 2.86 (br dd, J=5.3, 7.6 Hz, 1H), 2.55 (q, J=7.6 Hz, 2H), 2.06-1.91 (m, 3H), 1.40 (br t, J=5.2 Hz, 1H), 1.21-1.08 (m, 4H).

### Example 41E

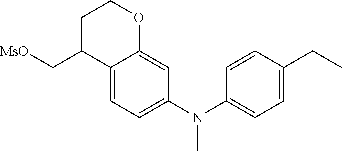 [SMILES: CCc1ccc(N(C)c2ccc3c(c2)OCCC3COS(C)(=O)=O)cc1]

1 (04241 To a solution of Example 41D (600 mg, 2.02 mmol, 1 eq) and triethylamine (245.3 mg, 2.42 mmol, 336 uL, 1.20 eq) in DCM (5 mL) was added MsCl (277.7 mg, 2.42 mmol, 187.6 uL, 1.20 eq). The mixture was stirred at 25° C. for 2 hour under N₂ atmosphere. The reaction was quenched with water and extracted with DCM (50 mL). The organic layer was concentrated to give the crude target compound (600 mg, 79.2%) as a yellow oil which was used in the next step immediately.

### Example 41F

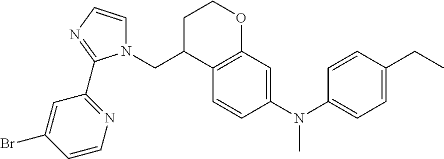 [SMILES: CCc1ccc(N(C)c2ccc3c(c2)OCCC3Cn2ccnc2-c2cc(Br)ccn2)cc1]

A mixture of Example 41E (600 mg, 1.60 mmol, 1 eq), 4-bromo-2-(1H-imidazol-2-yl)pyridine ([1211579-82-6], 358.5 mg, 1.60 mmol, 1 eq) and K₂CO₃ (221 mg, 1.60 mmol, 1 eq) in acetonitrile (5 mL) was stirred at 80° C. for 2 hour under N₂ atmosphere. The reaction was quenched with water and extracted with EtOAc (50 mL*2). The combined organic layers were concentrated to give the crude product which was purified by prep-TLC. The target compound (160 mg, 19.9%) was obtained as a yellow oil. LCMS MH+ calculated, 503.2, found, 503.1.

### Example 41

To a solution of Example 41F (160 mg, 317.8 umol, 1 eq) in MeOH (5 mL) was added Pd(dppf)C₁₂ (23.3 mg, 0.1 eq) and triethylamine (257.3 mg, 8 eq). The mixture was stirred at 80° C. for 10 hours under a CO atmosphere (50 psi). The solvent was removed in vacuo. The resulting residue was purified by prep-TLC to afford the target compound (80 mg, 52.2%) as a brown solid. LCMS MH+ calculated, 483.2, found, 483.1.

### Example 42

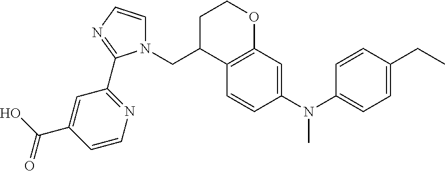 [SMILES: CCc1ccc(N(C)c2ccc3c(c2)OCCC3Cn2ccnc2-c2cc(C(=O)O)ccn2)cc1]

### Example 42

A mixture of Example 41 (80 mg, 165.8 umol, 1 eq) and NaOH (400 umol, 1.00 mL, 2.41 eq, 2 M) in MeOH (1 mL) was stirred at 25° C. for 0.5 hour under N₂ atmosphere. The MeOH was removed in vacuo and the mixture neutralized to pH 7. The resulting solid was collected by filtration and the filter cake washed with water. The solid was dried in vacuo to afford the target compound (20 mg, 25.8%) as a white solid. LCMS MH+ calculated, 469.2, found, 469.2. ¹H NMR (400 MHz, DMSO-d6) δ 8.81 (d, J=5.0 Hz, 1H), 8.50 (s, 1H), 7.74 (d, J=4.9 Hz, 1H), 7.50 (s, 1H), 7.23-7.07 (m, 3H), 7.01-6.78 (m, 3H), 6.35 (dd, J=2.3, 8.4 Hz, 1H), 6.15 (d, J=2.1 Hz, 1H), 5.16 (dd, J=5.5, 13.2 Hz, 1H), 4.54 (dd, J=10.2, 13.2 Hz, 1H), 4.22-3.97 (m, 2H), 3.14 (s, 4H), 2.57 (q, J=7.7 Hz, 2H), 1.85-1.55 (m, 2H), 1.18 (t, J=7.6 Hz, 3H).

### Example 43

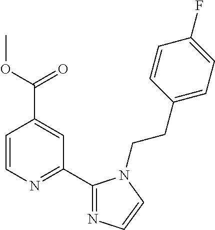 [SMILES: COC(=O)c1ccnc(-c2nccn2CCc2ccc(F)cc2)c1]

### Example 43A

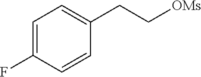 [SMILES: CS(=O)(=O)OCCc1ccc(F)cc1]

To a solution of 4-fluorophenethyl alcohol (1.40 g, 10 mmol, 1.25 mL, 1 eq), Et₃N (3.04 g, 30 mmol, 4.16 mL, 3 eq) in DCM (20 mL) was added MsCl (1.49 g, 13 mmol, 1.01 mL, 1.30 eq) at 0° C. The mixture was stirred at 0° C. for 30 min. Water (10 mL) was added to the mixture and the organic layer isolated. The water layer was further extracted with DCM (10 mL*2). The combined organic layers were dried over anhydrous sodium sulfate and concentrated in vacuo to afford the target compound as a yellow oil (1.8 g, 83.5%). ¹H NMR (400 MHz, CD₃OD) δ 7.33-7.25 (m, 2H), 7.07-7.00 (m, 2H), 4.40 (t, J=6.8 Hz, 2H), 3.02 (t, J=6.8 Hz, 2H), 2.95 (s, 3H).

### Example 43B

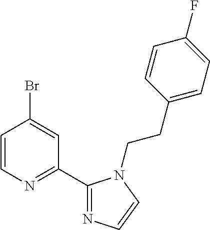 [SMILES: Fc1ccc(CCn2ccnc2-c2cc(Br)ccn2)cc1]

To a solution of Example 43A (759.7 mg, 3.48 mmol, 1.30 eq) and 4-bromo-2-(1H-imidazol-2-yl)pyridine ([1211579-82-6], 600 mg, 2.68 mmol, 1 eq) in DMF (8 mL) was added K₂CO₃ (925.3 mg, 6.69 mmol, 2.5 eq). The mixture was heated to 80° C. for 8 hours. The reaction mixture was filtered and the filtrate was evaporated under reduced pressure to afford the crude product. The crude product was purified by flash chromatography on silica gel to give the target compound as a yellow solid (780 mg, 84.1%). LCMS MH+ calculated, 346.0, found, 345.9. ¹H NMR (400 MHz, CD₃OD) δ=8.45 (d, J=5.2 Hz, 1H), 7.95 (d, J=1.6 Hz, 1H), 7.54 (dd, J=2.0, 5.2 Hz, 1H), 7.19 (d, J=1.2 Hz, 1H), 7.04 (d, J=1.2 Hz, 1H), 6.99-6.93 (m, 2H), 6.89-6.83 (m, 2H), 4.80 (t, J=7.2 Hz, 2H), 3.02 (t, J=7.2 Hz, 2H).

### Example 43

To a solution of Example 43B (780 mg, 2.25 mmol, 1 eq) in Et₃N (1.82 g, 18 mmol, 2.50 mL, 8 eq) and MeOH (20 mL) was added Pd(dppf)C₁₂ (247.3 mg, 338 umol, 0.15 eq). The mixture was purged with carbon monoxide 6 times and stirred for 12 hours under a carbon monoxide (50 psi.) atmosphere at 80° C. After cooling, the solid was filtered and the filtrate evaporated under reduced pressure to afford the crude product. The crude product was purified by flash chromatography on silica gel to obtain the target compound as a white solid (650 mg, 88.8%). LCMS MH+ calculated, 326.1, found, 326.0. ¹H NMR (400 MHz, CD₃OD) δ=8.79 (dd, J=0.4, 5.0 Hz, 1H), 8.28 (d, J=0.8 Hz, 1H), 7.83 (dd, J=1.6, 5.2 Hz, 1H), 7.23 (d, J=1.2 Hz, 1H), 7.08 (d, J=1.2 Hz, 1H), 7.01-6.94 (m, 2H), 6.88-6.81 (m, 2H), 4.83 (t, J=7.2 Hz, 2H), 4.02-3.97 (m, 3H), 3.04 (t, J=7.2 Hz, 2H).

### Example 44

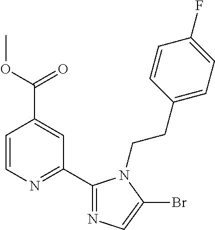 [SMILES: COC(=O)c1ccnc(-c2ncc(Br)n2CCc2ccc(F)cc2)c1]

### Example 44

To a solution of Example 43 (600 mg, 1.84 mmol, 1 eq) in DCM (20 mL) was added NBS (328.2 mg, 1.84 mmol, 1 eq). The mixture was stirred at 25° C. for 4 hours. After removal of the solvent in vacuo, the crude product was purified by flash chromatography on silica gel to give the target compound as a white solid (0.6 g, 80.7%). LCMS MH+ calculated, 404.0, found, 403.9. ¹H NMR (400 MHz, CDCl₃) δ 8.75 (dd, J=0.4, 5.0 Hz, 1H), 8.57 (d, J=0.8 Hz, 1H), 7.81 (dd, J=1.6, 5.2 Hz, 1H), 7.19 (s, 1H), 7.11-7.05 (m, 2H), 6.95-6.88 (m, 2H), 4.90-4.82 (m, 2H), 3.98 (s, 3H), 3.08-3.02 (m, 2H).

### Example 45

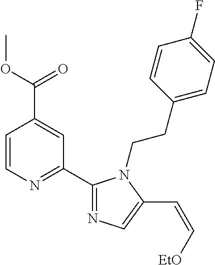 [SMILES: CCO/C=C\c1cnc(-c2cc(C(=O)OC)ccn2)n1CCc1ccc(F)cc1]

### Example 45

To a solution of Example 44 (120 mg, 296.9 umol, 1 eq) and (Z)-tributyl(2-ethoxyvinyl)stannane (128.7 mg, 356.2 umol, 1.20 eq) in toluene (2 mL) was added Pd(PPh₃)₄ (34.3 mg, 29.7 umol, 0.10 eq). The mixture was stirred at 120° C. under N₂ atmosphere for 12 hours. After cooling to room temperature, aqueous KF (5 mL) was added to the reaction and stirring continued for 1 hour. The resulting solution was extracted with EtOAc (5 mL*3). The combined organic layers were dried over sodium sulfate and the solvent removed in vacuo to give the crude product. The crude product was purified by Prep-TLC to afford the target compound as a yellow oil (90 mg, 76.7%). LCMS MH+ calculated, 396.2, found, 396.2. ¹H NMR (400 MHz, CD₃OD) δ 8.72 (d, J=5.2 Hz, 1H), 8.12 (s, 1H), 7.76 (d, J=5.2 Hz, 1H), 7.43 (s, 1H), 6.91-6.84 (m, 2H), 6.82-6.73 (m, 2H), 6.53 (d, J=6.8 Hz, 1H), 5.35 (d, J=6.8 Hz, 1H), 4.80 (br t, J=6.8 Hz, 2H), 4.10 (q, J=7.2 Hz, 2H), 3.96 (s, 3H), 2.94 (br t, J=6.8 Hz, 2H), 1.43-1.38 (m, 3H).

### Example 46

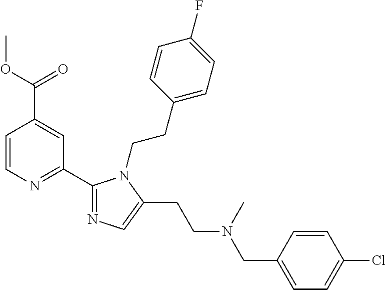 [SMILES: COC(=O)c1ccnc(-c2ncc(CCN(C)Cc3ccc(Cl)cc3)n2CCc2ccc(F)cc2)c1]

### Example 46

To a solution of Example 45 (30 mg, 75.9 umol, 1 eq) in CH₃CN (1 mL) was added TMSCl (24.7 mg, 227.6 umol, 28.8 uL, 3 eq) and NaI (34.1 mg, 227.6 umol, 3 eq) successively. The mixture was stirred at 25° C. for 15 min. 1-(4-chlorophenyl)-N-methyl-methanamine (18.9 mg, 121.4 umol, 1.20 eq) was then added and stirring continued for a further 10 min. NaBH(OAc)₃ (64.3 mg, 303.5 umol, 3 eq) was added and stirring continued for 2 hours. The residue was diluted with water (10 mL) and extracted with EtOAc (5 mL*3). The combined organic layers were dried and the solvent removed in vacuo to afford the crude product. The crude product was purified by Prep-TLC on silica gel to give the target compound as a yellow solid (30 mg, 58.5%). LCMS MH+ calculated, 507.2, found, 507.2. ¹H NMR (400 MHz, CD₃OD) δ 8.81-8.71 (m, 1H), 8.19 (br s, 1H), 7.86 (br d, J=4.4 Hz, 1H), 7.54-7.46 (m, 8H), 7.39 (d, J=8.4 Hz, 2H), 4.75 (br t, J=6.8 Hz, 2H), 4.21 (s, 4H), 4.06 (s, 2H), 4.00-3.93 (m, 3H), 3.21-3.12 (m, 2H), 3.05-2.92 (m, 4H).

### Example 47

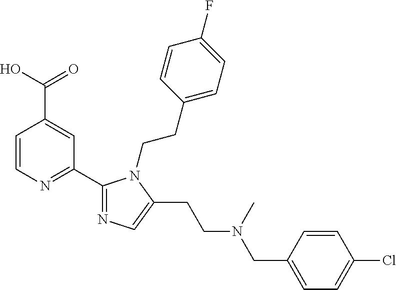 [SMILES: CN(CCc1cnc(-c2cc(C(=O)O)ccn2)n1CCc1ccc(F)cc1)Cc1ccc(Cl)cc1]

### Example 47

To a solution of Example 46 (30 mg, 59.2 umol, 1 eq) in MeOH (2 mL) was added 1 mL NaOH (2 M) solution. The mixture was stirred at 25° C. for 2 hours. After adjusting the pH to 7 by addition of HCl (2 M) solution, the resulting residue was purified by Prep-HPLC to obtain the target compound as a yellow solid (15 mg, 51.4%). LCMS MH+ calculated, 493.2, found, 493.3. ¹H NMR (400 MHz, CD₃OD) δ 8.69 (br d, J=4.4 Hz, 1H), 8.36 (s, 1H), 7.79 (br d, J=4.0 Hz, 1H), 7.31 (s, 4H), 7.04-6.98 (m, 2H), 6.96-6.89 (m, 2H), 6.84 (s, 1H), 4.65 (br t, J=7.2 Hz, 2H), 3.56 (s, 2H), 2.92 (br t, J=7.2 Hz, 2H), 2.66 (s, 4H), 2.29 (s, 3H).

### Example 48

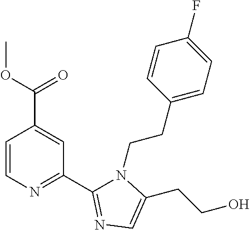 [SMILES: COC(=O)c1ccnc(-c2ncc(CCO)n2CCc2ccc(F)cc2)c1]

### Example 48

To a solution of Example 45 (150 mg, 379.3 umol, 1 eq) in CH₃CN (2 mL) was added TMSCl (123.6 mg, 1.14 mmol, 143.7 uL, 3 eq) and NaI (170.6 mg, 1.14 mmol, 3 eq) successively. The mixture was stirred at 25° C. for 30 min, then NaBH(OAc)₃ (241 mg, 1.14 mmol, 3 eq) was added to the solution. The mixture was stirred for a further 2 hours. The reaction was quenched by adding 5 mL water. The mixture was extracted with EtOAc (5 mL*3) and the combined organic layers dried over sodium sulfate. The solvent was removed in vacuo to afford the crude product which was purified by Prep-TLC on silica gel to give the target compound as a white solid (50 mg, 35.7%). LCMS MH+ calculated, 370.2, found, 370.0. ¹H NMR (400 MHz, CD₃OD) δ 8.77 (br d, J=11.2 Hz, 1H), 8.26-8.14 (m, 1H), 7.91-7.76 (m, 1H), 7.09 (s, 1H), 7.04-6.89 (m, 3H), 6.86-6.75 (m, 2H), 4.81 (br t, J=7.2 Hz, 2H), 3.98 (s, 3H), 3.85 (t, J=6.4 Hz, 2H), 2.97 (br t, J=6.8 Hz, 2H), 2.84 (t, J=6.4 Hz, 2H).

### Example 49

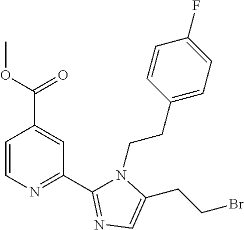 [SMILES: COC(=O)c1ccnc(-c2ncc(CCBr)n2CCc2ccc(F)cc2)c1]

### Example 49

To a solution of Example 48 (60 mg, 162.4 umol, 1 eq) and CBr₄ (215.5 mg, 649.7 umol, 4 eq) in DCM (3 mL) was added PPh₃ (170.4 mg, 649.7 umol, 4 eq). The mixture was stirred at 35° C. for 36 hours. After removal of the solvent in vacuo, the residue was purified by Prep-TLC on silica gel to give the target compound as a yellow solid (50 mg, 71.2%). LCMS MH+ calculated, 432.1, found, 432.0. ¹H NMR (400 MHz, CD₃OD) δ 8.76 (d, J=4.8 Hz, 1H), 8.19 (s, 1H), 7.85-7.80 (m, 1H), 7.04 (s, 1H), 6.92-6.86 (m, 2H), 6.84-6.74 (m, 2H), 4.80 (t, J=6.8 Hz, 2H), 3.98 (s, 3H), 3.68 (t, J=7.2 Hz, 2H), 3.15 (t, J=7.2 Hz, 2H), 2.97 (t, J=6.8 Hz, 2H).

### Example 50

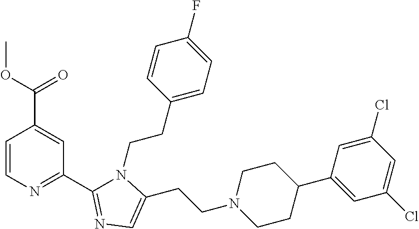 [SMILES: COC(=O)c1ccnc(-c2ncc(CCN3CCC(c4cc(Cl)cc(Cl)c4)CC3)n2CCc2ccc(F)cc2)c1]

### Example 50

To a solution of Example 49 (60 mg, 138.8 umol, 1 eq) and 4-(3,5-dichlorophenyl)piperidine (Bavetsias et al., J. Med. Chem. 2016, 59, 1388-1409, 51.1 mg, 222 umol, 1.60 eq) in CH₃CN (1 mL) was added K₂CO₃ (57.6 mg, 416.4 umol, 3 eq). The mixture was stirred at 80° C. for 12 hours. After cooling to room temperature, the reaction mixture was concentrated. The residue was purified by Prep-TLC on silica gel to afford the target compound as a white solid (25 mg, 30.5%). LCMS MH+ calculated, 581.2, found, 581.2.

### Example 51

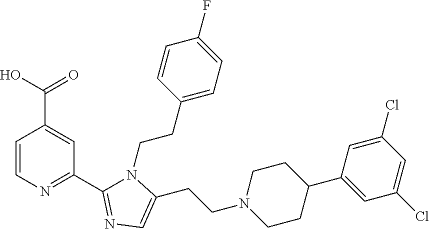 [SMILES: O=C(O)c1ccnc(-c2ncc(CCN3CCC(c4cc(Cl)cc(Cl)c4)CC3)n2CCc2ccc(F)cc2)c1]

### Example 51

To a solution of Example 50 (25 mg, 43 umol, 1 eq) in MeOH (1 mL) was added NaOH (4 M, 430 uL, 40 eq) solution. The mixture was stirred at 25° C. for 1 hour. After adjusting the pH to 7 by adding 2 M HCl solution, the residue was concentrated in vacuo. The residue was purified by Prep-HPLC to give the target compound as a pink solid (20 mg, 82.0%). LCMS MH+ calculated, 567.2, found, 567.0. ¹H NMR (400 MHz, CD₃OD) δ 8.68 (br s, 1H), 8.31 (br s, 1H), 7.80 (br d, J=4.0 Hz, 1H), 7.31 (s, 1H), 7.27 (d, J=1.6 Hz, 2H), 6.98 (br t, J=6.8 Hz, 3H), 6.91-6.83 (m, 2H), 4.75 (br t, J=6.8 Hz, 2H), 3.61 (br d, J=11.6 Hz, 2H), 3.26 (br s, 2H), 2.99-2.83 (m, 7H), 2.11-1.92 (m, 4H).

### Example 52

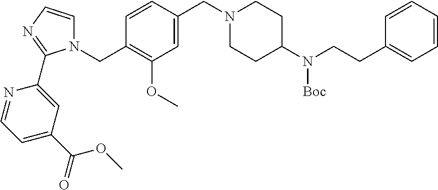 [SMILES: COC(=O)c1ccnc(-c2nccn2Cc2ccc(CN3CCC(N(CCc4ccccc4)C(=O)OC(C)(C)C)CC3)cc2OC)c1]

### Example 52A

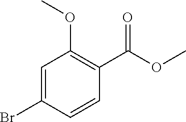

To a solution of 4-bromo-2-methoxybenzoic acid (1.00 g, 4.33 mmol, 1 eq) in MeOH (30 mL) was added H₂SO₄ (0.3 mL). The mixture was stirred at 90° C. for 4 hr. The reaction mixture was concentrated under reduced pressure to give a residue. The target compound (1.00 g, crude) was obtained as a light yellow oil. ¹H NMR (400 MHz, CDCl₃) δ 7.69 (d, J=8.4 Hz, 1H), 7.32-7.22 (m, 1H), 7.16-7.11 (m, 2H), 3.91 (s, 3H), 3.89 (s, 3H).

### Example 52B

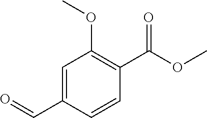

A mixture of Example 52A (200 mg, 816 umol, 1 eq), Pd(PPh₃)₂Cl₂ (85.9 mg, 122.4 umol, 0.15 eq) and HCOONa.2H₂O (424.5 mg, 4.08 mmol, 5 eq) in DMF (8 mL) was stirred at 70° C. for 24 hr under a CO atmosphere. The reaction mixture was diluted with EtOAc (30 mL) and washed with brine (15 mL*3), dried over sodium sulfate, filtered and concentrated in vacuo. The residue was purified by prep-TLC. The target compound (30 mg, 18.9%) was obtained as a yellow oil. ¹H NMR (400 MHz, CDC₃) δ 10.04 (s, 1H), 7.90 (d, J=8.0 Hz, 1H), 7.49 (m, 2H), 7.30-7.26 (m, 1H), 3.98 (s, 3H), 3.93 (s, 3H).

### Example 52C

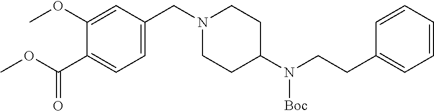 [SMILES: COC(=O)c1ccc(CN2CCC(N(CCc3ccccc3)C(=O)OC(C)(C)C)CC2)cc1OC]

To a solution of Example 52B (153 mg, 788.4 umol, 1.20 eq) in DCE (2 mL) was added AcOH (394.5 ug, 6.57 umol, 0.38 uL, 0.01 eq) and tert-butyl phenethyl(piperidin-4-yl)carbamate ([934695-79-1], 200 mg, 657 umol, 1 eq). The mixture was stirred at 50° C. for 3 hr then cooled to 40° C. NaBH(OAc)₃ (348 mg, 1.64 mmol, 2.5 eq) was added and the mixture was stirred at 40° C. for 8 hr. The reaction mixture was diluted with DCM (35 mL) and filtered. The filtrate was washed with saturated NaHCO₃ (10 mL) and brine (20 mL*3). The organic phase was dried over sodium sulfate and concentrated. The crude product was purified by prep-TLC to afford the target compound (150 mg, 47.3%) as a yellow oil. LCMS MH+ calculated, 483.3, found, 483.3.

### Example 52D

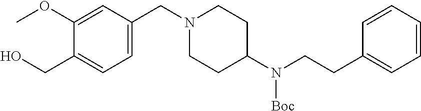 [SMILES: COc1cc(CN2CCC(N(CCc3ccccc3)C(=O)OC(C)(C)C)CC2)ccc1CO]

To a solution of Example 52C (150 mg, 310.8 umol, 1 eq) in THF (3 mL) was added LiAlH₄ (23.6 mg, 621.6 umol, 2 eq). The mixture was stirred at 0° C. for 2 hr. The reaction mixture was quenched by addition of H₂O (0.1 mL), followed by 15% NaOH aqueous solution (0.3 mL) and finally additional H₂O (0.1 mL). To the mixture was added anhydrous MgSO₄ and the mixture was stirred at room temperature for 0.5 hr, filtered and concentrated. The target compound (130 mg, crude) was obtained as a light yellow oil. LCMS MH+ calculated, 455.3, found, 455.3.

### Example 52E

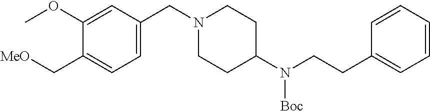 [SMILES: COCc1ccc(CN2CCC(N(CCc3ccccc3)C(=O)OC(C)(C)C)CC2)cc1OC]

To a solution of Example 52D (400 mg, 879.9 umol, 1 eq) in DCM (2 mL) was added MsCl (151.2 mg, 1.32 mmol, 102 uL, 1.50 eq) and Et₃N (178 mg, 1.76 mmol, 243.9 uL, 2 eq). The mixture was stirred at 20° C. for 2 hr. The reaction mixture was diluted with DCM (20 mL) and washed with brine (10 mL*3). The organic phase was dried over sodium sulfate and concentrated. The target compound (500 mg, crude) was obtained as a light yellow oil and used directly in the next reaction.

### Example 52F

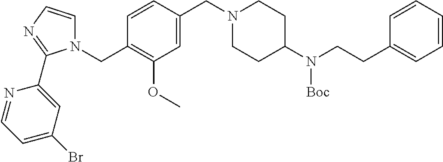 [SMILES: COc1cc(CN2CCC(N(CCc3ccccc3)C(=O)OC(C)(C)C)CC2)ccc1Cn1ccnc1-c1cc(Br)ccn1]

To a solution of Example 52E (500 mg, 938.6 umol, 1 eq) in DMF (5 mL) was added 4-bromo-2-(1H-imidazol-2-yl)pyridine ([1211579-82-6], 126.2 mg, 563.2 umol, 0.60 eq) and K₂CO₃ (259.5 mg, 1.88 mmol, 2 eq). The mixture was stirred at 82° C. for 10 hr. The reaction mixture was filtered, the filtrate was diluted with DCM (30 mL) and washed with brine (20 mL*3). The organic phase was dried over sodium sulfate and concentrated. The residue was purified by prep-TLC. The target compound (180 mg, 29.0%) was obtained as a light yellow oil. LCMS MH+ calculated, 660.3, found, 660.2.

### Example 52

To a solution of Example 52F (180 mg, 272.5 umol, 1 eq) in MeOH (5 mL) was added Et₃N (275.7 mg, 2.7 mmol, 377.7 uL, 10 eq) and Pd(dppf)Cl₂ (19.9 mg, 27.3 umol, 0.10 eq). The mixture was stirred at 70° C. for 7 hr under a CO (50 psi) atmosphere. The reaction mixture was filtered and concentrated. The residue was purified by prep-TLC. The target compound (160 mg, 91.8%) was obtained as a brown oil. LCMS MH+ calculated, 640.3, found, 640.4.

### Example 53

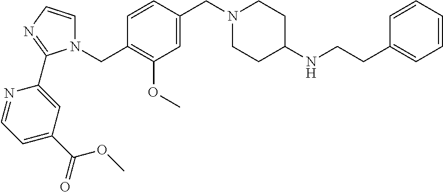 [SMILES: COC(=O)c1ccnc(-c2nccn2Cc2ccc(CN3CCC(NCCc4ccccc4)CC3)cc2OC)c1]

### Example 53

To a solution of Example 52 (160 mg, 250 umol, 1 eq) in DCM (3 mL) was added TFA (285 mg, 2.50 mmol, 185 uL, 10 eq). The mixture was stirred at 20° C. for 1 hr. The reaction mixture was filtered and concentrated. The target compound (150 mg, crude) was obtained as a green oil. LCMS MH+ calculated, 540.3, found, 540.3.

### Example 54

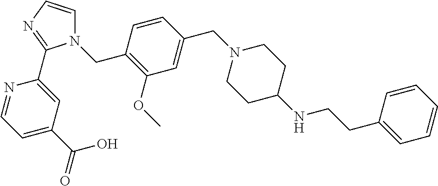 [SMILES: COc1cc(CN2CCC(NCCc3ccccc3)CC2)ccc1Cn1ccnc1-c1cc(C(=O)O)ccn1]

### Example 54

To a solution of Example 53 (150 mg, 278 umol, 1 eq) in H₂O (1 mL) and MeOH (2 mL) was added NaOH (22.2 mg, 555.9 umol, 2 eq). The mixture was stirred at 20° C. for 1 hr. The reaction mixture was filtered. The filtrate was adjusted to pH 8 with 4M HCl aqueous solution, then concentrated. The residue was purified by prep-HPLC. The target compound (86.7 mg, 59.4%) was obtained as a pink solid. LCMS MH+ calculated, 526.3, found, 526.3. ¹H NMR (400 MHz, CD₃OD) δ 8.62 (d, J=4.8 Hz, 1H), 8.31 (s, 1H), 7.77 (d, J=4.8 Hz, 1H), 7.33-7.26 (m, 2H), 7.25-7.16 (m, 4H), 7.05 (s, 1H), 6.90 (s, 1H), 6.83-6.77 (m, 1H), 6.72 (d, J=8.4 Hz, 1H), 5.68 (s, 2H), 3.72 (s, 3H), 3.45 (s, 2H), 3.00-2.90 (m, 2H), 2.84 (m, 4H), 2.65 (br s, 1H), 2.00 (br t, J=12. Hz, 2H), 1.90 (br d, =11.6 Hz, 2H), 1.50-1.38 (m, 2H).

### Example 55

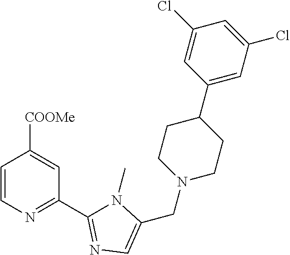 [SMILES: COC(=O)c1ccnc(-c2ncc(CN3CCC(c4cc(Cl)cc(Cl)c4)CC3)n2C)c1]

### Examples 55A and 57A

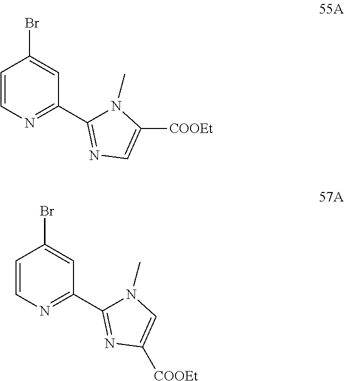 [SMILES: CCOC(=O)c1cnc(-c2cc(Br)ccn2)n1C ; CCOC(=O)c1cn(C)c(-c2cc(Br)ccn2)n1]

To a stirred solution of Example 14B (12 g, 0.040 mol) in DMF (120 mL) was added MeI (6.28 g, 0.044 mol) and K₂CO. (6.62 g, 0.048 mol) at 0° C. and stirred for 2 hours at the same temperature. After completion of the reaction, ice water was added to the reaction and stirring continued for 20 min. The precipitated solid was filtered and dried in vacuo. The crude product obtained was purified by column chromatography using basic alumina (gradient elution with 5 to 10% EtOAc/Hexane) to give 55A (2.5 g) as a pale yellow solid and 57A (5 g) as a pale yellow solid. LCMS MH+ calculated, 310.0, found, 310.1.

### Example 55B

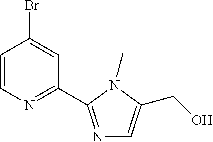

A suspension of LiAlH₄ (660 mg, 0.017 mol) in dry THF (100 mL) was cooled to 0° C. and Example 55A (2.5 g, 0.008 mol) dissolved in dry THF (75 mL) was added drop wise, maintaining the temperature at 0° C. The reaction was stirred for 30 min. The reaction was quenched with EtOAc (60 mL) followed by water (10 mL). The mixture was filtered through a celite pad and the pad washed with EtOAc (100 mL). The filtrate was concentrated and purified by column chromatography using basic alumina (gradient elution with 1% MeOH in DCM) to afford the target compound (1.2 g, 55.5%) as a white solid. LCMS MH+ calculated, 268.0, found, 268.1.

### Example 55C

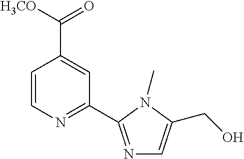 [SMILES: COC(=O)c1ccnc(-c2ncc(CO)n2C)c1]

To a stirred solution of Example 55B (180 mg, 0.671 mmol) in methanol (6 mL) and triethylamine (1.0 mL 10.07 mmol) at room temperature was added Pd(dppf)Cl₂ (73 mg, 0.10 mmol) and the temperature was raised to 80° C. under CO (50 psi) and stirred for 2 h. After cooling, the mixture was filtered through a celite pad and the pad washed with methanol (5 mL). The filtrate was concentrated and purified by column chromatography using basic alumina (gradient elution with 1% MeOH in DCM) to afford the target compound (100 mg, 60.3%) as a light brown solid. LCMS MH+ calculated, 248.1, found, 248.2.

### Example 55D

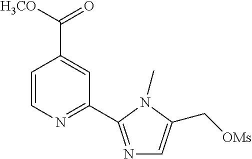 [SMILES: COC(=O)c1ccnc(-c2ncc(COS(C)(=O)=O)n2C)c1]

To a stirred solution of Example 55C (100 mg, 0.40 mmol) in DCM (4 mL) was added drop wise triethylamine (0.16 ml, 1.20 mmol) and methane sulfonyl chloride (92 mg, 0.809 mmol) at 0° C. and the reaction stirred for 2 h. The reaction was quenched with water (10 mL) and extracted with DCM (2×10 ml). The combined organic layers were dried over anhydrous sodium sulfate, concentrated and purified by alumina (basic) column chromatography (gradient elution with 1% MeOH in DCM) to afford the target compound (130 mg, 61.9%) as a pale yellow semi-solid which was used directly in the next reaction.

### Example 55

To a stirred solution of 4-(3,5-dichlorophenyl)piperidine (Bavetsias et al., J. Med. Chem. 2016, 59, 1388-1409, 59 mg, 0.258 mmol) in dry DMF (2 mL) was added potassium carbonate (59.3 mg, 0.43 mmol) at room temperature under inert atmosphere. Example 55D (70 mg, 0.215 mmol) was added and the reaction heated to 80° C. for 3 h. The reaction mixture was cooled to room temperature and filtered. The filtrate was concentrated and purified by alumina (basic) column chromatography (gradient elution with 1% MeOH in DCM) to afford the target compound (70 mg, 72.2%) as a yellow semi-solid. LCMS MH+ calculated, 459.1, found, 459.2.

### Example 56

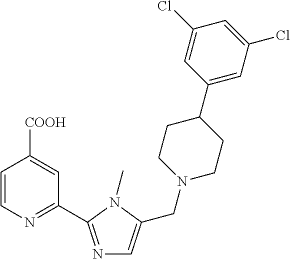 [SMILES: Cn1c(CN2CCC(c3cc(Cl)cc(Cl)c3)CC2)cnc1-c1cc(C(=O)O)ccn1]

### Example 56

To a stirred solution of Example 55 (70 mg, 0.155 mmol) in a mixture of THF (2 mL) and H₂O (0.5 mL) was added LiOH.H₂O (32 mg, 0.75 mmol) at 0° C. The reaction was stirred at room temperature for 2 h. The reaction mixture was concentrated in vacuo to remove THF and the aqueous phase acidified to pH 2 with 1M HCl and then concentrated. The resulting crude product was purified by preparative HPLC and lyophilized to furnish the TFA salt of the target compound (18.3 mg, 12.7/o) as a pink solid. LCMS MH+ calculated, 445.1, found, 445.0. ¹H NMR (300 MHz, DMSO-d6) δ 8.85 (1H, J=3.6 Hz, d), 8.51 (1H, s), 7.83 (1H, J=3.6 Hz, d), 7.52 (1H, s), 7.43-7.22 (3H, m), 4.51 (2H, s), 4.10 (3H, s), 3.62-3.28 (2H, m), 3.18-2.78 (3H, m), 2.10-1.72 (4H, m).

### Example 57

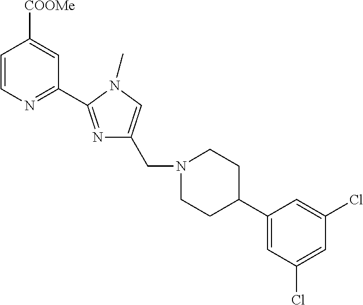 [SMILES: COC(=O)c1ccnc(-c2nc(CN3CCC(c4cc(Cl)cc(Cl)c4)CC3)cn2C)c1]

Example 57 was prepared from Example 57A in a manner analogous to that of Example 55. The target compound was isolated as a yellow semi-solid. LCMS MH+ calculated, 459.1, found, 459.2.

### Example 58

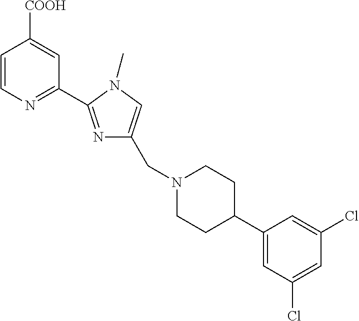 [SMILES: Cn1cc(CN2CCC(c3cc(Cl)cc(Cl)c3)CC2)nc1-c1cc(C(=O)O)ccn1]

Example 58 was prepared from Example 57 in a manner analogous to that of Example 56. The target compound was isolated as a pink solid. LCMS MH+ calculated, 445.1, found, 445.0. ¹H NMR (300 MHz, DMSO-d6) δ 8.81 (1H, d), 8.58 (1H, s), 7.81 (1H, d), 7.59 (1H, s), 7.49 (1H, s), 7.25 (2H, s), 4.30 (2H, s), 4.10 (3H, s), 3.62-3.51 (2H, m), 3.18-3.02 (2H, m), 2.96-2.81 (1H, m), 2.10-1.79 (4H, m).

### Example 59

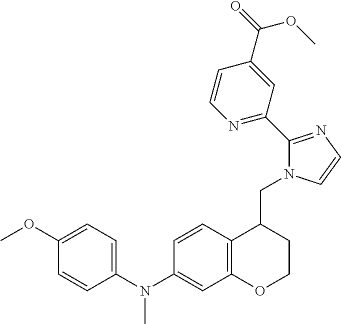 [SMILES: COC(=O)c1ccnc(-c2nccn2CC2CCOc3cc(N(C)c4ccc(OC)cc4)ccc32)c1]

### Example 59A

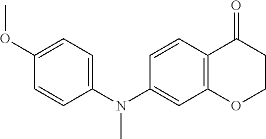 [SMILES: COc1ccc(N(C)c2ccc3c(c2)OCCC3=O)cc1]

To a stirred solution of 4-methoxy-N-methylaniline ([5961-59-1], 3.6 g, 26.27 mmol) and 7-bromochroman-4-one ([18442-22-3], 7.15 g, 31.53 mmol) in toluene (40 mL) were added Cs₂CO₃ (17.07 g, 52.54 mmol), BINAP (1.31 g, 2.10 mmol) and Pd(OAc)₂ (0.47 g, 2.10 mmol). The reaction was stirred at 100° C. for 12 h under inert atmosphere. The mixture was partitioned between water and EtOAc. The organic layer was concentrated and purified by silica-gel column chromatography [gradient elution with 10% EtOAc in Hexane] to afford the target compound (2.9 g, 40.0%) as a brown solid. LCMS MH+ calculated, 284.1, found, 284.2.

### Example 59B

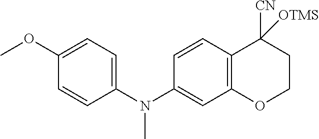

To a stirred solution of Example 59A (2 g, 7.06 mmol) in toluene (15 ml) was added trimethylsilyl cyanide (2.53 ml, 20.19 mmol) and zinc iodide (90 mg, 0.28 mmol). The reaction was heated at 60° C. for 2 h. The mixture was partitioned between saturated NaHCO₃ and EtOAc. The organic layer was dried and concentrated to give the target compound (1.91 g, 71%) as brown oil, which was used in the next step without further purification. LCMS MH+ calculated, 383.2, found, 383.3.

### Example 59C

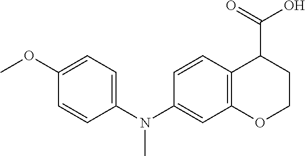 [SMILES: COc1ccc(N(C)c2ccc3c(c2)OCCC3C(=O)O)cc1]

To a stirred solution of Example 578B (1.3 g, 3.3 mmol) in AcOH (4 mL) was added SnCl₂.2H₂O (3.0 g, 13.5 mmol) and conc. HCl (4 mL). The reaction was stirred for 12 h at 100° C. under an inert atmosphere. The mixture was partitioned between water and DCM. The organic layer was concentrated and purified by silica-gel column chromatography [gradient elution with 2% MeOH in DCM] to afford the target compound (500 mg, 50%) as a brown solid.

### Example 59D

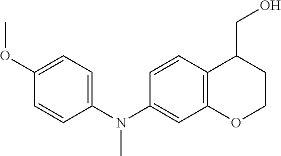 [SMILES: COc1ccc(N(C)c2ccc3c(c2)OCCC3CO)cc1]

To a stirred solution of Example 59C (500 mg, 1.59 mmol) in THF (5 ml) was added LiAlHₐ(242 mg, 6.38 mmol) portion wise. The reaction was stirred at 0° C. for 1 h under inert atmosphere. The mixture was partitioned between water and EtOAc. The organic layer was concentrated and purified by silica gel column chromatography [gradient elution with 3% MeOH in DCM] to afford the target compound (330 mg, 69.1%) as a white solid. LCMS MH+ calculated, 300.2, found, 300.3.

### Example 59E

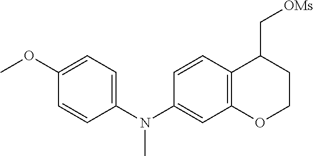 [SMILES: COc1ccc(N(C)c2ccc3c(c2)OCCC3COS(C)(=O)=O)cc1]

To a stirred solution of Example 59D (250 mg, 0.83 mmol) and TEA (0.23 ml, 1.67 mmol) in DCM (10 ml) was added methane sulfonylchloride (0.07 ml, 1.00 mmol). The reaction was stirred at room temperature for 30 min under inert atmosphere. The mixture was partitioned between water and DCM. The organic layer was concentrated to give the target compound (300 mg, 95%) as a yellow liquid, which was directly in the next step.

### Example 59F

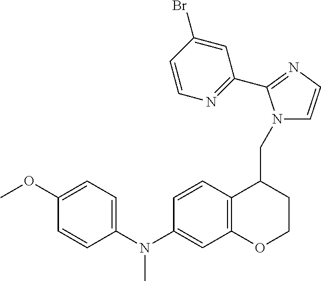 [SMILES: COc1ccc(N(C)c2ccc3c(c2)OCCC3Cn2ccnc2-c2cc(Br)ccn2)cc1]

To a stirred solution of Example 59E (210 mg, 0.557 mmol) and 4-bromo-2-(1H-imidazol-2-yl)pyridine ([1211579-82-6], 136 mg, 0.61 mmol) in dry DMF (5 ml) was added potassium carbonate (230 mg, 0.167 mmol). The reaction was stirred at 80° C. for 2 h. After cooling, the reaction mixture was filtered and concentrated. The mixture was purified by alumina [basic] column chromatography [gradient elution with 3% MeOH in Hexane] to give the target compound (150 mg, 46%) as a brown liquid. LCMS MH+ calculated, 505.1, found, 505.3.

### Example 59

To a stirred solution of Example 59F (150 mg, 0.28 mmol) in methanol (15 mL) was added Et₃N (480 mg, 4.45 mmol) and Pd(dppf)Cl₂ (32 mg, 0.043 mmol). The reaction was stirred at 80° C. under an atmosphere of CO (50 psi) for 2 h. The reaction was cooled and filtered through a celite pad. The filtrate was concentrated and purified by alumina [basic] column chromatography [gradient elution with 2% MeOH in DCM] to afford the target compound (80 mg, 55%) as a pale yellow solid. LCMS MH+ calculated, 485.2, found, 485.4.

### Example 60

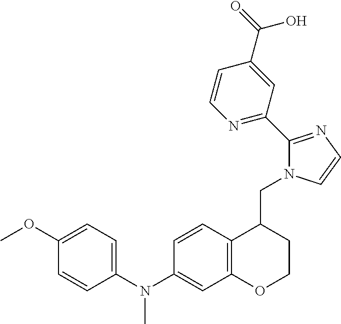 [SMILES: COc1ccc(N(C)c2ccc3c(c2)OCCC3Cn2ccnc2-c2cc(C(=O)O)ccn2)cc1]

To a stirred solution of Example 59 (80 mg, 0.165 mmol) in methanol (3 mL) was added LiOH.H₂O (2 M, 0.14 mL, 0.294 mmol). The reaction was stirred at room temperature for 1 h. The mixture was concentrated and the residue adjusted to pH 5 with 1M HCl solution. The crude material was purified by preparative HPLC and lyophilized to furnish the target compound (25 mg, 329%) as a pink solid. ¹H NMR (300 MHz, DMSO-d6) δ 8.9 (S, 1H), 8.39 (S, 1H), 7.9 (d, J=11.7 Hz, 2H), 7.64 (s, 1H), 7 (d, J=8.7 Hz, 2H), 6.92 (d, J=9 Hz, 2H),6.55 (d, J=8.4 Hz, 1H), 6.12 (d, J=2.1 Hz, 1H), 5.87 (s, 1H), 5.22-5.15 (m, 1H), 4.64-4.60 (m, 1H), 4.06 (d, J=5.7 Hz, 2H), 3.74 (s, 3H), 3.16 (s, 1H), 3.07 (s, 3H), 1.8-1.7 (m, 2H).

### Example 61

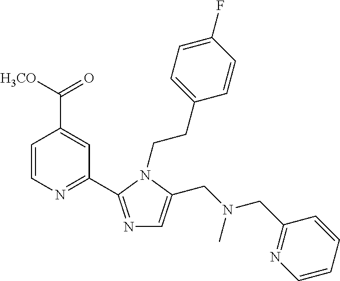 [SMILES: COC(=O)c1ccnc(-c2ncc(CN(C)Cc3ccccn3)n2CCc2ccc(F)cc2)c1]

### Examples 61A and 63A

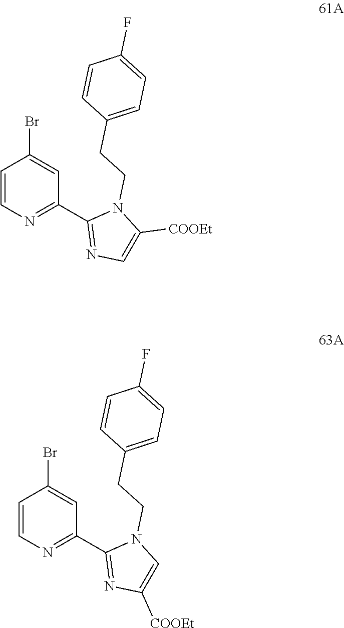 [SMILES: CCOC(=O)c1cnc(-c2cc(Br)ccn2)n1CCc1ccc(F)cc1 ; CCOC(=O)c1cn(CCc2ccc(F)cc2)c(-c2cc(Br)ccn2)n1]

To a stirred solution of Example 14B (7.8 g, 0.0263 mol) in DMF (78 mL) was added Example 43A (6.28 g, 0.0289 mol) and K₂CO₃ (7.27 g, 0.0526 mol). The reaction was stirred at 80° C. for 2 hours. The reaction mixture was filtered, concentrated and purified by alumina (basic) column chromatography [gradient elution with 4 to 70/o EtOAc/Hexane] to afford Example 61A (2.4 g) as a white solid and Example 63A (3 g) as an off white solid. LCMS MH+ calculated, 418.1, found, 418.2.

¹HNMR of 61A (500 MHz, CD₃OD) δ 8.59 (d, J=5.5 Hz, 1H), 7.99 (d, J=1.0 Hz, 1H), 7.80 (s, 1H), 7.75 (dd, J=2.0, 5.5 Hz, 1H), 7.02-6.96 (m, 4H), 5.09 (t, J=7.0 Hz, 2H), 4.32 (q, J=7.5, 14.5 Hz, 2H), 2.99 (t, J=6.5 Hz, 2H), 1.32 (t, J=7.0 Hz, 3H).

¹HNMR of 63A (500 MHz, CD₃OD) δ 8.56 (d, J=5.5 Hz, 1H), 8.09 (s, 2H), 7.72 (dd, J=2.5, 5.5 Hz, 1H), 7.11 (t, J=5.5 Hz, 2H), 7.03 (t, J=18 Hz, 2H), 4.79 (t, J=14.5 Hz, 2H), 4.26 (q, J=7.0, 4.5 Hz, 2H), 3.02 (t, J=14.5 Hz, 2H), 1.29 (t, J=14.0 Hz, 3H).

### Example 61B

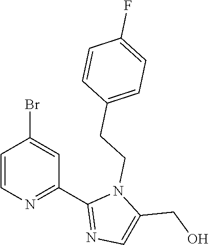 [SMILES: OCc1cnc(-c2cc(Br)ccn2)n1CCc1ccc(F)cc1]

A suspension of LiAlH₄ (77 mg, 2.037 mmol) in dry THF (6 mL) was cooled to 0° C. and a solution of Example 61A (850 mg, 2.037 mmol) in dry THF (8 mL) was added drop wise, maintaining the temperature at 0° C. The reaction was stirred for 30 min. The reaction was quenched with EtOAc (6 mL) followed by water (1 mL). The mixture was filtered through a celite pad and the pad washed with EtOAc (10 mL). The filtrate was concentrated in vacuo and purified by alumina (basic) column chromatography (gradient elution with 1% MeOH/DCM) to afford the target compound (400 mg, 52.3%) as a white solid. LCMS MH+ calculated, 376.0, found, 376.2. ¹HNMR (300 MHz, DMSO-d₆) δ 8.54 (d, J=5.4 Hz, 1H), 8.08 (s, 1H), 7.63 (d, J=5.4 Hz, 1H), 7.14-7.01 (m, 51H), 5.30 (t, J=4.8 Hz, 1H), 4.74 (t, J=7.2 Hz, 2H), 4.47 (d, J=5.1 Hz, 2H), 3.01 (t, J=7.5 Hz, 2H).

### Example 61C

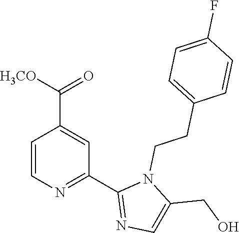 [SMILES: COC(=O)c1ccnc(-c2ncc(CO)n2CCc2ccc(F)cc2)c1]

To a stirred solution of Example 61B (1.2 g, 0.0032 mol) in methanol (30 mL) was added triethylamine (6.68 mL 0.048 mol) and Pd(dppf)Cl₂ (350 mg, 0.00048 mol). The reaction was stirred at 80° C. under CO pressure (50 psi) for 2 h. The mixture was filtered through a celite pad and the pad washed with methanol (15 mL). The filtrate was concentrated and purified by alumina (basic) column chromatography (gradient elution with 1% MeOH/DCM) to afford the target compound (860 mg, 78.1%) as a light brown solid. LCMS MH+ calculated, 356.1, found, 356.2. ¹HNMR (300 MHz, DMSO-d₆) δ 8.85 (d, J=4.8 Hz, 1H), 8.39 (s, 1H), 7.77 (d, J=4.2 Hz, 1H), 7.15-6.99 (m, 5H), 5.31 (t, J=5.1 Hz, 1H), 4.76 (t, J=7.2 Hz, 2H), 4.49 (d, J=4.8 Hz, 2H), 3.93 (s, 3H), 3.03 (t, J=7.2 Hz, 2H).

### Example 61D

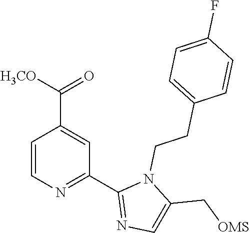

To a stirred solution of Example 61C (150 mg, 0.4225 mmol) in DCM (8 mL) was added drop wise triethyl amine (0.17 ml, 1.267 mmol). Methane sulfonyl chloride (58 mg, 0.507 mmol) was added at 0° C. and the reaction stirred at this temperature for 2 h. The mixture was partitioned between water and DCM. The organic phase was dried, concentrated under reduced pressure and purified by alumina (basic) column chromatography (gradient elution with 1% MeOH in DCM) to furnish the target compound (120 mg, 65.9%) as a brown semi-solid.

### Example 61

To a stirred solution of N-methyl-1-(pyridin-2-yl)methanamine ([21035-59-6], 42 mg, 0.346 mmol) in dry DMF (4 mL) was added potassium carbonate (48 mg, 0.346 mmol). Example 61D (150 mg, 0.346 mmol) was added and the reaction stirred at 80° C. for 2 h. The reaction mixture was cooled to room temperature and filtered. The filtrate was concentrated and purified by column chromatography using basic alumina (gradient elution with 1% MeOH in DCM) to afford the target compound (80 mg, 50%) as a brown semi-solid. LCMS MH+ calculated, 460.2, found, 460.4.

### Example 62

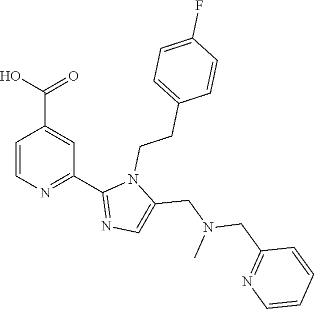 [SMILES: CN(Cc1ccccn1)Cc1cnc(-c2cc(C(=O)O)ccn2)n1CCc1ccc(F)cc1]

To a stirred solution of Example 61 (80 mg, 0.174 mmol) in methanol (1 mL) was added LiOH (2 M in water, 0.17 mL, 0.348 mmol) at 0° C. The reaction was stirred at room temperature for 1 h. The reaction mixture was concentrated to remove MeOH and acidified to pH 5 with 1M HCl. The resulting aqueous phase was purified by preparative HPLC and lyophilized to furnish the target compound (25 mg, 32.4%) as an off-white solid. LCMS MH+ calculated, 446.2, found, 446.4. ¹H NMR (300 MHz, DMSO-d6) δ 13.90 (br, 1H), 8.84 (d, J=4.8 Hz, 1H), 8.62 (d, J=4.2 Hz, 1H), 8.31 (s, 1H), 7.92 (t, J=7.5 Hz, 1H), 7.81 (d, J=4.5 Hz, 1H), 7.52-7.43 (m, 2H), 7.39 (s, 1H) 6.96-6.86 (m, 4H), 4.86 (t. J=5.7 Hz, 2H), 4.45 (s, 2H), 4.25 (s, 2H), 3.16 (s, 1H), 2.85 (t, J=5.7 Hz, 1H), 2.73 (s, 3H).

### Example 63

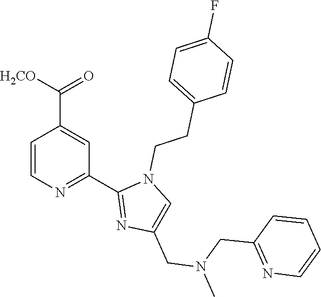

Example 63 was prepared from Example 63A in a manner analogous to Example 61. Example 63 (80 mg, 41.4%) was isolated as a brown semi solid. LCMS MH+ calculated, 460.2, found, 460.4.

### Example 64

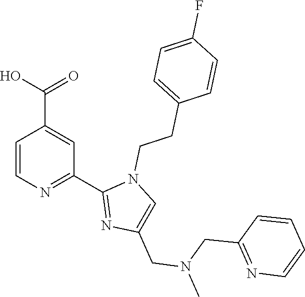 [SMILES: CN(Cc1ccccn1)Cc1cn(CCc2ccc(F)cc2)c(-c2cc(C(=O)O)ccn2)n1]

Example 64 was prepared from Example 63 in a manner analogous to Example 62. Example 64 (25 mg, 32.4%) was isolated as an off white solid. LCMS MH+ calculated, 446.2, found, 446.4. ¹H NMR (300 MHz, DMSO-d6) δ 8.85 (d, J=5.1 Hz, 1H), 8.70 (d, J=3.9 Hz, 1H), 8.38 (s, 1H), 8.01-7.98 (m, 1H), 7.84 (dd, J=1.5, 5.1 Hz, 1H), 7.69 (d, J=7.8 Hz, 1H), 7.63 (s, 1H), 7.55-7.50 (m, 1H), 7.13-7.08 (m, 2H), 6.96 (t, J=17.7 Hz, 2H), 5.93 (brs, 1H), 4.83 (t, J=14.1 Hz, 2H), 4.46 (s, 2H), 4.35 (s, 2H), 3.05-3.01 (t, J=13.8 Hz, 2H), 2.73 (s, 3H).

### Example 65

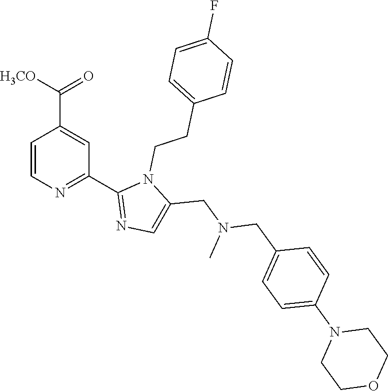 [SMILES: COC(=O)c1ccnc(-c2ncc(CN(C)Cc3ccc(N4CCOCC4)cc3)n2CCc2ccc(F)cc2)c1]

Example 65 was prepared from N-methyl-1-(4-morpholinophenyl)methanamine [179328-22-4] in a manner analogous to Example 61. The target compound (80 mg, 53.3%) was isolated as a brown semi-solid. LCMS MH+ calculated, 544.3, found, 544.5.

### Example 66

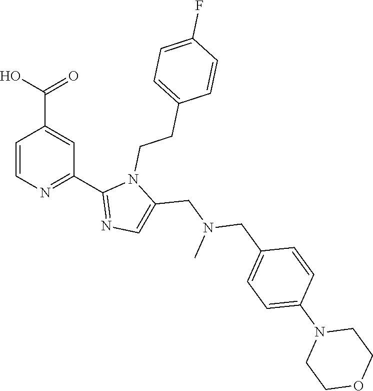 [SMILES: CN(Cc1ccc(N2CCOCC2)cc1)Cc1cnc(-c2cc(C(=O)O)ccn2)n1CCc1ccc(F)cc1]

Example 66 was prepared from Example 65 in a manner analogous to Example 62. The target compound (20 mg, 25.6%) was isolated as a pale pink solid. LCMS MH+ calculated, 530.3, found, 530.4. ¹H NMR (300 MHz, DMSO-d6) δ9.80 (br, 1H), 8.84 (d, J=4.8 Hz, 1H), 8.15 (s, 1H), 7.85 (d, J=4.2 Hz, 1H), 7.48 (s, 1H), 7.40 (d, J=8.1 Hz, 2H), 7.05 (d, J=8.1 Hz, 2H), 6.86 (t, J=8.4 Hz, 2H), 6.75 (t, J=4.8 Hz, 2H), 4.27 (t, J=12.3 Hz, 4H), 3.75 (s, 4H), 3.69-3.65 (m, 2H), 3.16-3.10 (m, 4H), 2.74 (t, J=5.4 Hz, 2H), 2.70 (s, 3H).

### Example 67

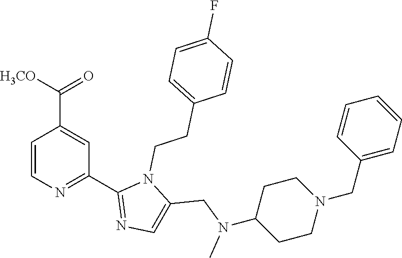 [SMILES: COC(=O)c1ccnc(-c2ncc(CN(C)C3CCN(Cc4ccccc4)CC3)n2CCc2ccc(F)cc2)c1]

Example 67 was prepared from 1-benzyl-N-methylpiperidin-4-amine [7006-50-0] in a manner analogous to Example 61. The target compound (80 mg, 42.6%) was isolated as a yellow semi-solid. LCMS MH+ calculated, 542.3, found, 542.4.

### Example 68

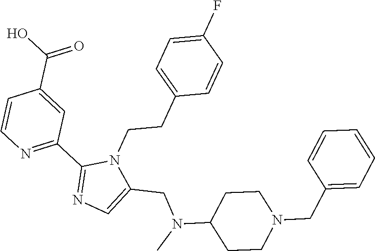 [SMILES: CN(Cc1cnc(-c2cc(C(=O)O)ccn2)n1CCc1ccc(F)cc1)C1CCN(Cc2ccccc2)CC1]

Example 68 was prepared from Example 67 in a manner analogous to Example 62. The target compound (20 mg, 25.6°) was isolated as an off white solid. LCMS MH+ calculated, 528.3, found, 528.4. ¹H NMR (300 MHz, DMSO-d6) δ8.70 (d, J=4.5 Hz, 1H), 8.32 (s, 1H), 7.71 (d, J=4.5 Hz, 1H), 7.40-7.30 (m, 5H), 7.12 (t, J=6.9 Hz, 2H), 7.03 (t, J=8.4 Hz, 2H), 6.96 (s, 1H), 4.71 (t, J=6.6 Hz, 2H), 3.86 (s, 2H), 3.54 (s, 2H), 3.17 (s, 3H), 3.14-3.10 (m, 1H), 2.97 (t, J=6.9 Hz, 2H), 2.14 (s, 4H), 1.73-1.65 (m, 4H).

### Example 69

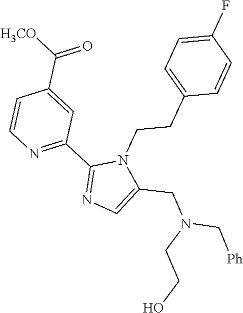 [SMILES: COC(=O)c1ccnc(-c2ncc(CN(CCO)Cc3ccccc3)n2CCc2ccc(F)cc2)c1]

Example 69 was prepared from 2-(benzylamino)ethan-1-ol [104-63-2] in a manner analogous to Example 61. The target compound (70 mg, 51.8%) was isolated as a yellow semi-solid. LCMS MH+ calculated, 489.2, found, 489.4.

### Example 70

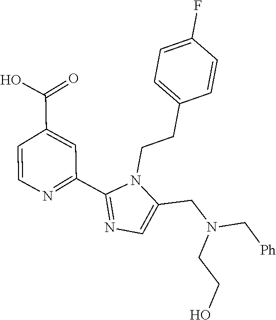 [SMILES: O=C(O)c1ccnc(-c2ncc(CN(CCO)Cc3ccccc3)n2CCc2ccc(F)cc2)c1]

Example 70 was prepared from Example 69 in a manner analogous to Example 62. The target compound (20 mg, 29.4%) was isolated as a pale pink solid. LCMS MH+ calculated, 475.2, found, 475.3. ¹H NMR (300 MHz, DMSO-d6) δ 8.85 (d, J=4.2 Hz, 1H), 8.11 (s, 1H), 7.90-7.85 (d, J=4.8 Hz, H), 7.51 (t, J=4.2 Hz, 6H), 6.84 (t, J=8.1 Hz, 2H), 6.72 (t, J=6.3 Hz, 2H), 4.68 (br s, 1H), 4.35-4.22 (m, 4H), 3.90-3.75 (m, 4H), 3.20-3.05 (m, 2H), 2.80-2.70 (m, 2H).

### Example 71

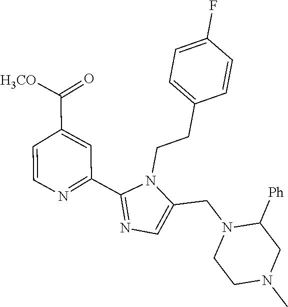 [SMILES: COC(=O)c1ccnc(-c2ncc(CN3CCN(C)CC3c3ccccc3)n2CCc2ccc(F)cc2)c1]

Example 71 was prepared from 1-methyl-3-phenylpiperazine [5271-27-2] in a manner analogous to Example 61. The target compound (80 mg, 37.2%) was isolated as a brown semi-solid. LCMS MH+ calculated, 514.3, found, 514.4.

### Example 72

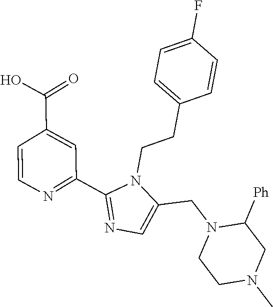 [SMILES: CN1CCN(Cc2cnc(-c3cc(C(=O)O)ccn3)n2CCc2ccc(F)cc2)C(c2ccccc2)C1]

Example 72 was prepared from Example 71 in a manner analogous to Example 62. The target compound (50 mg, 64.9%) was isolated as a pink solid. LCMS MH+ calculated, 500.2, found, 500.4. ¹H NMR (300 MHz, DMSO-d6) δ 13.90 (br s, 1H), 9.95 (br s, 1H), 8.80 (d, J=3.9 Hz 1H), 8.30 (s, 1H), 7.78 (d, J=3.6 Hz, 1H), 7.46 (s, 5H), 7.14 (s, 1H), 6.9 (t, J=8.1 Hz, 2H), 6.76 (t, J=5.7 Hz, 2H), 4.79-4.73 (m, 1H), 4.58-4.47 (m, 1H), 3.55-3.25 (m, 4H), 3.18 (s, 3H), 3.01 (t, J=13.5 Hz, 2H), 2.85-2.75 (m, 4H), 2.43-2.32 (m, 1H).

### Example 73

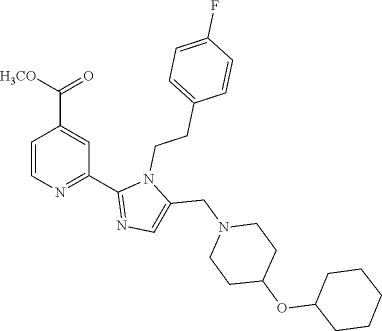 [SMILES: COC(=O)c1ccnc(-c2ncc(CN3CCC(OC4CCCCC4)CC3)n2CCc2ccc(F)cc2)c1]

Example 73 was prepared from 4-(cyclohexyloxy)piperidine [303975-02-2] in a manner analogous to Example 61. The target compound (90 mg, 37.5%) was isolated as a yellow liquid. LCMS MH+ calculated, 521.3, found, 521.5.

### Example 74

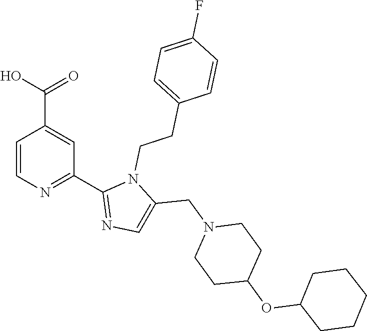 [SMILES: O=C(O)c1ccnc(-c2ncc(CN3CCC(OC4CCCCC4)CC3)n2CCc2ccc(F)cc2)c1]

Example 74 was prepared from Example 73 in a manner analogous to Example 62. The target compound (15 mg, 17%) was isolated as a pale pink solid. LCMS MH+ calculated, 507.3, found, 507.5. ¹H NMR (300 MHz, DMSO-d6) δ 9.65-9.40 (brs, 1H), 8.83 (d, J=4.5 Hz, 1H), 8.11 (s, 1H), 7.83 (d, J=4.2 Hz, 1H), 7.44 (s, 1H), 6.88-6.82 (m, 4H), 4.93 (s, 2H), 4.42 (s, 2H), 1.91-1.47 (m, 9H), 1.22 (d, J=6.9 Hz, 5H).

### Example 75

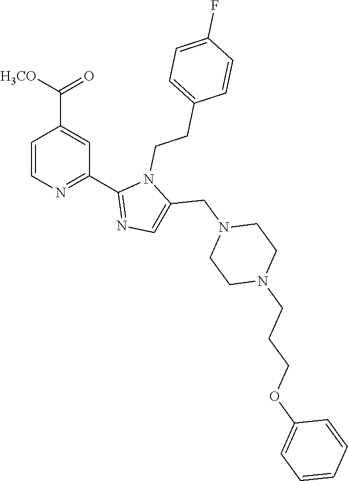 [SMILES: COC(=O)c1ccnc(-c2ncc(CN3CCN(CCCOc4ccccc4)CC3)n2CCc2ccc(F)cc2)c1]

Example 75 was prepared from 1-(3-phenoxypropyl)piperazine [41298-49-1] in a manner analogous to Example 61. The target compound (70 mg, 36.4%) was isolated as a yellow semi-solid. LCMS MH+ calculated, 558.3, found, 558.5.

### Example 76

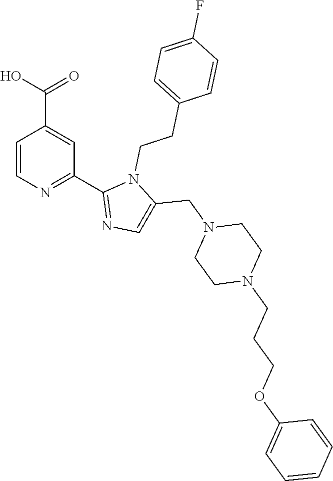 [SMILES: O=C(O)c1ccnc(-c2ncc(CN3CCN(CCCOc4ccccc4)CC3)n2CCc2ccc(F)cc2)c1]

Example 76 was prepared from Example 75 in a manner analogous to Example 62. The target compound (20 mg, 29.4%) was isolated as a pale pink solid. LCMS MH+ calculated, 544.3, found, 544.5. ¹H NMR (300 MHz, DMSO-d6) δ 13.92 (br s, 1H), 9.55 (br s, 1H), 8.88 (d, J=5.1 Hz, 1H), 8.34 (s, 1H), 7.82 (d, J=1.5 Hz, 1H), 7.32-7.25 (m, 3H), 7.16-7.13 (m, 2H), 7.11 (m, 2H), 7.05-6.91 (m, 3H), 4.79 (t, J=6.9 Hz, 2H), 4.03 (t, J=6.0 Hz, 3H), 3.65 (s, 2H), 3.56 (d, J=11.1 Hz, 2H), 3.31-3.06 (m, 6H), 2.50 (m, 1H), 2.49 (m, 2H).

### Example 77

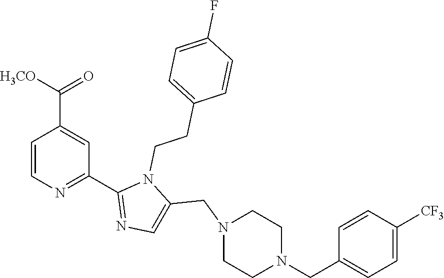 [SMILES: COC(=O)c1ccnc(-c2ncc(CN3CCN(Cc4ccc(C(F)(F)F)cc4)CC3)n2CCc2ccc(F)cc2)c1]

Example 77 was prepared from 1-(4-(trifluoromethyl)benzyl)piperazine [107890-32-4] in a manner analogous to Example 61. The target compound (100 mg, 40.9%) was isolated as a yellow semi-solid. LCMS MH+ calculated, 582.2, found, 582.5.

### Example 78

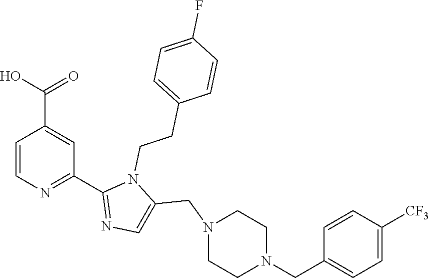 [SMILES: O=C(O)c1ccnc(-c2ncc(CN3CCN(Cc4ccc(C(F)(F)F)cc4)CC3)n2CCc2ccc(F)cc2)c1]

Example 78 was prepared from Example 77 in a manner analogous to Example 62. The target compound (40 mg, 41.2%) was isolated as a pale pink solid. LCMS MH+ calculated, 568.2, found, 568.4. ¹H NMR (300 MHz, DMSO-d6) δ 13.79 (br s, 1H), 9.96 (br s, 1H), 8.84 (d, J=4.8 Hz, 1H), 8.36 (s, 1H), 7.83-7.76 (m, 3H), 7.69 (m, 2H), 7.11-7.10 (m, 3H), 7.02 (t, J=8.7 Hz, 2H), 4.78 (s, 2H), 4.30 (s, 2H), 3.58-3.42 (m, 3H), 3.42-3.01 (m, 9H).

### Example 79

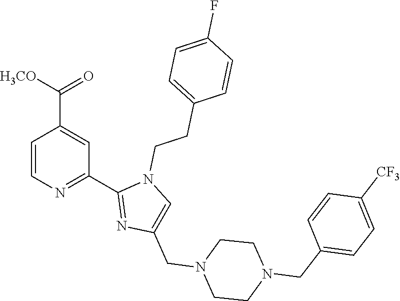 [SMILES: COC(=O)c1ccnc(-c2nc(CN3CCN(Cc4ccc(C(F)(F)F)cc4)CC3)cn2CCc2ccc(F)cc2)c1]

Example 79 was prepared from 1-(4-(trifluoromethyl)benzyl)piperazine [107890-32-4] in a manner analogous to Example 63. The target compound (70 mg, 40.2%) was isolated as a yellow semi-solid. LCMS MH+ calculated, 582.2, found, 582.5.

### Example 80

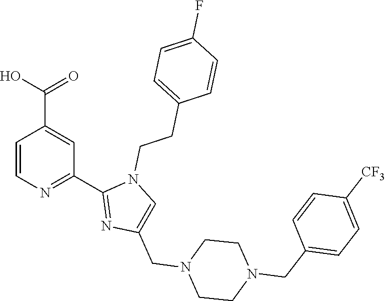 [SMILES: O=C(O)c1ccnc(-c2nc(CN3CCN(Cc4ccc(C(F)(F)F)cc4)CC3)cn2CCc2ccc(F)cc2)c1]

Example 80 was prepared from Example 79 in a manner analogous to Example 64. The target compound (30 mg, 44.1%) was isolated as an off white solid. LCMS MH+ calculated, 568.2, found, 568.4. ¹H NMR (400 MHz, DMSO-d6) δ 8.85 (d, 1H), 8.35 (s, 1H), 7.85 (d, 1H), 7.75 (d, 2H), 7.65-7.55 (d, 2H), 7.45 (s, 1H), 7.10-7.00 (d, 2H), 7.00-6.90 (d, 2H), 4.85-4.75 (t, 2H), 4.20 (s, 2H), 3.25-.3.05 (m, 6H), 3.05-2.95 (t, 2H), 2.90-2.65 (m, 4H).

### Example 81

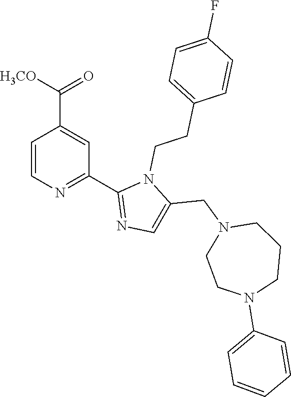 [SMILES: COC(=O)c1ccnc(-c2ncc(CN3CCCN(c4ccccc4)CC3)n2CCc2ccc(F)cc2)c1]

Example 81 was prepared from 1-phenyl-1,4-diazepane [61903-27-3] in a manner analogous to Example 61. The target compound (90 mg, 41.8%) was isolated as a brown semi-solid. LCMS MH+ calculated, 514.3, found, 514.4.

### Example 82 &

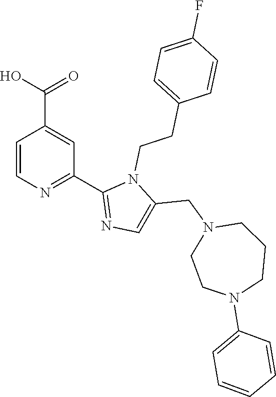 [SMILES: O=C(O)c1ccnc(-c2ncc(CN3CCCN(c4ccccc4)CC3)n2CCc2ccc(F)cc2)c1]

Example 82 was prepared from Example 81 in a manner analogous to Example 62. The target compound (30 mg, 34.4%) was isolated as an off-white solid. LCMS MH+ calculated, 500.2, found, 500.5. ¹H NMR (300 MHz, DMSO-d6) δ 11.92 (br s, 1H), 8.95 (d, J=5.1 Hz 1H), 8.17 (d, J=10.8 Hz, 2H), 7.98 (d, J=4.5 Hz, 1H), 7.21 (t, J=7.8 Hz, 2H), 6.97-6.85 (m, 4H), 6.81-6.71 (m, 2H), 6.67 (t, J=7.2 Hz, 1H), 5.06 (d, J=6.3 Hz, 2H), 4.61 (s, 2H), 3.86 (s, 2H), 3.72-3.41 (m, 6H), 2.90-2.80 (m, 2H), 2.30-2.20 (m, 2H).

### Example 83

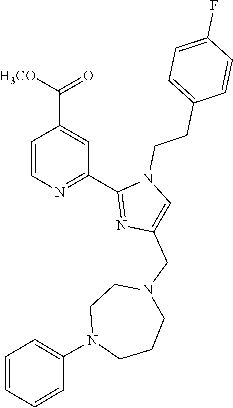 [SMILES: COC(=O)c1ccnc(-c2nc(CN3CCCN(c4ccccc4)CC3)cn2CCc2ccc(F)cc2)c1]

Example 83 was prepared from 1-phenyl-1,4-diazepane [61903-27-3] in a manner analogous to Example 63. The target compound (80 mg, 37.2%) was isolated as a pale yellow semi-solid. LCMS MH+ calculated, 514.3, found, 514.4.

### Example 84

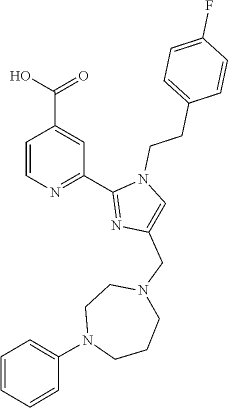 [SMILES: O=C(O)c1ccnc(-c2nc(CN3CCCN(c4ccccc4)CC3)cn2CCc2ccc(F)cc2)c1]

Example 84 was prepared from Example 83 in a manner analogous to Example 64. The target compound (40 mg, 51.9%) was isolated as a pink solid. LCMS MH+ calculated, 500.2, found, 500.4. ¹H NMR (300 MHz, DMSO-d6) δ 12.05 (br s, 1H), 8.81 (d, J=5.1 Hz, 1H), 8.39 (s, 1H), 7.79 (dd, J=1.8, 5.1 Hz, 1H), 7.34 (s, 1H), 7.17 (t, J=8.4 Hz, 2H), 7.10-7.06 (m, 2H), 6.97 (t, J=9.0 Hz, 2H), 6.74 (d, J=8.4 Hz, 2H), 6.63 (t, J=7.2 Hz, 1H), 4.79 (t, J=6.9 Hz, 2H), 4.13 (s, 2H), 3.67 (s, 2H), 3.43 (d, J=5.7 Hz, 2H), 3.20-2.49 (m, 6H), 2.11-2.08 (m, 2H).

### Example 85

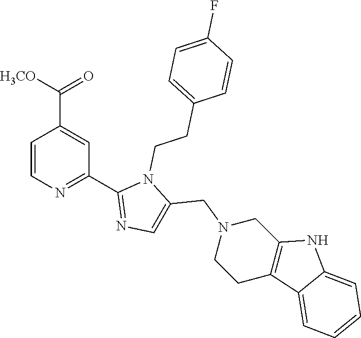 [SMILES: COC(=O)c1ccnc(-c2ncc(CN3CCc4c([nH]c5ccccc45)C3)n2CCc2ccc(F)cc2)c1]

Example 85 was prepared from 2,3,4,9-tetrahydro-H-pyrido[3,4-b]indole [16502-01-5] in a manner analogous to Example 61. The target compound (70 mg, 49.6%) was isolated as a yellow semi-solid. LCMS MH+ calculated, 510.2, found, 510.4.

### Example 86

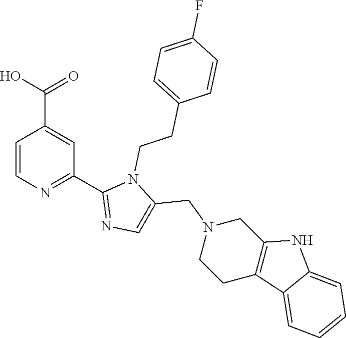 [SMILES: O=C(O)c1ccnc(-c2ncc(CN3CCc4c([nH]c5ccccc45)C3)n2CCc2ccc(F)cc2)c1]

Example 86 was prepared from Example 85 in a manner analogous to Example 62. The target compound (13 mg, 19.1%) was isolated as a brown solid. LCMS MH+ calculated, 496.2, found, 496.4. ¹H NMR (300 MHz, DMSO-d6) δ 11.05 (br s, 1H), 10.50 (br s, 1H), 8.85 (d, J=5.1 Hz, 1H), 8.15 (s, 1H), 7.85 (d, J=4.8 Hz, 1H), 7.56 (s, 1H), 7.51 (d, J=7.8 Hz, 2H), 7.15 (t, J=7.2 Hz, 1H), 7.06 (t, J=7.5 Hz, 1H), 6.90-6.82 (m, 4H), 4.96 (t, J=6.0 Hz, 2H), 4.66 (s, 2H), 4.51 (s, 2H), 3.83-3.72 (m, 2H), 3.15-2.98 (m, 2H), 2.89 (t, J=6.3 Hz, 2H).

### Example 87

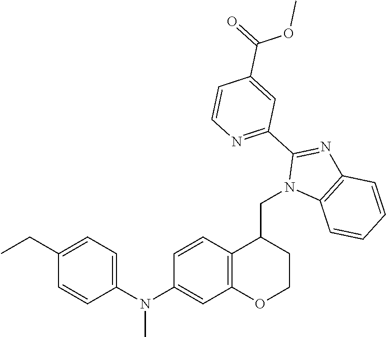 [SMILES: CCc1ccc(N(C)c2ccc3c(c2)OCCC3Cn2c(-c3cc(C(=O)OC)ccn3)nc3ccccc32)cc1]

### Example 87A

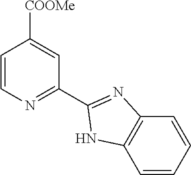 [SMILES: COC(=O)c1ccnc(-c2nc3ccccc3[nH]2)c1]

### Example 87A

A pressure vessel was charged with a mixture of Example 19A (400 mg, 1.4 mmol), Pd(dppf)Cl₂ (143 mg, 0.2 mmol) and Et₃N (1.6 mL, 11.6 mmol) in methanol (30 mL) and stirred under CO gas (50 psi) at 80° C. for 18 h. The reaction mixture was filtered through a celite pad. The filtrate was concentrated and purified by silica gel column chromatography (gradient elution with 20% EtOAc in Hexane) to afford the target compound (320 mg, 86.9%) as a white solid. LCMS MH+ calculated, 254.1, found, 254.1. ¹H-NMR [300 MHz, CDCl₃] δ 10.50 (br s, 1H), 8.96 (s, 1H), 8.98 (d, J=5.1 Hz, 1H), 7.93 (dd, J=5.1 Hz, 1.5 Hz, 1H), 7.89-7.87 (m, 1H), 7.55-7.52 (m, 1H), 7.32-7.34 (m, 2H), 4.00 (s, 3H).

### Example 87B

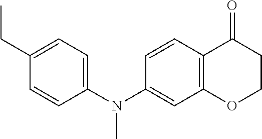 [SMILES: CCc1ccc(N(C)c2ccc3c(c2)OCCC3=O)cc1]

### Example 87B

To a stirred solution of 4-ethyl-N-methylaniline ([37846-06-3], 0.5 g, 3.70 mmol) and 7-bromochroman-4-one ([18442-22-3], 1.0 g, 4.44 mmol) in toluene (10 mL) was added Cs₂CO₃ (2.4 g, 7.40 mmol), BINAP (184 mg, 0.29 mmol) and Pd(OAc)₂ (66 mg, 0.29 mmol). The reaction was stirred at 100° C. for 12 h under inert atmosphere. The mixture was partitioned between water and EtOAc. The organic layer was concentrated and purified by silica-gel column chromatography [gradient elution with 10% EtOAc in Hexane] to afford the target compound (800 mg, 80.0%) as a brown solid. LCMS MH+ calculated, 282.1, found, 282.2.

### Example 87C

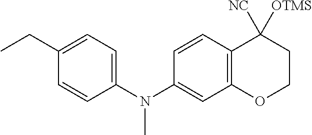

### Example 87C

To a stirred solution of Example 87B (500 mg, 1.77 mmol) in toluene (7 mL) was added trimethylsilyl cyanide (0.73 mL, 5.08 mmol) and zinc iodide (23 mg, 0.07 mmol). The reaction was heated at 60° C. for 2 h. The mixture was partitioned between saturated aqueous NaHCO₃ and EtOAc. The organic layer was dried and concentrated to give the target compound (630 mg, 93.3/0) as brown oil, which was used in the next step without further purification. LCMS MH+ calculated, 381.2, found, 381.5.

### Example 87D

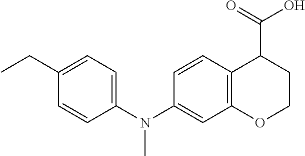 [SMILES: CCc1ccc(N(C)c2ccc3c(c2)OCCC3C(=O)O)cc1]

### Example 87D

To a stirred solution of Example 87C (630 mg, 1.66 mmol) in AcOH (1.6 mL) was added SnCl₂.2H₂O (1.49 g, 6.64 mmol) and conc. HCl (1.6 mL). The reaction was stirred for 12 h at 100° C. under inert atmosphere. The mixture was partitioned between water and DCM. The organic layer was concentrated and purified by silica-gel column chromatography [gradient elution with 30% EtOAc in hexane] to afford the target compound (340 mg, 93.3%) as a yellow solid.

### Example 87E

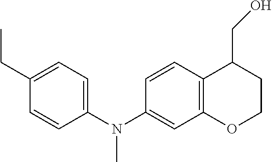 [SMILES: CCc1ccc(N(C)c2ccc3c(c2)OCCC3CO)cc1]

### Example 87E

To a stirred solution of Example 87D (240 mg, 0.77 mmol) in THF (5 ml) was added LiAlH₄ (117 mg, 3.08 mmol) portion wise. The reaction was stirred at 0° C. for 1 h under inert atmosphere. The mixture was partitioned between water and EtOAc. The organic layer was concentrated and purified by silica gel column chromatography [gradient elution with 20% EtOAc in hexane] to afford the target compound (150 mg, 65.2%) as a white solid LCMS MH+ calculated, 298.2, found, 298.3.

### Example 87F

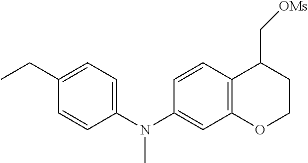 [SMILES: CCc1ccc(N(C)c2ccc3c(c2)OCCC3COS(C)(=O)=O)cc1]

### Example 87F

To a stirred solution of Example 87E (170 mg, 0.57 mmol) and triethylamine (0.16 mL, 1.14 mmol) in DCM (10 ml) was added methane sulfonylchloride (0.05 ml, 0.68 mmol). The reaction was stirred at room temperature for 30 min under inert atmosphere. The mixture was partitioned between water and DCM. The organic layer was concentrated to afford the target compound (182 mg, 85%) as a yellow liquid, which was used directly in the next step.

### Example 87

To a mixture of Example 87A (94 mg, 0.37 mmol) and Example 87F (182 mg, 0.48 mmol) in DMF (5 mL) was added Cs₂CO₃ (243 mg, 0.74 mmol). The reaction was stirred for 2 h at 80° C. The mixture was partitioned between water and EtOAc. The organic layer was dried, concentrated and purified by preparative HPLC to obtain the target compound (25 mg, 12.7%) as a brown solid. LCMS MH+ calculated, 533.3, found, 533.4.

### Example 88

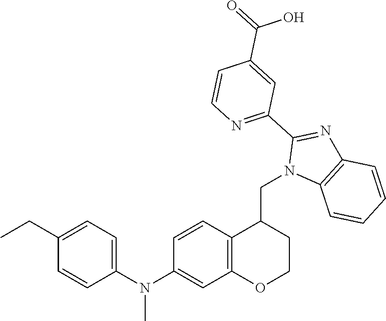 [SMILES: CCc1ccc(N(C)c2ccc3c(c2)OCCC3Cn2c(-c3cc(C(=O)O)ccn3)nc3ccccc32)cc1]

### Example 88

To a stirred solution of Example 87 (25 mg, 0.046 mmol) in a [3:1] mixture of THF in H₂O (2 mL) was added LiOH.H₂O (3 mg, 0.07 mmol). The reaction was stirred for 30 min at room temperature. The mixture was acidified with 2N aq. HCl and concentrated. The residue was purified by preparative HPLC to furnish the target compound (9 mg, 37%) as a light brown solid. LCMS MH+ calculated, 519.2, found, 519.4. ¹H-NMR: [300 MHz, DMSO-d₆] δ 8.89 (d, J=5.1 Hz, 1H), 8.65 (s, 1H), 7.86 (d, J=4.8 Hz, 1H), 7.79 (t, J=6.9 Hz, 2H), 7.39-7.28 (m, 2H), 7.12 (d, J=8.1 Hz, 2H), 6.88 (d, J=8.1 Hz, 2H), 6.47 (d, J=7.8 Hz, 1H), 6.20 (dd, J=8.1, 1.8 Hz, 1H), 6.06 (s, 1H), 5.49-5.42 (m, 1H), 4.95-4.87 (m, 1H), 4.22 (t, J=12.0 Hz, 1H), 4.08-4.04 (m, 1H), 3.23 (s, 1H), 3.09 (s, 3H), 2.57-2.50 (m, 2H), 1.75 (s, 1H), 1.64-1.59 (m, 1H), 1.17 (t, J=7.5 Hz, 3H).

### Example 89

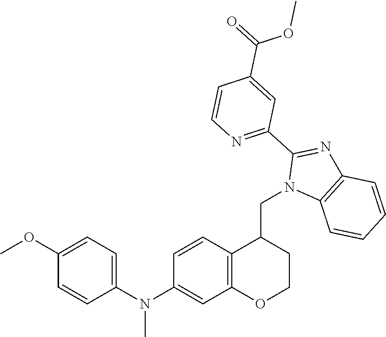 [SMILES: COC(=O)c1ccnc(-c2nc3ccccc3n2CC2CCOc3cc(N(C)c4ccc(OC)cc4)ccc32)c1]

### Example 89

To a mixture of Example 87A (129 mg, 0.50 mmol) and Example 59E (250 mg, 0.66 mmol) in DMF (5 mL) was added Cs₂CO₃ (335 mg, 1.00 mmol). The reaction was stirred for 2 h at 80° C. The mixture was partitioned between water and EtOAc. The organic layer was dried, concentrated and purified by preparative HPLC to obtain the target compound (70 mg, 25%) as a brown solid. LCMS MH+ calculated, 535.2, found, 535.5.

### Example 90

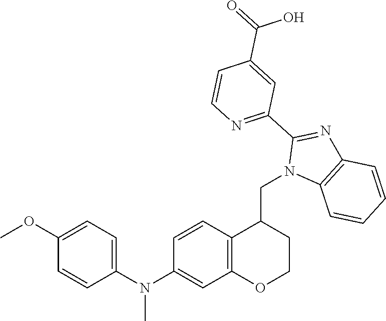 [SMILES: COc1ccc(N(C)c2ccc3c(c2)OCCC3Cn2c(-c3cc(C(=O)O)ccn3)nc3ccccc32)cc1]

### Example 90

To a stirred solution of Example 89 (60 mg, 0.11 mmol) in a [3:1] mixture of THF in H₂O (2 mL) was added LiOH.H₂O (7 mg, 0.16 mmol). The reaction was stirred for 30 min at room temperature. The reaction was acidified with 2N aq. HCl and concentrated. The residue was purified by preparative HPLC to furnish the target compound (35 mg, 60.34%) as a light brown solid. LCMS MH+ calculated, 521.2, found, 521.4. ¹H-NMR (300 MHz, DMSO-d₆) δ 8.88 (d, J=5.1 Hz, 1H), 8.62 (s, 10H), 7.86 (dd, J=5.1, 1.5 Hz, 1H), 7.79 (t, J=7.8 Hz, 2H), 7.28-7.39 (m, 2H), 6.98 (d, J=9.0 Hz, 2H), 6.91 (d, J=9.0 Hz, 2H), 6.42 (d, J=8.4 Hz, 1H), 6.06 (dd, J=8.4, 2.4 Hz, 1H), 5.89 (d, J=2.4 Hz, 1H), 5.47-5.40 (m, 1H), 4.92-4.85 (m, 1H), 4.18 (t, J=10.8 Hz, 1H), 4.05-4.01 (m, 1H), 3.75 (s, 3H), 3.19-3.16 (m, 1H), 3.06 (s, 3H), 1.75-1.69 (m, 1H), 1.59 (d, 1H).

### Example 91

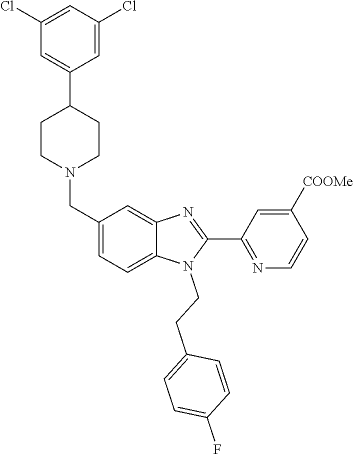 [SMILES: COC(=O)c1ccnc(-c2nc3cc(CN4CCC(c5cc(Cl)cc(Cl)c5)CC4)ccc3n2CCc2ccc(F)cc2)c1]

### Example 91A

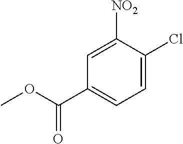

To the solution of 4-chloro-3-nitrobenzoic acid (2.2 gm, 0.01094 mol) in methanol (20 ml) was added thionyl chloride (0.95 ml, 0.01313 mol) at 0° C. The reaction was stirred at 100° C. for 3 h. After cooling, the mixture was partitioned between water and ethyl acetate. The organic layer was dried, filtered and concentrated. The crude product was obtained as an off-white solid and was used in the next step without further purification.

### Example 91B

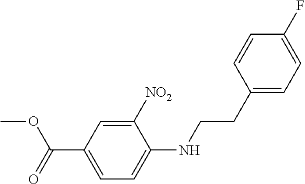 [SMILES: COC(=O)c1ccc(NCCc2ccc(F)cc2)c([N+](=O)[O-])c1]

A stirred mixture of Example 91A (2.0 g, 0.00925 mol) and 2-(4-fluorophenyl)ethanamine (5.1 g, 0.0370 mol) in a 10 mL sealed tube was heated to 80-90° C. for 20 min. Upon cooling, a solid formed. The solid was filtered to afford the target compound (2.0 g, 67.3%) as a yellow solid. LCMS MH+ calculated, 319.1, found, 319.2.

### Example 91C

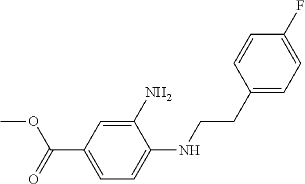 [SMILES: COC(=O)c1ccc(NCCc2ccc(F)cc2)c(N)c1]

To a solution of Example 91B (2.5 gm, 0.00786 mol) in a [1:3] mixture of ethanol:water (100 ml) was added powdered iron (3.1 g, 0.558 mol) followed by ammonium chloride (3.0 g, 0.0558 mol). The reaction was heated to 50° C. and stirring continued for 2 h. After cooling, the mixture was filtered through a pad of celite and the filtrate concentrated under reduced pressure. The crude product was purified by silica gel column chromatography [gradient elution with 15-20% EtOAc in Hexane] to afford the target compound (1.12 g, 49.5%) as an off-white solid. LCMS MH+ calculated, 289.1, found, 289.2.

### Example 91D

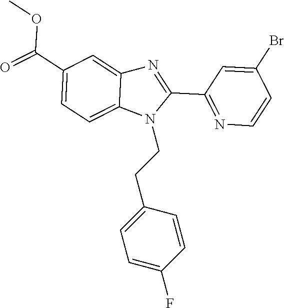 [SMILES: COC(=O)c1ccc2c(c1)nc(-c1cc(Br)ccn1)n2CCc1ccc(F)cc1]

To a stirred solution of 4-bromopicolinic acid (1.05 g, 0.005198 mol) in dry DCM (100 mL) was added EDC.HCl (1.98 g, 0.01036 mol) and HOBt (0.870 g, 0.005686 mol) at 0° C. DIPEA (3.0 mL, 0.026 mol) and Example 91C (1.5 g, 0.005208 mol) were added and the reaction was stirred at room temperature overnight. The mixture was diluted with ice water and extracted with EtOAc (3×100 mL). The combined organic layers were dried and concentrated under reduced pressure. The crude product was purified by silica gel column chromatography using 10-30% EtOAc/hexanes to obtain an off white-solid. The amide was dissolved in a solution of 10% TFA in acetonitrile (45 mL) at 0° C. The reaction was stirred at reflux for 1 h. The mixture was concentrated under reduced pressure. The crude product was purified by silica gel column chromatography [gradient elution with 15-20% EtOAc in hexane] to afford the target compound (1.09 g, 46.2%) as an off-white solid. LCMS MH+ calculated, 454.1, found, 454.2.

### Example 91E

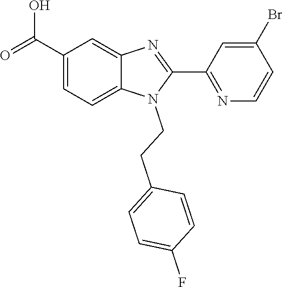 [SMILES: O=C(O)c1ccc2c(c1)nc(-c1cc(Br)ccn1)n2CCc1ccc(F)cc1]

To a stirred solution of Example 91 D (0.5 g, 1.10 mmol) in a [1:1] mixture of THF and H₂O (15 mL) was added LiOH.H₂O (0.067 g, 1.65 mmol). The reaction was stirred at room temperature overnight. The reaction was acidified to pH 5 with saturated citric acid solution and extracted with EtOAc (3×50 mL). The combined organic layers were dried, filtered and concentrated under reduced pressure to afford the target compound (0.400 g, 83.3%) as a yellow solid, which was used in next step without further purification. LCMS MH+ calculated, 440.0, found, 440.2.

### Example 91F

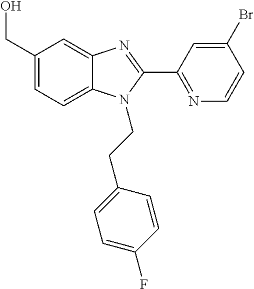 [SMILES: OCc1ccc2c(c1)nc(-c1cc(Br)ccn1)n2CCc1ccc(F)cc1]

To stirred solution of Example 91E (0.4 g, 0.909 mol) in dry THF (10 mL) was added triethylamine (0.18, 0.999 mol) and isobutyl chlorofomate (0.136 g, 0.999 mol) at 0° C. The reaction was stirred for 60 min at room temperature. To the reaction was added NaBH₄ (0.068 g, 1.818 mmol) portion wise at 0° C. and stirring continued for 30 min at room temperature. The mixture was diluted with water and extracted with EtOAc (3×20 mL). The combined organic layers were dried and concentrated under reduced pressure to provide the target compound (0.300 g, 79.0%) as an off white solid, which was used in next step without further purification. LCMS MH+ calculated, 426.1, found, 426.2.

### Example 91G

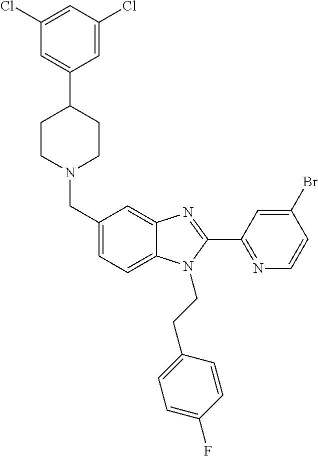 [SMILES: Fc1ccc(CCn2c(-c3cc(Br)ccn3)nc3cc(CN4CCC(c5cc(Cl)cc(Cl)c5)CC4)ccc32)cc1]

To a stirred solution of Example 91F (0.3 g, 0.704 mmol) in dry DCM (2 ml) was added triethylamine (0.1 mL, 0.845 mmol) and DMAP (0.005 g, 0.0422 mmol). The reaction was cooled to 0° C. and methyl sulfonylchloride (0.16 g, 1.408 mmol) was added. The reaction was stirred overnight at room temperature. Chilled water (50 mL) was added and the mixture extracted with DCM (3×50 mL). The combined organic layers were dried, filtered and concentrated under reduced pressure. The crude product was purified by silica gel column chromatography [gradient elution with 10-30% EtOAc in hexane] to provide a pale yellow liquid.

To a stirred solution of 4-(3,5-dichlorophenyl)piperidine (Bavetsias et al., J. Med. Chem. 2016, 59, 1388-1409; 0.109 g; 0.476 mmol) in DMF (5 mL) was added Cs₂CO₃ (0.25 g, 0.793 mmol) at 0° C. The mesylate prepared above (0.200 g, 0.396 mmol) was added to the reaction mixture and the reaction temperature slowly raised to 60-80° C. and stirred at this temperature for 12 h. After cooling, the reaction was diluted with water (50 mL) and extracted with EtOAc (3×50 mL). The combined organic extracts were washed with ice water (30 ml) and brine (50 mL), dried over Na₂SO₄ and evaporated. The crude product was purified by silica gel column chromatography [gradient elution with 10-30% EtOAc in hexane] to afford the target compound (0.180 g, 71.4%) as a pale yellow liquid. LCMS MH+ calculated, 637.1, found, 637.3.

### Example 91

To a stirred solution of Example 91G (430 mg, 0.671 mmol) in methanol (15 mL) was added Et₃N (1.0 mL 10.065 mmol) and Pd(dppf)C₂ (73 mg, 0.100 mmol). The reaction was heated at 80° C. under CO gas (50 psi) for 2 h. The mixture was filtered through a celite pad and the pad washed with methanol (10 mL). The filtrate was concentrated and purified by basic alumina column chromatography [gradient elution with 1% MeOH in DCM] to afford the target compound (0.400 g, 82.7%) as a light brown solid. LCMS MH+ calculated, 617.2, found, 617.2.

### Example 92

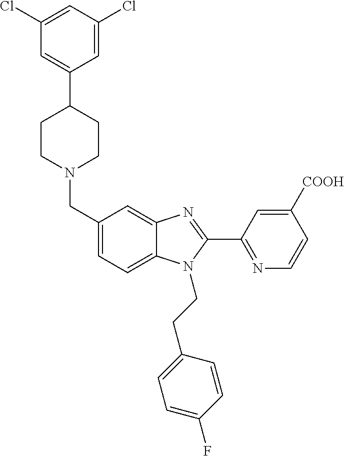 [SMILES: O=C(O)c1ccnc(-c2nc3cc(CN4CCC(c5cc(Cl)cc(Cl)c5)CC4)ccc3n2CCc2ccc(F)cc2)c1]

### Example 92

To a stirred solution of Example 91 (0.163 g, 2.369 mmol) in a [3:1] mixture of THF and H₂O (2 mL) was added LiOH.H₂O (0.025 g, 5.922 mmol). The reaction was stirred overnight. The reaction was acidified with 2N HCl solution, concentrated in vacuo and purified by preparative HPLC to afford the target compound (0.013 g, 8.40%) as a yellow solid. ¹H NMR (300 MHz, DMSO-d₆) δ 13.95 (br s, 1H), 9.60 (br s, 1H), 8.95 (d, J=4.8 Hz, 1H), 8.51 (s, 1H), 7.94-7.91 (m, 2H), 7.84 (d, J=8.4 Hz, 1H), 7.49 (s, 2H), 7.29 (d, J=1.8 Hz, 2H), 7.07-7.02 (m, 2H), 6.93 (t, J=9.0 Hz, 2H), 5.07 (t, J=6.9 Hz, 2H), 4.50 (s, 2H), 3.06 (t, J=6.6 Hz, 4H), 2.89 (t, J=15.9 Hz, 1H), 2.08-2.02 (m, 2H), 1.87 (t, J=12 Hz, 2H).

### Example 93

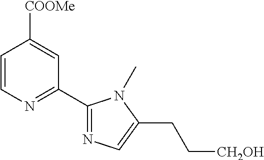 [SMILES: COC(=O)c1ccnc(-c2ncc(CCCO)n2C)c1]

### Example 93A

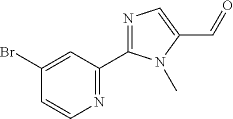

To a stirred solution of Example 55B (700 mg, 0.0028 mol) in dry DCM (14 mL) was added Dess-Martin periodinane (1.8 g, 0.0042 mol) portion wise over a period of 10 min at 0° C. The reaction was stirred at room temperature for 2 h. 10% hypo solution was added and the mixture extracted with EtOAc. The organic layer was further washed with sat NaHCO₃ solution and the separated organic layer dried over anhydrous sodium sulfate and concentrated in vacuo. The crude product was purified by silica gel column chromatography (gradient elution with 1% MeOH in DCM) to afford the target compound (500 mg, 66.4%) as an off white solid. LCMS MH+ calculated, 266.0, found, 266.1.

### Example 93B

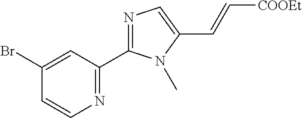 [SMILES: CCOC(=O)/C=C/c1cnc(-c2cc(Br)ccn2)n1C]

To a stirred solution of Example 93A (500 mg, 0.00187 mol) in dry toluene (20 mL) was added ethyl (triphenylphosphoranylidene)acetate (2.0 g, 0.00563). The reaction was heated to 80° C. for 16 h. The mixture was concentrated and purified by silica gel column chromatography (gradient elution with 15% EtOAc in hexane) to afford the target compound (300 mg, 47.5%) as a white solid. LCMS MH+ calculated, 336.0, found, 336.1.

### Example 93C

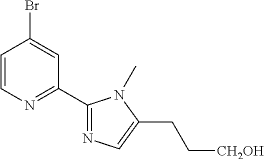 [SMILES: Cn1c(CCCO)cnc1-c1cc(Br)ccn1]

To a stirred solution of Example 93B (200 mg, 0.595 mmol) in EtOH (10 mL) was added NaBH₄ (300 mg, 0.00892 mmol) at 0° C. The reaction was stirred for 16 h at RT. Ice water was added to the reaction and stirring continued for 20 min. The mixture was extracted with EtOAc. The organic layer was dried over anhydrous sodium sulfate, concentrated and purified by silica gel chromatography (gradient elution with 1% MeOH in DCM) to afford the target compound (150 mg, 85%0) as an off white solid. LCMS MH+ calculated, 296.0, found, 296.1.

### Example 93D

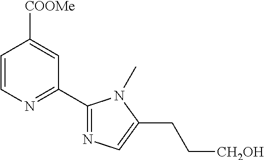 [SMILES: COC(=O)c1ccnc(-c2ncc(CCCO)n2C)c1]

To a stirred solution of Example 93C (250 mg, 0.844 mmol) in methanol (8 mL) and triethylamine (1.0 mL 12.66 mmol) at room temperature was added Pd(dppf)Cl₂ (93 mg, 0.126 mmol). The temperature was raised to 80° C. and the reaction stirred under CO (50 psi) for 2 h. After cooling, the reaction was filtered through a celite pad and the pad washed with methanol (20 mL). The filtrate was concentrated and purified by alumina (basic) column chromatography (gradient elution with 1% MeOH in DCM) to afford the target compound (150 mg, 64%) as a light brown solid. LCMS MH+ calculated, 276.1, found, 276.2.

### Example 93E

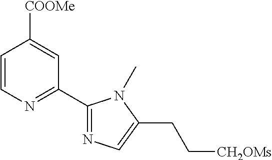

To a stirred solution of Example 93D (100 mg, 0.36 mmol) in DCM (4 mL) at 0° C. was added drop wise triethylamine (0.16 ml, 1.09 mmol) and methane sulfonylchloride (82 mg, 0.727 mmol). The reaction was stirred for 2 h. The reaction was quenched with water (10 mL) and extracted with DCM (2×10 ml). The combined organic layers were dried over anhydrous sodium sulfate, concentrated and purified by alumina (basic) column chromatography (gradient elution with 1% MeOH in DCM) to afford the target compound (90 mg, 70%) as a pale yellow semi-solid.

### Example 93

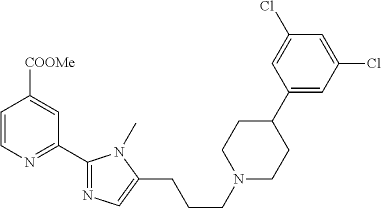 [SMILES: COC(=O)c1ccnc(-c2ncc(CCCN3CCC(c4cc(Cl)cc(Cl)c4)CC3)n2C)c1]

### Example 93

To a stirred solution of 4-(3,5-dichlorophenyl)piperidine (Bavetsias et al., J. Med. Chem. 2016, 59, 1388-1409; 77.8 mg; 0.33 mmol) in dry DMF (2 mL) was added potassium carbonate (77 mg, 0.56 mmol) and Example 93E (100 mg, 0.28 mmol). The reaction was heated to 80° C. for 2 h. The reaction mixture was cooled to room temperature and filtered. The filtrate was concentrated and purified by alumina (basic) column chromatography (gradient elution with 2% MeOH in DCM) to afford the target compound (90 mg, 65.6%) as a pale yellow semi-solid. LCMS MH+ calculated, 487.2, found, 487.3.

### Example 94

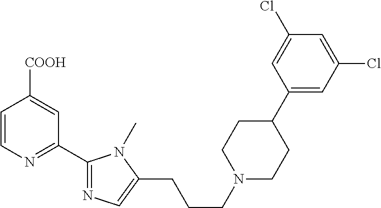 [SMILES: Cn1c(CCCN2CCC(c3cc(Cl)cc(Cl)c3)CC2)cnc1-c1cc(C(=O)O)ccn1]

### Example 94

To a stirred solution of Example 93 (60 mg, 0.123 mmol) in a mixture of THF (2 mL) and H₂O (0.5 mL) at 0° C. was added LiOH.H₂O (25 mg, 0.616 mmol). The reaction was stirred at 25° C. for 2 h. The reaction mixture was concentrated in vacuo to remove THF and the residue acidified to pH 2 with 1M HCl solution. The mixture was concentrated, purified by preparative HPLC and lyophilized to afford the target compound (10 mg, 17%) as a pink solid (TFA salt). LCMS MH+ calculated, 473.1, found, 473.3.

### Example 95

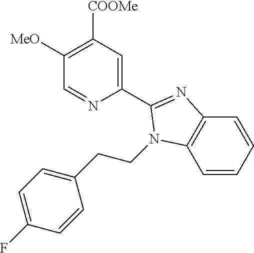 [SMILES: COC(=O)c1cc(-c2nc3ccccc3n2CCc2ccc(F)cc2)ncc1OC]

### Example 95A

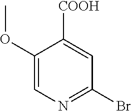

To a stirred solution of 2-bromo-5-methoxypyridine (1.0 g, 0.0053 mol) in dry THF (10 mL) at −78° C. was added LDA (2.12 mL, 2.5M in THF) drop wise over 10 min. The reaction was stirred for 1.5 h. Dry ice was added to the reaction mixture at −78° C. and stirring continued for 10 min. The reaction mixture was slowly warmed to RT and basified with 5% w/v. aq. NaOH solution. The mixture was washed with DCM to remove organic impurities. The aqueous phase was acidified to pH 2 using 6N HCl. A solid formed which was filtered to afford the target compound (540 mg, 40.84%) as an off-white solid. ¹HNMR (400 MHz, DMSO-d6) δ 8.38 (1, s), 7.78 (1H, s), 3.97 (3H, s), 3.82 (3H, s).

### Example 95B

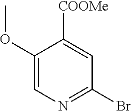

To a stirred solution of Example 95A (500 mg, 2.155 mmol) in DMF (20 mL) at 0° C. was added methyl iodide (455 mg, 3.23 mmol) and K₂CO₃ (445 g, 3.23 mmol). The reaction was stirred at 25° C. for 16 hours. Ice water was added and stirring continued for 20 min. The precipitated solid was filtered and dried in vacuo. The crude product obtained was purified by alumina (basic) column chromatography (gradient elution with 1% MeOH in DCM) to afford the target compound (400 mg, 75%) as a pale yellow solid. LCMS MH+ calculated, 246.0, found, 246.0.

### Example 95C

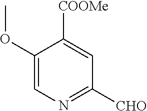

To a stirred solution of Example 95B (100 mg, 0.406 mmol) in DMF (2 mL) was added Et₃SiH (94 mg, 0.13 mmol), Na₂CO₃ (43 mg, 406 mmol) and Pd(dppf)C₁₂ (7.0 mg, 0.01 mmol). The reaction was heated to 80° C. for 3 h under CO (40 psi). The reaction mixture was filtered through a celite pad and the pad washed with methanol (5 mL). The filtrate was concentrated and purified by alumina (basic) column chromatography (gradient elution with 1% MeOH in DCM) to afford the target compound (80 mg, 40.8%) as a light brown solid. LCMS MH+ calculated, 196.1, found, 196.0.

### Example 95D

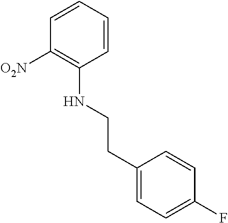 [SMILES: O=[N+]([O-])c1ccccc1NCCc1ccc(F)cc1]

To a stirred solution of 1-bromo-2-nitrobenzene (1 g, 0.00709 mol) and 4-fluorophenethylamine (985 mg, 0.00709 mol) in DMF (15 mL) was added DIEPA (1.26 ml, 0.00709 mol). The reaction was heated to 100° C. for 4 h. After cooling, water was added to the mixture and a precipitate formed. The solid was filtered and dried to afford the target compound (850 mg, 46.2%) as a yellow solid. LCMS MH+ calculated, 261.1, found, 261.2. ¹H NMR (400 MHz, DMSO-d₆) δ 8.18 (1H, J=8.6 Hz, d), 8.09 (1H, s), 7.43 (1H, J=7.3 Hz, t), 7.31-7.18 (2H, m), 7.03 (21, 1=8.6 Hz, t), 6.82 (1H, J=8.6 Hz, d), 6.64 (1H, J=7.3 Hz, t), 3.54 (2H, J=6.9 Hz, 12.4 Hz, q), 3.00 (2H, J=6.9 Hz, t).

### Example 95E

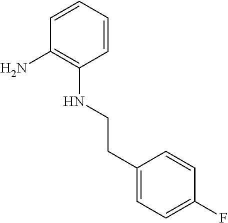 [SMILES: Nc1ccccc1NCCc1ccc(F)cc1]

To a stirred solution of Example 95D (500 mg, 0.00192 mol) in a [1:1] mixture of MeOH and AcOH (2 mL) was added Fe powder (590 mg, 0.01057 mol) portion wise over a period of 10 min. The reaction was heated to 50° C. for 2 h. After cooling, the reaction mixture was filtered. The filtrate was concentrated and purified by silica gel column chromatography (gradient elution with 20% EtOAc in hexane) to afford the target compound (300 mg, 46.2%) as an off white solid. LCMS MH+ calculated, 231.1, found, 231.2. ¹H NMR (400 MHz, DMSO-d6) δ 7.20 (2H, J=8.6 Hz, t), 6.98 (2H, J=8.6 Hz, t), 6.91-6.80 (1H, m), 6.78-6.62 (3H, m), 3.39 (2H, J-=6.9 Hz, t), 2.95 (2H, J=6.9 Hz, t).

### Example 95

To a stirred solution of Example 95E (200 mg, 2.5 mmol) in EtOH (2 mL) was added Example 93C (280 mg, 2.5 mmol) and AcOH (0.2 mL). The reaction was heated in a microwave reactor at 120° C. for 30 min. The reaction mixture was concentrated and the crude product was dissolved in EtOAc and washed with water and brine. The organic layer was dried over anhydrous sodium sulfate, concentrated and purified by silica gel column chromatography (gradient elution with 20% EtOAc in hexane) to afford the target compound (150 mg, 42%) as an off-white solid. LCMS MH+ calculated, 406.2, found, 406.0.

### Example 96

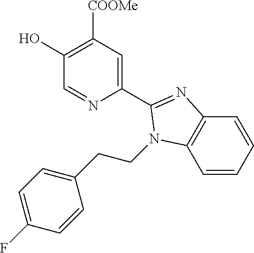 [SMILES: COC(=O)c1cc(-c2nc3ccccc3n2CCc2ccc(F)cc2)ncc1O]

### Example 96

Example 95 (100 mg, 0.24 mmol) was stirred in 30% HBr in AcOH (1 mL) at room temperature for 16 h. The reaction was diluted with ice water and extracted with EtOAc. The organic layer was dried over sodium sulfate and concentrated to afford the target compound (50 mg, 48.5%) which was used in the next step without any further purification. LCMS MH+ calculated, 392.1, found, 392.3.

### Example 97

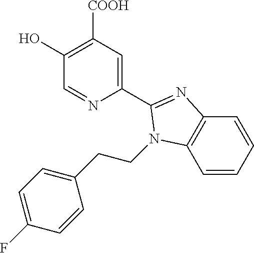 [SMILES: O=C(O)c1cc(-c2nc3ccccc3n2CCc2ccc(F)cc2)ncc1O]

### Example 97

Example 96 (50 mg) was stirred in concentrated HCl (1 mL) overnight at room temperature. The reaction mixture was concentrated and purified by preparative HPLC to afford the target compound (20 mg, 43.0%) as a pale pink solid (HCl salt). LCMS MH+ calculated, 378.1, found, 378.3. ¹H NMR (300 MHz, DMSO-d₆) δ 8.43 (1H, s), 8.24 (1H, s), 7.67-7.55 (2H, m), 7.31-7.15 (4H, m), 7.05 (2H, J=8.7 Hz, t), 4.90 (2H, J=7.2 Hz, t), 3.05 (2H, =7.2 Hz, t).

### Example 98

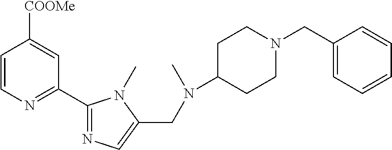 [SMILES: COC(=O)c1ccnc(-c2ncc(CN(C)C3CCN(Cc4ccccc4)CC3)n2C)c1]

### Example 98

To a stirred solution of 1-benzyl-N-methylpiperidin-4-amine ([7006-50-0], 68 mg, 0.33 mmol) in dry DMF (2 mL) was added potassium carbonate (76 mg, 0.553 mmol). Example 55D (90 mg, 0.276 mmol) was added and the reaction heated to 80° C. for 3 h. The reaction mixture was cooled to room temperature and filtered. The filtrate was concentrated and purified by alumina (basic) column chromatography (gradient elution with 1% MeOH in DCM) to afford the target compound (90 mg, 75%) as a yellow semi-solid. LCMS MH+ calculated, 434.3, found, 434.5.

### Example 99

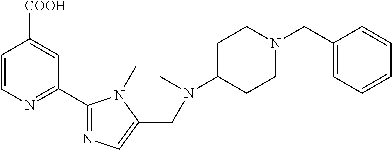 [SMILES: CN(Cc1cnc(-c2cc(C(=O)O)ccn2)n1C)C1CCN(Cc2ccccc2)CC1]

### Example 99

To a stirred solution of Example 98 (90 mg, 0.207 mmol) in a mixture of THF (2 mL) and H₂O (0.5 mL) was added LiOH.H₂O (43 mg, 1.038 mmol) at 0° C. The reaction was stirred at room temperature for 2 h. The reaction mixture was concentrated in vacuo to remove THF. The residue was acidified to pH 2 with 1M HCl and then concentrated. The crude product was purified by preparative HPLC and lyophilized to afford the target compound (25 mg, 29%) as a pink solid (TFA salt). LCMS MH+ calculated, 420.2, found, 420.5. ¹H NMR (300 MHz, DMSO-d6) δ 8.87 (1H, J=4.5 Hz, d), 8.50 (1H, s), 7.86 (1H, J=4.5 Hz, d), 7.56-7.42 (6H, m), 4.31 (4H, s), 4.08 (3H, s), 3.12-2.90 (2H, m), 2.76-2.70 (2H, m), 2.60 (3H, s), 2.32-2.11 (2H, m), 2.07-1.83 (2H, m).

### Example 100

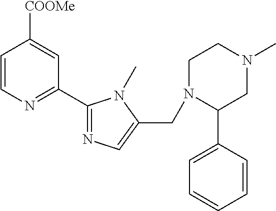 [SMILES: COC(=O)c1ccnc(-c2ncc(CN3CCN(C)CC3c3ccccc3)n2C)c1]

Example 100 was prepared from 1-methyl-3-phenylpiperazine [5271-27-2] in a manner analogous to Example 98. The target compound (90 mg, 80%) was isolated as a yellow semi-solid. LCMS MH+ calculated, 406.2, found, 406.6.

### Example 101

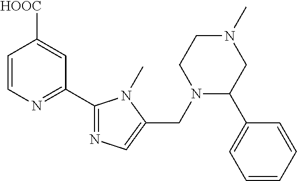 [SMILES: CN1CCN(Cc2cnc(-c3cc(C(=O)O)ccn3)n2C)C(c2ccccc2)C1]

Example 101 was prepared from Example 100 in a manner analogous to Example 99. The target compound (11 mg, 12.7%) was isolated as a pink solid (TFA salt). LCMS MH+ calculated, 392.2, found, 392.0. ¹H NMR (300 MHz, DMSO-d6) δ 8.95 (1H, J=4.34 Hz, d), 8.40 (1H, s), 7.99 (1H, J=4.34 Hz, d), 7.57-7.33 (6H, m), 3.91 (3H, s), 3.66-3.08 (9H, m), 2.82 (3H, s).

### Example 102

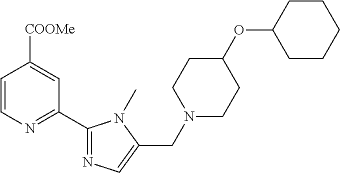 [SMILES: COC(=O)c1ccnc(-c2ncc(CN3CCC(OC4CCCCC4)CC3)n2C)c1]

Example 102 was prepared from 4-(cyclohexyloxy)piperidine [303975-02-2] in a manner analogous to Example 98. The target compound (90 mg, 74.3%) was isolated as a brown semi-solid.

### Example 103

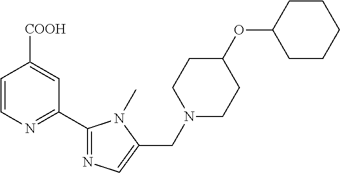 [SMILES: Cn1c(CN2CCC(OC3CCCCC3)CC2)cnc1-c1cc(C(=O)O)ccn1]

Example 103 was prepared from Example 102 in a manner analogous to Example 99. The target compound (30 mg, 34%) was isolated as a pink solid (TFA salt). LCMS MH+ calculated, 399.2, found, 399.3. ¹H NMR (400 MHz, DMSO-d6) δ 8.88 (1H, s), 8.50 (1H, s), 7.84 (1H, s), 7.38 (1H, s), 4.51 (2H, s), 4.08 (3H, s), 3.78-3.60 (2H, m), 3.42-3.11 (4H, m), 2.19-1.60 (8H, m), 1.54-1.41 (1H, m), 1.36-1.02 (5H, m).

### Example 104

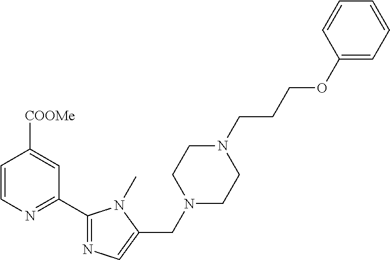 [SMILES: COC(=O)c1ccnc(-c2ncc(CN3CCN(CCCOc4ccccc4)CC3)n2C)c1]

Example 104 was prepared from 1-(3-phenoxypropyl)piperazine [41298-49-1] in a manner analogous to Example 98, The target compound (80 mg, 57.9%) was isolated as a brown semi-solid. LCMS MH+ calculated, 450.2, found, 450.5.

### Example 105

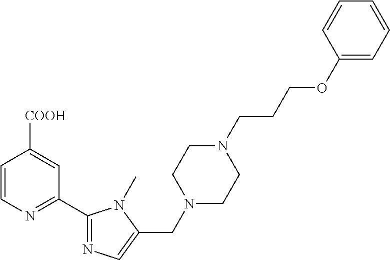 [SMILES: Cn1c(CN2CCN(CCCOc3ccccc3)CC2)cnc1-c1cc(C(=O)O)ccn1]

Example 105 was prepared from Example 104 in a manner analogous to Example 99. The target compound (20 mg, 26%) was isolated as a pink solid (TFA salt). LCMS MH+ calculated, 436.2, found, 436.5. ¹H NMR (400 MHz, DMSO-d6) δ 8.97 (1H, J=4.53 Hz, d), 8.40 (1H, s), 8.03 (1H, J=4.53 Hz, d), 7.61 (1H, s), 7.32-7.23 (2H, m), 6.99-6.88 (3H, m), 4.19-3.89 (711, m), 3.79-3.71 (2H, m), 3.53-3.38 (2H, m), 3.26 (2H, J=6.98 Hz, t), 3.16-2.89 (4H, m), 2.11 (2H, J=6.98 Hz, m).

### Example 106

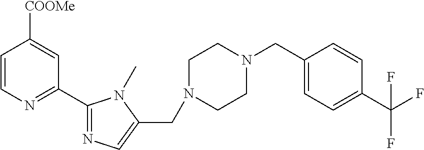 [SMILES: COC(=O)c1ccnc(-c2ncc(CN3CCN(Cc4ccc(C(F)(F)F)cc4)CC3)n2C)c1]

Example 106 was prepared from 1-(4-(trifluoromethyl)benzyl)piperazine [107890-32-4] in a manner analogous to Example 98. The target compound (80 mg, 809) was isolated as a yellow semi-solid. LCMS MH+ calculated, 474.2, found, 474.4.

### Example 107

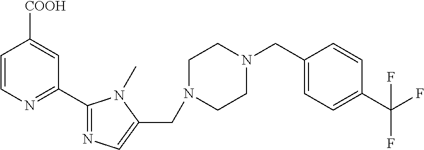 [SMILES: Cn1c(CN2CCN(Cc3ccc(C(F)(F)F)cc3)CC2)cnc1-c1cc(C(=O)O)ccn1]

Example 107 was prepared from Example 106 in a manner analogous to Example 99. The target compound (15 mg, 13.7%) was isolated as a pink solid (TFA salt). ¹H NMR (300 MHz, DMSO-d6) δ 9.02 (1H, J=4.5 Hz, d), 8.44 (1H, s), 8.08 (1H, J=4.5 Hz, d), 7.85 (2H, J=7.9 Hz, d), 7.72 (2H, J=7.9 Hz, d), 7.65 (1H, s), 4.37 (4H, s), 4.07 (3H, s), 3.86-3.78 (4H, m), 3.28-3.13 (4H, m).

The compounds disclosed herein may have one or more chiral centers and can be synthesized as stereoisomeric mixtures, isomers of identical constitution that differ in the arrangement of their atoms in space. The compounds may be used as mixtures or the individual components/isomers may be separated using reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of disclosed herein may be synthesized using optically pure, chiral reagents and intermediates to prepare individual isomers or entantiomers. Unless otherwise indicated, all chiral (enantiomeric and diastereomeric) and racemic forms are within the scope of the invention.

Example 108 This Example illustrates that exemplary compounds disclosed herein inhibit KDM5B enzymatic activity.

Ten point dose-response curves for compounds shown in Table 1 were determined using a homogeneous time resolved fluorescence (HTRF) assay (Reaction Biology Corp, Malvern, Pa.). This assay combines fluorescence resonance energy transfer (FRET) with time resolve (TR) measurements (TR-FRET). The KDM5B α-ketoglutarate-dependent demethylase activity is calculated by measuring demethylation of a biotin labeled histone tri-methylated H3K4 substrate using an europium-cryptate (Eu)-labeled antibody donor specific for dimethylated histone H3K4 and a Streptavidin-XL665 acceptor that binds to the biotin group of substrate, and detecting FRET by exciting the reaction mixture at 320 nm and reading dual emissions at 615 nm and 665 nm.

Briefly, compounds disclosed herein were solubilized in DMSO and a series of 10, three-fold serial dilutions were made for each compound in 100% DMSO. The initial starting concentration for the serial dilutions of each compound was 10 μM or 100 μM. Control samples lacking compound, KDM5B enzyme or various reaction components also were prepared and processed in parallel with compound test samples.

An aliquot of each serial dilution of test compound was added to a 384 well plate (Corning Cat #3572) containing 1.2 nM KDM5B enzyme suspended in 50 mM Hepes, pH7.5, 50 mM NaCl, 0.01% Tween 20, 0.1% BSA, and 1% DMSO (final concentrations) in a 10 microliter reaction volume using a LABCYTE ECHO liquid handler. The samples were mixed, subjected to centrifugation and the plate was pre-incubated at room temperature for 15 min, to which 30 nM Biotin-H3K4Me3 1-21 substrate (Anaspec Cat #64192), 20 μM Fe(III) and 20 μM α-ketoglutarate co-factors, and 100 μM Ascorbate were added to initiate the enzymatic reaction. The reaction mixture was incubated at room temperature for 45 minutes. A 10 μl aliquot of a detection mixture of Eu-labeled anti-histone H3K4Me2 antibody (CisBIO Cat #610AXLB) and Streptavidin-XL665 (CisBIO Cat #610 SAXLB) in 200 mM potassium fluoride and 10 mM EDTA was added and kinetic measurements were read at 5 minute intervals for a period of 30 minutes using an Envision Multiplate Reader (PerkinElmer Model 2102; excitation at 320 nm and emmission reads at 615 nm and 665 nm). The IC₅₀ value for each compound was determined from the 665/615 ratio obtained for each 10 point dose-response curve using GraphPad Prism 4 software with a sigmodial dose response. The results for exemplary compounds of Formula (I) are shown in Table 1. Key: “A”≤100 nM; “B”>100 nM-≤500 nM; “C”>500 nM and N/D is not determined.

TABLE 1
Inhibition of KDM5B Activity by
Exemplary Compounds of Formula (I)

| Example Number | IC₅₀ (nM) |
|---|---|
| 1 | B |
| 2 | A |
| 3 | B |
| 4 | A |
| 5 | C |
| 6 | B |
| 7 | B |
| 8 | C |
| 9 | B |
| 10 | B |
| 11 | C |
| 12 | A |
| 13 | C |
| 14 | B |
| 15 | A |
| 16 | B |
| 28 | B |
| 29 | A |
| 31 | B |
| 34 | B |
| 36 | C |
| 42 | B |
| 47 | A |
| 51 | A |
| 54 | B |
| 62 | B |
| 66 | B |
| 68 | A |
| 70 | B |
| 72 | A |
| 74 | A |
| 76 | A |
| 78 | A |
| 82 | A |
| 86 | A |
| 88 | C |
| 90 | C |
| 101 | A |
| 107 | A |

EXAMPLE 109 This Example illustrates methodology for testing exemplary compounds for inhibition of growth of tumor cell lines that express KDM5B.

The ZR-75-1 cell line cell line was established from the mammary gland of a 63-year-old female, derived from a metastatic site, and has been shown to be sensitive to inhibitors of KDM5B.

Inhibition of KDM5B-mediated cellular proliferation by compounds of Formula (I) is measured in a CellTiter Glo luminescence assay (Promega Corp, Madison, Wis.), which determines the number of viable cells by quantitating the amount of ATP, using a BMG LabTech CLARIOStar instrument in accordance with the manufacturer's instructions. Briefly, ZR-75-1 cells are plated at a density of 1500 cells/90 μl/well in 96 well culture plates and cultured in RPMI 1640 medium (Gibco) supplemented with 10% fetal bovine serum (FBS) and 1% penicillin and 1% streptomycin at 37° C. A series of 3-fold serial dilutions of each test compound of Formula (I) is prepared in complete RPMI 1640 medium and added to the cells at final concentrations ranging from 10 μM to 0.0015 nM. Control samples lacking test compound or cells are processed in parallel. The plates are incubated at 37° C. for four days and thereafter 50 μl fresh medium containing the same concentration of test compound is added. The plates are incubated for an additional three days (Day 7), at which time 50 uL is removed from each well and replaced with 50 uL fresh medium containing the same concentration of test compound, and plates are incubated for an additional three days (Day 10). A baseline measurement, as described below, is taken for a time zero point at Day 0.

At Day 10, the supernatant is removed by aspiration and the plate is allowed to equilibrate to room temperature (˜15 min). The cells are lysed using 30 μl (30 μl for Day 0) of Cell Titer Glo reagent (Promega Corp, Madison, Wis.). The plates are shaken for two minutes and incubated at room temperature for 30 minutes protected from light. The degree of inhibition of cell viability is determined using a spectrophotometric readout by measuring the luminescence at 340 nm and the EC₅₀ concentration for each compound is calculated using Graph Pad Prism 4 software.

The foregoing description discloses only exemplary embodiments of the invention.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

1. A compound of formula (I): 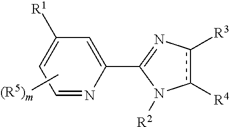
or a pharmaceutical salt thereof:
wherein:
R¹ is —COOR⁶, —C(O)N(H)CN, —C(O)N(H)OH, or tetrazolyl;
R² is C₀-C₆ alkyl-R⁷;
R³ and R⁴ are each independently selected from hydrogen, halogen, alkyl, alkoxy, —(CH₂)ₙOH, —(CH₂)ₙC(O)NHR⁶, —(CH₂)ₙC(O)NHR⁵, —(CH₂)ₙ C(O)NHR⁷, —(CH₂)ₙ C(O)NR⁶R⁷, —(CH₂), N(R⁶)C(O)R⁵, (CH₂)ₙN(R⁶)C(O)R⁷, or —(CH₂)ₙNHC(O)R⁵, —(CH₂)ₙ NHC(O)R⁷, carbocyclyl, heterocyclyl, aryl, heteroaryl, alkylcarbocyclyl, alkylheterocyclyl, alkylaryl, alkylheteroaryl, wherein each of the carbocyclyl, heterocyclyl, aryl, heteroaryl, alkylcarbocyclyl, alkylheterocyclyl, alkylaryl, alkylheteroaryl is optionally substituted with one or more R;
or R³ and R⁴ and the carbon atoms to which they are attached form a 5-7 membered unsaturated, partial unsaturated or saturated ring system optionally containing 1-3 heteroatoms selected from N, O or S, and further optionally substituted with one or more R⁵;
R⁵ is alkyl, alkenyl, alkynyl, halogen, haloalkyl, alkoxy, cyano, amino, —COOR⁶, C(O)NHR⁶, C(O)N(R⁶)₂, N(R⁶)C(O)R³, NHC(O)R³ aryloxy, or optionally substituted heterocyclyl;
R⁶ is hydrogen or alkyl;
R⁷ is hydrogen, carbocyclyl, heterocyclyl, aryl, or heteroaryl, wherein each of the carbocyclyl, heterocyclyl, aryl, or heteroaryl may be optionally substituted with one or more R⁵; or
R⁶ and R⁷ and the nitrogen atom to which they are attached form a 4-7 membered unsaturated, partial unsaturated or saturated ring system optionally containing 1-3 heteroatoms selected from N, O or S, and further optionally substituted with one or more R; and
m and n are each independently zero or an integer between 1 and 3.

2. The compound of claim 1, wherein R¹ is —COOR⁶.

3. The compound of claim 2, wherein R⁶ is hydrogen.

4. The compound of claim 2, wherein R² is C₁-C₃ alkyl-R⁷.

5. The compound of claim 4, wherein R⁷ is selected from the group consisting of aryl or heteroaryl, wherein each of the aryl or the heteroaryl may be optionally substituted with one or more R⁵.

6. The compound of claim 4, wherein the aryl is phenyl or 1,2,3,4-tetrahydronaphthalyl.

7. The compound of claim 5, wherein R⁵ is alkyl, halogen, haloalkyl, alkoxy or cyano.

8. The compound of claim 5, wherein R⁴ is hydrogen, —C(O)NH₂ or —(CH₂)ₙOH, wherein n is zero or an integer between 1 and 3.

9. The compound of claim 1, wherein the compound is selected from the group consisting of:

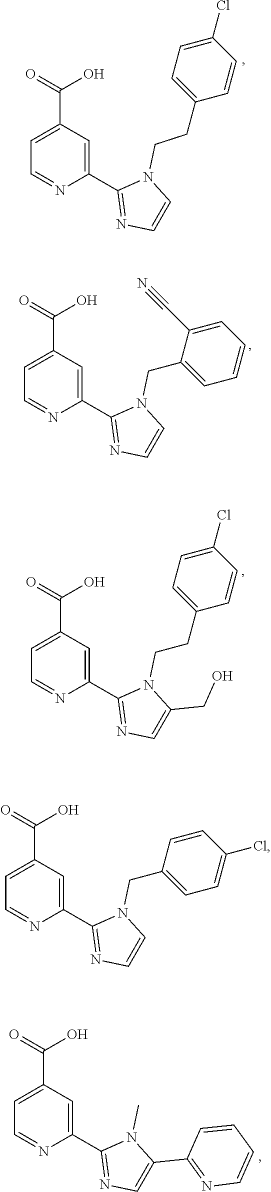 [SMILES: O=C(O)c1ccnc(-c2nccn2CCc2ccc(Cl)cc2)c1 ; N#Cc1ccccc1Cn1ccnc1-c1cc(C(=O)O)ccn1 ; O=C(O)c1ccnc(-c2ncc(CO)n2CCc2ccc(Cl)cc2)c1 ; O=C(O)c1ccnc(-c2nccn2Cc2ccc(Cl)cc2)c1 ; Cn1c(-c2ccccn2)cnc1-c1cc(C(=O)O)ccn1]

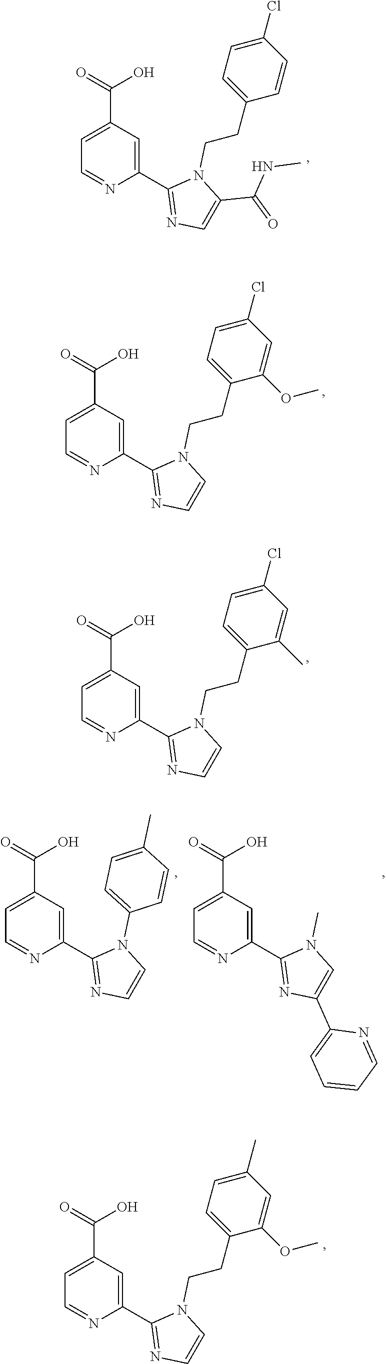

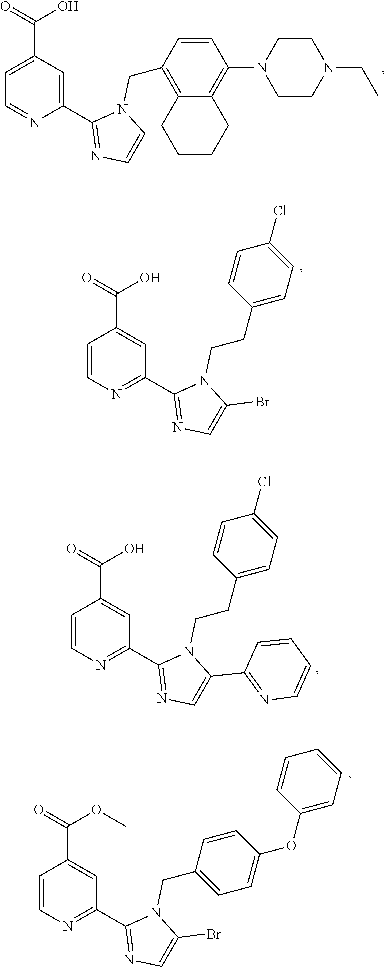 [SMILES: CCN1CCN(c2ccc(Cn3ccnc3-c3cc(C(=O)O)ccn3)c3c2CCCC3)CC1 ; O=C(O)c1ccnc(-c2ncc(Br)n2CCc2ccc(Cl)cc2)c1 ; O=C(O)c1ccnc(-c2ncc(-c3ccccn3)n2CCc2ccc(Cl)cc2)c1 ; COC(=O)c1ccnc(-c2ncc(Br)n2Cc2ccc(Oc3ccccc3)cc2)c1]

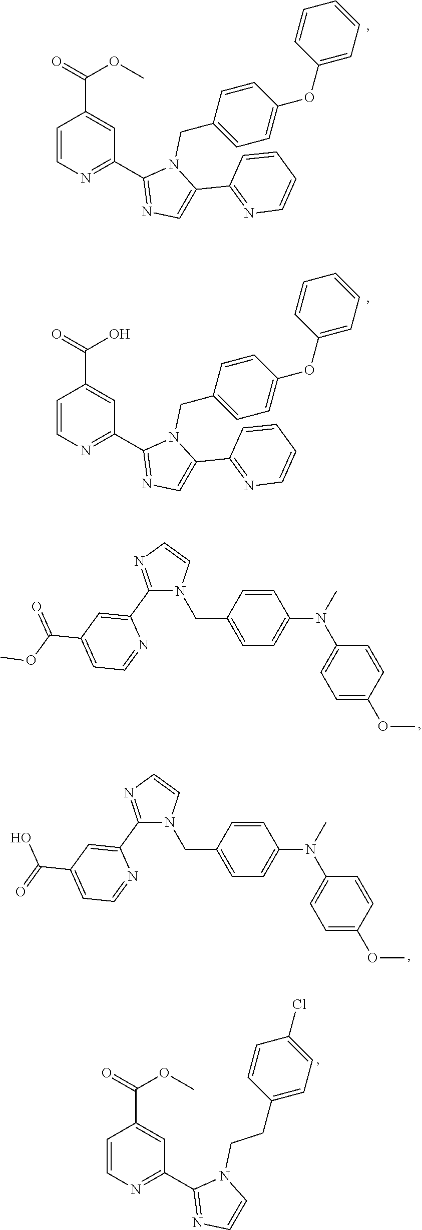

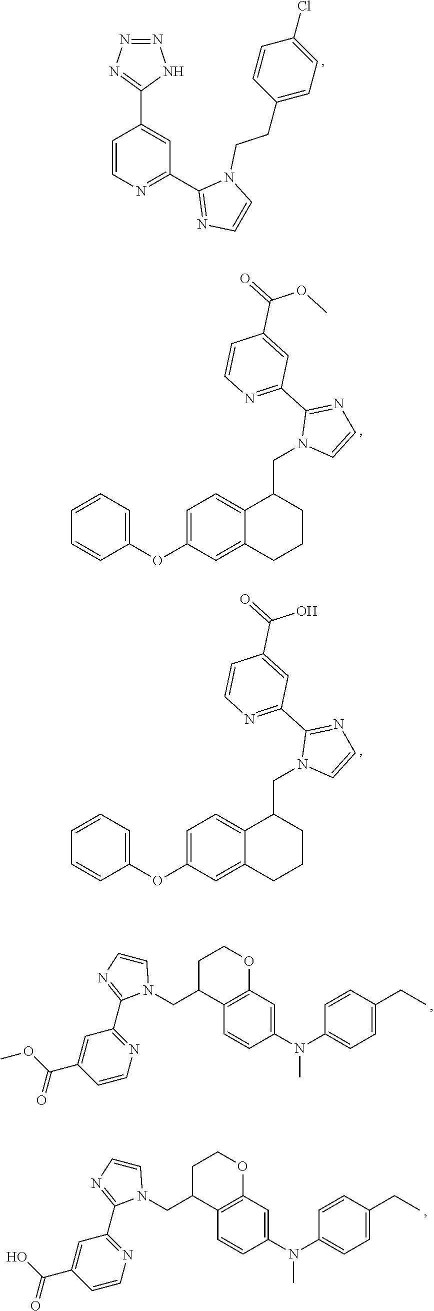 [SMILES: Clc1ccc(CCn2ccnc2-c2cc(-c3nnn[nH]3)ccn2)cc1 ; COC(=O)c1ccnc(-c2nccn2CC2CCCc3cc(Oc4ccccc4)ccc32)c1 ; O=C(O)c1ccnc(-c2nccn2CC2CCCc3cc(Oc4ccccc4)ccc32)c1 ; CCc1ccc(N(C)c2ccc3c(c2)OCCC3Cn2ccnc2-c2cc(C(=O)OC)ccn2)cc1 ; CCc1ccc(N(C)c2ccc3c(c2)OCCC3Cn2ccnc2-c2cc(C(=O)O)ccn2)cc1]

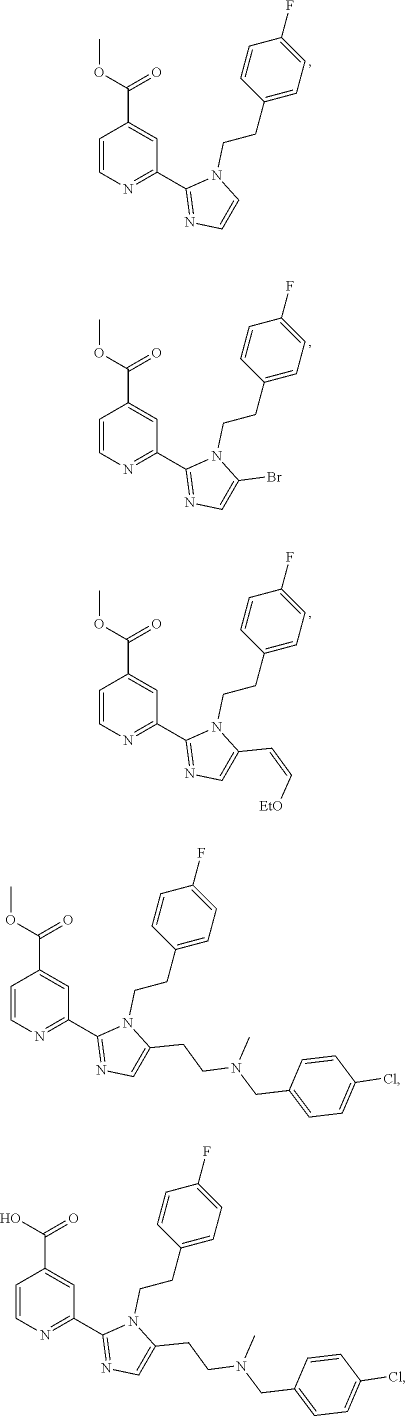 [SMILES: COC(=O)c1ccnc(-c2nccn2CCc2ccc(F)cc2)c1 ; COC(=O)c1ccnc(-c2ncc(Br)n2CCc2ccc(F)cc2)c1 ; CCO/C=C\c1cnc(-c2cc(C(=O)OC)ccn2)n1CCc1ccc(F)cc1 ; COC(=O)c1ccnc(-c2ncc(CCN(C)Cc3ccc(Cl)cc3)n2CCc2ccc(F)cc2)c1 ; CN(CCc1cnc(-c2cc(C(=O)O)ccn2)n1CCc1ccc(F)cc1)Cc1ccc(Cl)cc1]

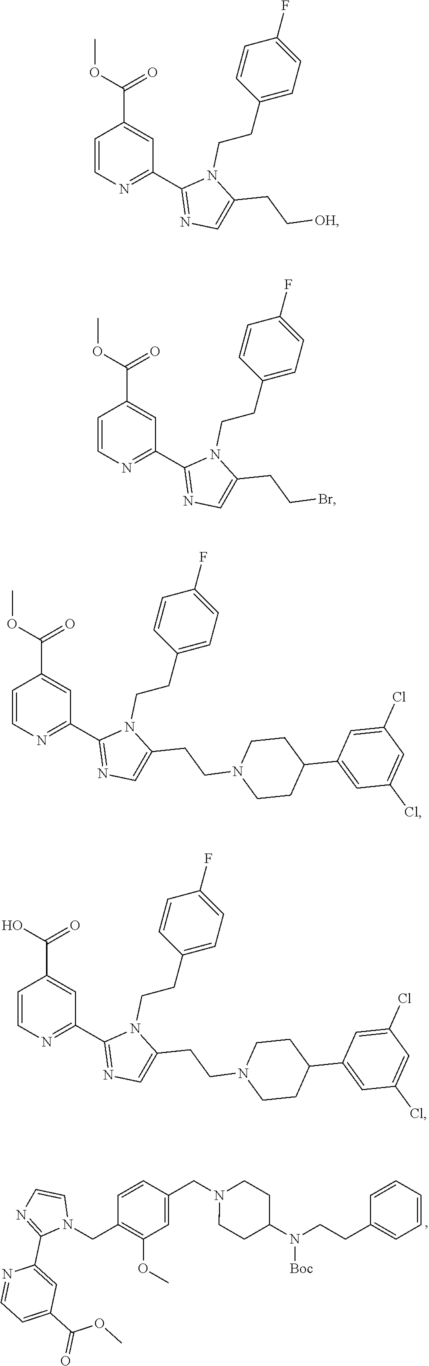 [SMILES: COC(=O)c1ccnc(-c2ncc(CCO)n2CCc2ccc(F)cc2)c1 ; COC(=O)c1ccnc(-c2ncc(CCBr)n2CCc2ccc(F)cc2)c1 ; COC(=O)c1ccnc(-c2ncc(CCN3CCC(c4cc(Cl)cc(Cl)c4)CC3)n2CCc2ccc(F)cc2)c1 ; O=C(O)c1ccnc(-c2ncc(CCN3CCC(c4cc(Cl)cc(Cl)c4)CC3)n2CCc2ccc(F)cc2)c1 ; COC(=O)c1ccnc(-c2nccn2Cc2ccc(CN3CCC(N(CCc4ccccc4)C(=O)OC(C)(C)C)CC3)cc2OC)c1]

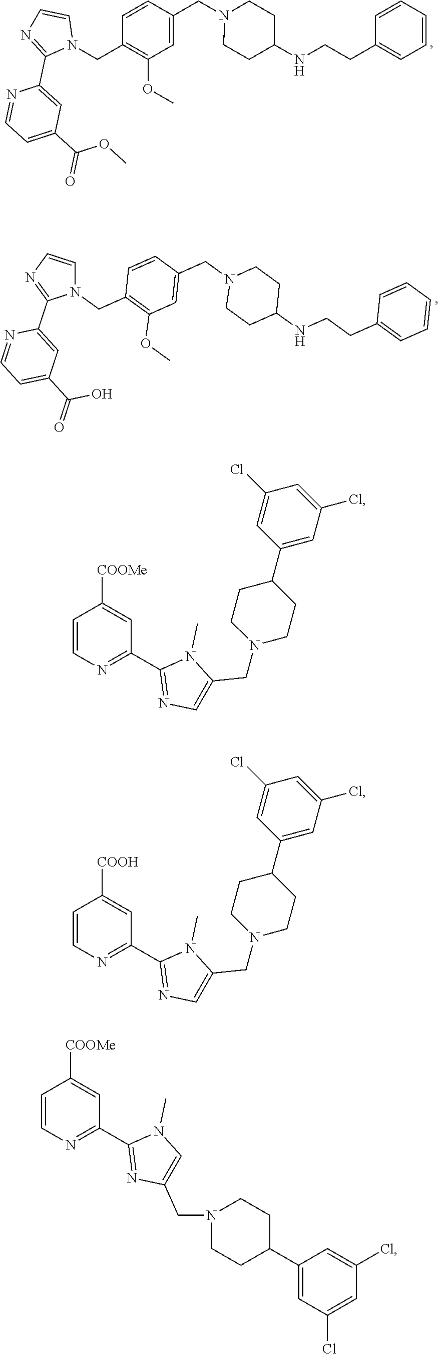 [SMILES: COC(=O)c1ccnc(-c2nccn2Cc2ccc(CN3CCC(NCCc4ccccc4)CC3)cc2OC)c1 ; COc1cc(CN2CCC(NCCc3ccccc3)CC2)ccc1Cn1ccnc1-c1cc(C(=O)O)ccn1 ; COC(=O)c1ccnc(-c2ncc(CN3CCC(c4cc(Cl)cc(Cl)c4)CC3)n2C)c1 ; Cn1c(CN2CCC(c3cc(Cl)cc(Cl)c3)CC2)cnc1-c1cc(C(=O)O)ccn1 ; COC(=O)c1ccnc(-c2nc(CN3CCC(c4cc(Cl)cc(Cl)c4)CC3)cn2C)c1]

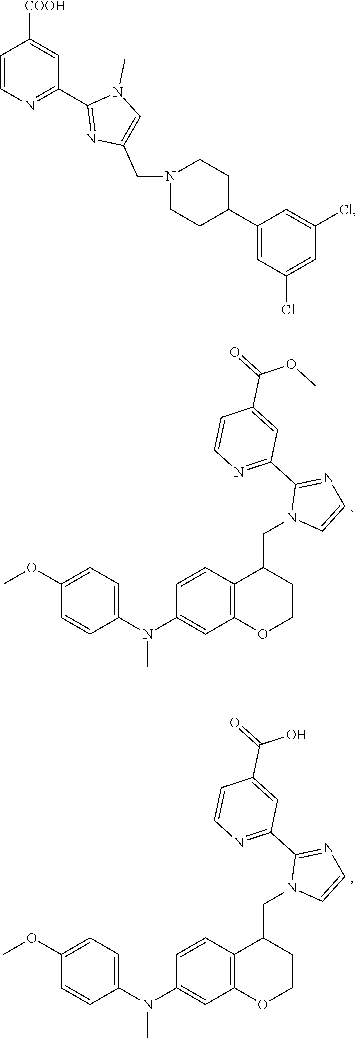 [SMILES: Cn1cc(CN2CCC(c3cc(Cl)cc(Cl)c3)CC2)nc1-c1cc(C(=O)O)ccn1 ; COC(=O)c1ccnc(-c2nccn2CC2CCOc3cc(N(C)c4ccc(OC)cc4)ccc32)c1 ; COc1ccc(N(C)c2ccc3c(c2)OCCC3Cn2ccnc2-c2cc(C(=O)O)ccn2)cc1]

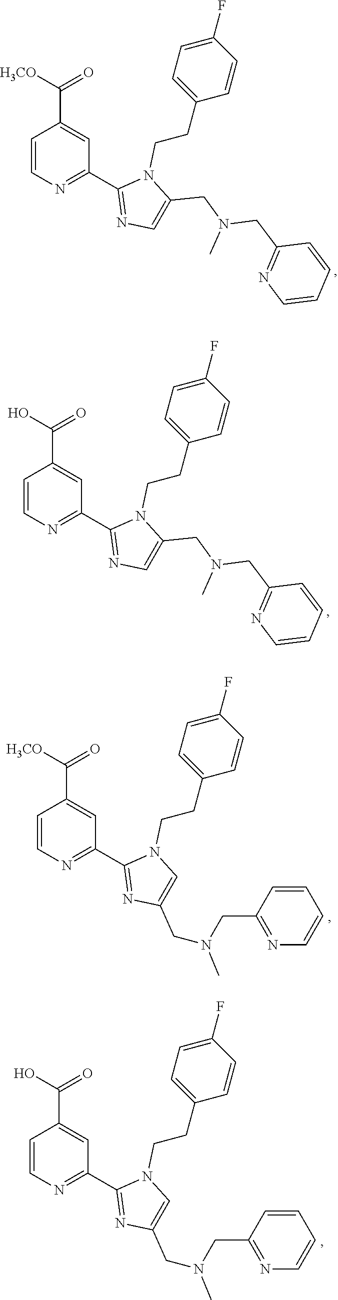

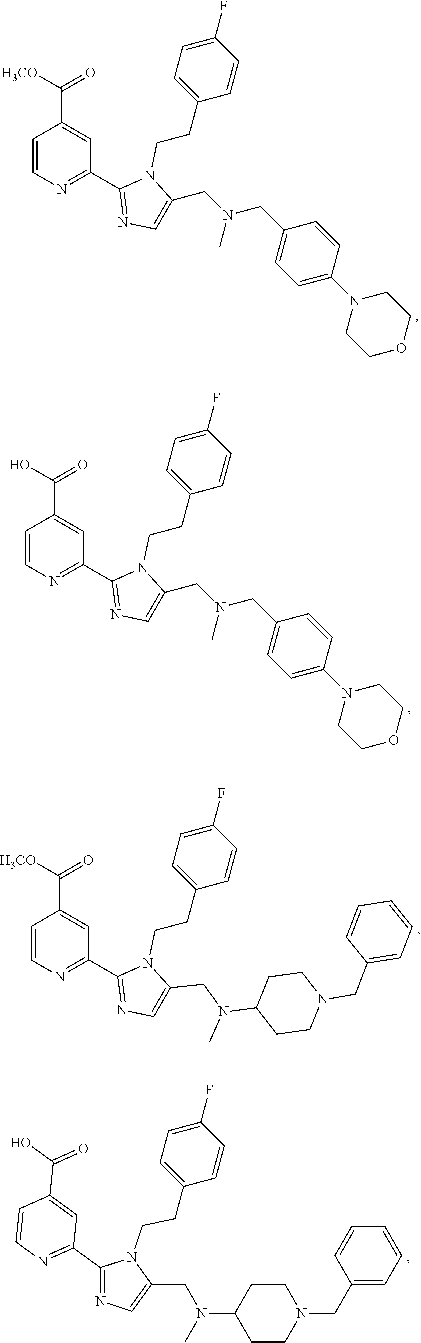 [SMILES: COC(=O)c1ccnc(-c2ncc(CN(C)Cc3ccc(N4CCOCC4)cc3)n2CCc2ccc(F)cc2)c1 ; CN(Cc1ccc(N2CCOCC2)cc1)Cc1cnc(-c2cc(C(=O)O)ccn2)n1CCc1ccc(F)cc1 ; COC(=O)c1ccnc(-c2ncc(CN(C)C3CCN(Cc4ccccc4)CC3)n2CCc2ccc(F)cc2)c1 ; CN(Cc1cnc(-c2cc(C(=O)O)ccn2)n1CCc1ccc(F)cc1)C1CCN(Cc2ccccc2)CC1]

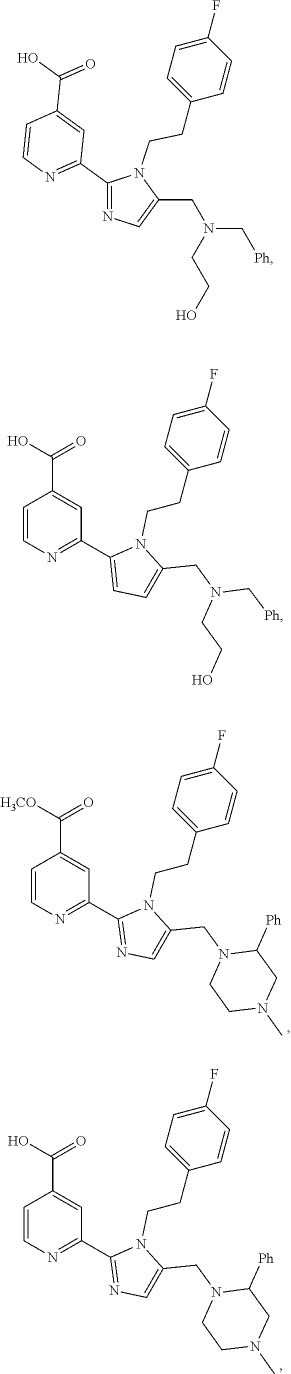 [SMILES: O=C(O)c1ccnc(-c2ncc(CN(CCO)Cc3ccccc3)n2CCc2ccc(F)cc2)c1 ; O=C(O)c1ccnc(-c2ccc(CN(CCO)Cc3ccccc3)n2CCc2ccc(F)cc2)c1 ; COC(=O)c1ccnc(-c2ncc(CN3CCN(C)CC3c3ccccc3)n2CCc2ccc(F)cc2)c1 ; CN1CCN(Cc2cnc(-c3cc(C(=O)O)ccn3)n2CCc2ccc(F)cc2)C(c2ccccc2)C1]

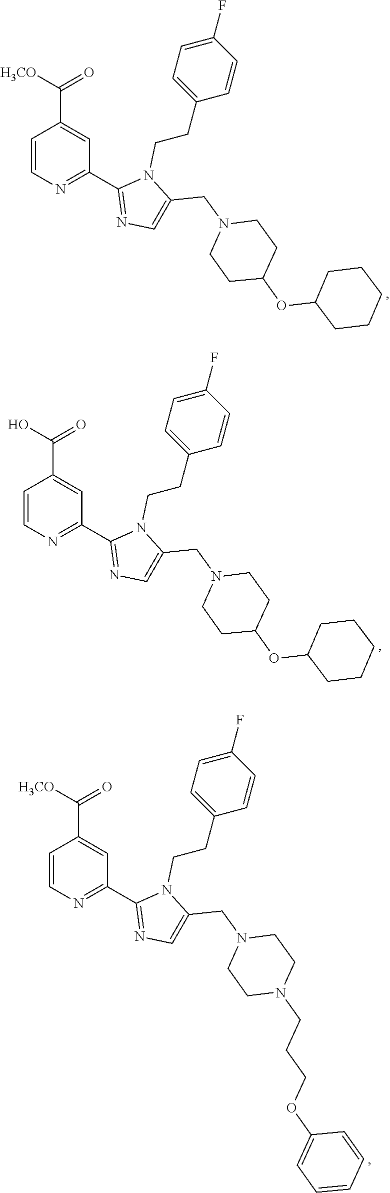 [SMILES: COC(=O)c1ccnc(-c2ncc(CN3CCC(OC4CCCCC4)CC3)n2CCc2ccc(F)cc2)c1 ; O=C(O)c1ccnc(-c2ncc(CN3CCC(OC4CCCCC4)CC3)n2CCc2ccc(F)cc2)c1 ; COC(=O)c1ccnc(-c2ncc(CN3CCN(CCCOc4ccccc4)CC3)n2CCc2ccc(F)cc2)c1]

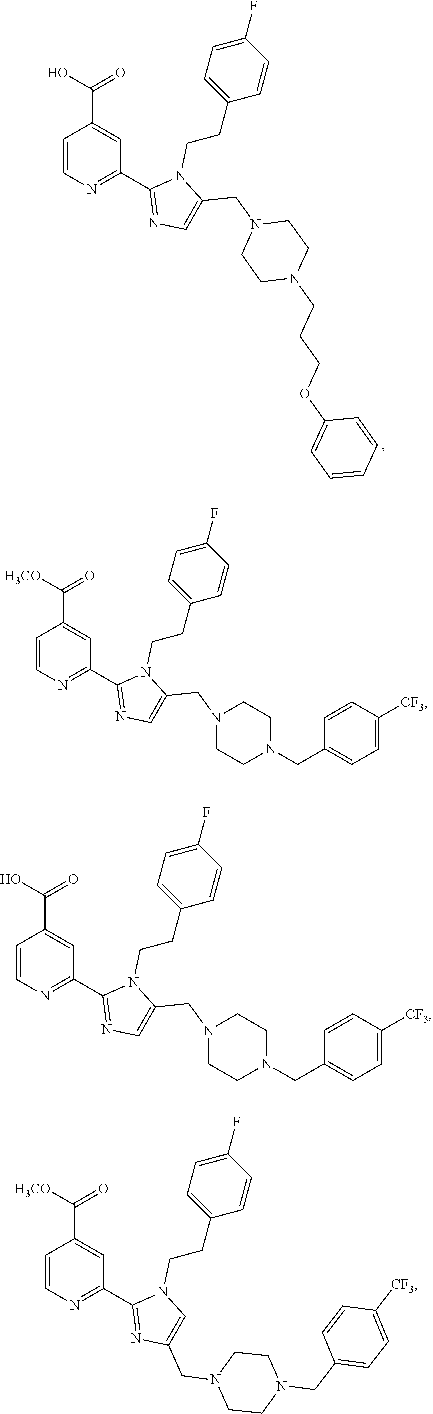

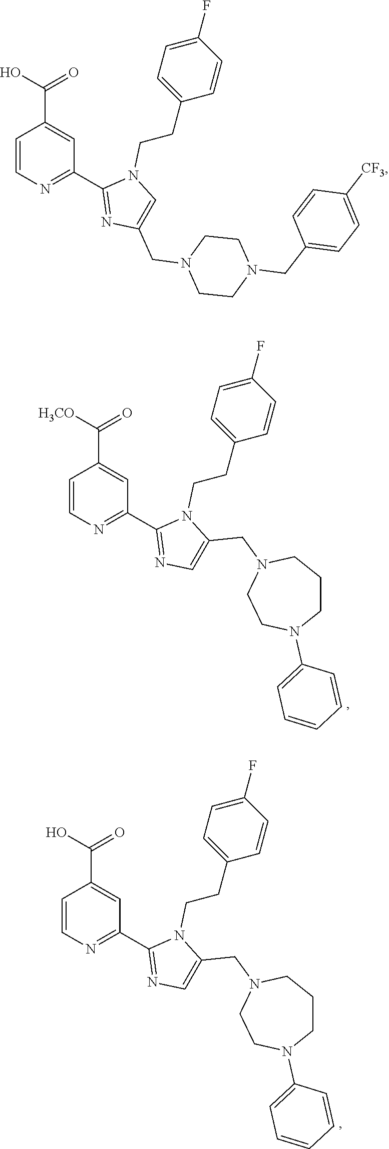 [SMILES: O=C(O)c1ccnc(-c2nc(CN3CCN(Cc4ccc(C(F)(F)F)cc4)CC3)cn2CCc2ccc(F)cc2)c1 ; COC(=O)c1ccnc(-c2ncc(CN3CCCN(c4ccccc4)CC3)n2CCc2ccc(F)cc2)c1 ; O=C(O)c1ccnc(-c2ncc(CN3CCCN(c4ccccc4)CC3)n2CCc2ccc(F)cc2)c1]

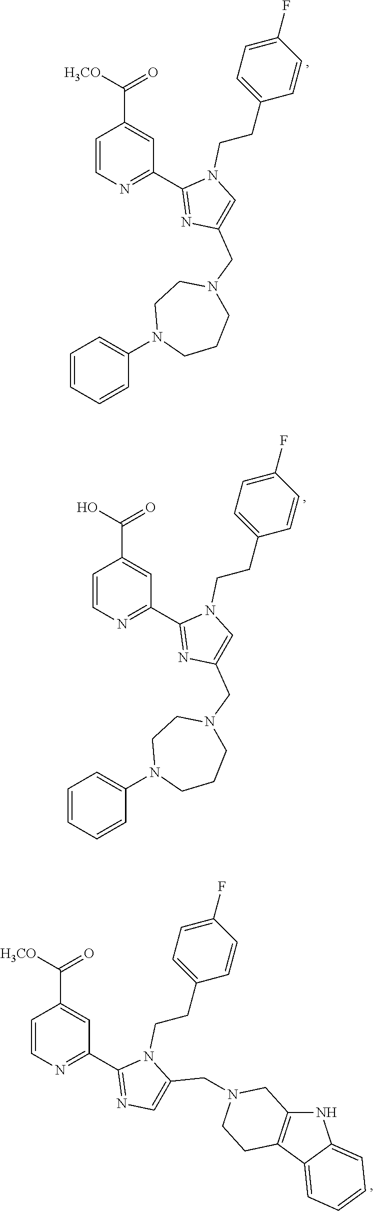 [SMILES: COC(=O)c1ccnc(-c2nc(CN3CCCN(c4ccccc4)CC3)cn2CCc2ccc(F)cc2)c1 ; O=C(O)c1ccnc(-c2nc(CN3CCCN(c4ccccc4)CC3)cn2CCc2ccc(F)cc2)c1 ; COC(=O)c1ccnc(-c2ncc(CN3CCc4c([nH]c5ccccc45)C3)n2CCc2ccc(F)cc2)c1]

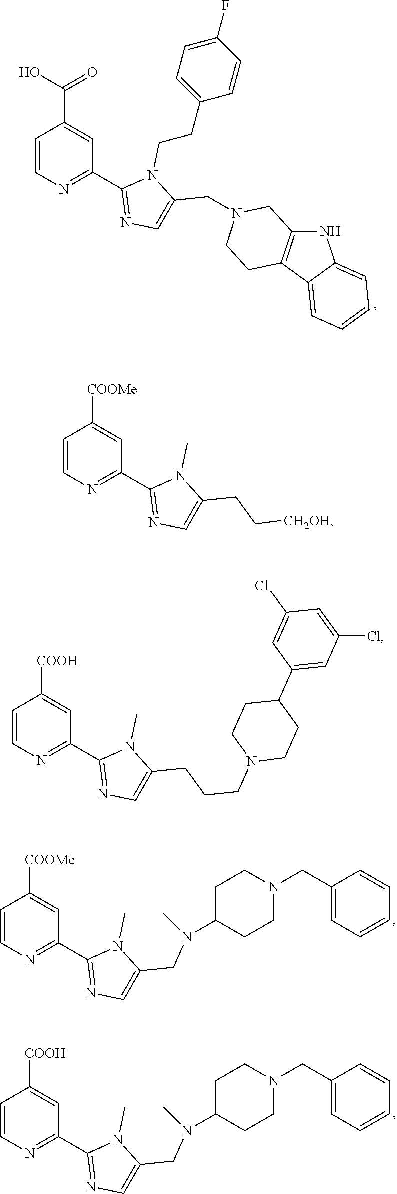 [SMILES: O=C(O)c1ccnc(-c2ncc(CN3CCc4c([nH]c5ccccc45)C3)n2CCc2ccc(F)cc2)c1 ; COC(=O)c1ccnc(-c2ncc(CCCO)n2C)c1 ; Cn1c(CCCN2CCC(c3cc(Cl)cc(Cl)c3)CC2)cnc1-c1cc(C(=O)O)ccn1 ; COC(=O)c1ccnc(-c2ncc(CN(C)C3CCN(Cc4ccccc4)CC3)n2C)c1 ; CN(Cc1cnc(-c2cc(C(=O)O)ccn2)n1C)C1CCN(Cc2ccccc2)CC1]

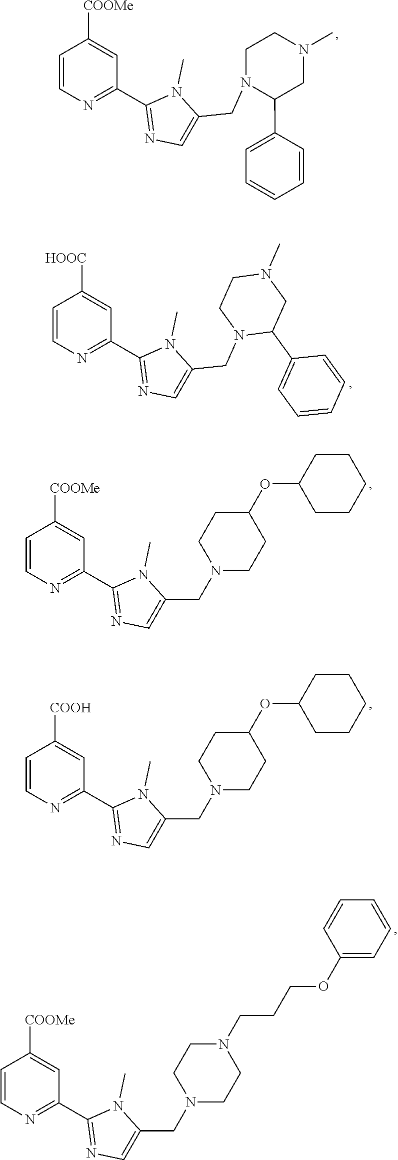

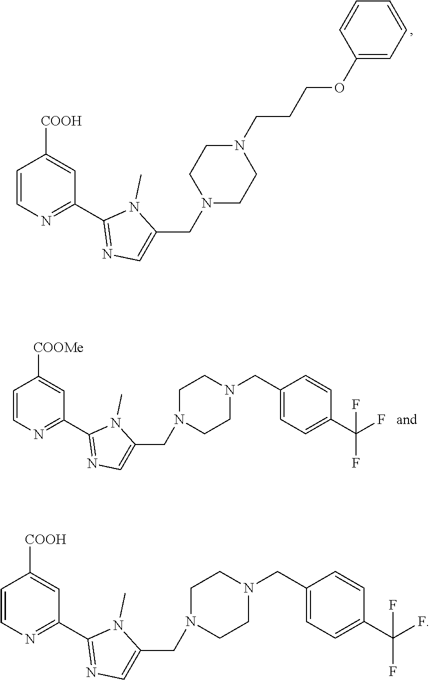 [SMILES: Cn1c(CN2CCN(CCCOc3ccccc3)CC2)cnc1-c1cc(C(=O)O)ccn1 ; COC(=O)c1ccnc(-c2ncc(CN3CCN(Cc4ccc(C(F)(F)F)cc4)CC3)n2C)c1 ; "and" ; Cn1c(CN2CCN(Cc3ccc(C(F)(F)F)cc3)CC2)cnc1-c1cc(C(=O)O)ccn1]

10. The compound of claim 1, wherein R³ and R⁴ and the carbon atoms to which they are attached form a 5-7 membered unsaturated, partial unsaturated or saturated ring system optionally containing 1-3 heteroatoms selected from N, O or S, and further optionally substituted with one or more R⁵.

11. The compound of claim 10, wherein R¹ is —COOR⁶.

12. The compound of claim 11, wherein R⁶ is hydrogen.

13. The compound of claim 11, wherein R² is C₁-C₃ alkyl-R⁷.

14. The compound of claim 13, wherein R⁷ is selected from the group consisting of aryl or heteroaryl, wherein each of the aryl or the heteroaryl may be optionally substituted with one or more R⁵.

15. The compound of claim 14, wherein the aryl is phenyl.

16. The compound of claim 14, wherein R⁵ is alkyl, halogen, haloalkyl, alkoxy or cyano.

17. The compound of claim 10, wherein the compound is selected from the group consisting of:

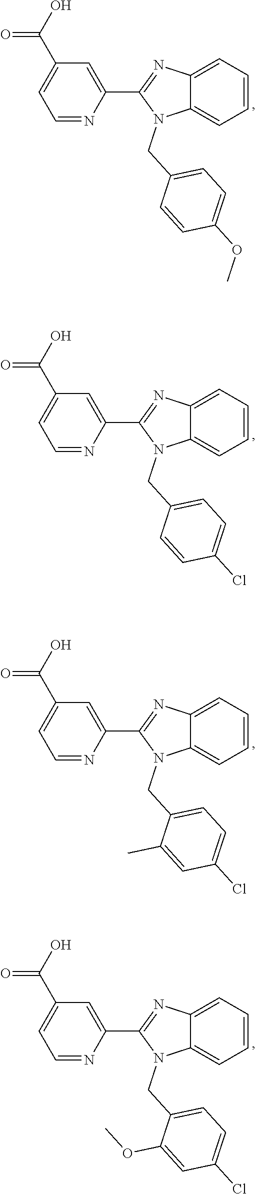 [SMILES: COc1ccc(Cn2c(-c3cc(C(=O)O)ccn3)nc3ccccc32)cc1 ; O=C(O)c1ccnc(-c2nc3ccccc3n2Cc2ccc(Cl)cc2)c1 ; Cc1cc(Cl)ccc1Cn1c(-c2cc(C(=O)O)ccn2)nc2ccccc21 ; COc1cc(Cl)ccc1Cn1c(-c2cc(C(=O)O)ccn2)nc2ccccc21]

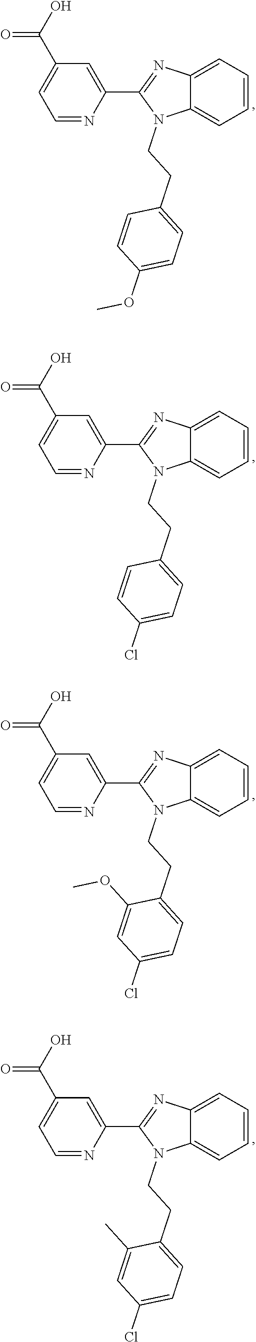 [SMILES: COc1ccc(CCn2c(-c3cc(C(=O)O)ccn3)nc3ccccc32)cc1 ; O=C(O)c1ccnc(-c2nc3ccccc3n2CCc2ccc(Cl)cc2)c1 ; COc1cc(Cl)ccc1CCn1c(-c2cc(C(=O)O)ccn2)nc2ccccc21 ; Cc1cc(Cl)ccc1CCn1c(-c2cc(C(=O)O)ccn2)nc2ccccc21]

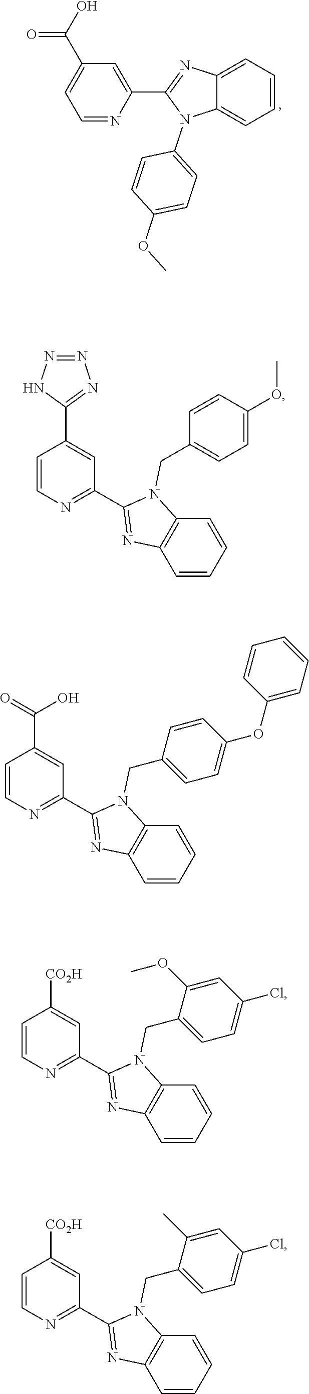 [SMILES: COc1ccc(-n2c(-c3cc(C(=O)O)ccn3)nc3ccccc32)cc1 ; COc1ccc(Cn2c(-c3cc(-c4nnn[nH]4)ccn3)nc3ccccc32)cc1 ; O=C(O)c1ccnc(-c2nc3ccccc3n2Cc2ccc(Oc3ccccc3)cc2)c1 ; COc1cc(Cl)ccc1Cn1c(-c2cc(C(=O)O)ccn2)nc2ccccc21 ; Cc1cc(Cl)ccc1Cn1c(-c2cc(C(=O)O)ccn2)nc2ccccc21]

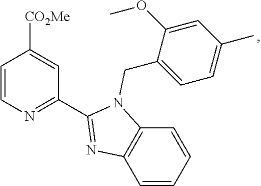 [SMILES: COC(=O)c1ccnc(-c2nc3ccccc3n2Cc2ccc(C)cc2OC)c1]

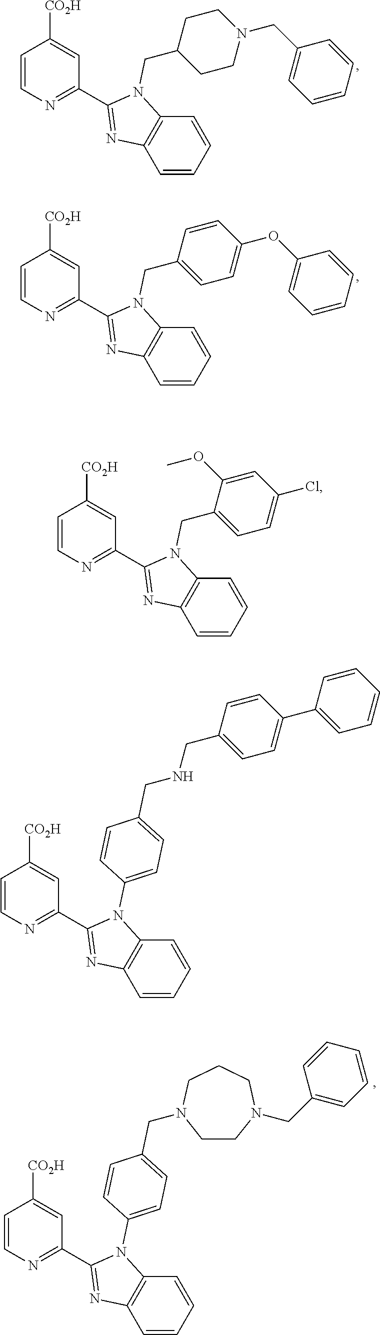 [SMILES: O=C(O)c1ccnc(-c2nc3ccccc3n2CC2CCN(Cc3ccccc3)CC2)c1 ; O=C(O)c1ccnc(-c2nc3ccccc3n2Cc2ccc(Oc3ccccc3)cc2)c1 ; COc1cc(Cl)ccc1Cn1c(-c2cc(C(=O)O)ccn2)nc2ccccc21 ; O=C(O)c1ccnc(-c2nc3ccccc3n2-c2ccc(CNCc3ccc(-c4ccccc4)cc3)cc2)c1 ; O=C(O)c1ccnc(-c2nc3ccccc3n2-c2ccc(CN3CCCN(Cc4ccccc4)CC3)cc2)c1]

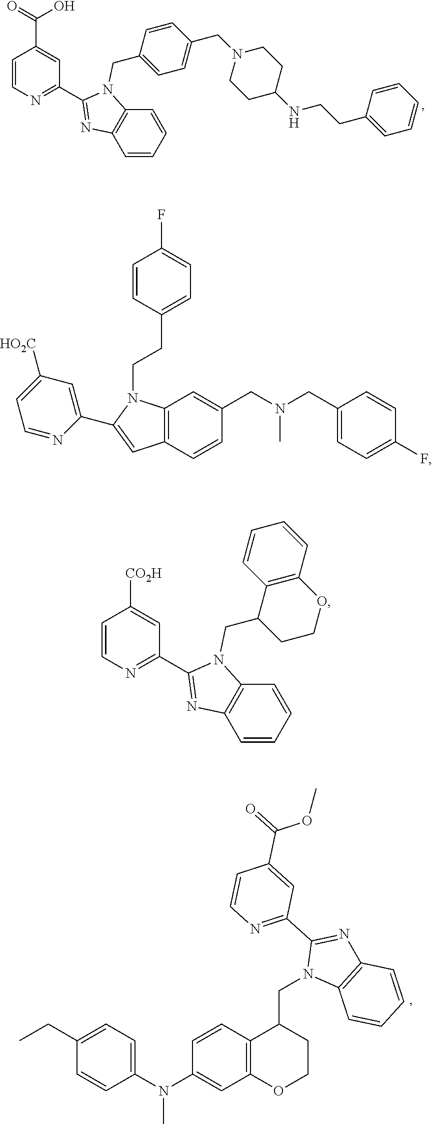 [SMILES: O=C(O)c1ccnc(-c2nc3ccccc3n2Cc2ccc(CN3CCC(NCCc4ccccc4)CC3)cc2)c1 ; CN(Cc1ccc(F)cc1)Cc1ccc2cc(-c3cc(C(=O)O)ccn3)n(CCc3ccc(F)cc3)c2c1 ; O=C(O)c1ccnc(-c2nc3ccccc3n2CC2CCOc3ccccc32)c1 ; CCc1ccc(N(C)c2ccc3c(c2)OCCC3Cn2c(-c3cc(C(=O)OC)ccn3)nc3ccccc32)cc1]

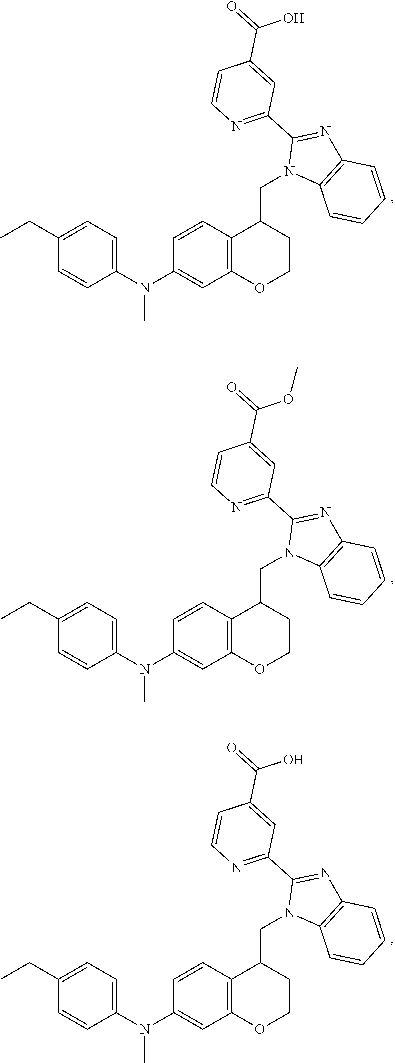 [SMILES: CCc1ccc(N(C)c2ccc3c(c2)OCCC3Cn2c(-c3cc(C(=O)O)ccn3)nc3ccccc32)cc1 ; CCc1ccc(N(C)c2ccc3c(c2)OCCC3Cn2c(-c3cc(C(=O)OC)ccn3)nc3ccccc32)cc1 ; CCc1ccc(N(C)c2ccc3c(c2)OCCC3Cn2c(-c3cc(C(=O)O)ccn3)nc3ccccc32)cc1]

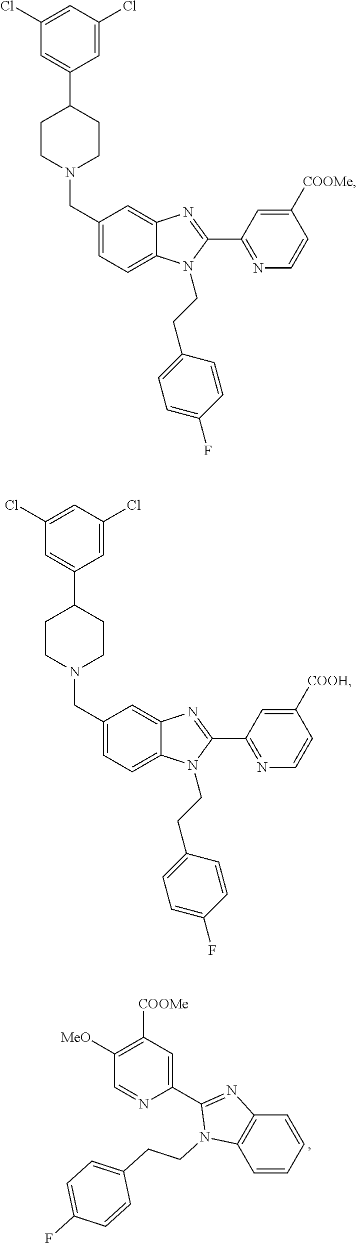 [SMILES: COC(=O)c1ccnc(-c2nc3cc(CN4CCC(c5cc(Cl)cc(Cl)c5)CC4)ccc3n2CCc2ccc(F)cc2)c1 ; O=C(O)c1ccnc(-c2nc3cc(CN4CCC(c5cc(Cl)cc(Cl)c5)CC4)ccc3n2CCc2ccc(F)cc2)c1 ; COC(=O)c1cc(-c2nc3ccccc3n2CCc2ccc(F)cc2)ncc1OC]

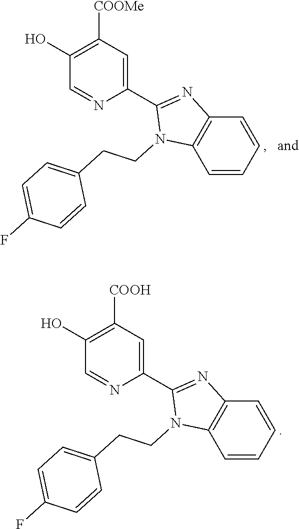 [SMILES: COC(=O)c1cc(-c2nc3ccccc3n2CCc2ccc(F)cc2)ncc1O ; O=C(O)c1cc(-c2nc3ccccc3n2CCc2ccc(F)cc2)ncc1O]

18. A pharmaceutical composition, comprising a therapeutically effective amount of a compound of formula (I) of claim 1, and a pharmaceutically acceptable excipient.

19. A method for inhibiting histone lysine demethylase activity in a cell, comprising contacting the cell in which inhibition of histone lysine demethylase activity is desired with a therapeutically effective amount of a compound of formula (I) or a pharmaceutical compositions containing the compound of formula (I) of claim 1, or pharmaceutically acceptable salts of said compound or said composition.

20. (canceled)

21. (canceled)

22. (canceled)

23. A method for treating cancer comprising administering to a patient having cancer a therapeutically effective amount of a compound of formula (I) of claim 1, or a pharmaceutically acceptable salt thereof, alone or combined with a pharmaceutically acceptable carrier, excipient or diluents.

24. (canceled)

25. (canceled)

26. (canceled)

27. (canceled)
